(19) United States
(12) Patent Application Publication
(10) Pub. No.: US 2026/0305057 A1
(43) Pub. Date: Oct. 1, 2026

(54) ORGANIC ELECTROLUMINESCENT MATERIAL AND ITS DEVICE

(71) Applicant: GUANGDONG AGLAIA OPTOELECTRONIC MATERIALS CO., LTD, Guangdong (CN)

(72) Inventors: Shaofu CHEN, Guangdong (CN); Lei DAI, Guangdong (CN); Lifei CAI, Guangdong (CN)

(73) Assignee: GUANGDONG AGLAIA OPTOELECTRONIC MATERIALS CO., LTD, Guangdong (CN)

(21) Appl. No.: 19/164,509

(22) PCT Filed: Jan. 18, 2024
(86) PCT No.: PCT/CN2024/072899

§ 371 Date: Sep. 11, 2025

(30) Foreign Application Priority Data
Mar. 13, 2023 (CN) 202310235871.0
Sep. 4, 2023 (CN) 202311128039.7

(51) Int. Cl. H10K 50/11; H10K 59/12; H10K 85/30; H10K 85/60

(52) U.S. Cl. CPC H10K 50/11; H10K 85/342; H10K 85/626; H10K 85/633; H10K 85/654; H10K 85/6572; H10K 85/6574; H10K 59/12

(57) ABSTRACT

An organic electroluminescent material and its device are provided. The organic electroluminescent material has a structure of formula (1). The organic electroluminescent material has the advantages of high optical and electrical stability, low sublimation temperature, low driving voltage, high light emitting efficiency, and long device service life, and can be used as a host material in an OLED device. As a host material, this material has the possibility of being applied to AMOLED industry.

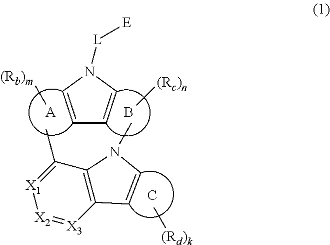

### TECHNICAL FIELD

The present disclosure relates to the technical field of organic electroluminescent technology, and in particular, to a compound formed by linking an indole- and pyrrole-fused aza-macrocyclic ring with an N-heterocycle and a similar structure thereof and an organic electroluminescent device comprising the compound.

### BACKGROUND

At present, an organic electroluminescent device (OLED), as a new generation of display technology, has attracted increasing attention in both display and lighting technologies, with very broad application prospects. However, compared with market application requirements, the light emitting efficiency, driving voltage, and service life of the OLED device remain to be further strengthened and improved.

In general, the basic structure of the OLED device is a thin film of an organic functional material with various functions sandwiched between metal electrodes, like a sandwich structure. Driven by a current, holes and electrons are injected from a cathode and an anode, respectively. After moving a certain distance, the holes and the electrons are recombined in a light emitting layer and are released as light or heat, so that the OLED emits light. However, the properties of a phosphorescent OLED are not only determined by a triplet luminophore used, but also by other types of materials, such as a host material, which are also very important. The host material has significant effects on reducing the driving voltage of the device, improving the light emitting efficiency of the device, and improving the service life of the device, etc.

Therefore, it is necessary to continue research and development of a novel host material to further improve the performance of the organic electroluminescent device.

Patent document 1 (CN108391433B) discloses that

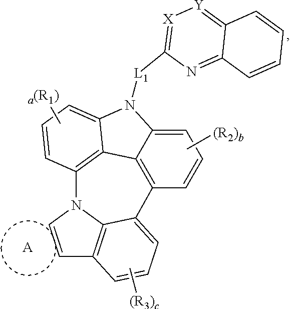

has an azaaromatic structural unit bonded to a particular site of quinazoline or quinoxaline, and is used as a red electroluminescent host material, but the thermal stability and device life of this type of material remain to be further improved. Patent document 2 (CN114591341A) discloses a host material of an indolocarbazole structural unit

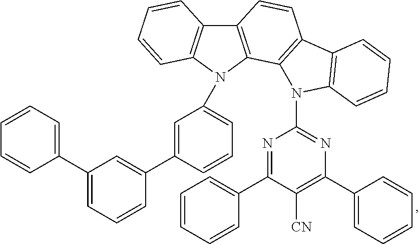

and the device efficiency and service life of this type of material also remain to be improved. Patent document 3 (US20220289681A1) discloses a red host material that is a combination of an indole-fused azamacrocyclic structural unit and triazine with a structure similar to

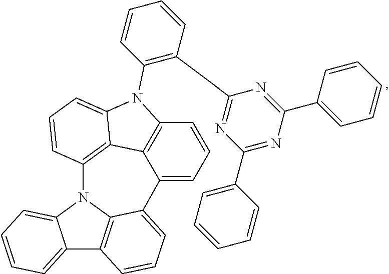

this structure is different from that in the present disclosure, and the efficiency of a device using this type of material as a host material remains to be further improved. Patent document 4 (KR101877961B1) discloses a bipolar host material of indolocarbazole with a structure similar to

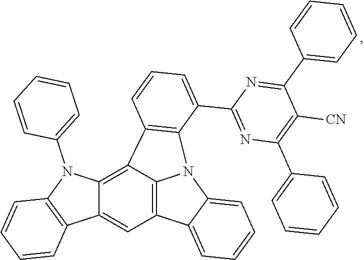 [SMILES: N#Cc1c(-c2ccccc2)nc(-c2cccc3c4c5c(cc6c7ccccc7n(c23)c64)c2ccccc2n5-c2ccccc2)nc1-c1ccccc1]

and the service life of a device using this type of material as a green electroluminescent host material remains to be further improved.

### SUMMARY

In order to solve the above detects, the present disclosure provides a compound formed by linking an indole- and pyrrole-fused aza-macrocyclic ring with an N-heterocycle and a similar structure thereof and an organic electroluminescent device comprising the compound.

An organic electroluminescent material in the present disclosure has a structure of formula (1). An organic electroluminescent material provided in the present disclosure has the advantages of high optical and electrical stability, low sublimation temperature, low driving voltage, high light emitting efficiency, and long device service life, can be used as a host material in an OLED device, further has a low melting point, and is conductive to material evaporation stability as a molten material. As a host material, this compound has the possibility of being applied to AMOLED industry.

An organic electroluminescent material has a structure of formula (1),

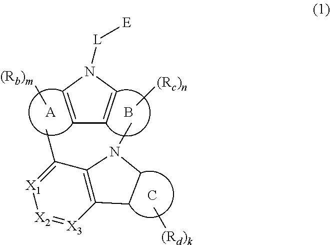

wherein L is selected from any one of a single bond, a substituted or unsubstituted C₆-C₃₀ aryl, or a substituted or unsubstituted C₃-C₂₀ heteroaryl;
X₁ to X₃ each independently represent N or CRₐ; ring A, ring B, and ring C are, identically or differently during each occurrence, selected from an aromatic ring with 5-18 carbon atoms or a heteroaromatic ring with 3-18 carbon atoms;
wherein E has a structure of formula (2):

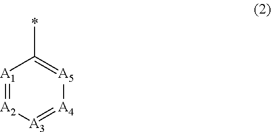

wherein A₁ to A₅ are identical or different; A₁ to A₅ are each independently selected from N or CRI; at least one of A₁ to A₅ is N, at least one thereof is C—CN; and * represents a linking site with formula (1);
wherein Rₐ, R_{b}, R_{c}, R_{d}, and R₁ are identical or different during each occurrence, and are each independently selected from a group consisting of hydrogen, deuterium, halogen, a substituted or unsubstituted alkyl with 1-10 carbon atoms, a substituted or unsubstituted cycloalkyl with 3-20 ring carbon atoms, a substituted or unsubstituted heteroalkyl with 1-10 carbon atoms, a substituted or unsubstituted heterocyclyl with 3-20 ring atoms, a substituted or unsubstituted arylalkyl with 7-30 carbon atoms, a substituted or unsubstituted alkoxy with 1-carbon atoms, a substituted or unsubstituted aryloxy with 6-30 carbon atoms, a substituted or unsubstituted alkenyl with 2-10 carbon atoms, a substituted or unsubstituted aryl with 6-30 carbon atoms, a substituted or unsubstituted heteroaryl with 3-30 carbon atoms, a substituted or unsubstituted alkylsilyl with 3-20 carbon atoms, a substituted or unsubstituted arylsilyl with 6-20 carbon atoms, a substituted or unsubstituted amino with 0-20 carbon atoms, a substituted or unsubstituted acyl with 0-20 carbon atoms, carbonyl, carboxyl, a substituted or unsubstituted ester group with 0-20 carbon atoms, cyano, isocyano, hydroxyl, sulfydryl, a substituted or unsubstituted sulfinyl with 0-20 carbon atoms, a substituted or unsubstituted sulfonyl with 0-carbon atoms, or phosphino; adjacent Rₐ, adjacent R_{b}, adjacent R_{c}, and adjacent R_{d} can each be linked to form a ring; and
the substitution refers to a substitution with deuterium, halogen, cyano, an alkyl with 1-6 carbon atoms, or an aryl with 6-30 carbon atoms,
wherein m, n, and k are each independently selected from an integer of 1-4; and
heteroatoms in the heteroaromatic ring, the heteroalkyl, the heterocyclyl, and the heteroaryl are each independently selected from O, S, N, Si, Ge, or P.

In some embodiments, the organic electroluminescent material has a structure of formula (3),

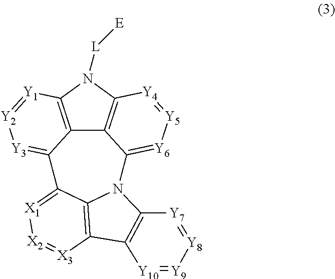

wherein X₁ to X₃ each independently represent N or CRₐ; Y₁ to Y₁₀ are identical or different, Y₁ to Y₁₀ are each independently selected from N or CR; and Rₐ is as defined above;
R is as defined as R_{b}, R_{c}, and R_{d} above, and R is identical or different during each occurrence, and is each independently selected from a group consisting of hydrogen, deuterium, halogen, a substituted or unsubstituted alkyl with 1-10 carbon atoms, a substituted or unsubstituted cycloalkyl with 3-20 ring carbon atoms, a substituted or unsubstituted heteroalkyl with 1-10 carbon atoms, a substituted or unsubstituted heterocyclyl with 3-20 ring atoms, a substituted or unsubstituted arylalkyl with 7-30 carbon atoms, a substituted or unsubstituted alkoxy with 1-20 carbon atoms, a substituted or unsubstituted aryloxy with 6-30 carbon atoms, a substituted or unsubstituted alkenyl with 2-10 carbon atoms, a substituted or unsubstituted aryl with 6-30 carbon atoms, a substituted or unsubstituted heteroaryl with 3-30 carbon atoms, a substituted or unsubstituted alkylsilyl with 3-20 carbon atoms, a substituted or unsubstituted arylsilyl with 6-20 carbon atoms, a substituted or unsubstituted amino with 0-20 carbon atoms, a substituted or unsubstituted acyl with 0-20 carbon atoms, carbonyl, carboxyl, a substituted or unsubstituted ester group with 0-20 carbon atoms, cyano, isocyano, hydroxyl, sulfydryl, a substituted or unsubstituted sulfinyl with 0-20 carbon atoms, a substituted or unsubstituted sulfonyl with 0-20 carbon atoms, or phosphino; adjacent R and the adjacent Rₐ can each be linked to form a ring, and the substitution refers to a substitution with deuterium, halogen, cyano, an alkyl with 1-6 carbon atoms, or an aryl with 6-30 carbon atoms.

In some embodiments, Y₁ to Y₁₀ each independently represent CR.

In some embodiments, A₁ to A₅ in the formula (2) comprise two N, and at least one of A₁ to A₅ is C—CN.

In some embodiments, the organic electroluminescent material has one of structures represented by formulas (4)-(27):

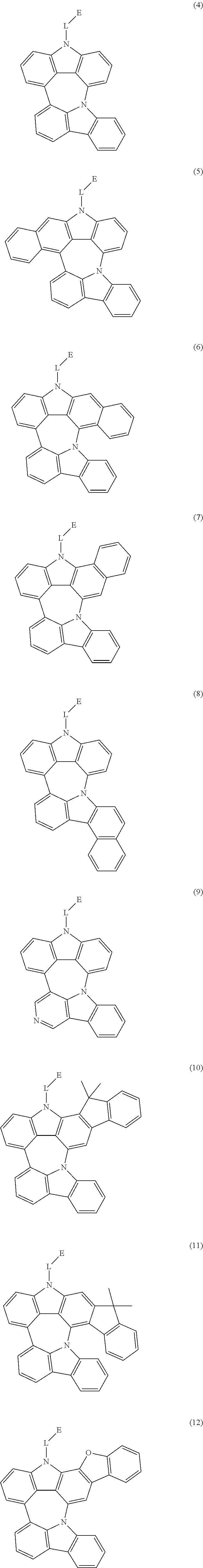

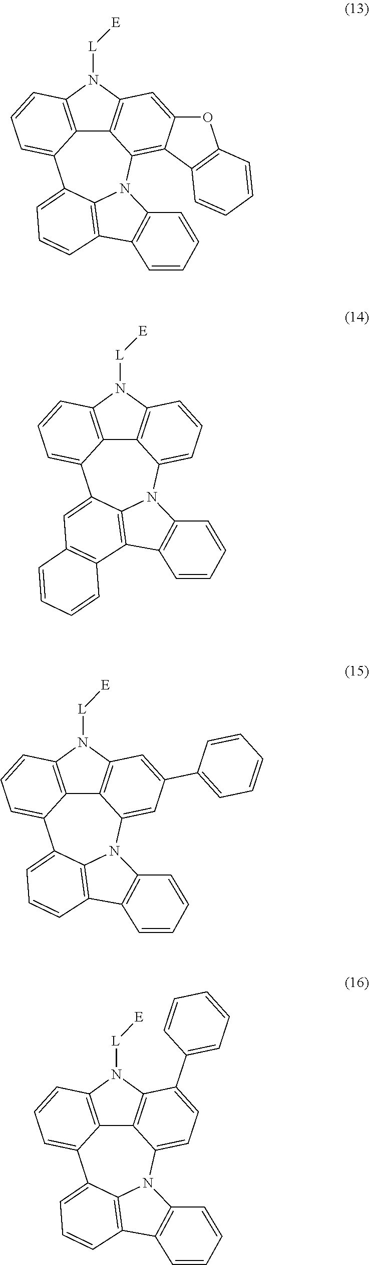

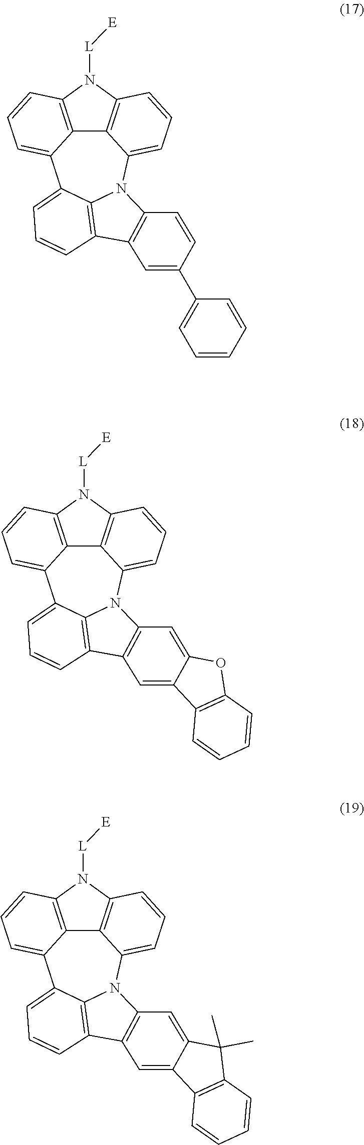

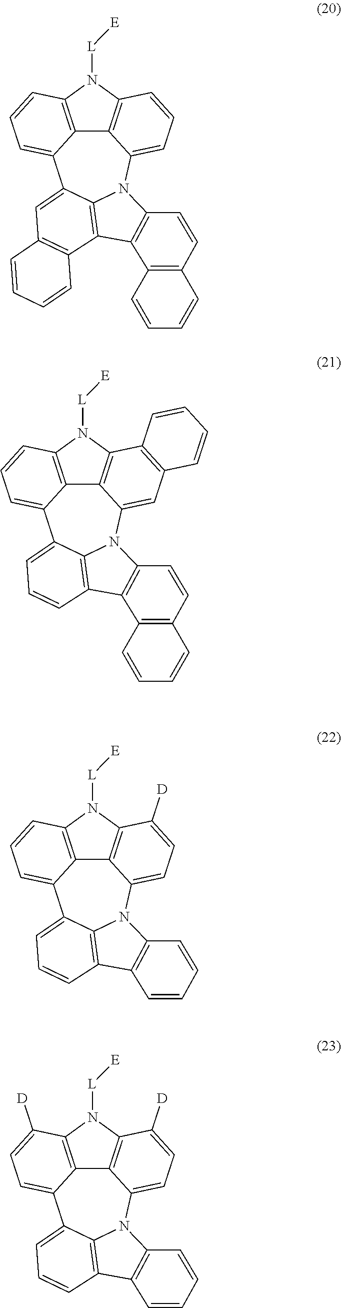

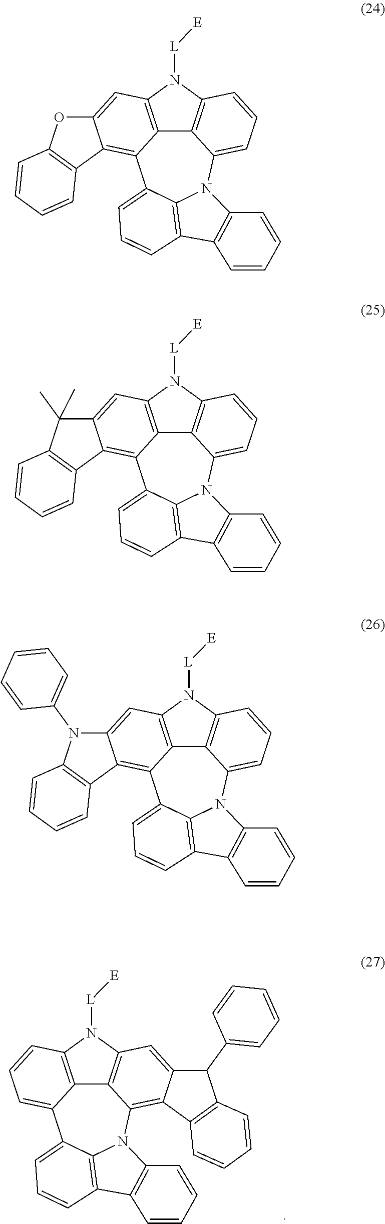

In some embodiments, E represents a structure represented by one of formulas (28)-(34) below:

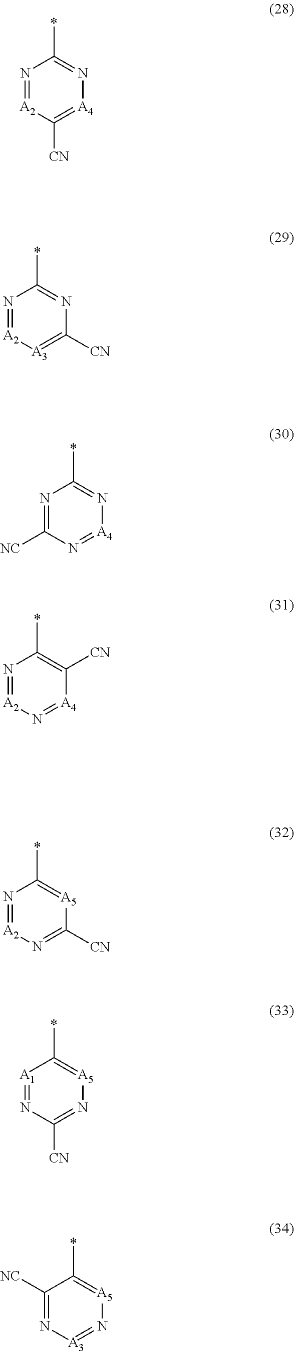

wherein A₁ to A₅ in the formulas (28)-(34) are CR₁, and R₁ is as defined above.

In some embodiments, L is a single bond or a substituted or unsubstituted C₆-C₁₀ aryl.

In some embodiments, L is a substituted or unsubstituted phenyl.

In some embodiments, R₁ is a substituted or unsubstituted aryl with 6-18 carbon atoms, or a substituted or unsubstituted heteroaryl with 3-18 carbon atoms.

In some embodiments, R₁ is, identically or differently during each occurrence, selected from a group consisting of phenyl, deuterated phenyl, methylphenyl, fluorophenyl, tert-butylphenyl, trideuterated methylphenyl, biphenyl, naphthyl, deuterated naphthyl, dibenzofuryl, dibenzothiophenyl, 9,9-dimethylfluorenyl, carbazolyl, pyridyl, pyrimidinyl, 4-cyanophenyl, 3-cyanophenyl, and a triphenylene.

In some embodiments, the organic electroluminescent material is one of following structural formulas, or is correspondingly partially or completely deuterated or fluorinated,

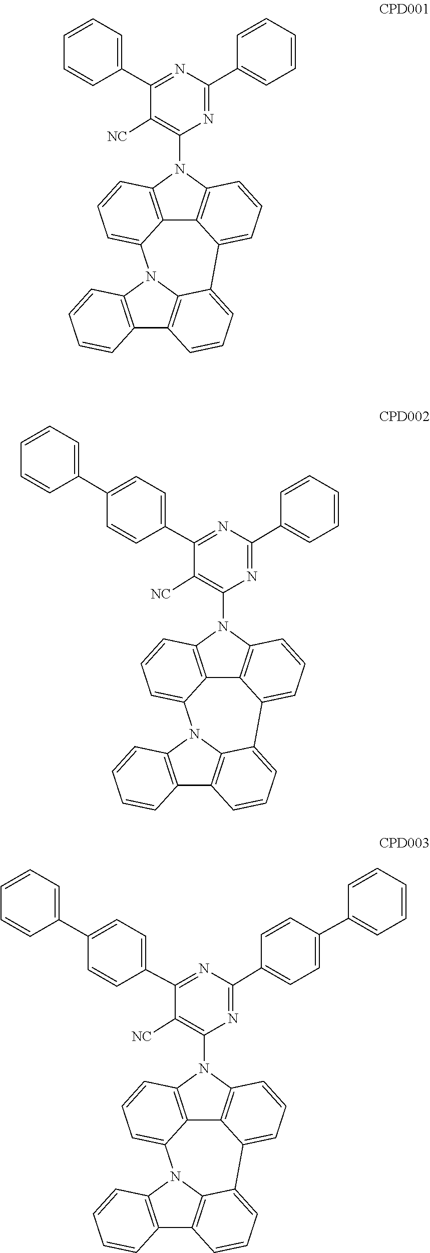 [SMILES: N#Cc1c(-c2ccccc2)nc(-c2ccccc2)nc1-n1c2cccc3c4cccc5c6ccccc6n(c6cccc1c6c32)c45 ; N#Cc1c(-c2ccc(-c3ccccc3)cc2)nc(-c2ccccc2)nc1-n1c2cccc3c4cccc5c6ccccc6n(c6cccc1c6c32)c45 ; N#Cc1c(-c2ccc(-c3ccccc3)cc2)nc(-c2ccc(-c3ccccc3)cc2)nc1-n1c2cccc3c4cccc5c6ccccc6n(c6cccc1c6c32)c45]

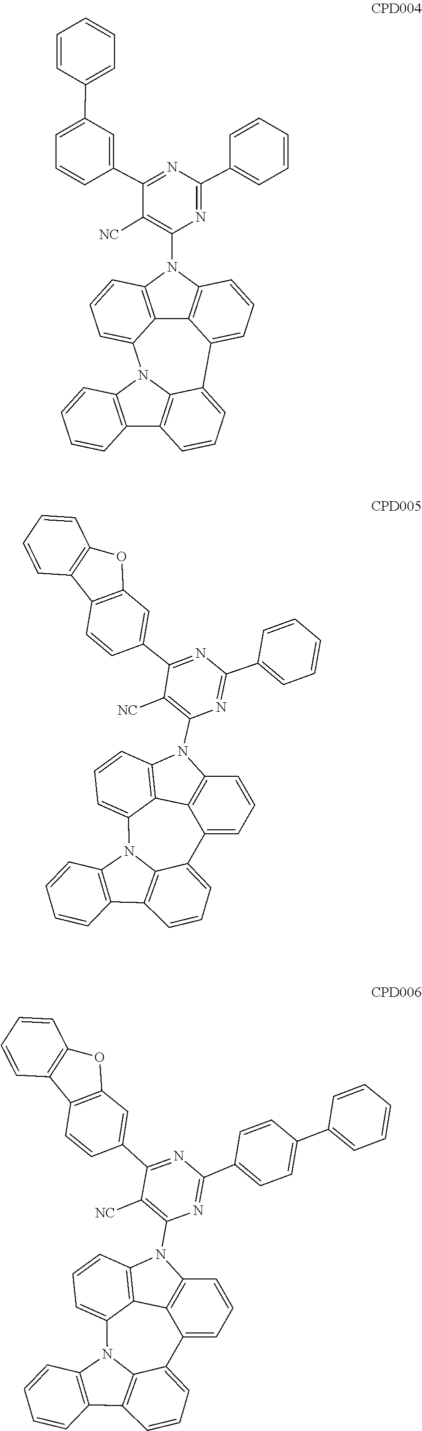 [SMILES: N#Cc1c(-c2cccc(-c3ccccc3)c2)nc(-c2ccccc2)nc1-n1c2cccc3c4cccc5c6ccccc6n(c6cccc1c6c32)c45 ; N#Cc1c(-c2ccc3c(c2)oc2ccccc23)nc(-c2ccccc2)nc1-n1c2cccc3c4cccc5c6ccccc6n(c6cccc1c6c32)c45 ; N#Cc1c(-c2ccc3c(c2)oc2ccccc23)nc(-c2ccc(-c3ccccc3)cc2)nc1-n1c2cccc3c4cccc5c6ccccc6n(c6cccc1c6c32)c45]

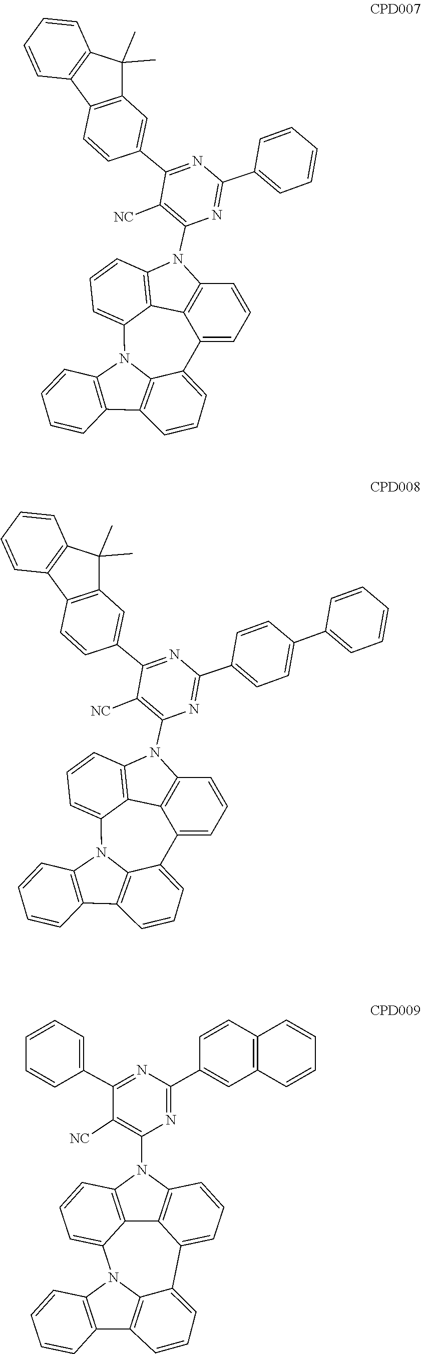 [SMILES: CC1(C)c2ccccc2-c2ccc(-c3nc(-c4ccccc4)nc(-n4c5cccc6c7cccc8c9ccccc9n(c9cccc4c9c65)c78)c3C#N)cc21 ; CC1(C)c2ccccc2-c2ccc(-c3nc(-c4ccc(-c5ccccc5)cc4)nc(-n4c5cccc6c7cccc8c9ccccc9n(c9cccc4c9c65)c78)c3C#N)cc21 ; N#Cc1c(-c2ccccc2)nc(-c2ccc3ccccc3c2)nc1-n1c2cccc3c4cccc5c6ccccc6n(c6cccc1c6c32)c45]

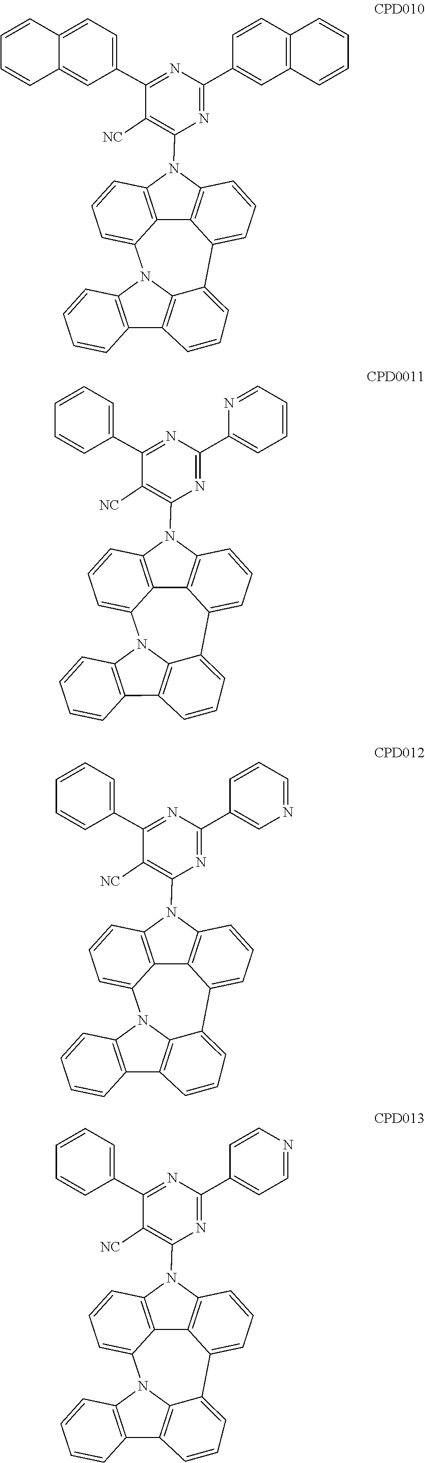

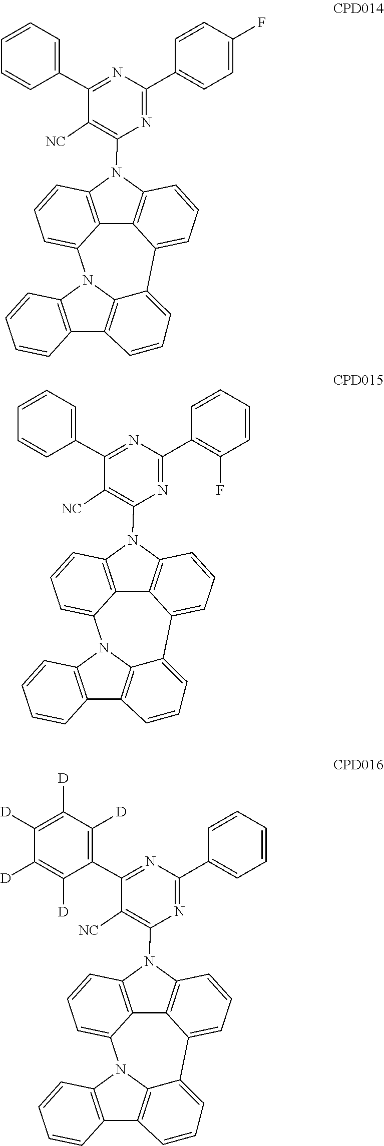

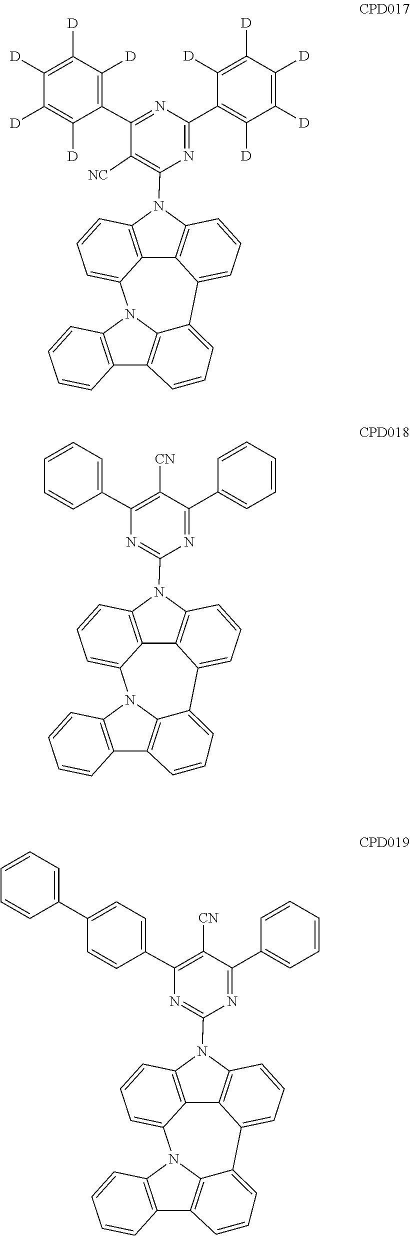 [SMILES: [2H]c1c([2H])c([2H])c(-c2nc(-c3c([2H])c([2H])c([2H])c([2H])c3[2H])c(C#N)c(-n3c4cccc5c6cccc7c8ccccc8n(c8cccc3c8c54)c67)n2)c([2H])c1[2H] ; N#Cc1c(-c2ccccc2)nc(-n2c3cccc4c5cccc6c7ccccc7n(c7cccc2c7c43)c56)nc1-c1ccccc1 ; N#Cc1c(-c2ccccc2)nc(-n2c3cccc4c5cccc6c7ccccc7n(c7cccc2c7c43)c56)nc1-c1ccc(-c2ccccc2)cc1]

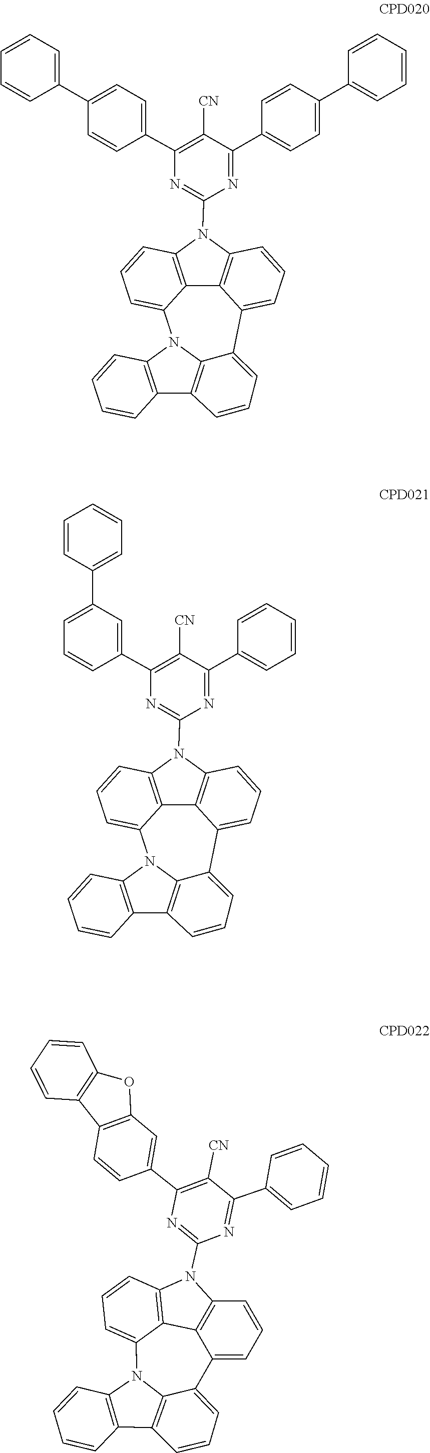 [SMILES: N#Cc1c(-c2ccc(-c3ccccc3)cc2)nc(-n2c3cccc4c5cccc6c7ccccc7n(c7cccc2c7c43)c56)nc1-c1ccc(-c2ccccc2)cc1 ; N#Cc1c(-c2ccccc2)nc(-n2c3cccc4c5cccc6c7ccccc7n(c7cccc2c7c43)c56)nc1-c1cccc(-c2ccccc2)c1 ; N#Cc1c(-c2ccccc2)nc(-n2c3cccc4c5cccc6c7ccccc7n(c7cccc2c7c43)c56)nc1-c1ccc2c(c1)oc1ccccc12]

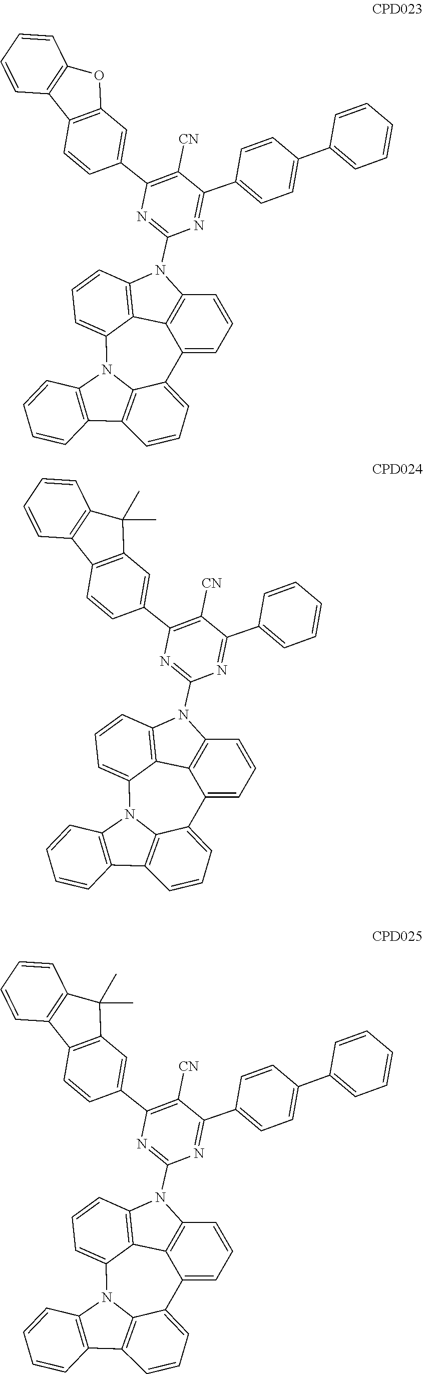

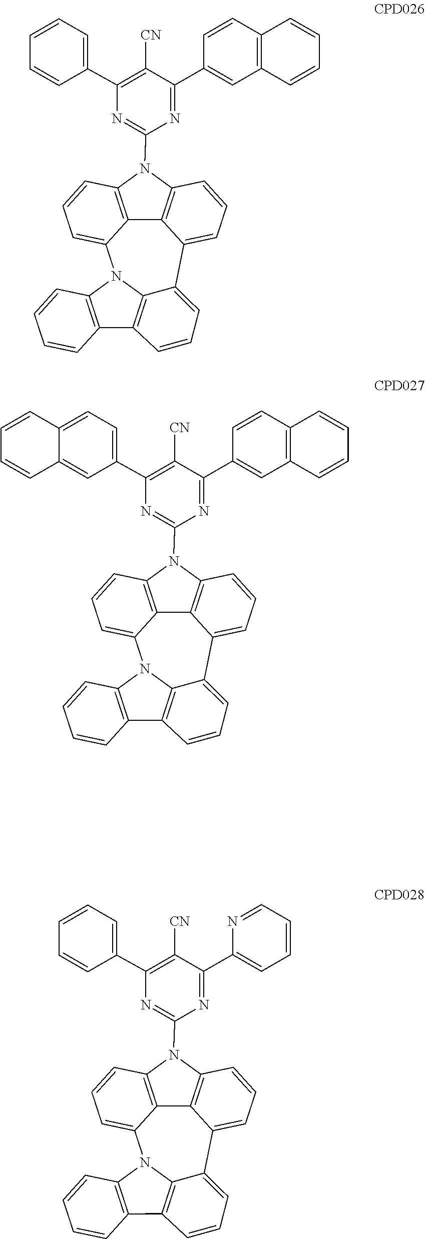 [SMILES: N#Cc1c(-c2ccccc2)nc(-n2c3cccc4c5cccc6c7ccccc7n(c7cccc2c7c43)c56)nc1-c1ccc2ccccc2c1 ; N#Cc1c(-c2ccc3ccccc3c2)nc(-n2c3cccc4c5cccc6c7ccccc7n(c7cccc2c7c43)c56)nc1-c1ccc2ccccc2c1 ; N#Cc1c(-c2ccccc2)nc(-n2c3cccc4c5cccc6c7ccccc7n(c7cccc2c7c43)c56)nc1-c1ccccn1]

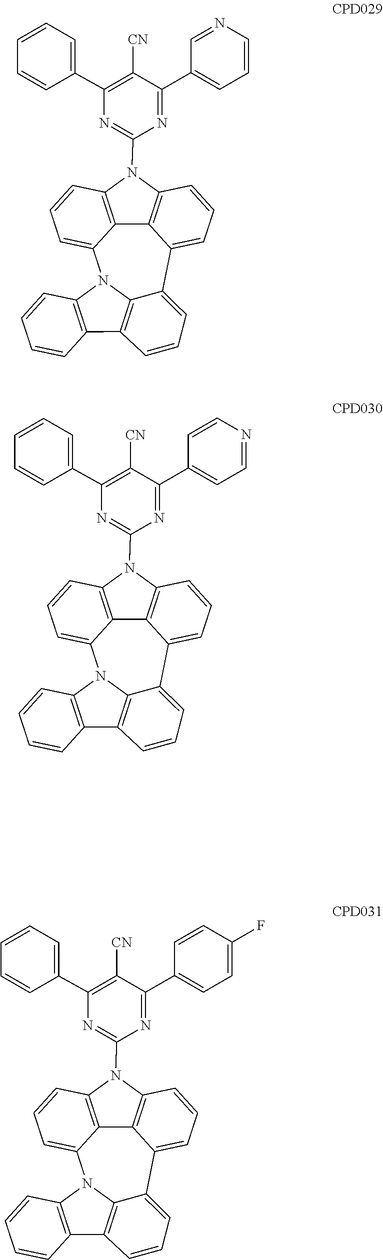 [SMILES: N#Cc1c(-c2ccccc2)nc(-n2c3cccc4c5cccc6c7ccccc7n(c7cccc2c7c43)c56)nc1-c1cccnc1 ; N#Cc1c(-c2ccccc2)nc(-n2c3cccc4c5cccc6c7ccccc7n(c7cccc2c7c43)c56)nc1-c1ccncc1 ; N#Cc1c(-c2ccccc2)nc(-n2c3cccc4c5cccc6c7ccccc7n(c7cccc2c7c43)c56)nc1-c1ccc(F)cc1]

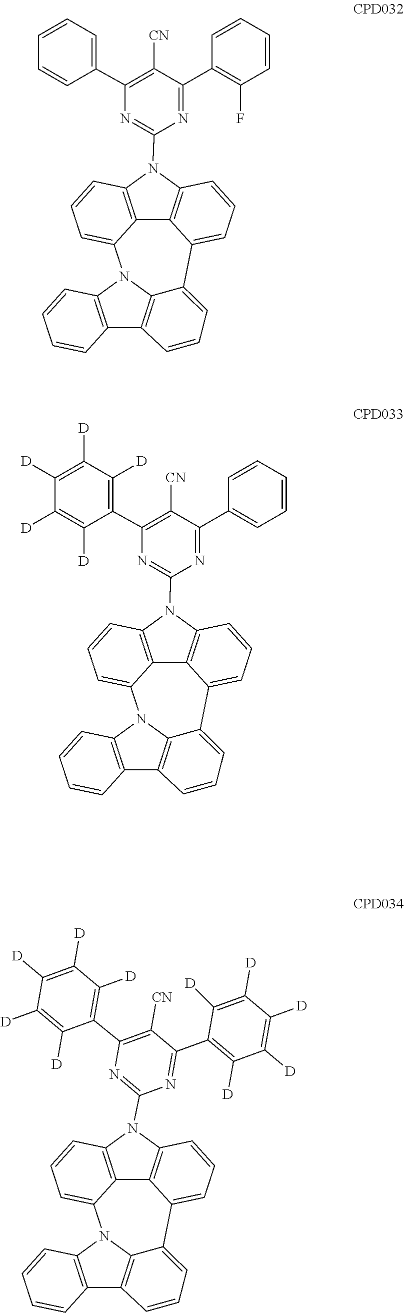 [SMILES: N#Cc1c(-c2ccccc2)nc(-n2c3cccc4c5cccc6c7ccccc7n(c7cccc2c7c43)c56)nc1-c1ccccc1F ; [2H]c1c([2H])c([2H])c(-c2nc(-n3c4cccc5c6cccc7c8ccccc8n(c8cccc3c8c54)c67)nc(-c3ccccc3)c2C#N)c([2H])c1[2H] ; [2H]c1c([2H])c([2H])c(-c2nc(-n3c4cccc5c6cccc7c8ccccc8n(c8cccc3c8c54)c67)nc(-c3c([2H])c([2H])c([2H])c([2H])c3[2H])c2C#N)c([2H])c1[2H]]

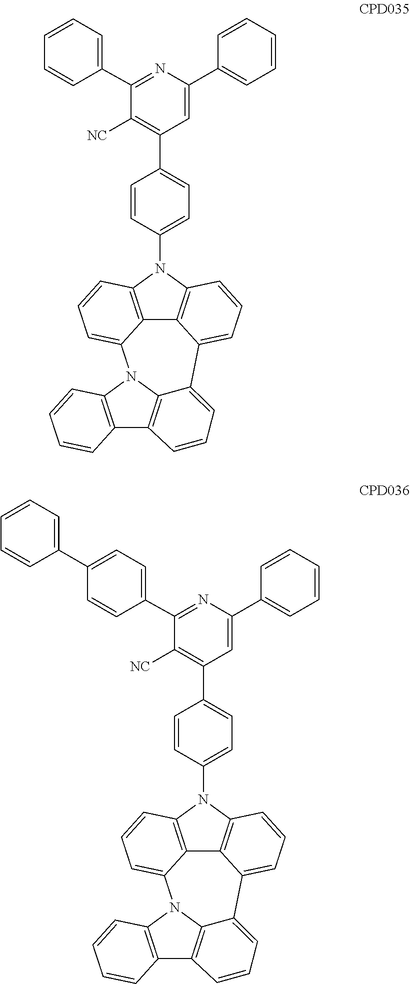 [SMILES: N#Cc1c(-c2ccc(-n3c4cccc5c6cccc7c8ccccc8n(c8cccc3c8c54)c67)cc2)cc(-c2ccccc2)nc1-c1ccccc1 ; N#Cc1c(-c2ccc(-n3c4cccc5c6cccc7c8ccccc8n(c8cccc3c8c54)c67)cc2)cc(-c2ccccc2)nc1-c1ccc(-c2ccccc2)cc1]

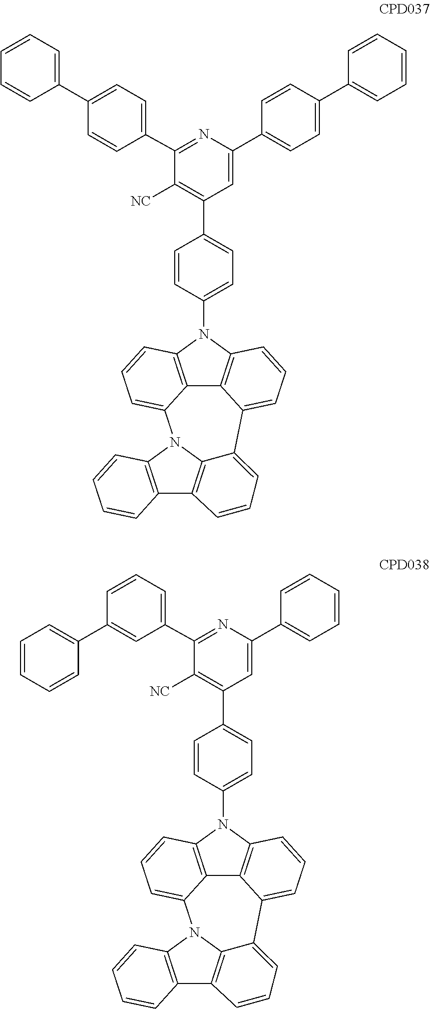 [SMILES: N#Cc1c(-c2ccc(-n3c4cccc5c6cccc7c8ccccc8n(c8cccc3c8c54)c67)cc2)cc(-c2ccc(-c3ccccc3)cc2)nc1-c1ccc(-c2ccccc2)cc1 ; N#Cc1c(-c2ccc(-n3c4cccc5c6cccc7c8ccccc8n(c8cccc3c8c54)c67)cc2)cc(-c2ccccc2)nc1-c1cccc(-c2ccccc2)c1]

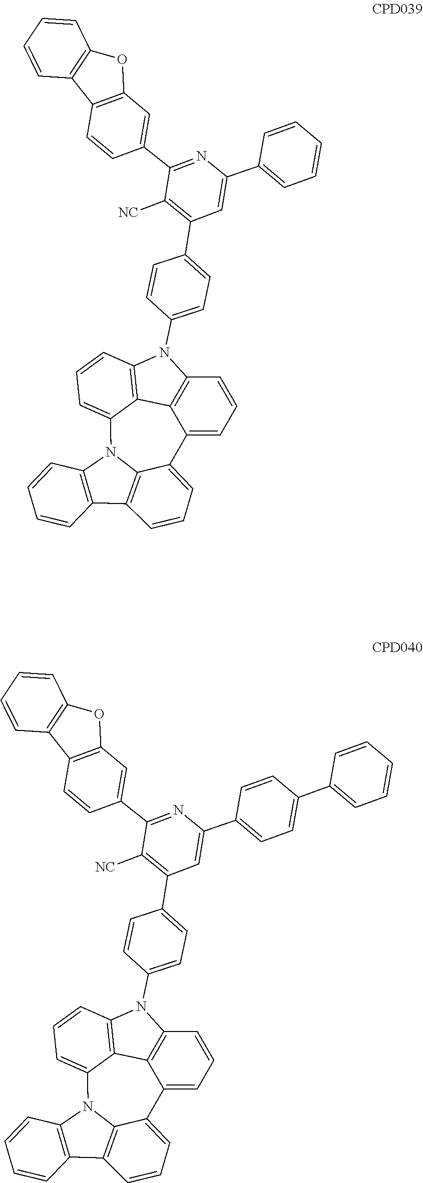 [SMILES: N#Cc1c(-c2ccc(-n3c4cccc5c6cccc7c8ccccc8n(c8cccc3c8c54)c67)cc2)cc(-c2ccccc2)nc1-c1ccc2c(c1)oc1ccccc12 ; N#Cc1c(-c2ccc(-n3c4cccc5c6cccc7c8ccccc8n(c8cccc3c8c54)c67)cc2)cc(-c2ccc(-c3ccccc3)cc2)nc1-c1ccc2c(c1)oc1ccccc12]

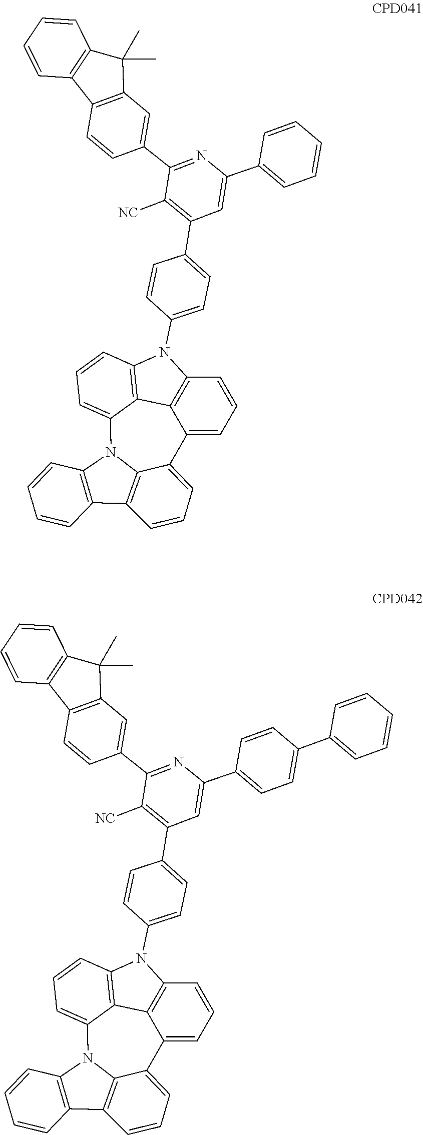 [SMILES: CC1(C)c2ccccc2-c2ccc(-c3nc(-c4ccccc4)cc(-c4ccc(-n5c6cccc7c8cccc9c%10ccccc%10n(c%10cccc5c%10c76)c89)cc4)c3C#N)cc21 ; CC1(C)c2ccccc2-c2ccc(-c3nc(-c4ccc(-c5ccccc5)cc4)cc(-c4ccc(-n5c6cccc7c8cccc9c%10ccccc%10n(c%10cccc5c%10c76)c89)cc4)c3C#N)cc21]

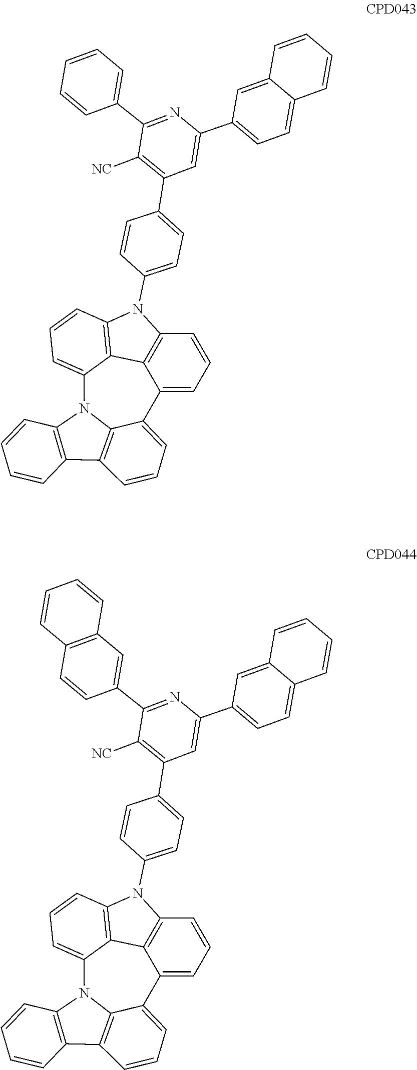 [SMILES: N#Cc1c(-c2ccc(-n3c4cccc5c6cccc7c8ccccc8n(c8cccc3c8c54)c67)cc2)cc(-c2ccc3ccccc3c2)nc1-c1ccccc1 ; N#Cc1c(-c2ccc(-n3c4cccc5c6cccc7c8ccccc8n(c8cccc3c8c54)c67)cc2)cc(-c2ccc3ccccc3c2)nc1-c1ccc2ccccc2c1]

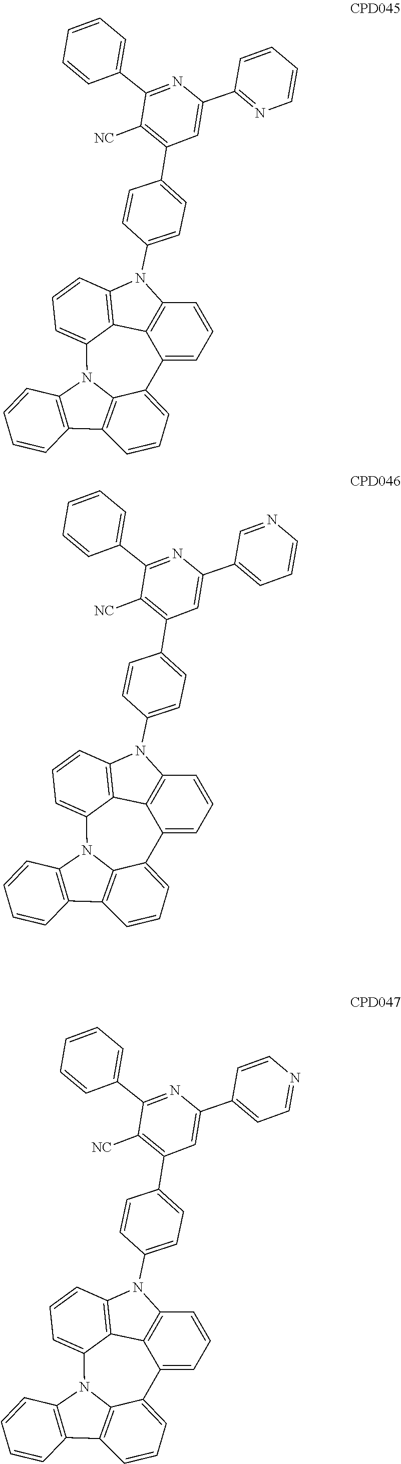

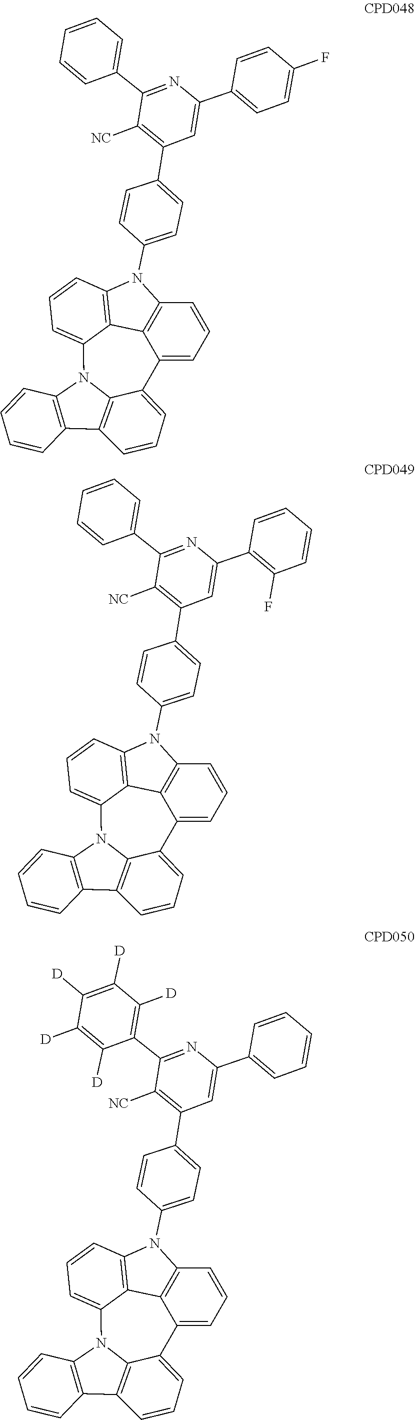

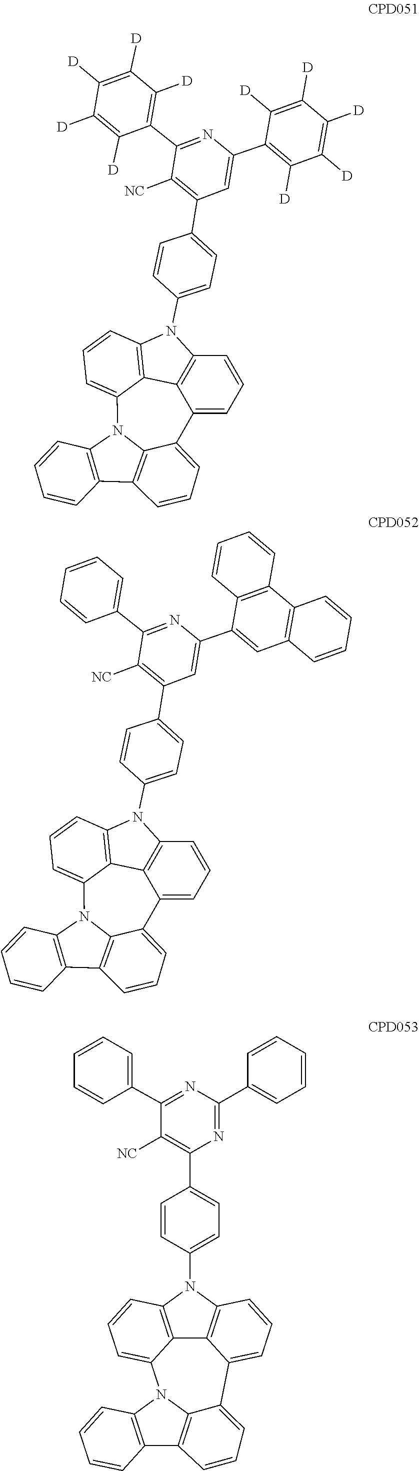 [SMILES: [2H]c1c([2H])c([2H])c(-c2cc(-c3ccc(-n4c5cccc6c7cccc8c9ccccc9n(c9cccc4c9c65)c78)cc3)c(C#N)c(-c3c([2H])c([2H])c([2H])c([2H])c3[2H])n2)c([2H])c1[2H] ; N#Cc1c(-c2ccc(-n3c4cccc5c6cccc7c8ccccc8n(c8cccc3c8c54)c67)cc2)cc(-c2cc3ccccc3c3ccccc23)nc1-c1ccccc1 ; N#Cc1c(-c2ccccc2)nc(-c2ccccc2)nc1-c1ccc(-n2c3cccc4c5cccc6c7ccccc7n(c7cccc2c7c43)c56)cc1]

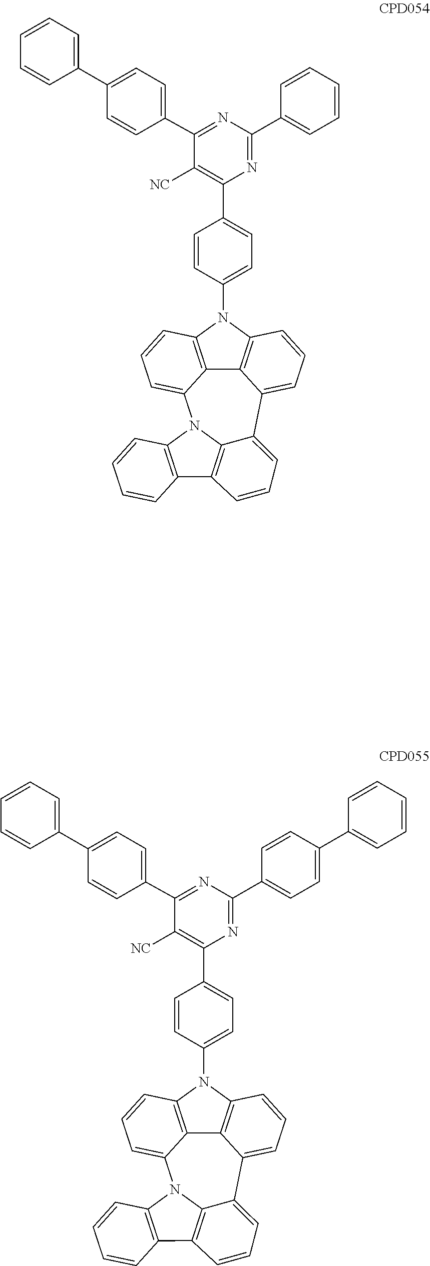 [SMILES: N#Cc1c(-c2ccc(-c3ccccc3)cc2)nc(-c2ccccc2)nc1-c1ccc(-n2c3cccc4c5cccc6c7ccccc7n(c7cccc2c7c43)c56)cc1 ; N#Cc1c(-c2ccc(-c3ccccc3)cc2)nc(-c2ccc(-c3ccccc3)cc2)nc1-c1ccc(-n2c3cccc4c5cccc6c7ccccc7n(c7cccc2c7c43)c56)cc1]

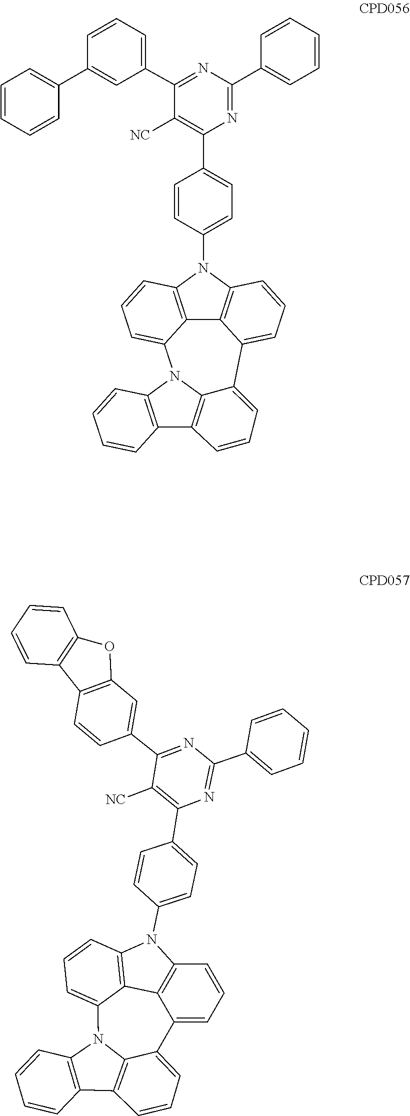 [SMILES: N#Cc1c(-c2ccc(-n3c4cccc5c6cccc7c8ccccc8n(c8cccc3c8c54)c67)cc2)nc(-c2ccccc2)nc1-c1cccc(-c2ccccc2)c1 ; N#Cc1c(-c2ccc(-n3c4cccc5c6cccc7c8ccccc8n(c8cccc3c8c54)c67)cc2)nc(-c2ccccc2)nc1-c1ccc2c(c1)oc1ccccc12]

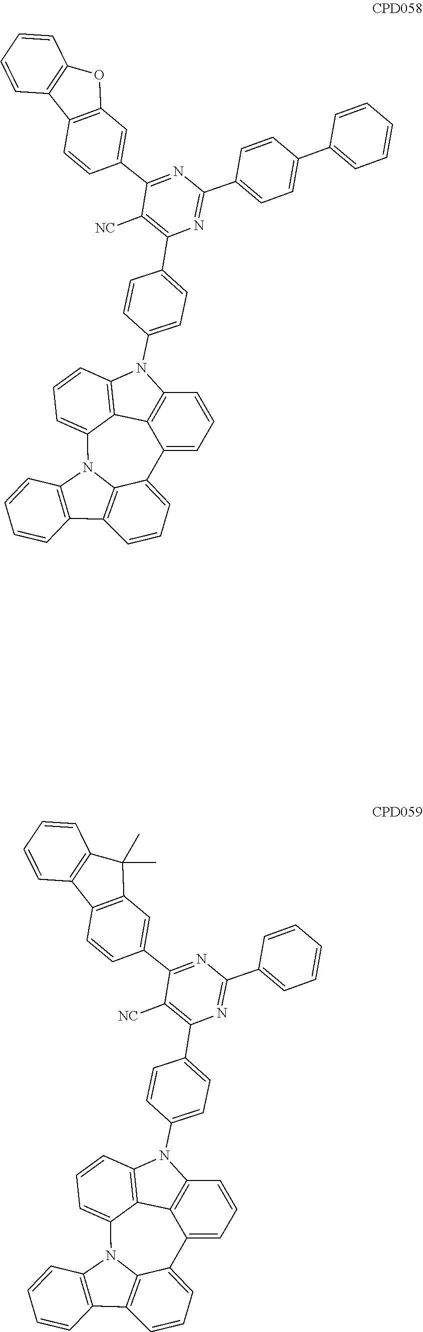 [SMILES: N#Cc1c(-c2ccc(-n3c4cccc5c6cccc7c8ccccc8n(c8cccc3c8c54)c67)cc2)nc(-c2ccc(-c3ccccc3)cc2)nc1-c1ccc2c(c1)oc1ccccc12 ; CC1(C)c2ccccc2-c2ccc(-c3nc(-c4ccccc4)nc(-c4ccc(-n5c6cccc7c8cccc9c%10ccccc%10n(c%10cccc5c%10c76)c89)cc4)c3C#N)cc21]

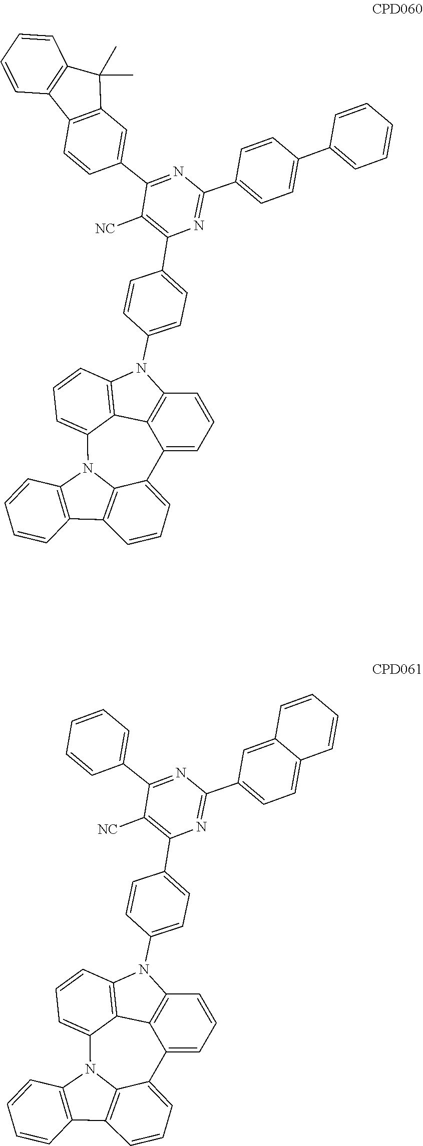 [SMILES: CC1(C)c2ccccc2-c2ccc(-c3nc(-c4ccc(-c5ccccc5)cc4)nc(-c4ccc(-n5c6cccc7c8cccc9c%10ccccc%10n(c%10cccc5c%10c76)c89)cc4)c3C#N)cc21 ; N#Cc1c(-c2ccccc2)nc(-c2ccc3ccccc3c2)nc1-c1ccc(-n2c3cccc4c5cccc6c7ccccc7n(c7cccc2c7c43)c56)cc1]

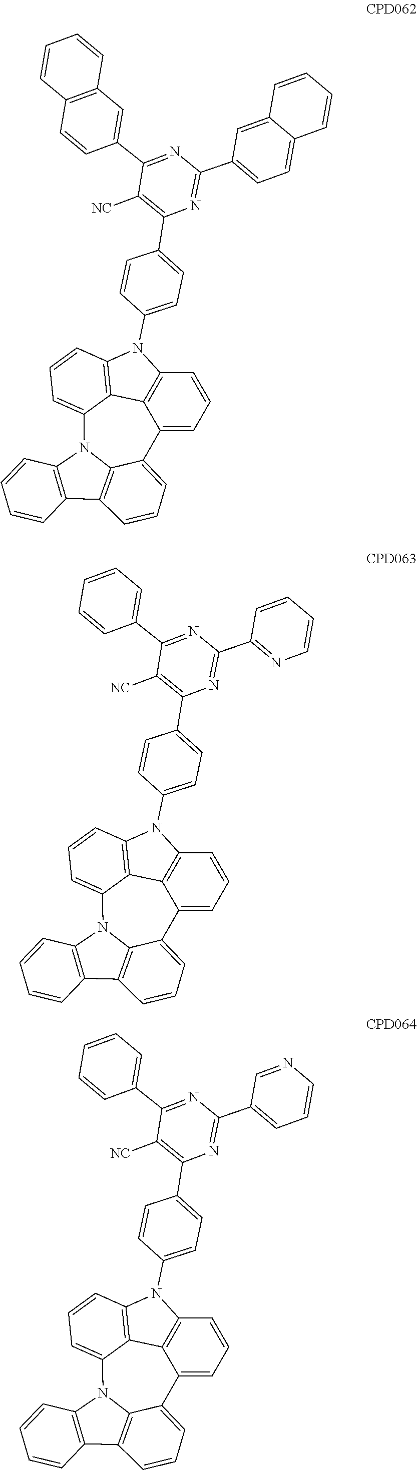

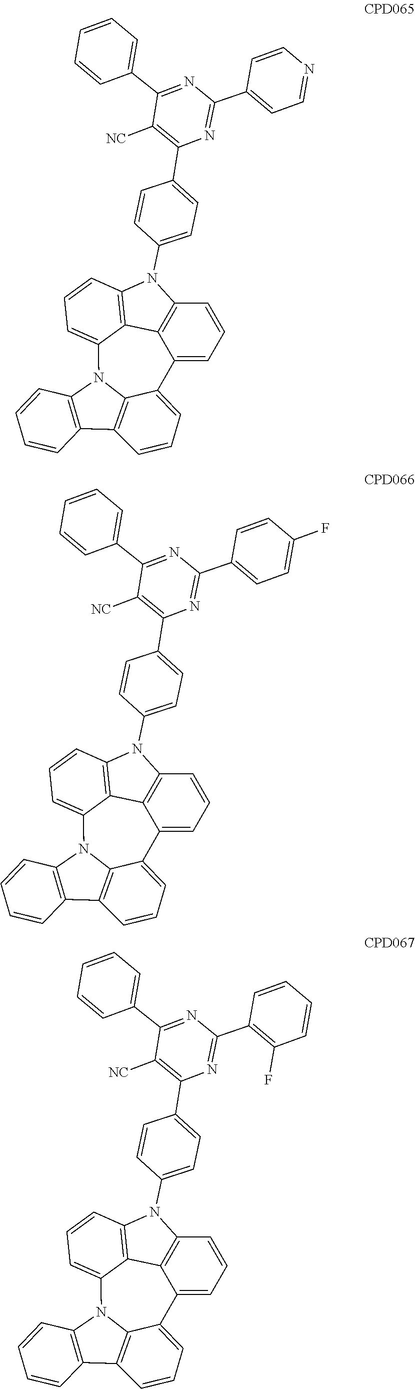

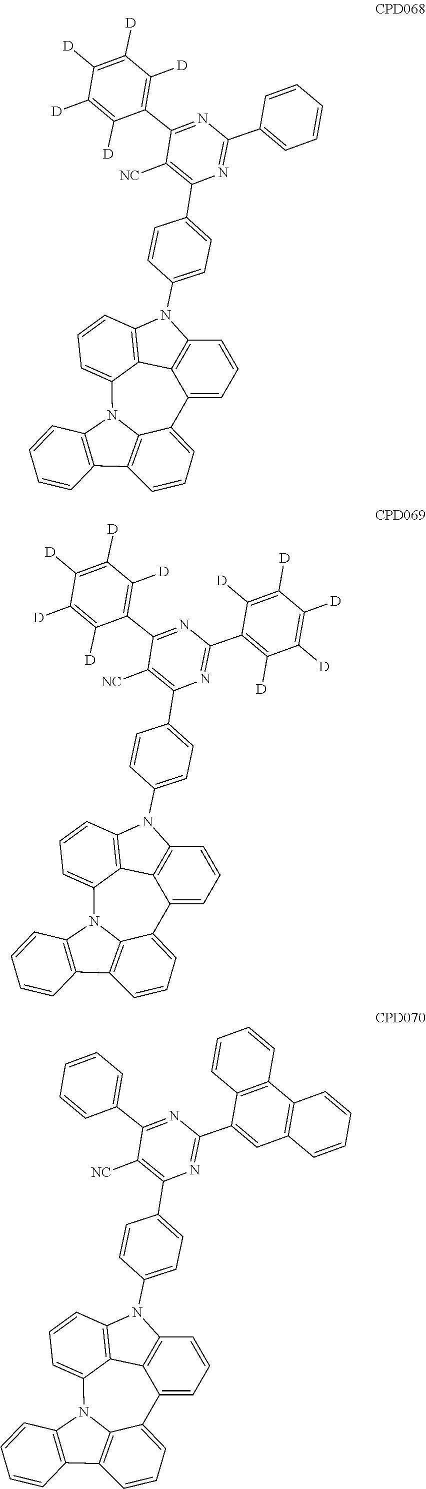 [SMILES: [2H]c1c([2H])c([2H])c(-c2nc(-c3ccccc3)nc(-c3ccc(-n4c5cccc6c7cccc8c9ccccc9n(c9cccc4c9c65)c78)cc3)c2C#N)c([2H])c1[2H] ; [2H]c1c([2H])c([2H])c(-c2nc(-c3ccc(-n4c5cccc6c7cccc8c9ccccc9n(c9cccc4c9c65)c78)cc3)c(C#N)c(-c3c([2H])c([2H])c([2H])c([2H])c3[2H])n2)c([2H])c1[2H] ; N#Cc1c(-c2ccccc2)nc(-c2cc3ccccc3c3ccccc23)nc1-c1ccc(-n2c3cccc4c5cccc6c7ccccc7n(c7cccc2c7c43)c56)cc1]

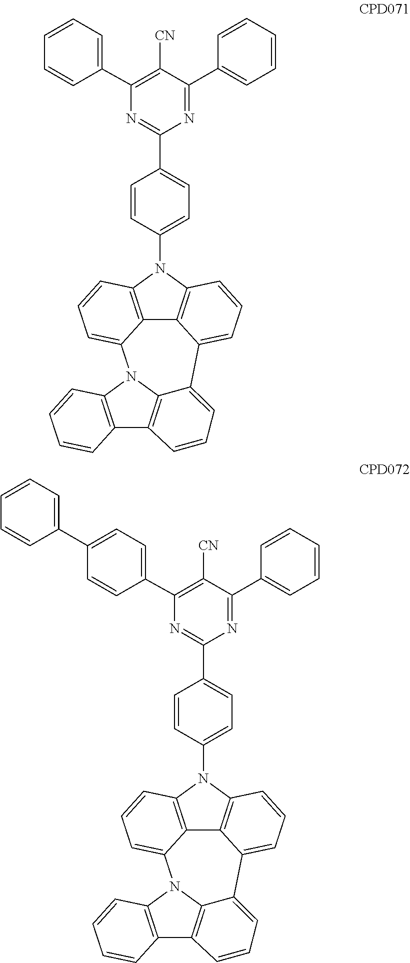

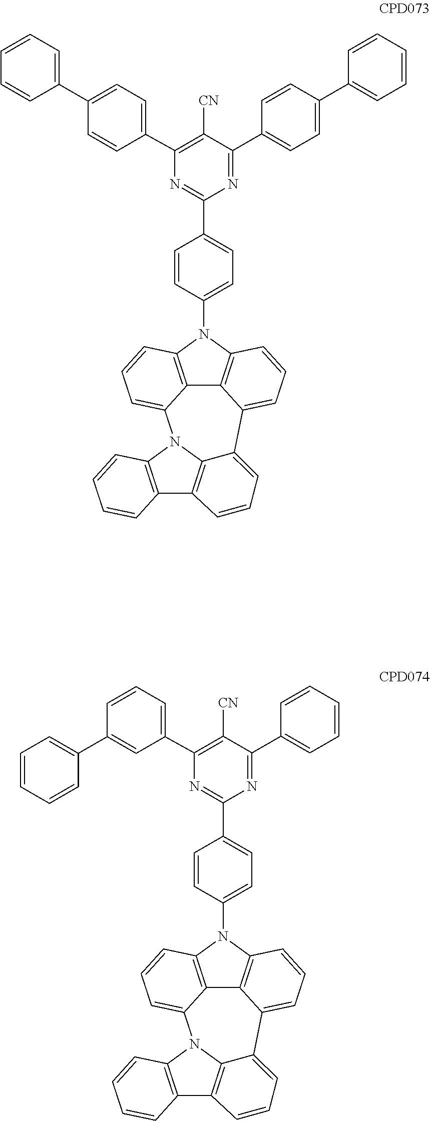 [SMILES: N#Cc1c(-c2ccc(-c3ccccc3)cc2)nc(-c2ccc(-n3c4cccc5c6cccc7c8ccccc8n(c8cccc3c8c54)c67)cc2)nc1-c1ccc(-c2ccccc2)cc1 ; N#Cc1c(-c2ccccc2)nc(-c2ccc(-n3c4cccc5c6cccc7c8ccccc8n(c8cccc3c8c54)c67)cc2)nc1-c1cccc(-c2ccccc2)c1]

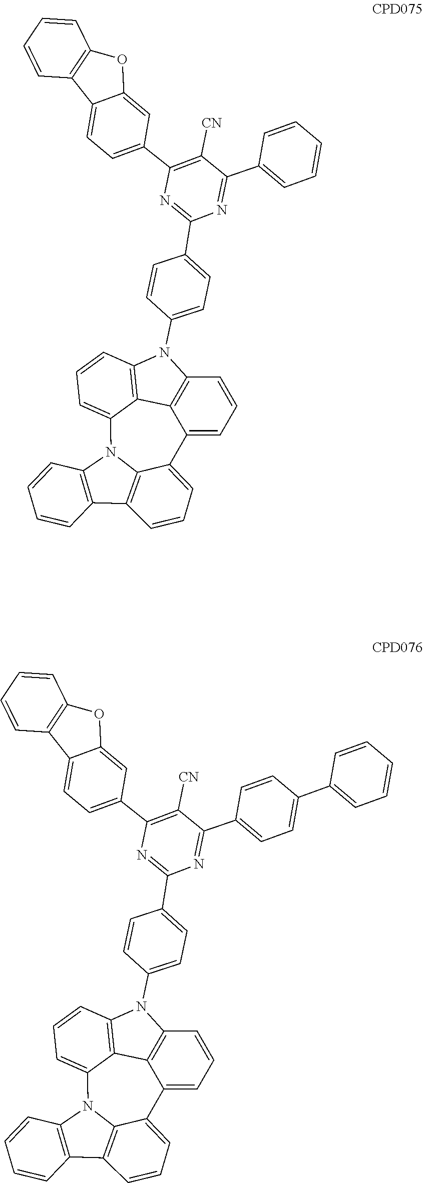 [SMILES: N#Cc1c(-c2ccccc2)nc(-c2ccc(-n3c4cccc5c6cccc7c8ccccc8n(c8cccc3c8c54)c67)cc2)nc1-c1ccc2c(c1)oc1ccccc12 ; N#Cc1c(-c2ccc(-c3ccccc3)cc2)nc(-c2ccc(-n3c4cccc5c6cccc7c8ccccc8n(c8cccc3c8c54)c67)cc2)nc1-c1ccc2c(c1)oc1ccccc12]

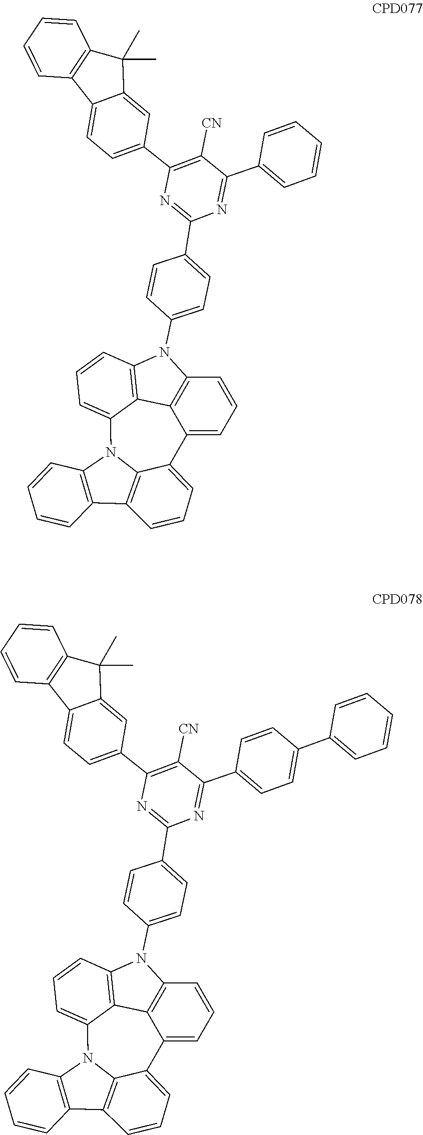 [SMILES: CC1(C)c2ccccc2-c2ccc(-c3nc(-c4ccc(-n5c6cccc7c8cccc9c%10ccccc%10n(c%10cccc5c%10c76)c89)cc4)nc(-c4ccccc4)c3C#N)cc21 ; CC1(C)c2ccccc2-c2ccc(-c3nc(-c4ccc(-n5c6cccc7c8cccc9c%10ccccc%10n(c%10cccc5c%10c76)c89)cc4)nc(-c4ccc(-c5ccccc5)cc4)c3C#N)cc21]

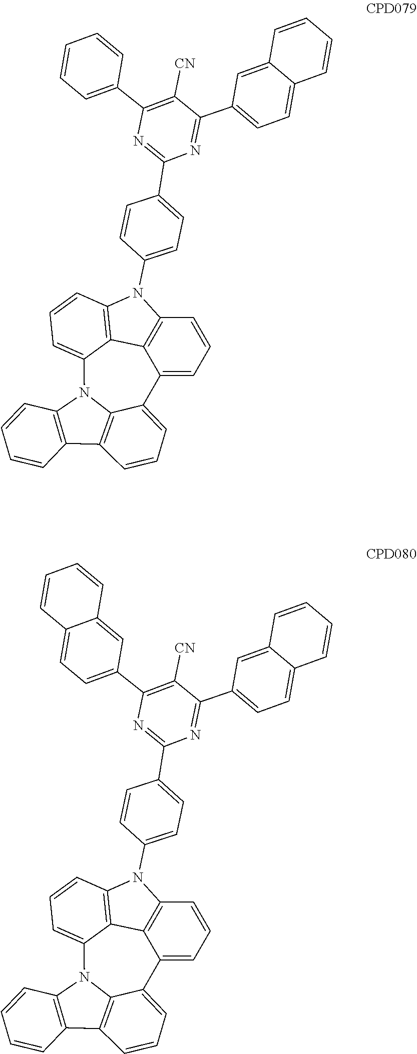 [SMILES: N#Cc1c(-c2ccccc2)nc(-c2ccc(-n3c4cccc5c6cccc7c8ccccc8n(c8cccc3c8c54)c67)cc2)nc1-c1ccc2ccccc2c1 ; N#Cc1c(-c2ccc3ccccc3c2)nc(-c2ccc(-n3c4cccc5c6cccc7c8ccccc8n(c8cccc3c8c54)c67)cc2)nc1-c1ccc2ccccc2c1]

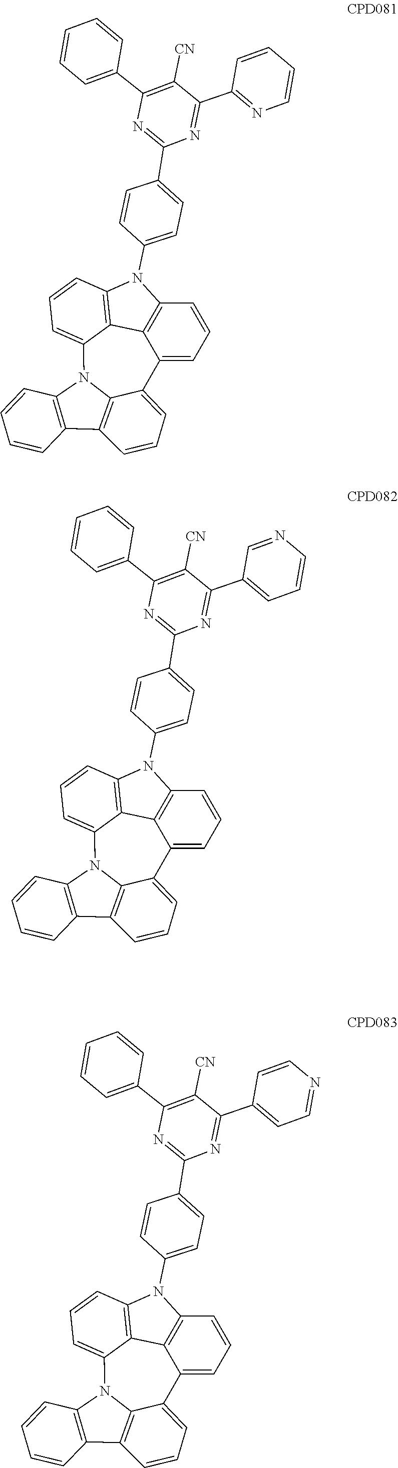 [SMILES: N#Cc1c(-c2ccccc2)nc(-c2ccc(-n3c4cccc5c6cccc7c8ccccc8n(c8cccc3c8c54)c67)cc2)nc1-c1ccccn1 ; N#Cc1c(-c2ccccc2)nc(-c2ccc(-n3c4cccc5c6cccc7c8ccccc8n(c8cccc3c8c54)c67)cc2)nc1-c1cccnc1 ; N#Cc1c(-c2ccccc2)nc(-c2ccc(-n3c4cccc5c6cccc7c8ccccc8n(c8cccc3c8c54)c67)cc2)nc1-c1ccncc1]

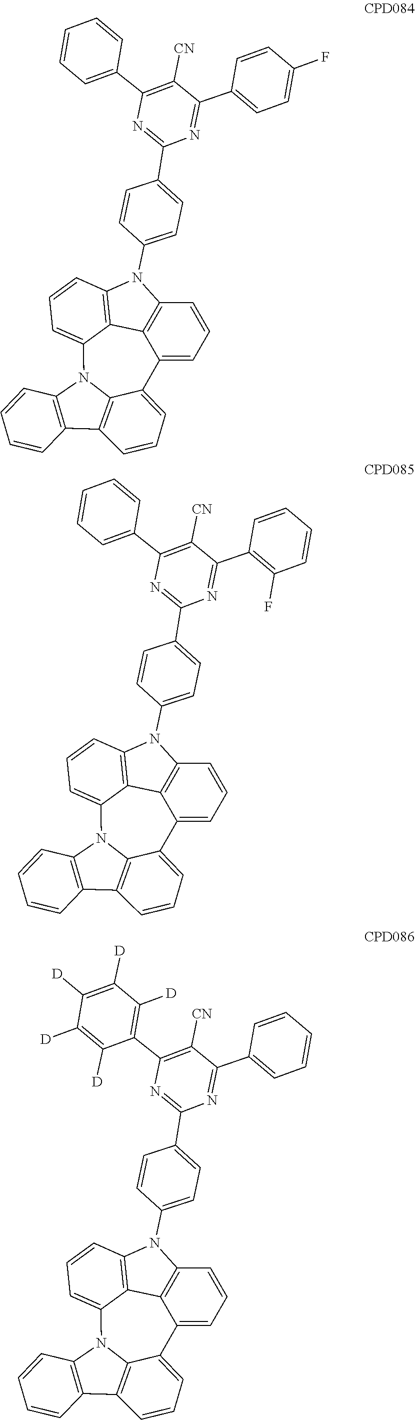

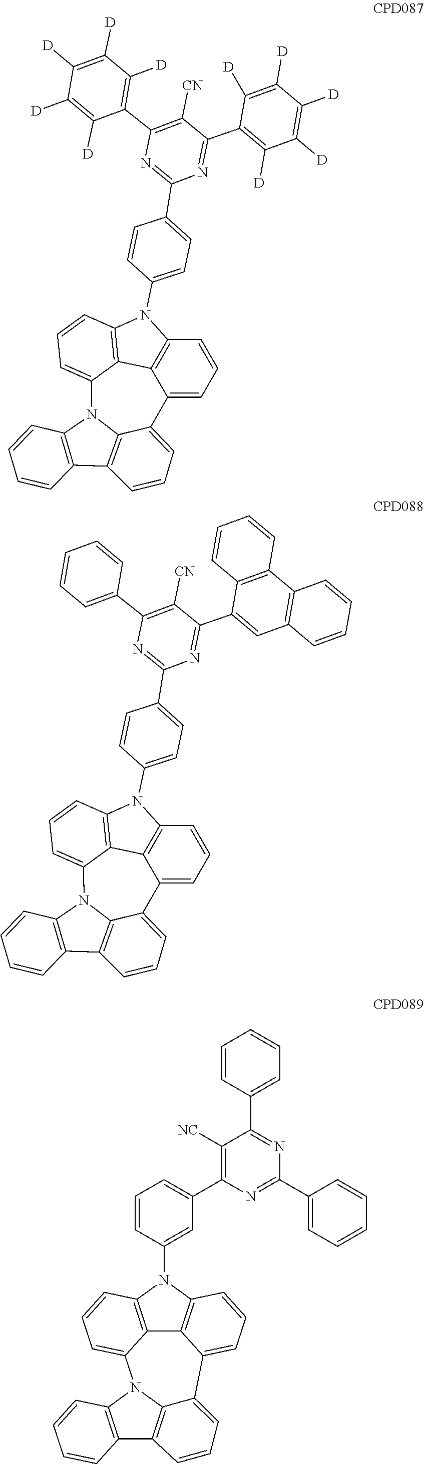 [SMILES: [2H]c1c([2H])c([2H])c(-c2nc(-c3ccc(-n4c5cccc6c7cccc8c9ccccc9n(c9cccc4c9c65)c78)cc3)nc(-c3c([2H])c([2H])c([2H])c([2H])c3[2H])c2C#N)c([2H])c1[2H] ; N#Cc1c(-c2ccccc2)nc(-c2ccc(-n3c4cccc5c6cccc7c8ccccc8n(c8cccc3c8c54)c67)cc2)nc1-c1cc2ccccc2c2ccccc12 ; N#Cc1c(-c2ccccc2)nc(-c2ccccc2)nc1-c1cccc(-n2c3cccc4c5cccc6c7ccccc7n(c7cccc2c7c43)c56)c1]

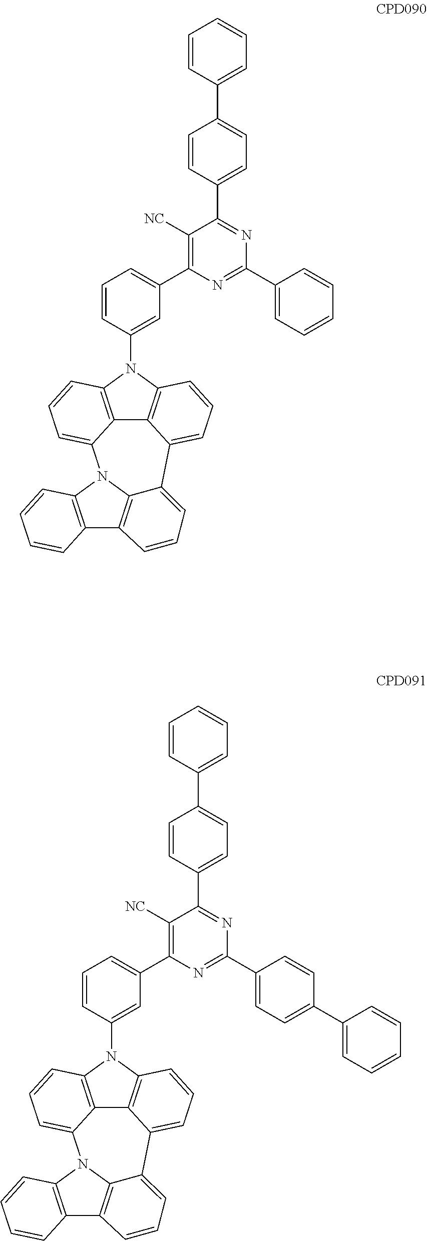 [SMILES: N#Cc1c(-c2ccc(-c3ccccc3)cc2)nc(-c2ccccc2)nc1-c1cccc(-n2c3cccc4c5cccc6c7ccccc7n(c7cccc2c7c43)c56)c1 ; N#Cc1c(-c2ccc(-c3ccccc3)cc2)nc(-c2ccc(-c3ccccc3)cc2)nc1-c1cccc(-n2c3cccc4c5cccc6c7ccccc7n(c7cccc2c7c43)c56)c1]

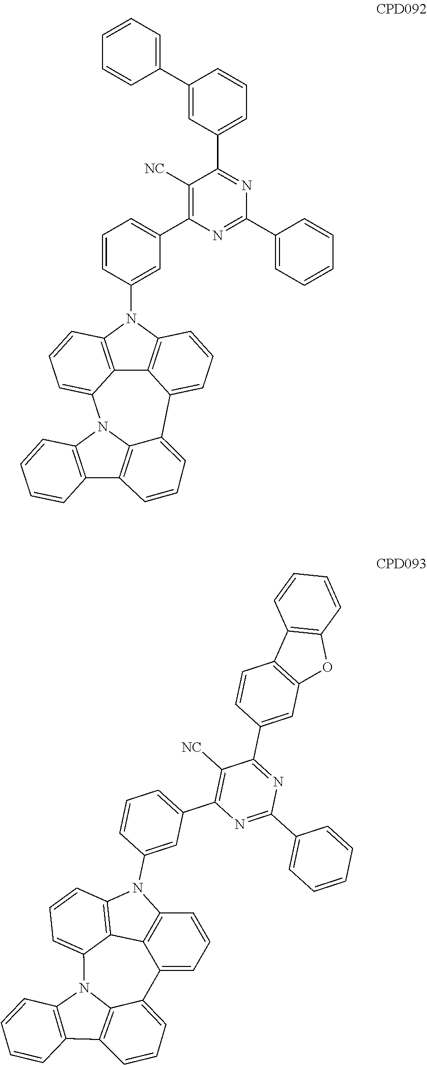 [SMILES: N#Cc1c(-c2cccc(-c3ccccc3)c2)nc(-c2ccccc2)nc1-c1cccc(-n2c3cccc4c5cccc6c7ccccc7n(c7cccc2c7c43)c56)c1 ; N#Cc1c(-c2cccc(-n3c4cccc5c6cccc7c8ccccc8n(c8cccc3c8c54)c67)c2)nc(-c2ccccc2)nc1-c1ccc2c(c1)oc1ccccc12]

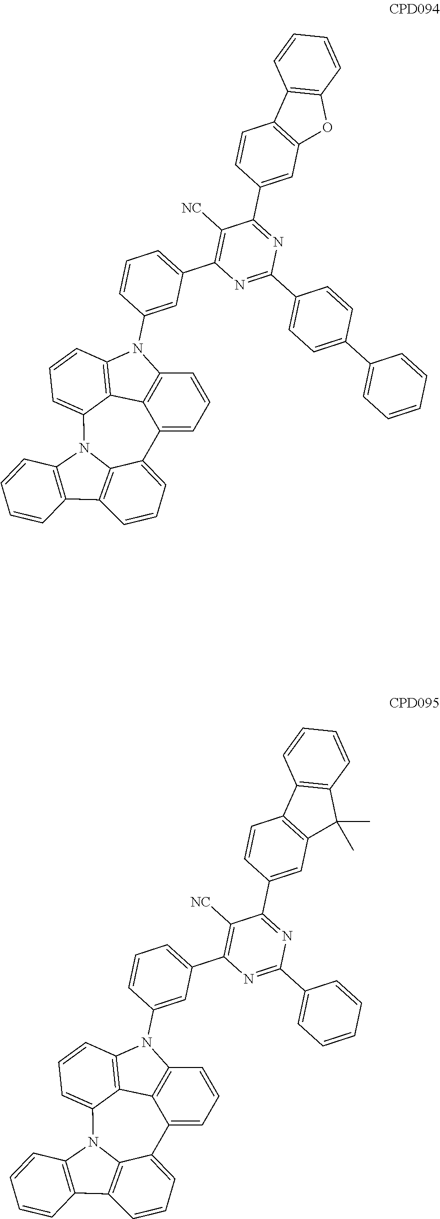 [SMILES: N#Cc1c(-c2cccc(-n3c4cccc5c6cccc7c8ccccc8n(c8cccc3c8c54)c67)c2)nc(-c2ccc(-c3ccccc3)cc2)nc1-c1ccc2c(c1)oc1ccccc12 ; CC1(C)c2ccccc2-c2ccc(-c3nc(-c4ccccc4)nc(-c4cccc(-n5c6cccc7c8cccc9c%10ccccc%10n(c%10cccc5c%10c76)c89)c4)c3C#N)cc21]

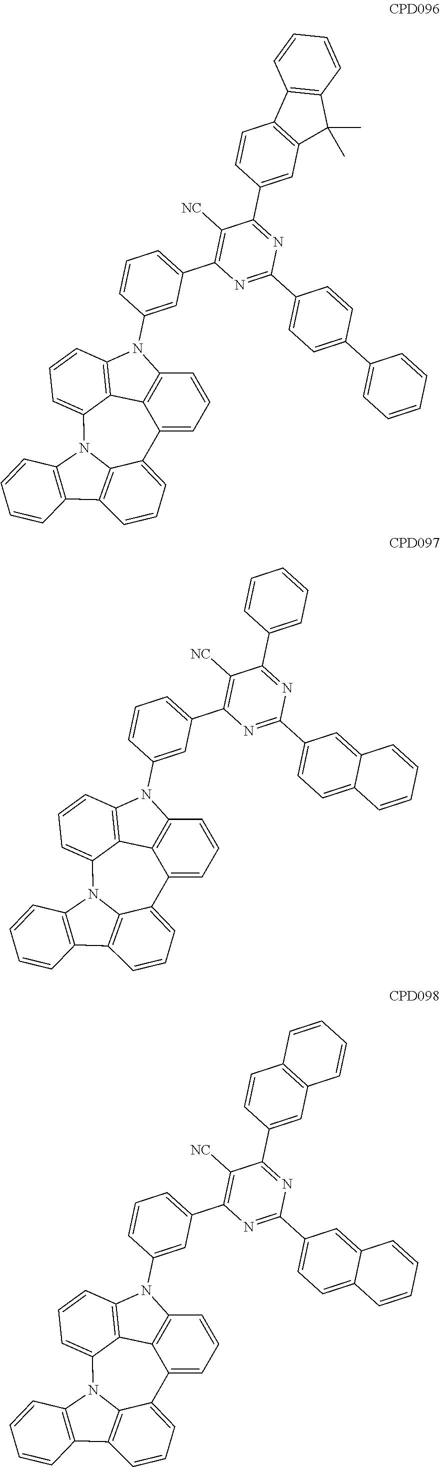 [SMILES: CC1(C)c2ccccc2-c2ccc(-c3nc(-c4ccc(-c5ccccc5)cc4)nc(-c4cccc(-n5c6cccc7c8cccc9c%10ccccc%10n(c%10cccc5c%10c76)c89)c4)c3C#N)cc21 ; N#Cc1c(-c2ccccc2)nc(-c2ccc3ccccc3c2)nc1-c1cccc(-n2c3cccc4c5cccc6c7ccccc7n(c7cccc2c7c43)c56)c1 ; N#Cc1c(-c2cccc(-n3c4cccc5c6cccc7c8ccccc8n(c8cccc3c8c54)c67)c2)nc(-c2ccc3ccccc3c2)nc1-c1ccc2ccccc2c1]

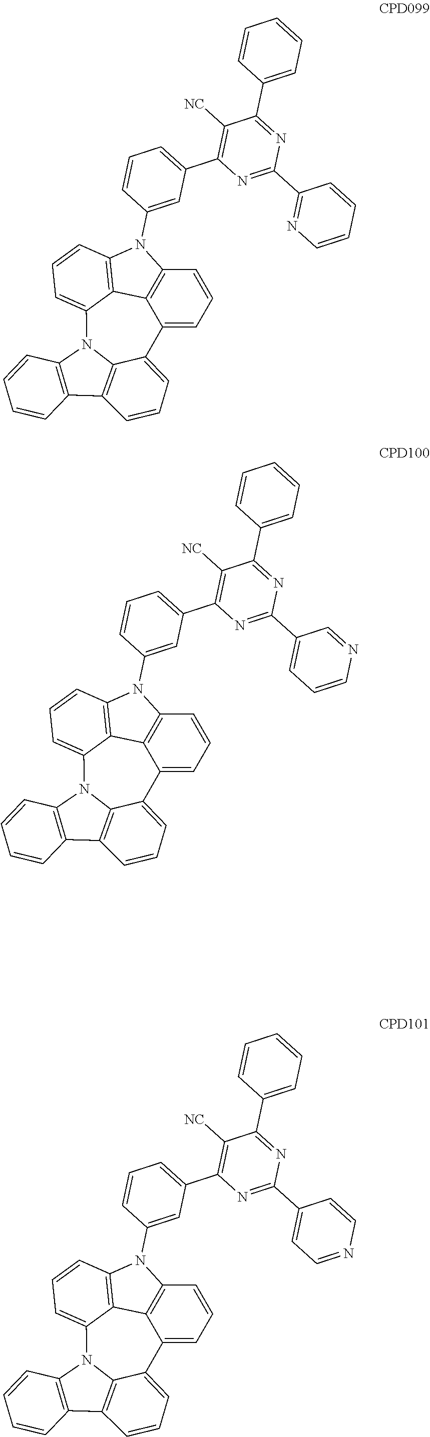 [SMILES: N#Cc1c(-c2ccccc2)nc(-c2ccccn2)nc1-c1cccc(-n2c3cccc4c5cccc6c7ccccc7n(c7cccc2c7c43)c56)c1 ; N#Cc1c(-c2ccccc2)nc(-c2cccnc2)nc1-c1cccc(-n2c3cccc4c5cccc6c7ccccc7n(c7cccc2c7c43)c56)c1 ; N#Cc1c(-c2ccccc2)nc(-c2ccncc2)nc1-c1cccc(-n2c3cccc4c5cccc6c7ccccc7n(c7cccc2c7c43)c56)c1]

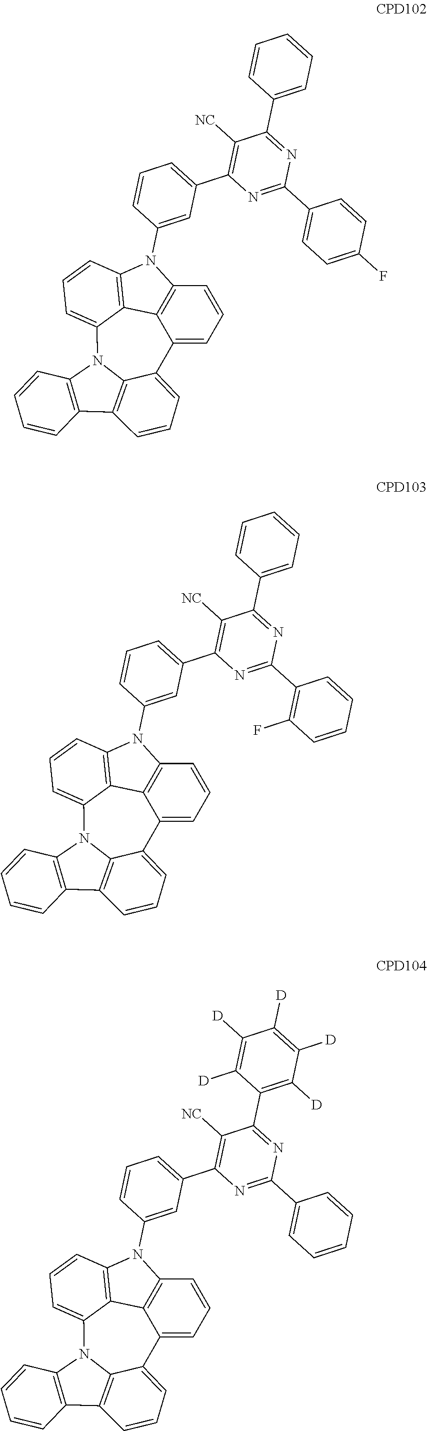 [SMILES: N#Cc1c(-c2ccccc2)nc(-c2ccc(F)cc2)nc1-c1cccc(-n2c3cccc4c5cccc6c7ccccc7n(c7cccc2c7c43)c56)c1 ; N#Cc1c(-c2ccccc2)nc(-c2ccccc2F)nc1-c1cccc(-n2c3cccc4c5cccc6c7ccccc7n(c7cccc2c7c43)c56)c1 ; [2H]c1c([2H])c([2H])c(-c2nc(-c3ccccc3)nc(-c3cccc(-n4c5cccc6c7cccc8c9ccccc9n(c9cccc4c9c65)c78)c3)c2C#N)c([2H])c1[2H]]

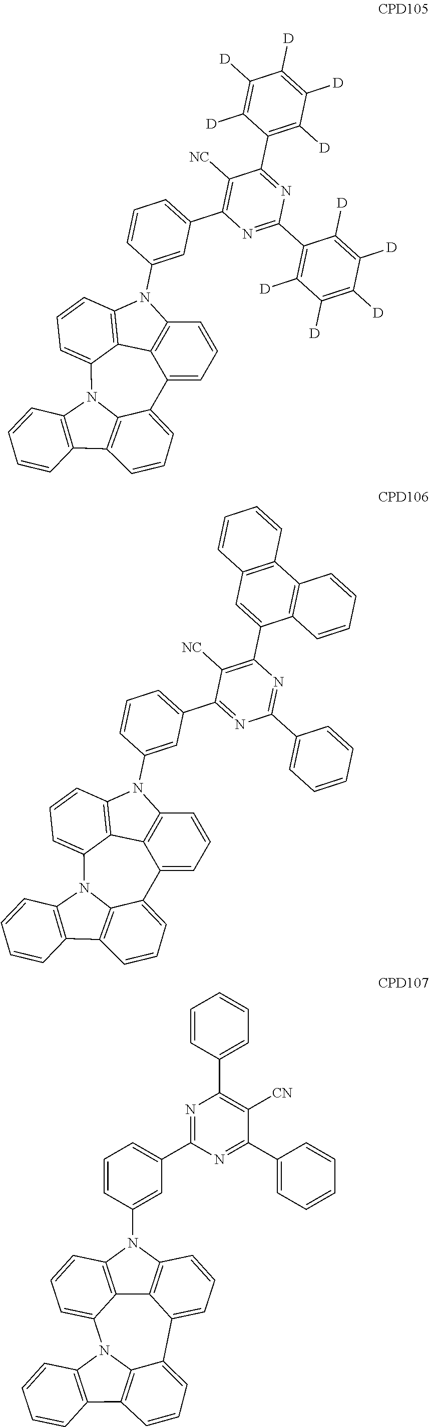 [SMILES: [2H]c1c([2H])c([2H])c(-c2nc(-c3cccc(-n4c5cccc6c7cccc8c9ccccc9n(c9cccc4c9c65)c78)c3)c(C#N)c(-c3c([2H])c([2H])c([2H])c([2H])c3[2H])n2)c([2H])c1[2H] ; N#Cc1c(-c2cccc(-n3c4cccc5c6cccc7c8ccccc8n(c8cccc3c8c54)c67)c2)nc(-c2ccccc2)nc1-c1cc2ccccc2c2ccccc12 ; N#Cc1c(-c2ccccc2)nc(-c2cccc(-n3c4cccc5c6cccc7c8ccccc8n(c8cccc3c8c54)c67)c2)nc1-c1ccccc1]

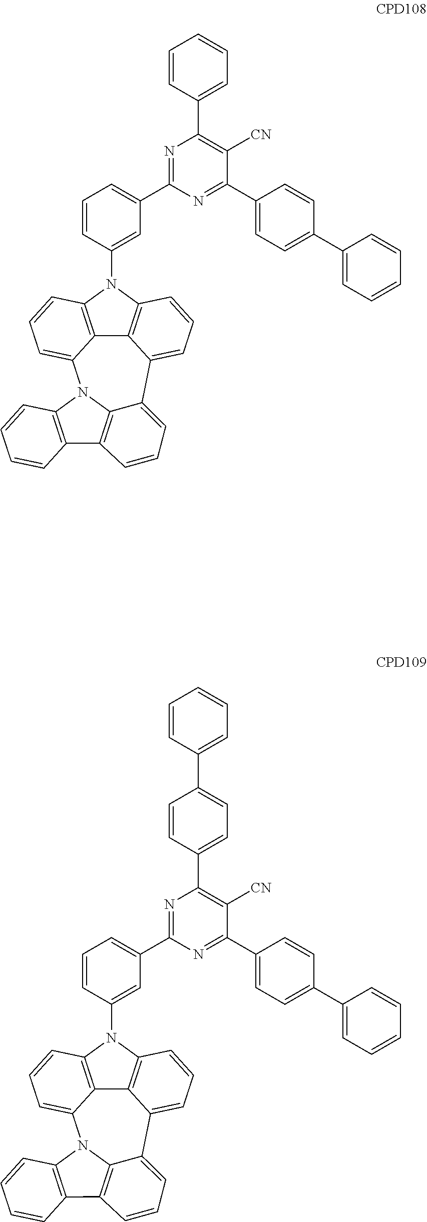 [SMILES: N#Cc1c(-c2ccccc2)nc(-c2cccc(-n3c4cccc5c6cccc7c8ccccc8n(c8cccc3c8c54)c67)c2)nc1-c1ccc(-c2ccccc2)cc1 ; N#Cc1c(-c2ccc(-c3ccccc3)cc2)nc(-c2cccc(-n3c4cccc5c6cccc7c8ccccc8n(c8cccc3c8c54)c67)c2)nc1-c1ccc(-c2ccccc2)cc1]

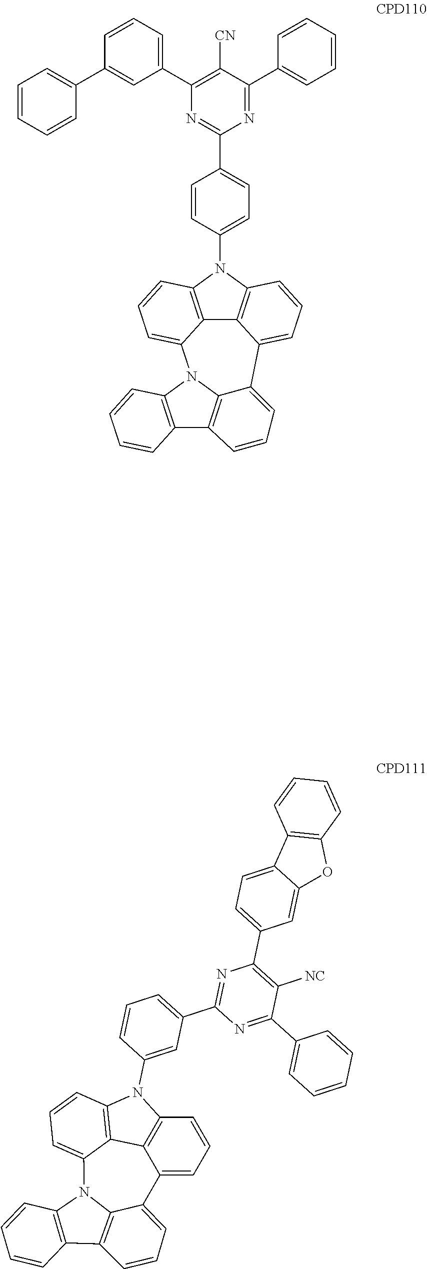

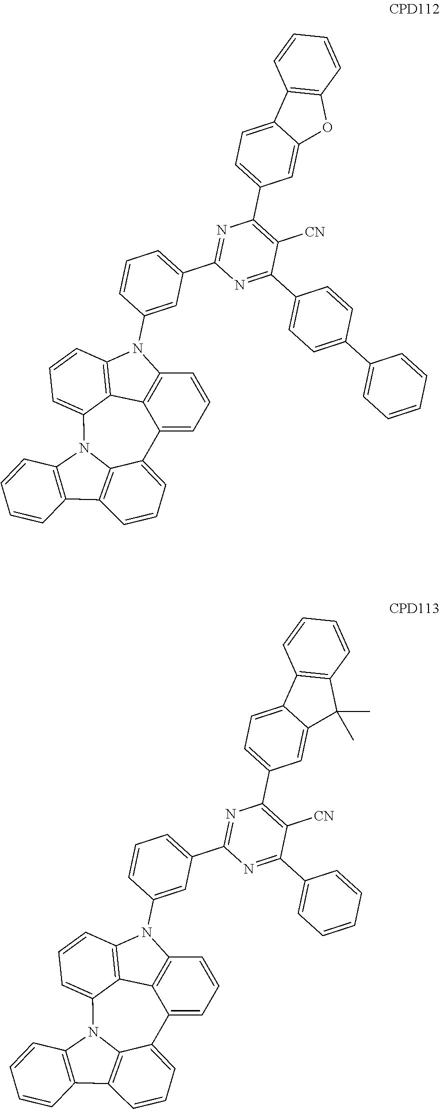 [SMILES: N#Cc1c(-c2ccc(-c3ccccc3)cc2)nc(-c2cccc(-n3c4cccc5c6cccc7c8ccccc8n(c8cccc3c8c54)c67)c2)nc1-c1ccc2c(c1)oc1ccccc12 ; CC1(C)c2ccccc2-c2ccc(-c3nc(-c4cccc(-n5c6cccc7c8cccc9c%10ccccc%10n(c%10cccc5c%10c76)c89)c4)nc(-c4ccccc4)c3C#N)cc21]

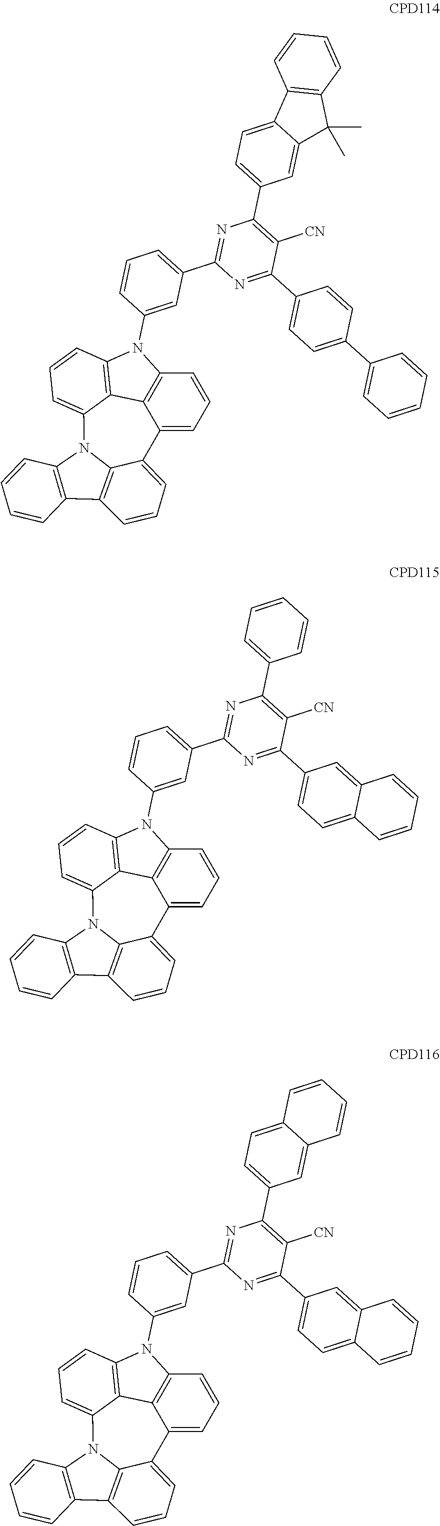 [SMILES: CC1(C)c2ccccc2-c2ccc(-c3nc(-c4cccc(-n5c6cccc7c8cccc9c%10ccccc%10n(c%10cccc5c%10c76)c89)c4)nc(-c4ccc(-c5ccccc5)cc4)c3C#N)cc21 ; N#Cc1c(-c2ccccc2)nc(-c2cccc(-n3c4cccc5c6cccc7c8ccccc8n(c8cccc3c8c54)c67)c2)nc1-c1ccc2ccccc2c1 ; N#Cc1c(-c2ccc3ccccc3c2)nc(-c2cccc(-n3c4cccc5c6cccc7c8ccccc8n(c8cccc3c8c54)c67)c2)nc1-c1ccc2ccccc2c1]

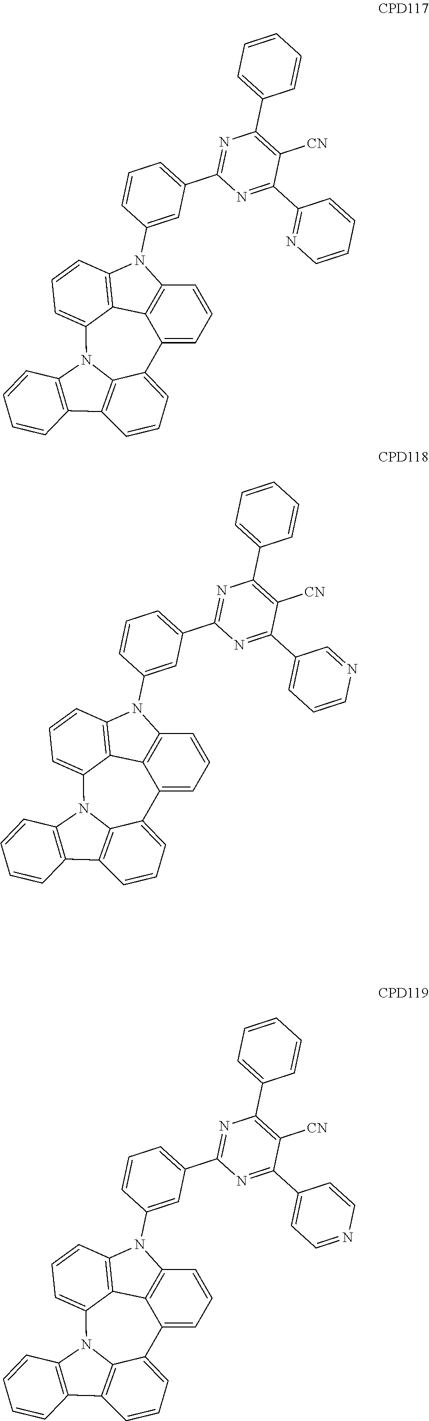 [SMILES: N#Cc1c(-c2ccccc2)nc(-c2cccc(-n3c4cccc5c6cccc7c8ccccc8n(c8cccc3c8c54)c67)c2)nc1-c1ccccn1 ; N#Cc1c(-c2ccccc2)nc(-c2cccc(-n3c4cccc5c6cccc7c8ccccc8n(c8cccc3c8c54)c67)c2)nc1-c1cccnc1 ; N#Cc1c(-c2ccccc2)nc(-c2cccc(-n3c4cccc5c6cccc7c8ccccc8n(c8cccc3c8c54)c67)c2)nc1-c1ccncc1]

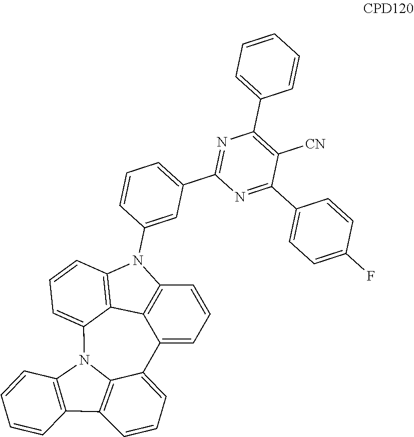 [SMILES: N#Cc1c(-c2ccccc2)nc(-c2cccc(-n3c4cccc5c6cccc7c8ccccc8n(c8cccc3c8c54)c67)c2)nc1-c1ccc(F)cc1]

[chemical structure]

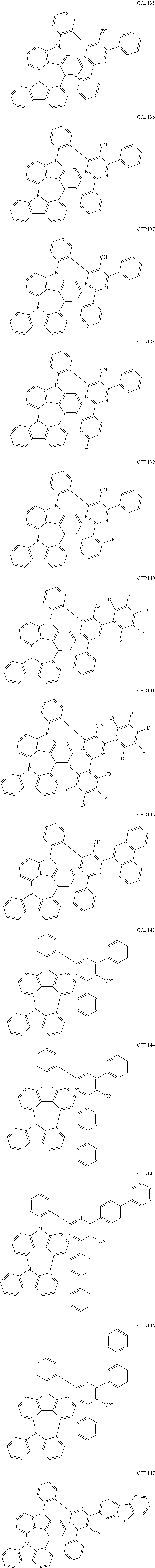

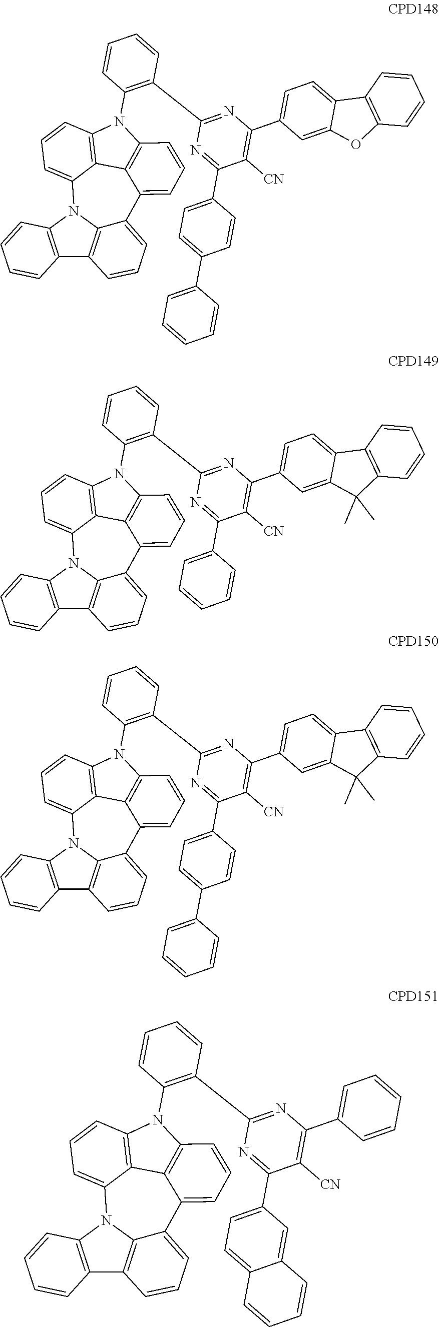 [SMILES: N#Cc1c(-c2ccc(-c3ccccc3)cc2)nc(-c2ccccc2-n2c3cccc4c5cccc6c7ccccc7n(c7cccc2c7c43)c56)nc1-c1ccc2c(c1)oc1ccccc12 ; CC1(C)c2ccccc2-c2ccc(-c3nc(-c4ccccc4-n4c5cccc6c7cccc8c9ccccc9n(c9cccc4c9c65)c78)nc(-c4ccccc4)c3C#N)cc21 ; CC1(C)c2ccccc2-c2ccc(-c3nc(-c4ccccc4-n4c5cccc6c7cccc8c9ccccc9n(c9cccc4c9c65)c78)nc(-c4ccc(-c5ccccc5)cc4)c3C#N)cc21 ; N#Cc1c(-c2ccccc2)nc(-c2ccccc2-n2c3cccc4c5cccc6c7ccccc7n(c7cccc2c7c43)c56)nc1-c1ccc2ccccc2c1]

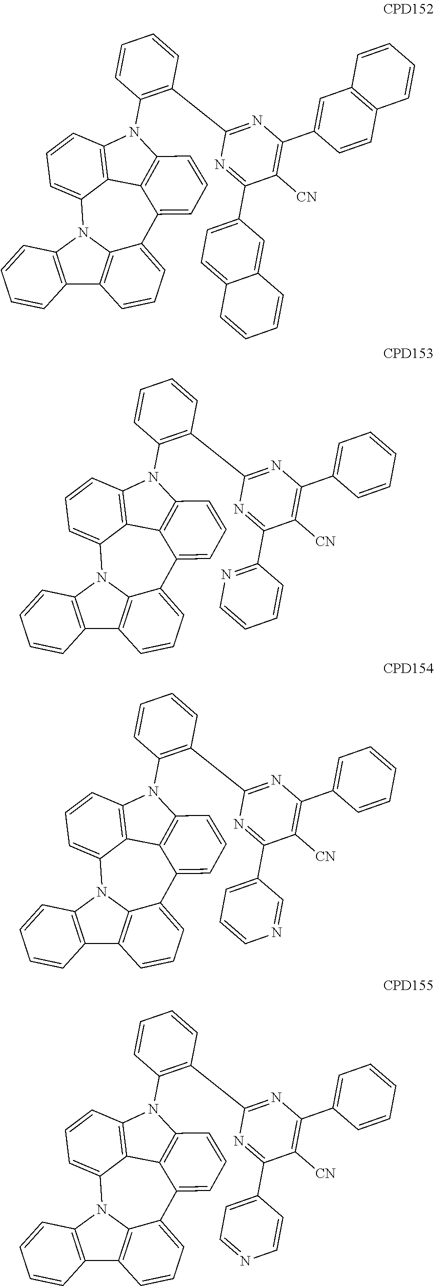 [SMILES: N#Cc1c(-c2ccc3ccccc3c2)nc(-c2ccccc2-n2c3cccc4c5cccc6c7ccccc7n(c7cccc2c7c43)c56)nc1-c1ccc2ccccc2c1 ; N#Cc1c(-c2ccccc2)nc(-c2ccccc2-n2c3cccc4c5cccc6c7ccccc7n(c7cccc2c7c43)c56)nc1-c1ccccn1 ; N#Cc1c(-c2ccccc2)nc(-c2ccccc2-n2c3cccc4c5cccc6c7ccccc7n(c7cccc2c7c43)c56)nc1-c1cccnc1 ; N#Cc1c(-c2ccccc2)nc(-c2ccccc2-n2c3cccc4c5cccc6c7ccccc7n(c7cccc2c7c43)c56)nc1-c1ccncc1]

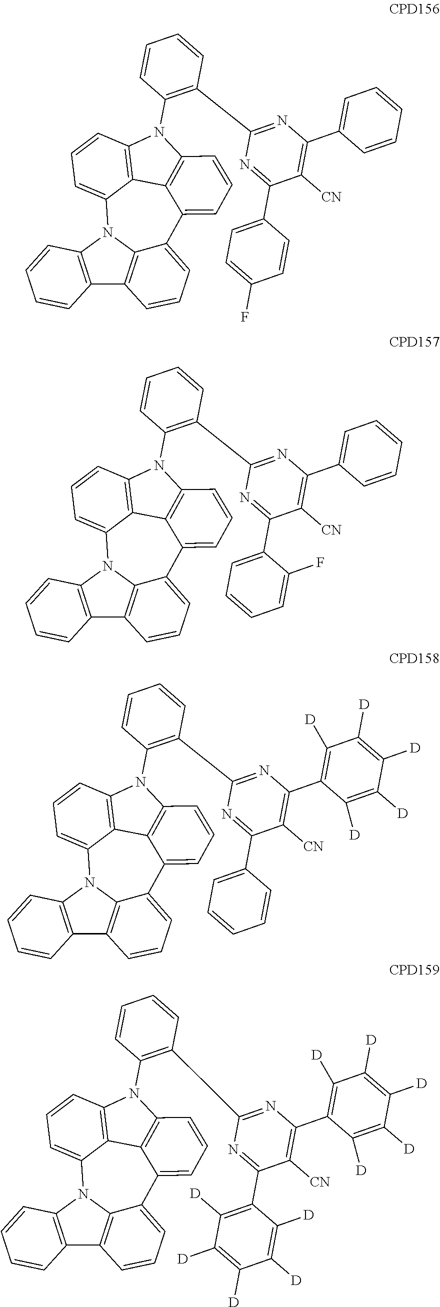

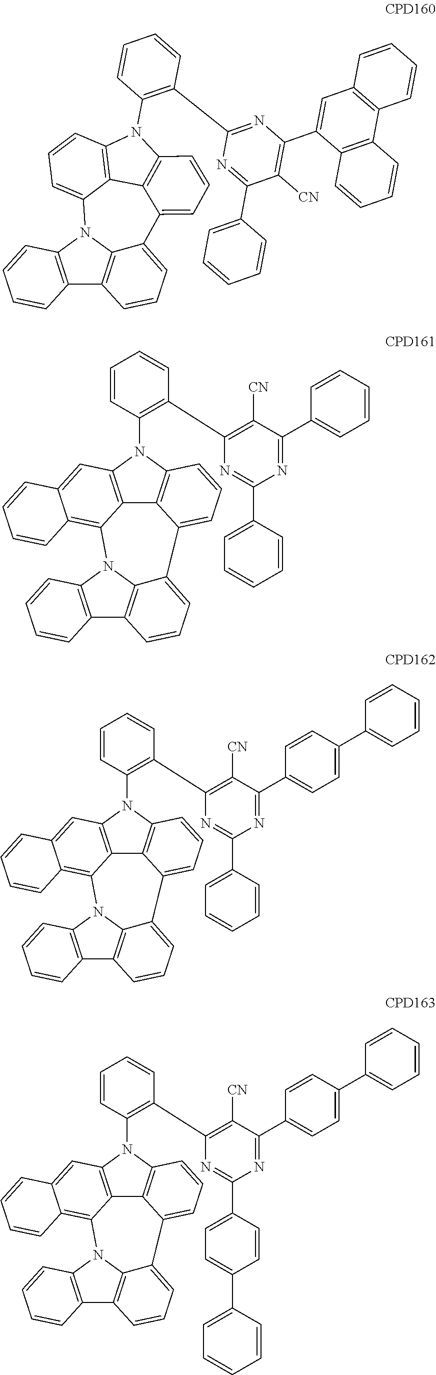

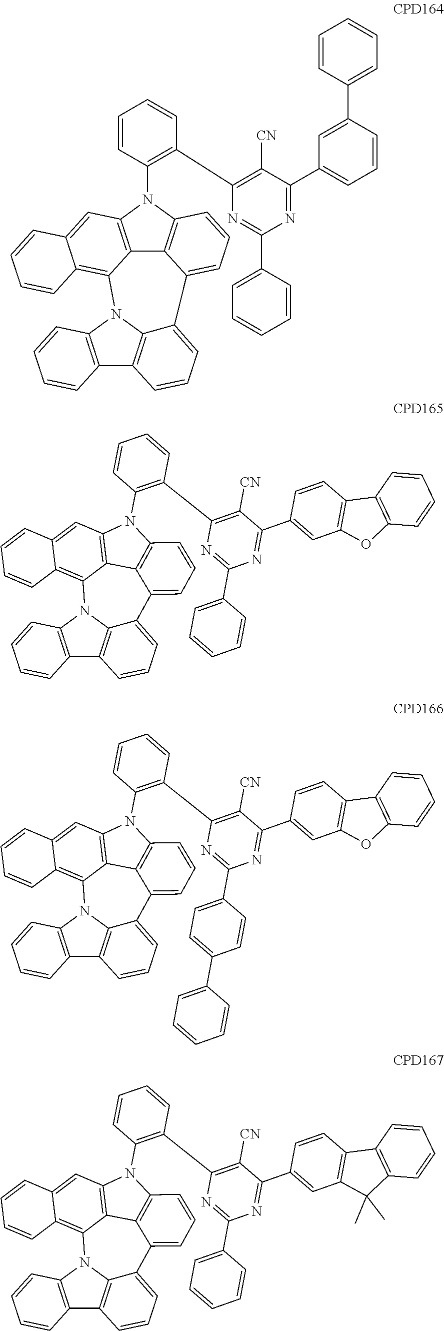 [SMILES: N#Cc1c(-c2cccc(-c3ccccc3)c2)nc(-c2ccccc2)nc1-c1ccccc1-n1c2cccc3c4cccc5c6ccccc6n(c45)c4c5ccccc5cc1c4c32 ; N#Cc1c(-c2ccc3c(c2)oc2ccccc23)nc(-c2ccccc2)nc1-c1ccccc1-n1c2cccc3c4cccc5c6ccccc6n(c45)c4c5ccccc5cc1c4c32 ; N#Cc1c(-c2ccc3c(c2)oc2ccccc23)nc(-c2ccc(-c3ccccc3)cc2)nc1-c1ccccc1-n1c2cccc3c4cccc5c6ccccc6n(c45)c4c5ccccc5cc1c4c32 ; CC1(C)c2ccccc2-c2ccc(-c3nc(-c4ccccc4)nc(-c4ccccc4-n4c5cccc6c7cccc8c9ccccc9n(c78)c7c8ccccc8cc4c7c65)c3C#N)cc21]

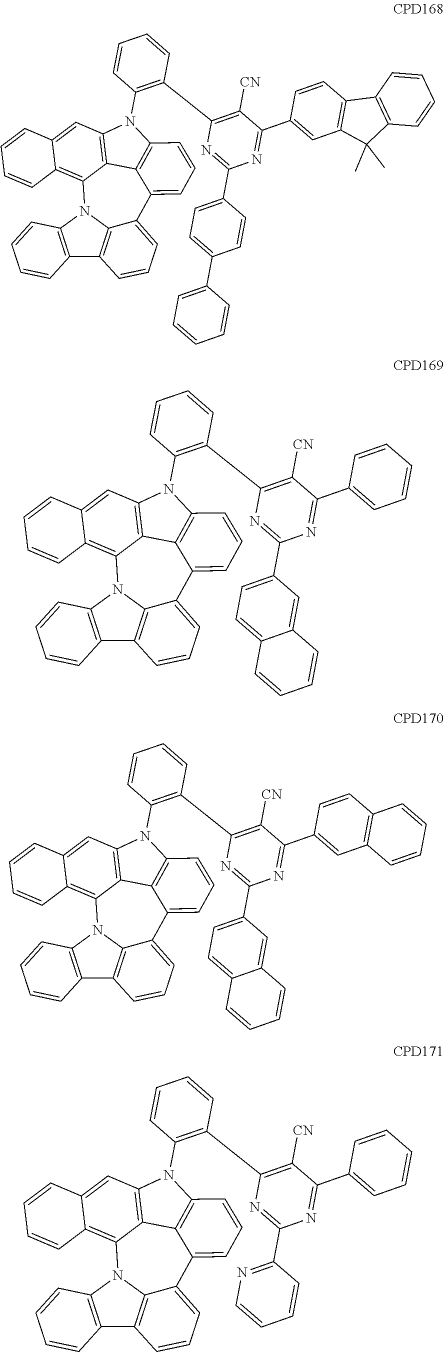 [SMILES: CC1(C)c2ccccc2-c2ccc(-c3nc(-c4ccc(-c5ccccc5)cc4)nc(-c4ccccc4-n4c5cccc6c7cccc8c9ccccc9n(c78)c7c8ccccc8cc4c7c65)c3C#N)cc21 ; N#Cc1c(-c2ccccc2)nc(-c2ccc3ccccc3c2)nc1-c1ccccc1-n1c2cccc3c4cccc5c6ccccc6n(c45)c4c5ccccc5cc1c4c32 ; N#Cc1c(-c2ccc3ccccc3c2)nc(-c2ccc3ccccc3c2)nc1-c1ccccc1-n1c2cccc3c4cccc5c6ccccc6n(c45)c4c5ccccc5cc1c4c32 ; N#Cc1c(-c2ccccc2)nc(-c2ccccn2)nc1-c1ccccc1-n1c2cccc3c4cccc5c6ccccc6n(c45)c4c5ccccc5cc1c4c32]

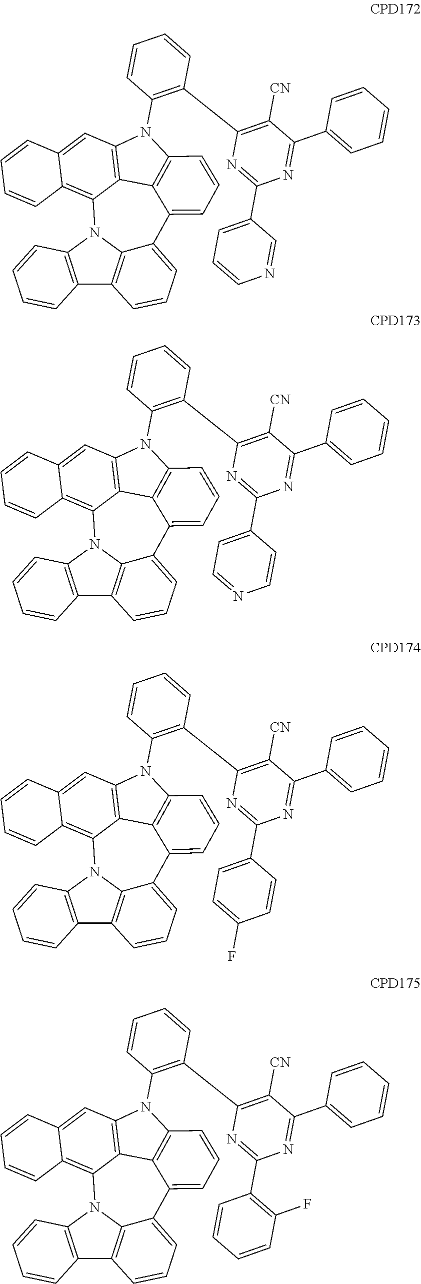

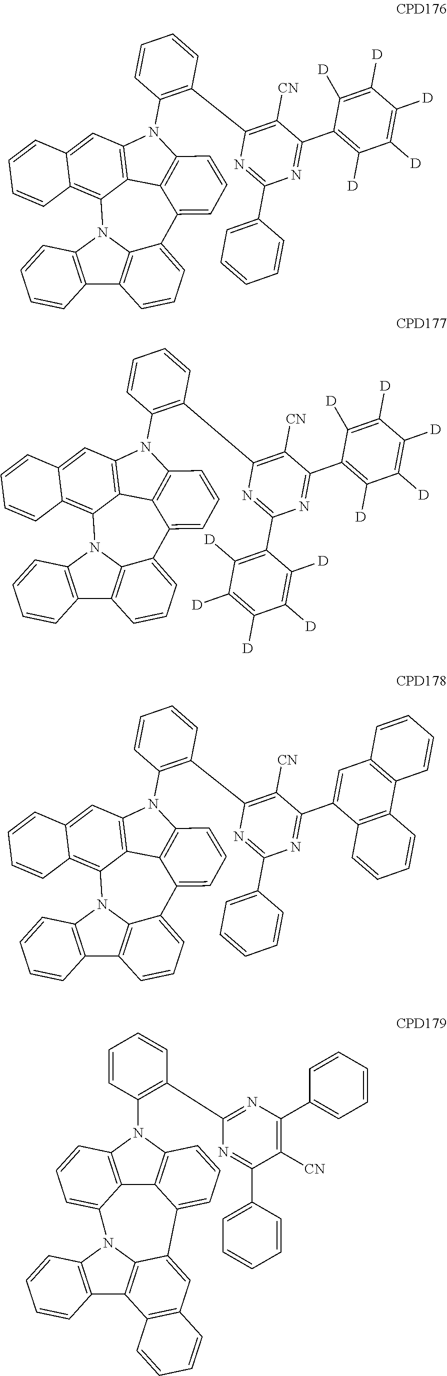

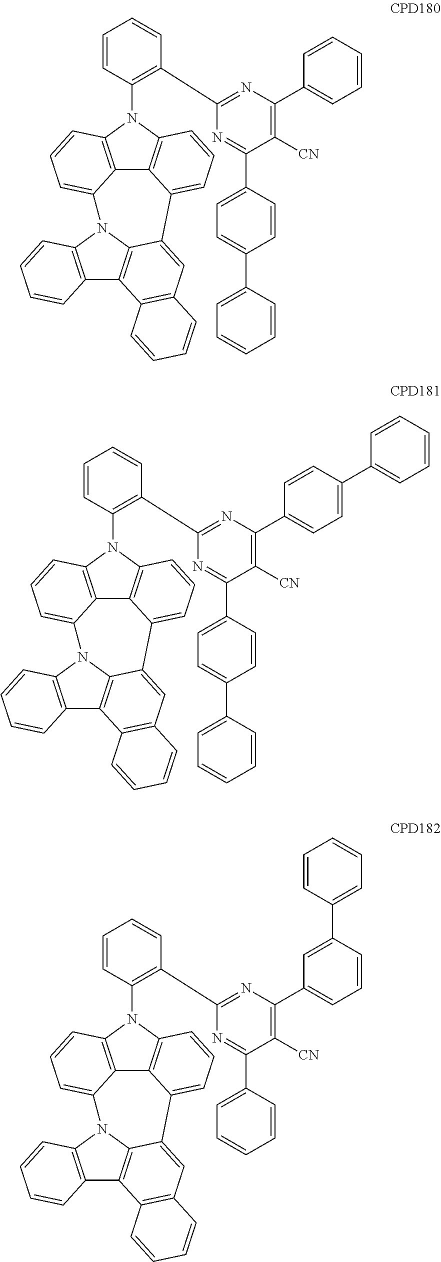 [SMILES: N#Cc1c(-c2ccccc2)nc(-c2ccccc2-n2c3cccc4c5cc6ccccc6c6c7ccccc7n(c7cccc2c7c43)c56)nc1-c1ccc(-c2ccccc2)cc1 ; N#Cc1c(-c2ccc(-c3ccccc3)cc2)nc(-c2ccccc2-n2c3cccc4c5cc6ccccc6c6c7ccccc7n(c7cccc2c7c43)c56)nc1-c1ccc(-c2ccccc2)cc1 ; N#Cc1c(-c2ccccc2)nc(-c2ccccc2-n2c3cccc4c5cc6ccccc6c6c7ccccc7n(c7cccc2c7c43)c56)nc1-c1cccc(-c2ccccc2)c1]

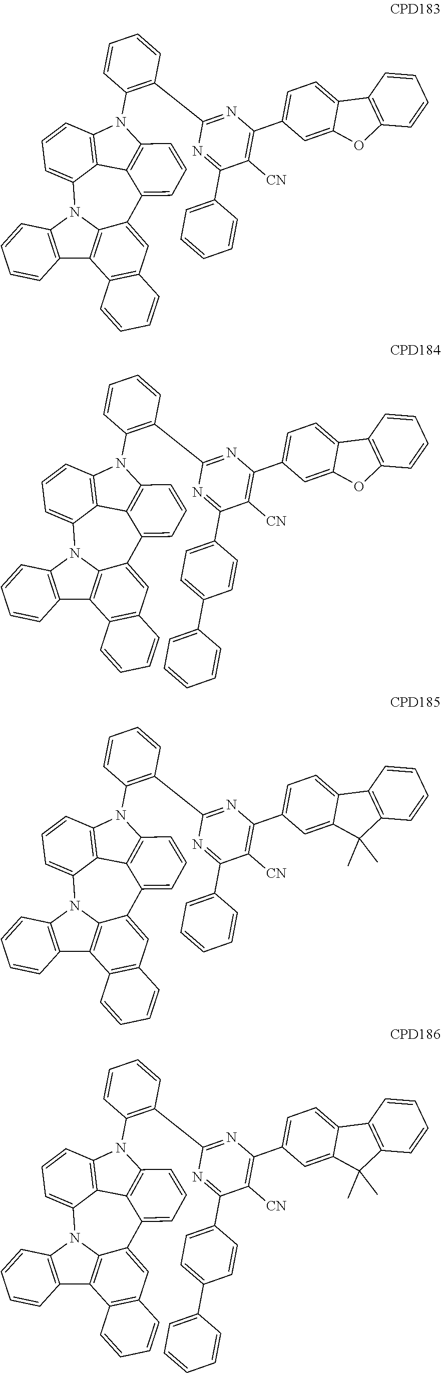

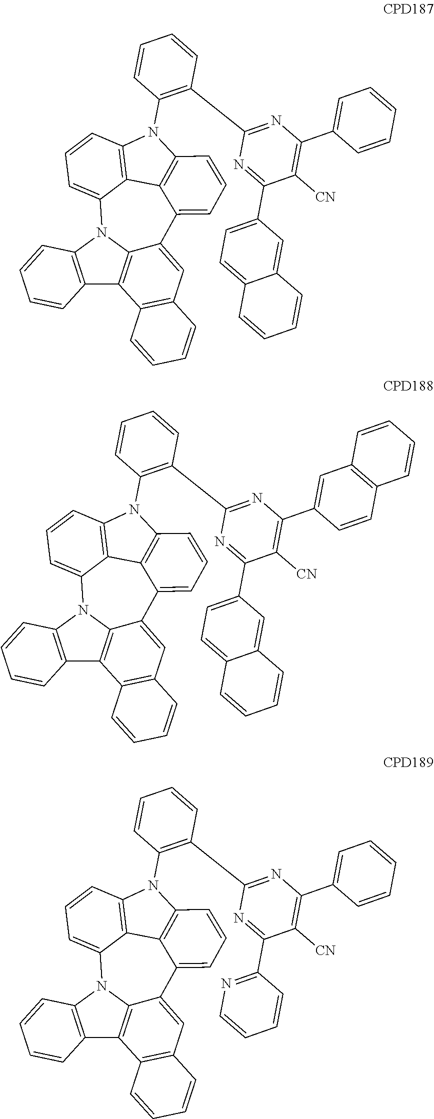

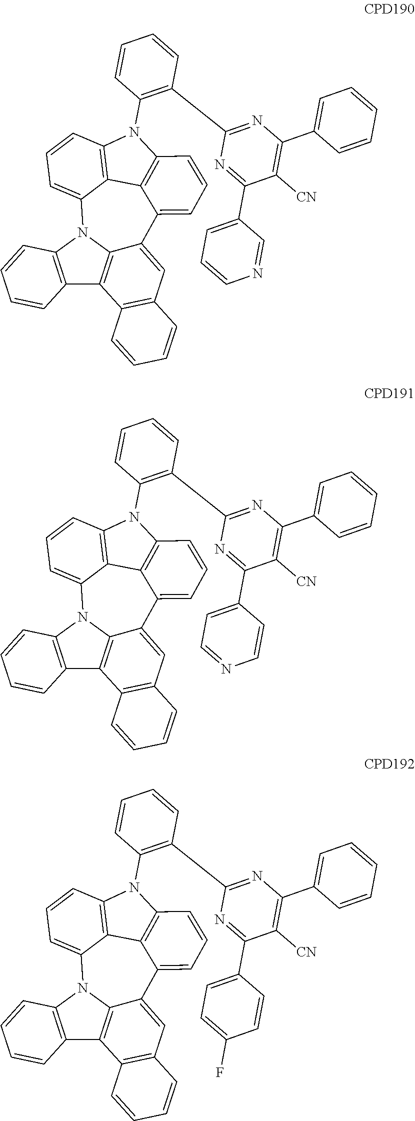

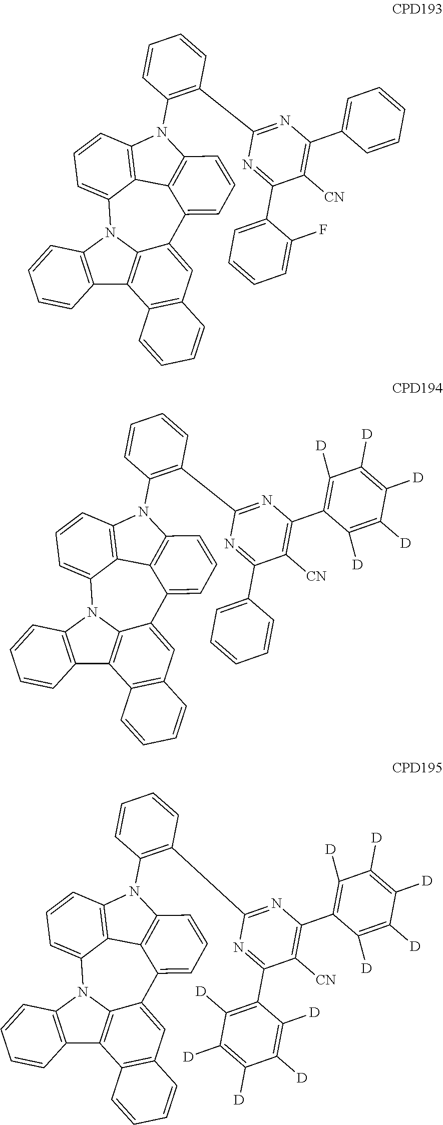 [SMILES: N#Cc1c(-c2ccccc2)nc(-c2ccccc2-n2c3cccc4c5cc6ccccc6c6c7ccccc7n(c7cccc2c7c43)c56)nc1-c1ccccc1F ; [2H]c1c([2H])c([2H])c(-c2nc(-c3ccccc3-n3c4cccc5c6cc7ccccc7c7c8ccccc8n(c8cccc3c8c54)c67)nc(-c3ccccc3)c2C#N)c([2H])c1[2H] ; [2H]c1c([2H])c([2H])c(-c2nc(-c3ccccc3-n3c4cccc5c6cc7ccccc7c7c8ccccc8n(c8cccc3c8c54)c67)nc(-c3c([2H])c([2H])c([2H])c([2H])c3[2H])c2C#N)c([2H])c1[2H]]

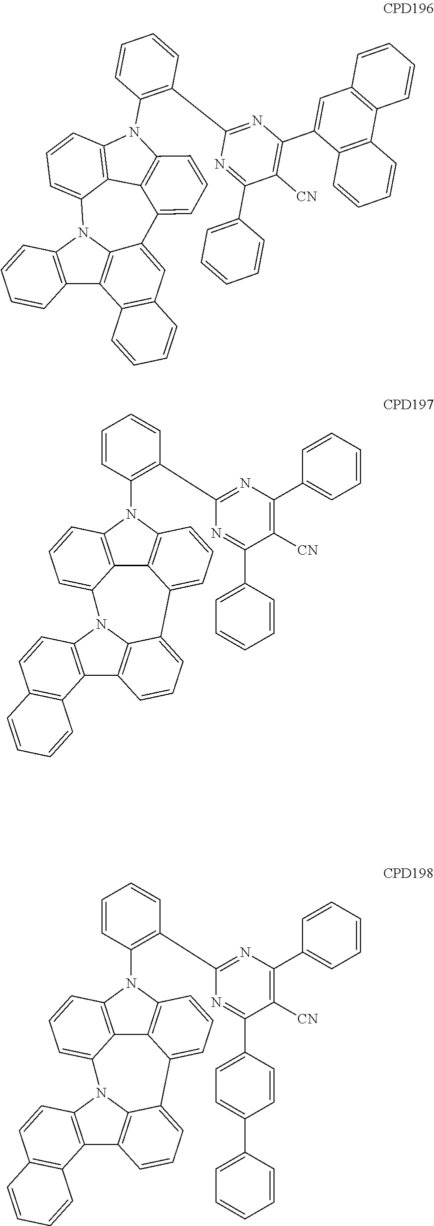 [SMILES: N#Cc1c(-c2ccccc2)nc(-c2ccccc2-n2c3cccc4c5cc6ccccc6c6c7ccccc7n(c7cccc2c7c43)c56)nc1-c1cc2ccccc2c2ccccc12 ; N#Cc1c(-c2ccccc2)nc(-c2ccccc2-n2c3cccc4c5cccc6c7c8ccccc8ccc7n(c7cccc2c7c43)c56)nc1-c1ccccc1 ; N#Cc1c(-c2ccccc2)nc(-c2ccccc2-n2c3cccc4c5cccc6c7c8ccccc8ccc7n(c7cccc2c7c43)c56)nc1-c1ccc(-c2ccccc2)cc1]

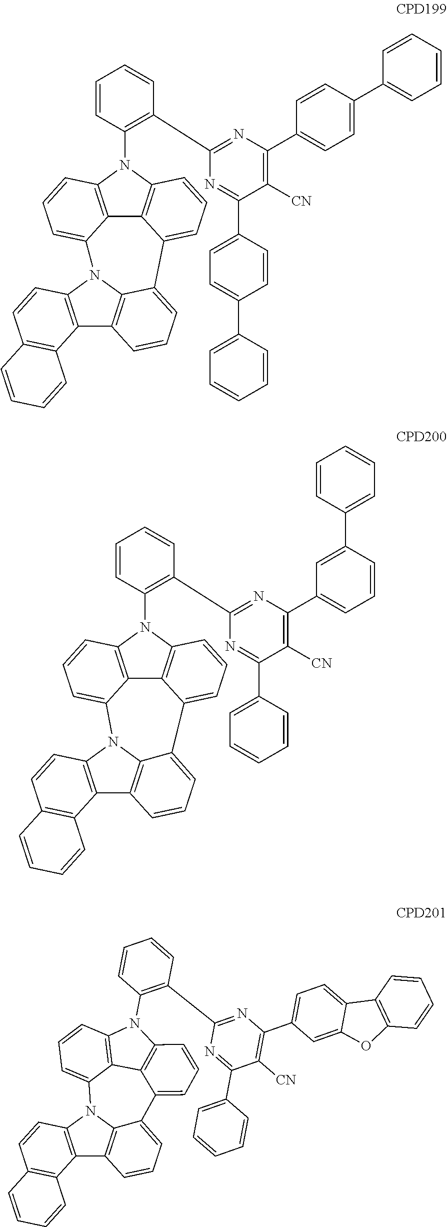 [SMILES: N#Cc1c(-c2ccc(-c3ccccc3)cc2)nc(-c2ccccc2-n2c3cccc4c5cccc6c7c8ccccc8ccc7n(c7cccc2c7c43)c56)nc1-c1ccc(-c2ccccc2)cc1 ; N#Cc1c(-c2ccccc2)nc(-c2ccccc2-n2c3cccc4c5cccc6c7c8ccccc8ccc7n(c7cccc2c7c43)c56)nc1-c1cccc(-c2ccccc2)c1 ; N#Cc1c(-c2ccccc2)nc(-c2ccccc2-n2c3cccc4c5cccc6c7c8ccccc8ccc7n(c7cccc2c7c43)c56)nc1-c1ccc2c(c1)oc1ccccc12]

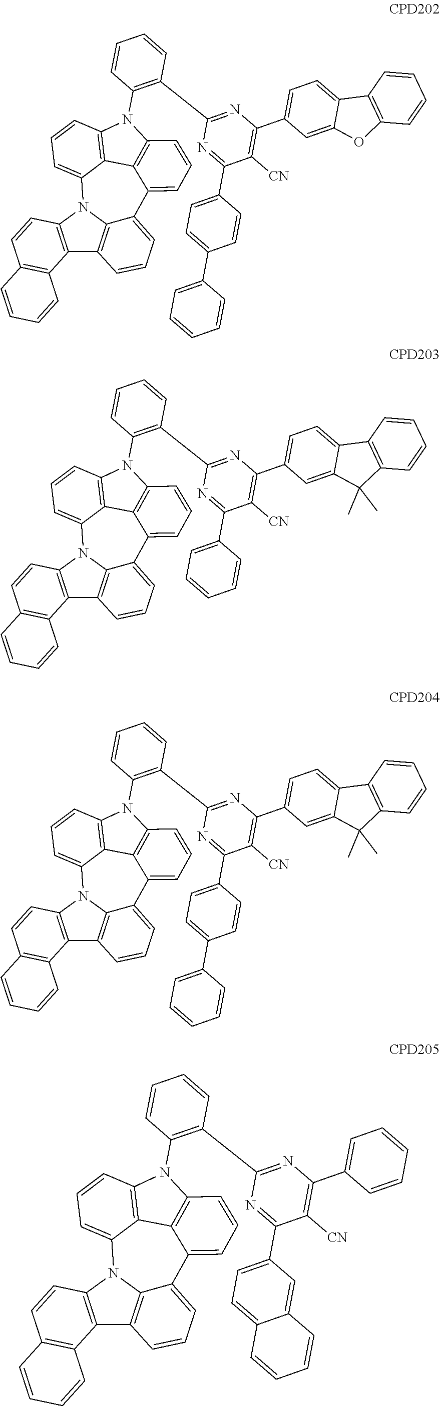 [SMILES: N#Cc1c(-c2ccc(-c3ccccc3)cc2)nc(-c2ccccc2-n2c3cccc4c5cccc6c7c8ccccc8ccc7n(c7cccc2c7c43)c56)nc1-c1ccc2c(c1)oc1ccccc12 ; CC1(C)c2ccccc2-c2ccc(-c3nc(-c4ccccc4-n4c5cccc6c7cccc8c9c%10ccccc%10ccc9n(c9cccc4c9c65)c78)nc(-c4ccccc4)c3C#N)cc21 ; CC1(C)c2ccccc2-c2ccc(-c3nc(-c4ccccc4-n4c5cccc6c7cccc8c9c%10ccccc%10ccc9n(c9cccc4c9c65)c78)nc(-c4ccc(-c5ccccc5)cc4)c3C#N)cc21 ; N#Cc1c(-c2ccccc2)nc(-c2ccccc2-n2c3cccc4c5cccc6c7c8ccccc8ccc7n(c7cccc2c7c43)c56)nc1-c1ccc2ccccc2c1]

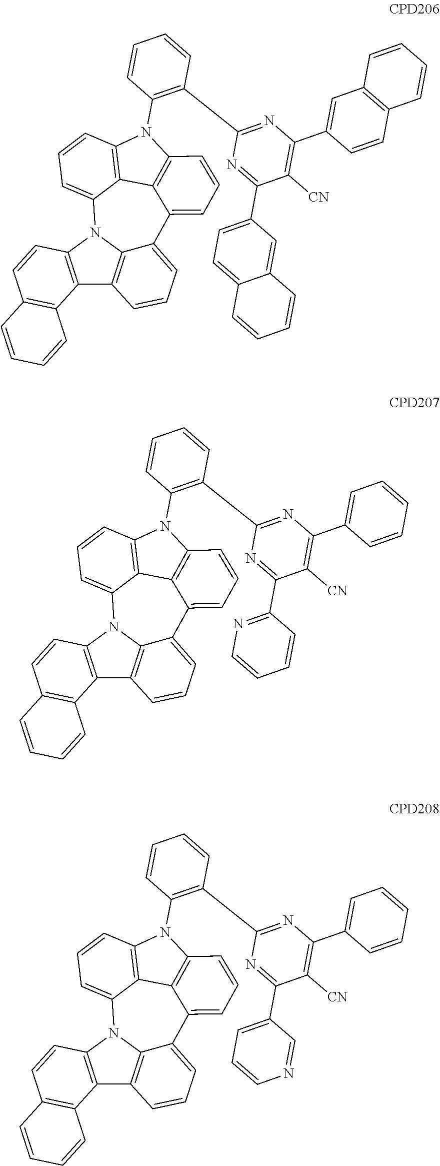 [SMILES: N#Cc1c(-c2ccc3ccccc3c2)nc(-c2ccccc2-n2c3cccc4c5cccc6c7c8ccccc8ccc7n(c7cccc2c7c43)c56)nc1-c1ccc2ccccc2c1 ; N#Cc1c(-c2ccccc2)nc(-c2ccccc2-n2c3cccc4c5cccc6c7c8ccccc8ccc7n(c7cccc2c7c43)c56)nc1-c1ccccn1 ; N#Cc1c(-c2ccccc2)nc(-c2ccccc2-n2c3cccc4c5cccc6c7c8ccccc8ccc7n(c7cccc2c7c43)c56)nc1-c1cccnc1]

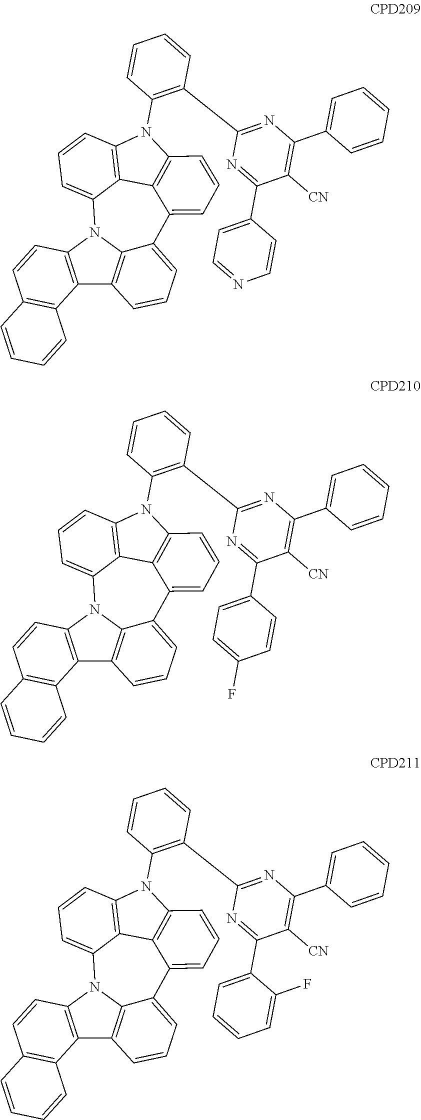 [SMILES: N#Cc1c(-c2ccccc2)nc(-c2ccccc2-n2c3cccc4c5cccc6c7c8ccccc8ccc7n(c7cccc2c7c43)c56)nc1-c1ccncc1 ; N#Cc1c(-c2ccccc2)nc(-c2ccccc2-n2c3cccc4c5cccc6c7c8ccccc8ccc7n(c7cccc2c7c43)c56)nc1-c1ccc(F)cc1 ; N#Cc1c(-c2ccccc2)nc(-c2ccccc2-n2c3cccc4c5cccc6c7c8ccccc8ccc7n(c7cccc2c7c43)c56)nc1-c1ccccc1F]

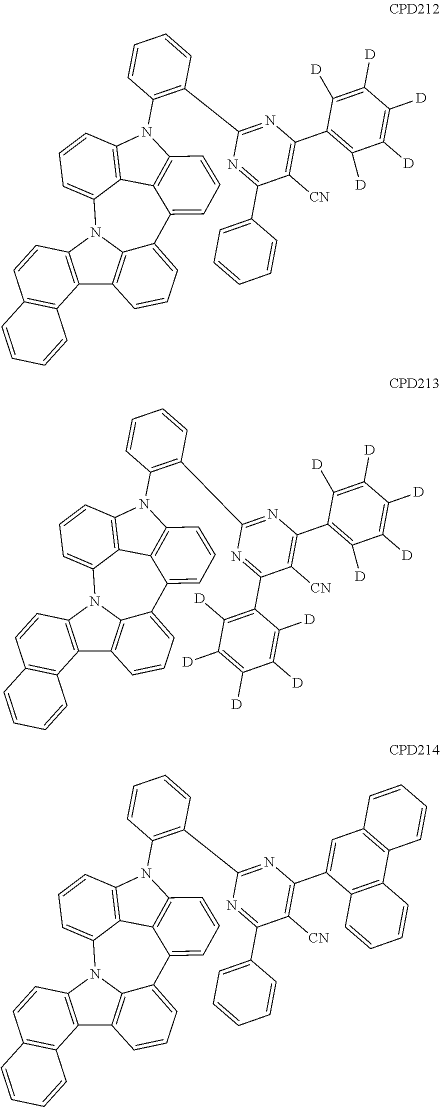 [SMILES: [2H]c1c([2H])c([2H])c(-c2nc(-c3ccccc3-n3c4cccc5c6cccc7c8c9ccccc9ccc8n(c8cccc3c8c54)c67)nc(-c3ccccc3)c2C#N)c([2H])c1[2H] ; [2H]c1c([2H])c([2H])c(-c2nc(-c3ccccc3-n3c4cccc5c6cccc7c8c9ccccc9ccc8n(c8cccc3c8c54)c67)nc(-c3c([2H])c([2H])c([2H])c([2H])c3[2H])c2C#N)c([2H])c1[2H] ; N#Cc1c(-c2ccccc2)nc(-c2ccccc2-n2c3cccc4c5cccc6c7c8ccccc8ccc7n(c7cccc2c7c43)c56)nc1-c1cc2ccccc2c2ccccc12]

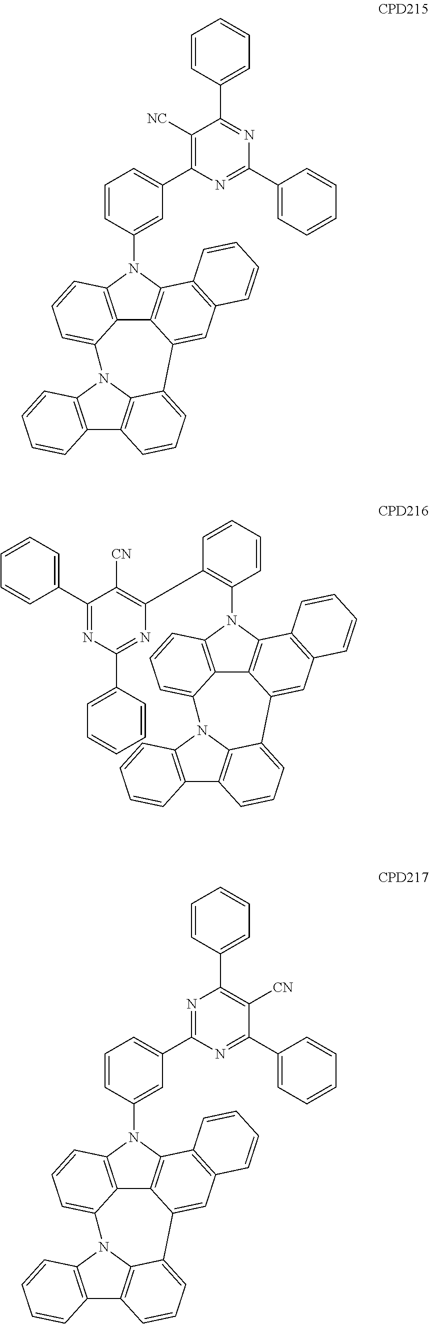 [SMILES: N#Cc1c(-c2ccccc2)nc(-c2ccccc2)nc1-c1cccc(-n2c3cccc4c3c3c(cc5ccccc5c32)c2cccc3c5ccccc5n4c23)c1 ; N#Cc1c(-c2ccccc2)nc(-c2ccccc2)nc1-c1ccccc1-n1c2cccc3c2c2c(cc4ccccc4c21)c1cccc2c4ccccc4n3c12 ; N#Cc1c(-c2ccccc2)nc(-c2cccc(-n3c4cccc5c4c4c(cc6ccccc6c43)c3cccc4c6ccccc6n5c34)c2)nc1-c1ccccc1]

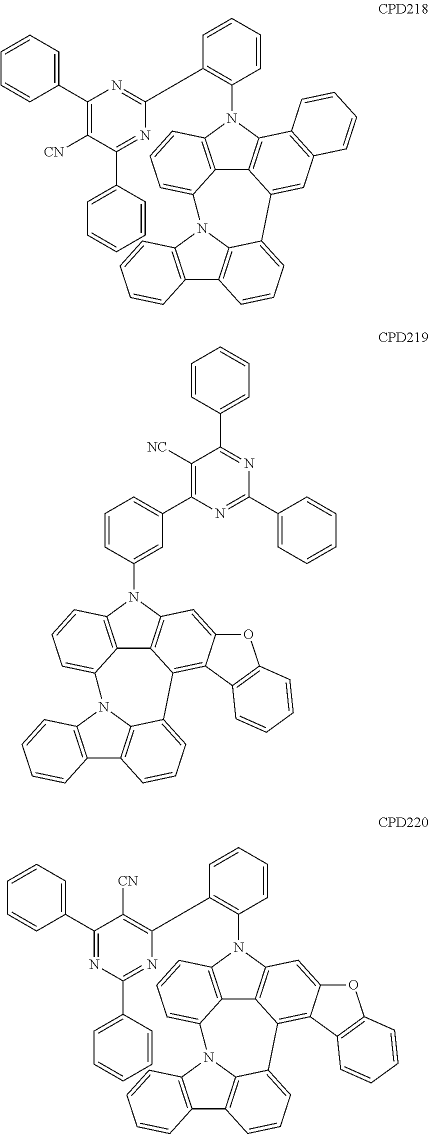

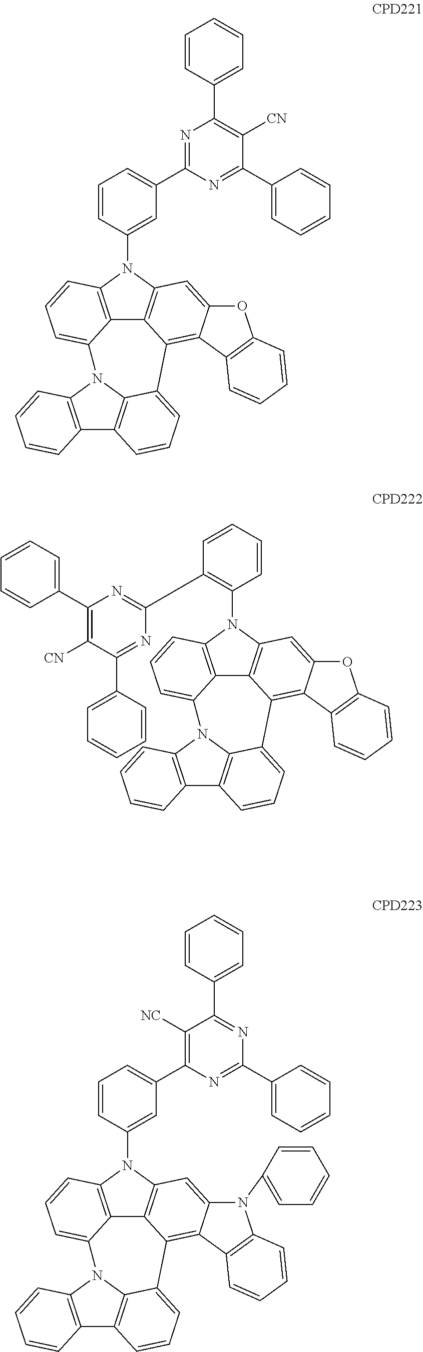

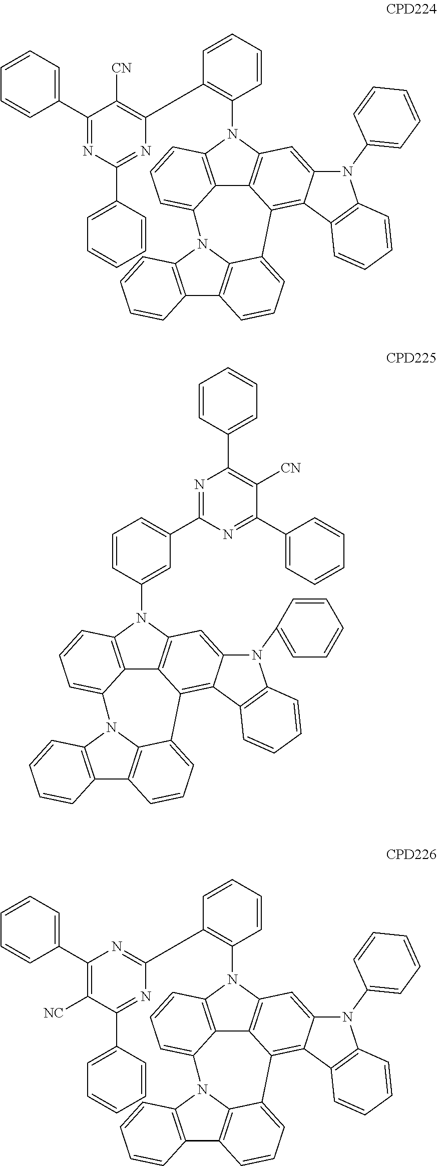 [SMILES: N#Cc1c(-c2ccccc2)nc(-c2ccccc2)nc1-c1ccccc1-n1c2cc3c(c4ccccc4n3-c3ccccc3)c3c4cccc5c6ccccc6n(c6cccc1c6c32)c54 ; N#Cc1c(-c2ccccc2)nc(-c2cccc(-n3c4cc5c(c6ccccc6n5-c5ccccc5)c5c6cccc7c8ccccc8n(c8cccc3c8c54)c76)c2)nc1-c1ccccc1 ; N#Cc1c(-c2ccccc2)nc(-c2ccccc2-n2c3cc4c(c5ccccc5n4-c4ccccc4)c4c5cccc6c7ccccc7n(c7cccc2c7c43)c65)nc1-c1ccccc1]

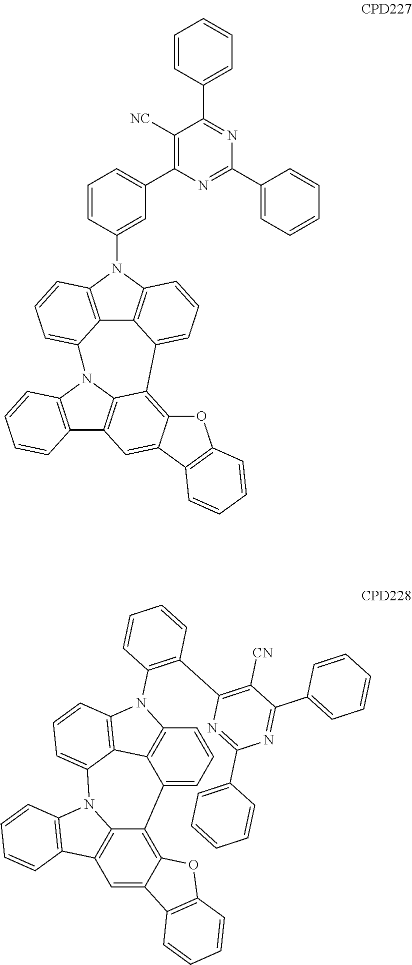 [SMILES: N#Cc1c(-c2ccccc2)nc(-c2ccccc2)nc1-c1cccc(-n2c3cccc4c3c3c2cccc3n2c3ccccc3c3cc5c6ccccc6oc5c4c32)c1 ; N#Cc1c(-c2ccccc2)nc(-c2ccccc2)nc1-c1ccccc1-n1c2cccc3c2c2c1cccc2n1c2ccccc2c2cc4c5ccccc5oc4c3c21]

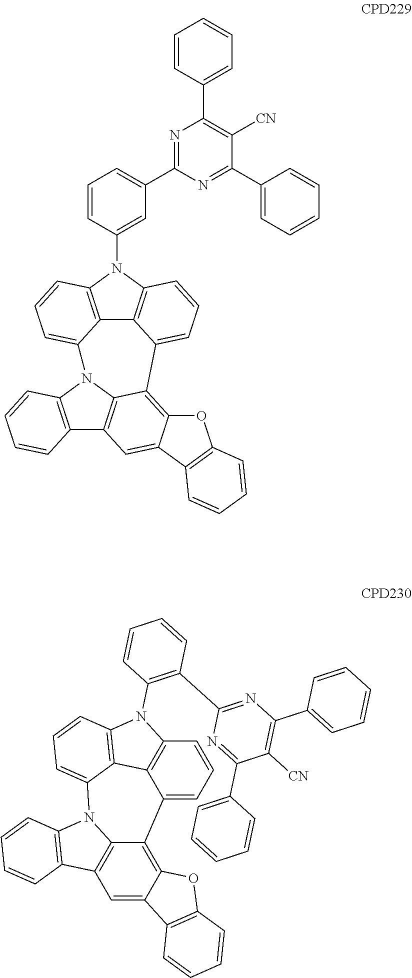 [SMILES: N#Cc1c(-c2ccccc2)nc(-c2cccc(-n3c4cccc5c4c4c3cccc4n3c4ccccc4c4cc6c7ccccc7oc6c5c43)c2)nc1-c1ccccc1 ; N#Cc1c(-c2ccccc2)nc(-c2ccccc2-n2c3cccc4c3c3c2cccc3n2c3ccccc3c3cc5c6ccccc6oc5c4c32)nc1-c1ccccc1]

An organic electroluminescent device comprises the above organic electroluminescent material.

The organic electroluminescent device comprises a light emitting layer, wherein the light emitting layer comprises the organic electroluminescent material.

The material in the present disclosure has the advantages of high optical and electrical stability, low sublimation temperature, low driving voltage, high light emitting efficiency, and long device service life, can be used as a host material in an OLED device, further has a low melting point, and is conductive to material evaporation stability as a molten material. As a host material, this material has the possibility of being applied to AMOLED industry.

### BRIEF DESCRIPTION OF DRAWINGS

FIG. 1 is ¹HNMR spectrum of compound CPD125; and

FIG. 2 is ¹HNMR spectrum of compound CPD143.

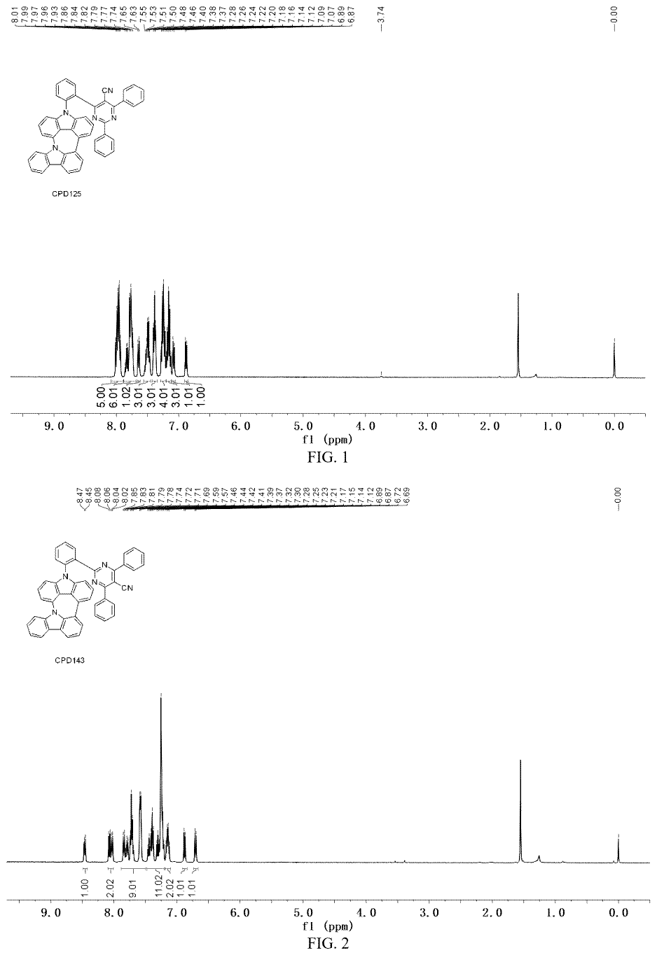

### DETAILED DESCRIPTION

A compound in the present disclosure is an organic electroluminescent material having a structure of formula (1),

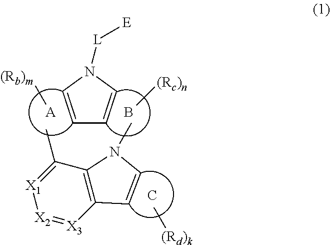

wherein L is selected from any one of a single bond, a substituted or unsubstituted C₆-C₃₀ aryl, or a substituted or unsubstituted C₃-C₂₀ heteroaryl;
X₁ to X₃ each independently represent N or CRₐ, ring A, ring B, and ring C are, identically or differently during each occurrence, selected from a carbocyclic ring with 5-18 carbon atoms or a heterocyclic ring with 3-18 carbon atoms;
wherein E has a structure represented by formula (2):

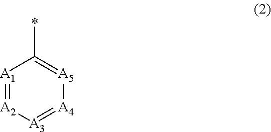

wherein A₁ to A₅ are selected identically or differently from the group consisting of N and CR₁; at least one of A₁ to A₅ is N, at least one thereof is C—CN; and * represents a linking site with formula (1);
wherein Rₐ, R_{b}, R_{c}, R_{d}, and R₁ are, identically or differently during each occurrence, selected from the group consisting of hydrogen, deuterium, halogen, a substituted or unsubstituted alkyl with 1-10 carbon atoms, a substituted or unsubstituted cycloalkyl with 3-20 ring carbon atoms, a substituted or unsubstituted heteroalkyl with 1-10 carbon atoms, a substituted or unsubstituted heterocyclyl with 3-20 ring atoms, a substituted or unsubstituted arylalkyl with 7-30 carbon atoms, a substituted or unsubstituted alkoxy with 1-20 carbon atoms, a substituted or unsubstituted aryloxy with 6-30 carbon atoms, a substituted or unsubstituted alkenyl with 2-10 carbon atoms, a substituted or unsubstituted aryl with 6-30 carbon atoms, a substituted or unsubstituted heteroaryl with 3-30 carbon atoms, a substituted or unsubstituted alkylsilyl with 3-20 carbon atoms, a substituted or unsubstituted arylsilyl with 6-20 carbon atoms, a substituted or unsubstituted amino with 0-20 carbon atoms, a substituted or unsubstituted acyl with 0-20 carbon atoms, carbonyl, carboxyl, an ester group, cyano, isocyano, hydroxyl, sulfydryl, a substituted or unsubstituted sulfinyl with 0-20 carbon atoms, a substituted or unsubstituted sulfonyl with 0-20 carbon atoms, phosphino, or a combination thereof, adjacent Rₐ, adjacent R_{b}, adjacent R_{c}, and adjacent R_{d} can be optionally linked to form a ring; and
the substitution refers to a substitution with deuterium, halogen, cyano, an alkyl with 1-6 carbon atoms, or an aryl with 6-30 carbon atoms,
wherein m, n, and k are each independently selected from an integer of 1-4; and
heteroatoms in the heteroaromatic ring, the heteroalkyl, the heterocyclyl, and the heteroaryl are each independently selected from O, S, N, Si, Ge, or P.

Examples of each group of the compound represented by formula (1) will be described below.

It should be noted that in this specification, “carbon number a-b” in the expression “substituted or unsubstituted X group with a carbon number a-b” means a carbon number in an unsubstituted X group, and does not include a carbon number of a substituent on a substituted X group.

The C₁-C₁₀ alkyl is a linear or branched alkyl, and is specifically, for example, methyl, ethyl, propyl, isopropyl, n-butyl, isobutyl, sec-butyl, tert-butyl, n-pentyl and an isomer thereof, n-hexyl and an isomer thereof, n-heptyl and an isomer thereof, n-octyl and an isomer thereof, n-nonyl and an isomer thereof, n-decyl and an isomer thereof, preferably methyl, ethyl, propyl, isopropyl, n-butyl, isobutyl, sec-butyl, tert-butyl, and more preferably propyl, isopropyl, isobutyl, sec-butyl, and tert-butyl.

Examples of the C₃-C₂₀ cycloalkyl may include, for example, cyclopropyl, cyclobutyl, cyclopentyl, cyclohexyl, 1-adamantyl, 2-adamantyl, 1-norbornyl, and 2-norbornyl, preferably cyclopentyl and cyclohexyl.

Examples of the C₂-C₁₀ alkenyl may include, for example, vinyl, propenyl, allyl, 1-butadienyl, 2-butadienyl, 1-hexatrienyl, 2-hexatrienyl, and 3-hexatrienyl, and are preferably propenyl and allyl.

The C₁-C₁₀ heteroalkyl is, for example, a linear or branched alkyl or cycloalkyl containing an atom other than carbon and hydrogen, and examples thereof may include, for example, thiomethylmethyl, methoxymethyl, ethoxymethyl, tert-butoxymethyl, N,N-dimethylmethyl, epoxybutyl, epoxypentyl, and epoxyhexyl, preferably methoxymethyl and epoxypentyl.

Specific examples of the aryl include, for example, phenyl, naphthyl, anthryl, phenanthryl, tetraphenyl, pyrenyl, chrysenyl, benzo[c]phenanthryl, benzo[g]chrysenyl, fluorenyl, benzofluorenyl, dibenzofluorenyl, biphenyl, terphenyl, tetraphenyl, and fluoranthenyl, preferably phenyl and naphthyl.

Specific examples of the heteroaryl may include, for example, pyrrolyl, pyrazinyl, pyridyl, pyrimidinyl, triazinyl, indolyl, isoindolyl, imidazolyl, furyl, benzofuryl, isobenzofuryl, dibenzofuryl, dibenzothiophenyl, azadibenzofuryl, azadibenzothiophenyl, diazadibenzofuryl, diazadibenzothiophenyl, quinolyl, isoquinolyl, quinoxalinyl, carbazolyl, phenanthridinyl, acridinyl, phenanthrolinyl, phenazinyl, phenothiazinyl, phenoxazinyl, oxazolinyl, oxadiazolyl, furazanyl, thienyl, benzothienyl, dihydroacridinyl, azacarbazolyl, diazacarbazolyl, and quinazolinyl, preferably pyridyl, pyrimidinyl, triazinyl, dibenzofuryl, dibenzothienyl, azadibenzofuryl, azadibenzothienyl, diazadibenzofuryl, diazadibenzothienyl, carbazolyl, azacarbazolyl, and diazacarbazolyl.

Adjacent substituents Rₐ, R_{b}, R_{c}, and R_{d} can optionally be linked to form a ring, which means that when adjacent ring atoms on rings A, B, or C are each linked to the group R_{b}, R_{c}, or R_{d} respectively, the group R_{b}, R_{c}, or R_{d} on the adjacent ring atoms can be linked to form a ring. When the adjacent ring atoms on the ring formed by X₁, X₂, and X₃ are linked to the group Rₐ, the group Rₐ on the adjacent ring atoms can be linked to form a ring.

The embodiments below are only for the convenience of understanding the technical invention, and should not be regarded as specific limitations to the present disclosure.

The starting materials and solvents involved in the synthesis of the compound in the present disclosure are all purchased from suppliers well-known to those skilled in the art, such as Alfa and Acros.

### Synthesis of Compound CPD001

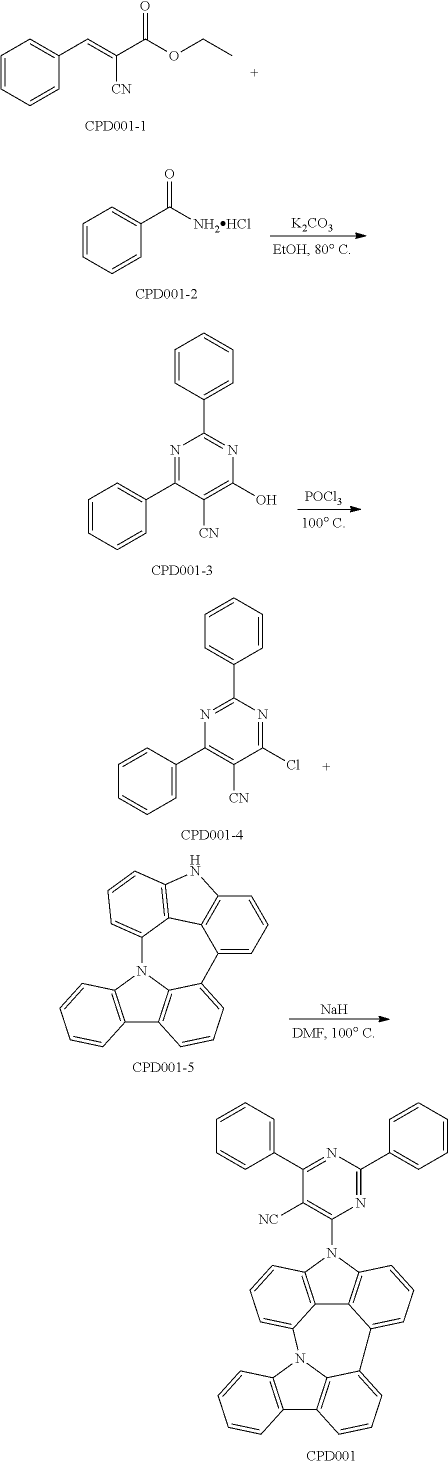

### Synthesis of Compound CPD001-3

Compounds CPD001-1 (40.00 g, 198.78 mmol), CPD001-2 (37.36 g, 238.54 mmol), potassium carbonate (54.95 g, 397.56 mmol), and ethanol (600 mL) were added into a 1,000 mL three-necked round bottomed flask, and after nitrogen replacement under vacuum three times, the mixture was heated to a temperature of 80° C. for reaction overnight. The complete consumption of the starting material CPD001-1 was monitored by TLC (ethyl acetate:n-hexane=1:5 as a developing solvent). After cooled to room temperature, the mixture was concentrated under reduced pressure at 65° C. to remove ethanol. 500 mL of deionized water was added and the mixture was slurried in an oil at 80° C. for 1 h. After suction filtration, the same operations were repeated. The mixture was vacuum dried at 100° C. overnight to provide the compound CPD001-3 as a white solid (46.21 g, purity: 99.41%, yield: 85.06%, mass spectrum: 274.10 (M+H)).

### Synthesis of Compound CPD001-4

The compound CPD001-3 (45.00 g, 164.66 mmol) and phosphorus oxychloride (150 mL, 397.56 mmol) were added into a 500 mL three-necked round bottomed flask, and the mixture was heated to a temperature of 100° C. for reaction for 4 h. The complete consumption of the starting material CPD001-3 was monitored by TLC (ethyl acetate:n-hexane=1:5 as a developing solvent). After cooled to room temperature, the mixture was concentrated under reduced pressure at 70° C. to remove phosphorus oxychloride. Dichloromethane (500 mL) was added to dissolve the materials, and then the above dichloromethane solution was slowly added dropwise to 1,000 mL of deionized water. The mixture was stirred at room temperature for 1 h, and left to stand for liquid separation. The organic phase was washed with deionized water (300 mL*3); purified by silica gel column chromatography (300 g, 200-300 mesh, ethyl acetate:n-hexane=1:10 as an eluent), eluted, and then concentrated under reduced pressure at 70° C. for 2 h to provide the compound CPD001-4 as a white solid (39.60 g, purity: 99.56%, yield: 82.44%, mass spectrum: 292.64 (M+H)).

### Synthesis of Compound CPD001

Compound CPD001-5 (20.38 g, 61.70 mmol) and N,N-dimethylformamide (300 mL) were added into a 500 mL three-necked round bottomed flask, and after nitrogen replacement under vacuum three times, sodium hydride (2.47 g, 61.70 mmol) with a mass fraction of 60% was slowly added while stirring at room temperature. After the mixture was stirred at room temperature for 30 min, the CPD001-4 (15.00 g, 51.42 mmol) was slowly added, and finally the mixture was heated to 100° C. for reaction overnight. The complete consumption of the starting material CPD001-4 was monitored by TLC (ethyl acetate:n-hexane=1:10 as a developing solvent). The reaction mixture was cooled to room temperature and slowly added dropwise to 1,000 mL of deionized water. The mixture was stirred at room temperature for 1.5 h, so that a large amount of yellow solid precipitated. After suction filtration, the filter cake was washed with 300 mL of deionized water, vacuum dried at 80° C. for 2 h, and crystallized twice with toluene and methanol, to provide the compound CPD001 as a yellow solid (15.06 g, purity: 99.95%, yield: 50.00%). 15.06 g of the crude CPD001 was purified by sublimation to provide sublimation-purified CPD001 (12.51 g, purity: 99.95%, yield: 83.07%, mass spectrum: 586.24 (M+H)). ¹H NMR (400 MHz, CDCl₃) δ 8.25-8.14 (m, 4H), 7.89-7.79 (m, 2H), 7.72 (dd, J=15.0, 2.9 Hz, 1H), 7.59-7.42 (m, 8H), 7.42-7.36 (m, 2H), 7.21-7.15 (m, 3H), 7.10-7.05 (m, 2H), 7.04-6.97 (m, 1H).

### Synthesis of Compound CPD009

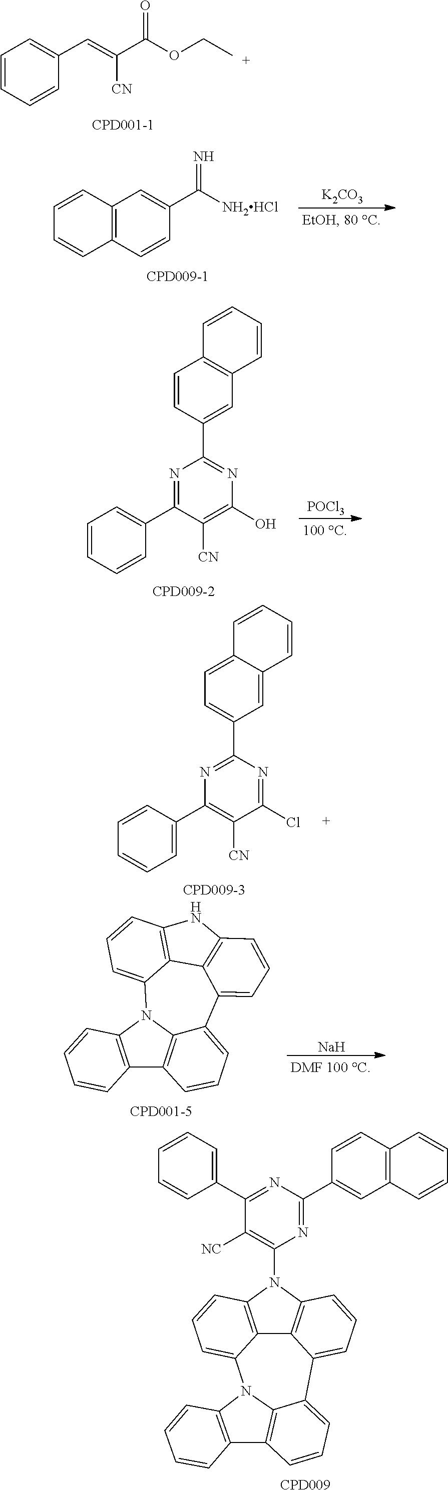

### Synthesis of Compound CPD009-2

With reference to the synthesis and purification methods for the compound CPD001-3, only the corresponding starting materials were replaced, to provide the target compound CPD009-2 as a white solid (32.55 g, purity: 99.31%, yield: 81.01%, mass spectrum: 324.26 (M+H)).

### Synthesis of Compound CPD009-3

With reference to the synthesis and purification methods for the compound CPD001-4, only the corresponding starting materials were replaced, to provide the target compound CPD009-3 as a white solid (27.63 g, purity: 99.59%, yield: 83.33%, mass spectrum: 342.07 (M+H)).

### Synthesis of Compound CPD009

With reference to the synthesis and purification methods for the compound CPD001, only the corresponding starting materials were replaced, to provide the target compound CPD009 as a yellow solid (14.25 g, purity: 99.94%, yield: 51.66%). 14.25 g of the crude CPD009 was purified by sublimation to provide sublimation-purified CPD009 (11.29 g, purity: 99.94%, yield: 79.23%, mass spectrum: 636.22 (M+H)). ¹H NMR (400 MHz, CDCl₃) δ 8.65 (s, 1H), 8.49 (dd, J=15.0, 3.0 Hz, 1H), 8.21-8.14 (m, 3H), 8.10-8.06 (m, 1H), 8.02-7.98 (m, 1H), 7.86-7.82 (m, 2H), 7.74-7.70 (m, 1H), 7.68-7.47 (m, 6H), 7.45-7.40 (m, 3H), 7.21-7.13 (m, 3H), 7.13-6.97 (m, 3H).

### Synthesis of Compound CPD019

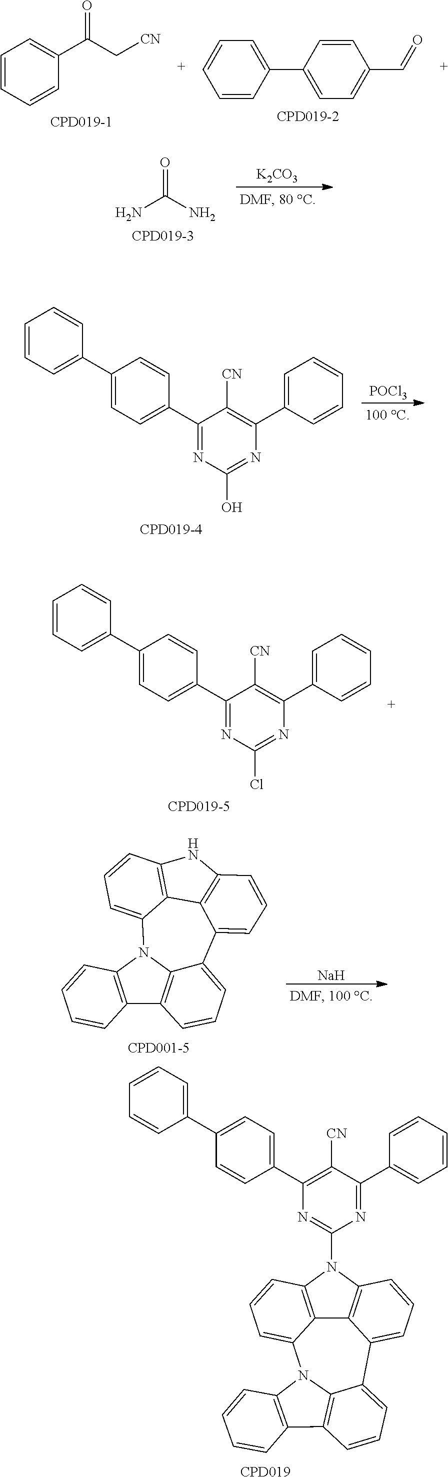

### Synthesis of Compound CPD019-4

Compounds CPD019-1 (30.00 g, 206.67 mmol), CPD019-2 (37.66 g, 206.67 mmol), CPD019-3 (37.23 g, 620.00 mmol), potassium carbonate (85.69 g, 620.00 mmol), and N,N-dimethylformamide (500 mL) were added into a 1,000 mL three-necked round bottomed flask, and after nitrogen replacement under vacuum three times, the mixture was heated to a temperature of 80° C. for reaction overnight. The complete consumption of the starting materials CPD019-1 and CPD019-2 was monitored by TLC (ethyl acetate:n-hexane=1:5 as a developing solvent). After cooled to room temperature, the mixture was concentrated under reduced pressure at 65° C. to remove N,N-dimethylformamide. 500 mL of deionized water was added and the mixture was slurried in an oil at 70° C. for 1 h. After suction filtration, the filter cake was washed with 500 mL of deionized water, washed with 100 mL of ethanol, and vacuum dried at 100° C. overnight to provide the compound CPD019-4 as a white solid (44.87 g, purity: 99.21%, yield: 62.14%, mass spectrum: 350.12 (M+H)).

### Synthesis of Compound CPD019-5

With reference to the synthesis and purification methods for the compound CPD001-4, only the corresponding starting materials were replaced, to provide the target compound CPD019-5 as a white solid (30.06 g, purity: 99.42%, yield: 80.09%, mass spectrum: 368.12 (M+H)).

### Synthesis of Compound CPD019

With reference to the synthesis and purification methods for the compound CPD001, only the corresponding starting materials were replaced, to provide the target compound CPD019 as a yellow solid (17.51 g, purity: 99.93%, yield: 53.85%). 17.51 g of the crude CPD019 was purified by sublimation to provide sublimation-purified CPD019 (14.20 g, purity: 99.93%, yield: 81.10%, mass spectrum: 662.24 (M+H)). ¹H NMR (400 MHz, CDCl₃) δ 8.34-8.26 (m, 2H), 8.22-8.15 (m, 2H), 7.90-7.79 (m, 4H), 7.79-7.67 (m, 3H), 7.61-7.34 (m, 10H), 7.26-7.11 (m, 3H), 7.11-6.95 (m, 3H).

### Synthesis of Compound CPD033

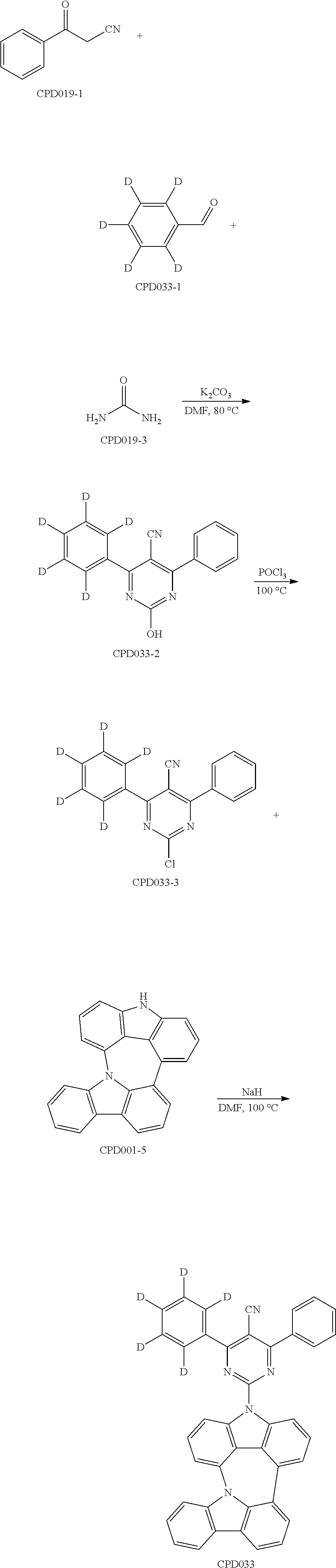

### Synthesis of Compound CPD033-2

With reference to the synthesis and purification methods for the compound CPD019-4, only the corresponding starting materials were replaced, to provide the target compound CPD033-2 as a white solid (18.88 g, purity: 99.32%, yield: 65.33%, mass spectrum: 279.22 (M+H)).

### Synthesis of Compound CPD033-3

With reference to the synthesis and purification methods for the compound CPD001-4, only the corresponding starting materials were replaced, to provide the target compound CPD033-3 as a white solid (15.23 g, purity: 99.55%, yield: 86.78%, mass spectrum: 297.08 (M+H)).

### Synthesis of Compound CPD033

With reference to the synthesis and purification methods for the compound CPD001, only the corresponding starting materials were replaced, to provide the target compound as a yellow solid CPD033 (11.44 g, purity: 99.95%, yield: 56.12%). 11.44 g of the crude CPD033 was purified by sublimation to provide sublimation-purified CPD033 (8.68 g, purity: 99.95%, yield: 75.88%, mass spectrum: 591.24 (M+H)). ¹H NMR (400 MHz, CDCl₃) δ 8.19 (dd, J=7.4, 1.5 Hz, 2H), 7.85-7.83 (m, 2H), 7.72 (dd, J=7.5, 1.4 Hz, 1H), 7.60-0.47 (m, 4H), 7.47-7.42 (m, 1H), 7.40 (dd, J=7.4, 1.7 Hz, 2H), 7.24-7.15 (m, 3H), 7.10 (t, J=7.5 Hz, 1H), 7.05 (t, J=7.5 Hz, 1H), 7.01-6.99 (m, 1H).

### Synthesis of Compound CPD053

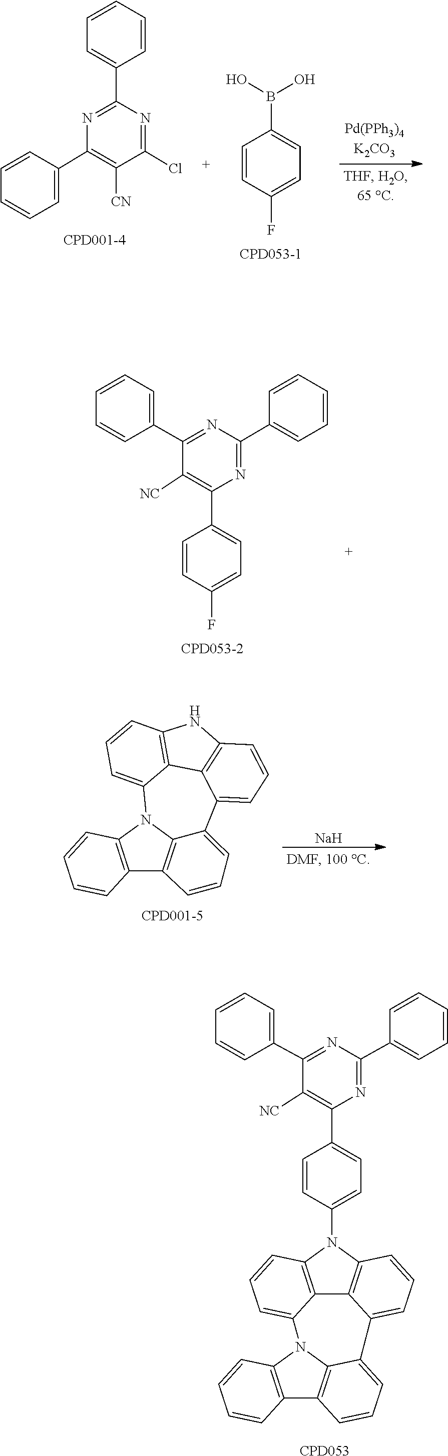

### Synthesis of Compound CPD053-2

Compounds CPD001-4 (20.00 g, 68.55 mmol), CPD053-1 (10.55 g, 75.41 mmol), tetrakis(triphenylphosphine)palladium (3.96 g, 3.43 mmol), potassium carbonate (18.95 g, 137.11 mmol), tetrahydrofuran (300 mL), and deionized water (100 mL) were added into a 1,000 mL three-necked round bottomed flask, and after nitrogen replacement under vacuum three times, the mixture was heated to a temperature of 65° C. for reaction for 4 h. The complete consumption of the starting material CPD001-4 was monitored by TLC (ethyl acetate:n-hexane=1:10 as a developing solvent). After cooled to room temperature, the mixture was concentrated under reduced pressure at 65° C. to remove the solvent. 500 mL of dichloromethane was added to dissolve the materials, and the mixture was washed with deionized water (250 mL*3), followed by liquid separation and concentration. The organic phase was purified by silica gel column chromatography (350 g, 200-300 mesh, ethyl acetate:n-hexane=1:20 as an eluent), eluted, and then concentrated under reduced pressure at 70° C. for 2 h to provide the compound CPD053-2 as a white solid (20.90 g, purity: 99.46%, yield: 86.78%, mass spectrum: 352.12 (M+H)).

### Synthesis of Compound CPD053

With reference to the synthesis and purification methods for the compound CPD001, only the corresponding starting materials were replaced, to provide the target compound as a yellow solid CPD053 (21.20 g, purity: 99.93%, yield: 75.06%). 21.20 g of the crude CPD053 was purified by sublimation to provide sublimation-purified CPD053 (17.54 g, purity: 99.95%, yield: 82.74%, mass spectrum: 662.13 (M+H)). ¹H NMR (400 MHz, CDCl₃) δ 8.41-8.29 (m, 2H), 8.25-8.15 (m, 2H), 7.95-7.90 (m, 4H), 7.88-7.79 (m, 2H), 7.72 (dd, J=15.0, 2.9 Hz, 1H), 7.61-7.45 (m, 7H), 7.45-7.35 (m, 3H), 7.21-7.18 (m, 3H), 7.14-6.97 (m, 3H).

### Synthesis of Compound CPD074

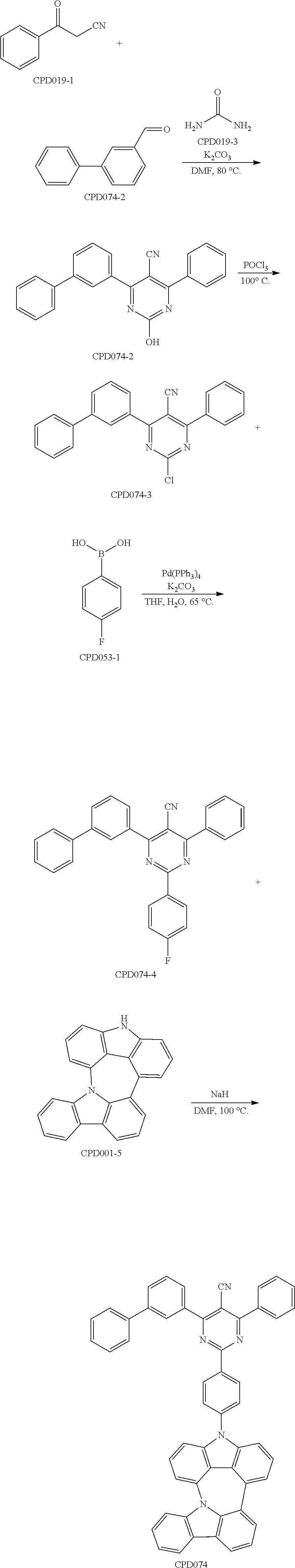

### Synthesis of Compound CPD074-2

With reference to the synthesis and purification methods for the compound CPD019-4, only the corresponding starting materials were replaced, to provide the target compound CPD074-2 as a white solid (15.23 g, purity: 99.33%, yield: 64.05%, mass spectrum: 350.22 (M+H)).

### Synthesis of Compound CPD074-3

With reference to the synthesis and purification methods for the compound CPD001-4, only the corresponding starting materials were replaced, to provide the target compound CPD074-3 as a white solid (14.55 g, purity: 99.56%, yield: 81.36%, mass spectrum: 368.24 (M+H)).

### Synthesis of Compound CPD074-4

With reference to the synthesis and purification methods for the compound CPD053-2, only the corresponding starting materials were replaced, to provide the target compound CPD074-4 as a white solid (15.65 g, purity: 99.46%, yield: 83.25%, mass spectrum: 428.16 (M+H)).

### Synthesis of Compound CPD074

With reference to the synthesis and purification methods for the compound CPD001, only the corresponding starting materials were replaced, to provide the target compound CPD074 as a yellow solid (16.86 g, purity: 99.94%, yield: 72.11%). 16.86 g of the crude CPD074 was purified by sublimation to provide sublimation-purified CPD074 (13.30 g, purity: 99.94%, yield: 78.89%, mass spectrum: 738.26 (M+H)). ¹H NMR (400 MHz, CDCl₃) δ 8.22-8.16 (m, 2H), 8.11 (t, J=2.9 Hz, 1H), 7.98-7.89 (m, 5H), 7.87-7.81 (m, 2H), 7.79-7.66 (m, 4H), 7.63-7.59 (m, 1H), 7.57-7.36 (m, 10H), 7.21-7.13 (m, 3H), 7.13-6.97 (m, 3H).

### Synthesis of Compound CPD089

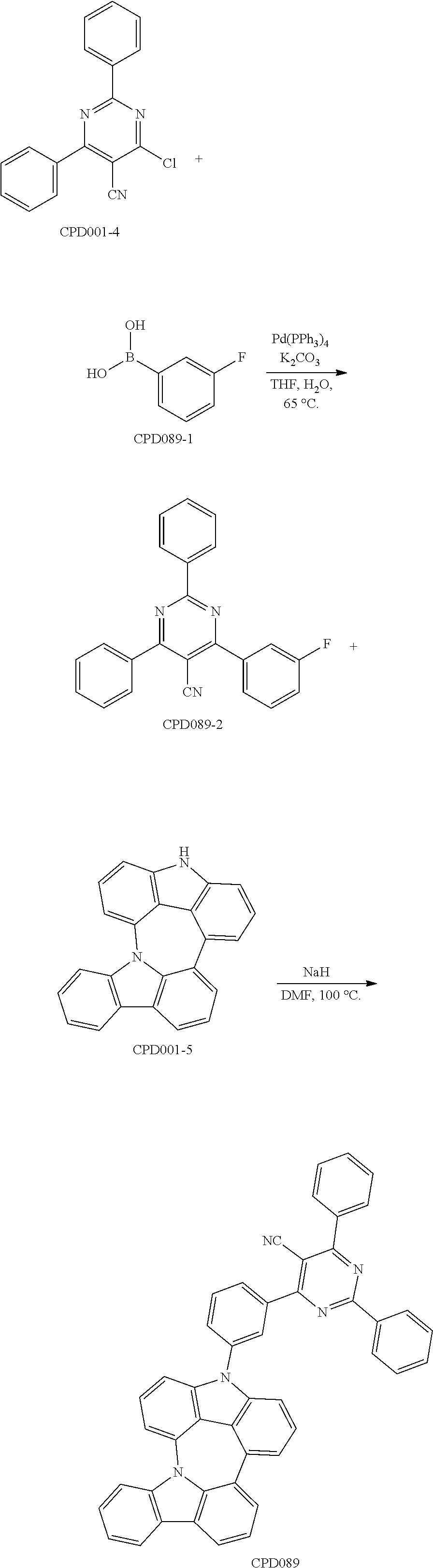

### Synthesis of Compound CPD089-2

With reference to the synthesis and purification methods for the compound CPD053-2, only the corresponding starting materials were replaced, to provide the target compound CPD089-2 as a white solid (18.25 g, purity: 99.53%, yield: 84.22%, mass spectrum: 352.16 (M+H)).

### Synthesis of Compound CPD089

With reference to the synthesis and purification methods for the compound CPD001, only the corresponding starting materials were replaced, to provide the target compound CPD089 as a yellow solid (17.47 g, purity: 99.96%, yield: 75.62%). 17.47 g of the crude CPD089 was purified by sublimation to provide sublimation-purified CPD089 (14.01 g, purity: 99.96%, yield: 80.20%, mass spectrum: 662.23 (M+H)). ¹H NMR (400 MHz, CDCl₃) δ 8.40-8.29 (m, 2H), 8.25-8.14 (m, 3H), 7.89-7.80 (m, 2H), 7.80-7.67 (m, 2H), 7.66-7.59 (m, 2H), 7.58-7.35 (m, 10H), 7.25-7.12 (m, 3H), 7.07 (dd, J=14.9, 10.4 Hz, 2H), 7.02-6.96 (m, 1H).

### Synthesis of Compound CPD100

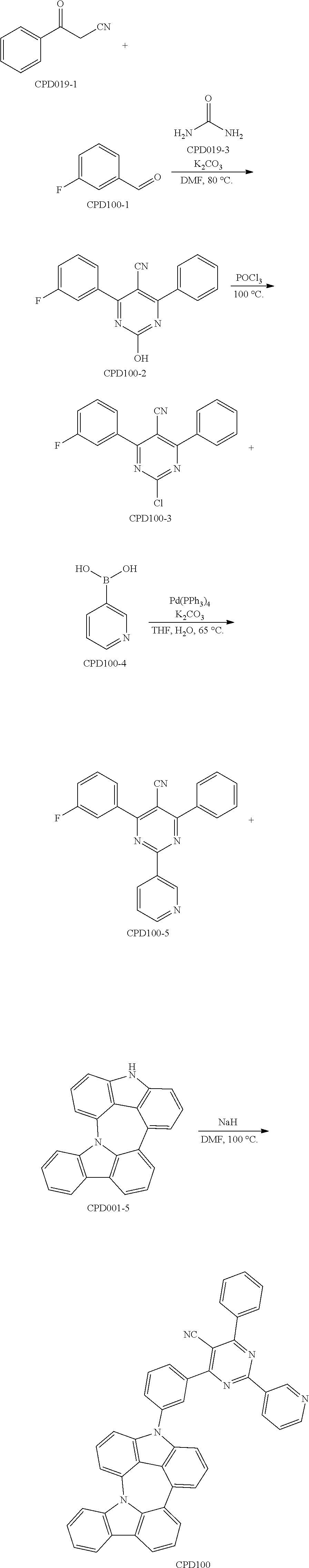

### Synthesis of Compound CPD100-2

With reference to the synthesis and purification methods for the compound CPD019-4, only the corresponding starting materials were replaced, to provide the target compound CPD100-2 as a white solid (19.45 g, purity: 99.42%, yield: 62.96%, mass spectrum: 292.06 (M+H)).

### Synthesis of Compound CPD100-3

With reference to the synthesis and purification methods for the compound CPD001-4, only the corresponding starting materials were replaced, to provide the target compound CPD100-3 as a white solid (18.08 g, purity: 99.51%, yield: 88.05%, mass spectrum: 310.05 (M+H)).

### Synthesis of Compound CPD100-5

With reference to the synthesis and purification methods for the compound CPD053-2, only the corresponding starting materials were replaced, to provide the target compound CPD100-5 as a white solid (17.06 g, purity: 99.63%, yield: 85.33%, mass spectrum: 353.21 (M+H)).

### Synthesis of Compound CPD100

With reference to the synthesis and purification methods for the compound CPD001, only the corresponding starting materials were replaced, to provide the target compound CPD100 as a yellow solid (18.96 g, purity: 99.93%, yield: 78.09%). 18.96 g of the crude CPD100 was purified by sublimation to provide sublimation-purified CPD100 (15.74 g, purity: 99.95%, yield: 83.02%, mass spectrum: 663.22 (M+H)). ¹H NMR (400 MHz, CDCl₃) δ 9.10 (s, 1H), 8.77-8.64 (m, 1H), 8.33 (dt, J=15.0, 3.0 Hz, 1H), 8.25-8.13 (m, 3H), 7.91-7.76 (m, 3H), 7.76-7.59 (m, 3H), 7.59-7.45 (m, 5H), 7.45-7.34 (m, 3H), 7.22-7.12 (m, 3H), 7.07 (dd, J=14.9, 10.4 Hz, 2H), 7.03-6.95 (m, 1H).

### Synthesis of Compound CPD125

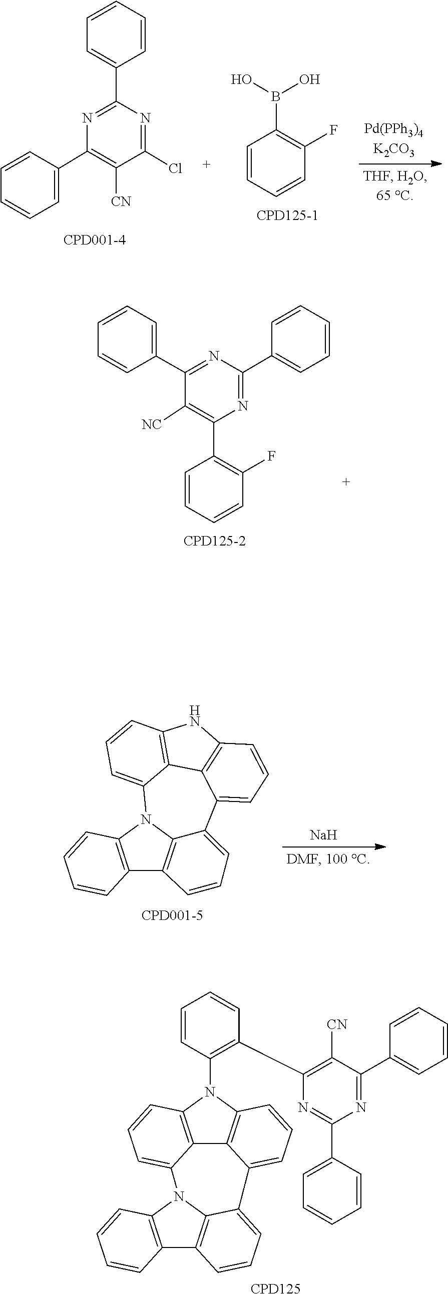

### Synthesis of Compound CPD125-2

With reference to the synthesis and purification methods for the compound CPD053-2, only the corresponding starting materials were replaced, to provide the target compound CPD125-2 as a white solid (17.63 g, purity: 99.40%, yield: 85.23%, mass spectrum: 352.12 (M+H)).

### Synthesis of Compound CPD125

With reference to the synthesis and purification methods for the compound CPD001, only the corresponding starting materials were replaced, to provide the target compound CPD125 as a yellow solid (15.05 g, purity: 99.96%, yield: 74.63%). 15.05 g of the crude CPD125 was purified by sublimation to provide sublimation-purified CPD125 (12.24 g, purity: 99.96%, yield: 81.33%, mass spectrum: 662.23 (M+H)). ¹H NMR (400 MHz, CDCl₃) δ 8.01-7.93 (m, 5H), 7.86-7.74 (m, 6H), 7.65 (d, J=7.7 Hz, 1H), 7.55-7.46 (m, 3H), 7.38 (t, J=6.6 Hz, 3H), 7.28-7.22 (m, 4H), 7.20-7.12 (m, 3H), 7.09 (d, J=8.0 Hz, 1H), 6.89 (d, J=8.0 Hz, 1H).

### Synthesis of Compound CPD127

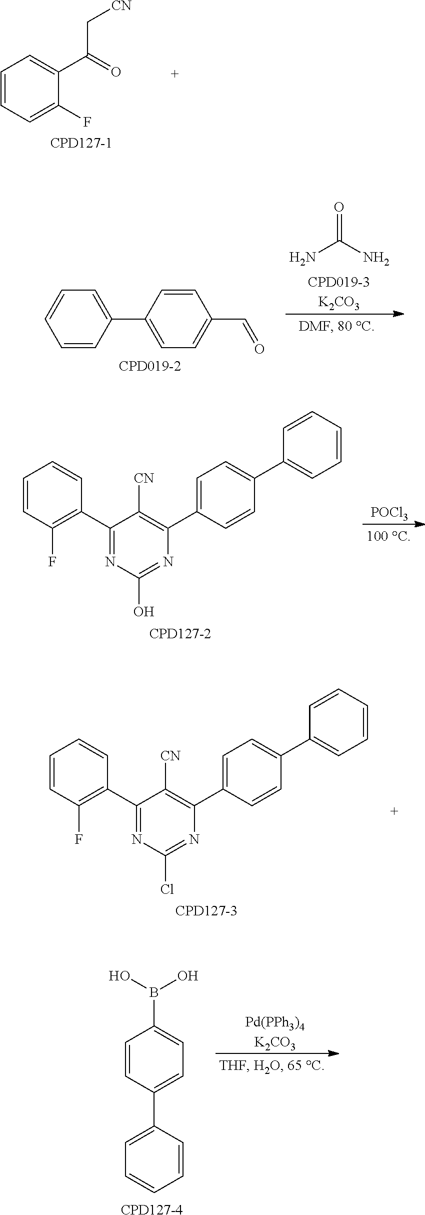

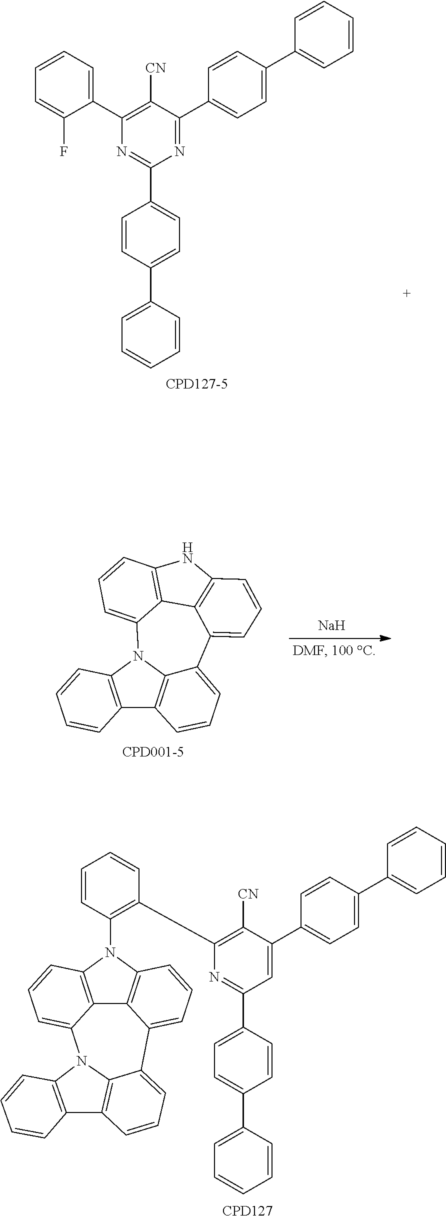

### Synthesis of Compound CPD127-2

With reference to the synthesis and purification methods for the compound CPD019-4, only the corresponding starting materials were replaced, to provide the target compound CPD127-2 as a white solid (19.89 g, purity: 99.36%, yield: 65.18%, mass spectrum: 368.11 (M+H)).

### Synthesis of Compound CPD127-3

With reference to the synthesis and purification methods for the compound CPD001-4, only the corresponding starting materials were replaced, to provide the target compound CPD127-3 as a white solid (17.85 g, purity: 99.54%, yield: 85.36%, mass spectrum: 386.08 (M+H)).

### Synthesis of Compound CPD127-5

With reference to the synthesis and purification methods for the compound CPD053-2, only the corresponding starting materials were replaced, to provide the target compound CPD127-5 as a white solid (16.63 g, purity: 99.71%, yield: 82.55%, mass spectrum: 504.08 (M+H)).

### Synthesis of Compound CPD127

With reference to the synthesis and purification methods for the compound CPD001, only the corresponding starting materials were replaced, to provide the target compound CPD127 as a yellow solid (16.52 g, purity: 99.94%, yield: 72.21%). 16.52 g of the crude CPD127 was purified by sublimation to provide sublimation-purified CPD127 (12.99 g, purity: 99.95%, yield: 78.64%, mass spectrum: 814.30 (M+H)). ¹H NMR (400 MHz, CDCl₃) δ 8.34-8.26 (m, 2H), 8.23-8.15 (m, 2H), 8.00-7.88 (m, 4H), 7.88-7.78 (m, 3H), 7.78-7.69 (m, 5H), 7.56-7.35 (m, 11H), 7.30-7.23 (m, 2H), 7.21-7.18 (m, 3H), 7.13-6.94 (m, 3H).

### Synthesis of Compound CPD143

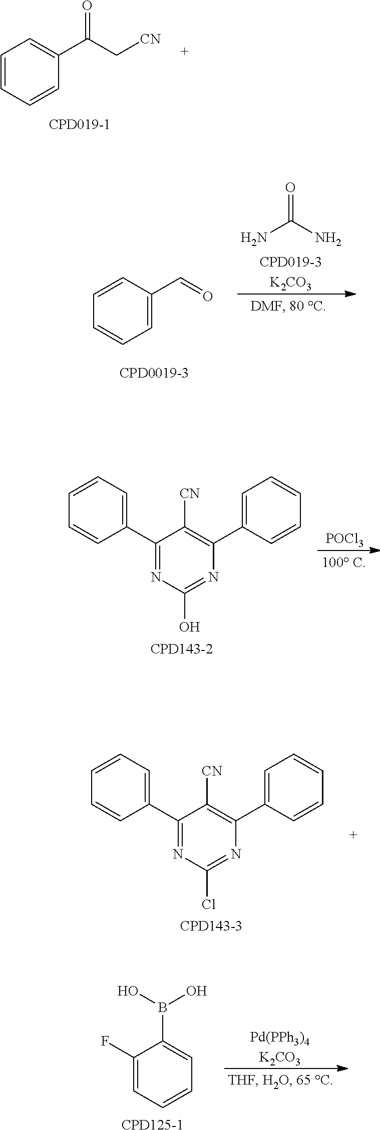

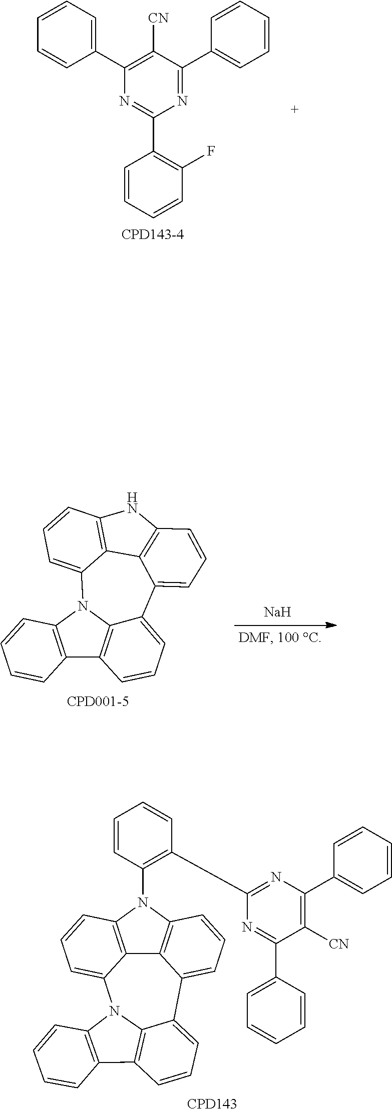

### Synthesis of Compound CPD143-2

With reference to the synthesis and purification methods for the compound CPD019-4, only the corresponding starting materials were replaced, to provide the target compound CPD143-2 as a white solid (25.36 g, purity: 99.21%, yield: 61.18%, mass spectrum: 274.08 (M+H)).

### Synthesis of Compound CPD143-3

With reference to the synthesis and purification methods for the compound CPD001-4, only the corresponding starting materials were replaced, to provide the target compound CPD143-3 as a white solid (24.58 g, purity: 99.61%, yield: 86.41%, mass spectrum: 292.06 (M+H)).

### Synthesis of Compound CPD143-4

With reference to the synthesis and purification methods for the compound CPD053-2, only the corresponding starting materials were replaced, to provide the target compound CPD143-4 as a white solid (20.35 g, purity: 99.68%, yield: 81.41%, mass spectrum: 352.08 (M+H)).

### Synthesis of Compound CPD143

With reference to the synthesis and purification methods for the compound CPD001, only the corresponding starting materials were replaced, to provide the target compound CPD143 as a yellow solid (14.04 g, purity: 99.95%, yield: 68.57%). 14.04 g of the crude CPD143 was purified by sublimation to provide sublimation-purified CPD143 (10.99 g, purity: 99.95%, yield: 78.27%, mass spectrum: 662.24 (M+H)). ¹H NMR (400 MHz, CDCl₃) δ 8.47 (d, J=7.1 Hz, 1H), 8.05 (dd, J=17.1, 8.0 Hz, 2H), 7.85-7.57 (m, 9H), 7.46-7.21 (m, 11H), 7.17-7.12 (m, 2H), 6.89 (d, J=8.0 Hz, 1H), 6.72 (d, J=8.0 Hz, 1H).

### Synthesis of Compound CPD159

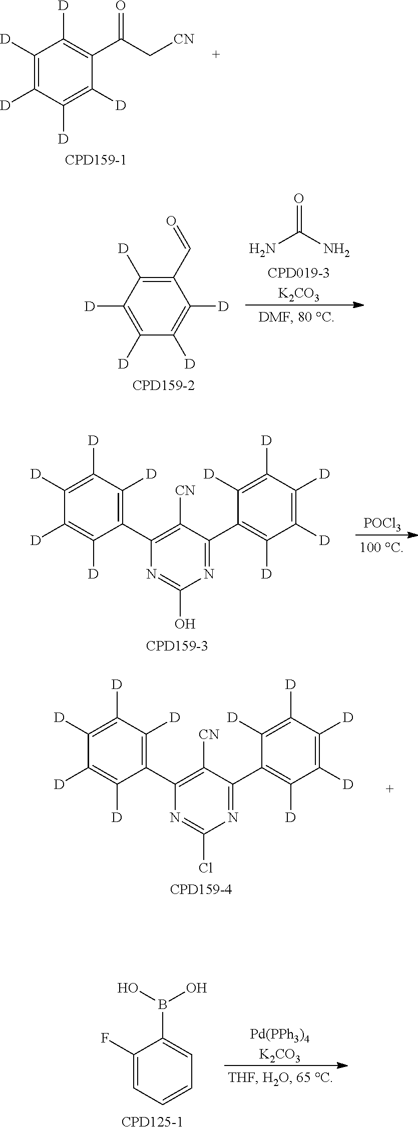

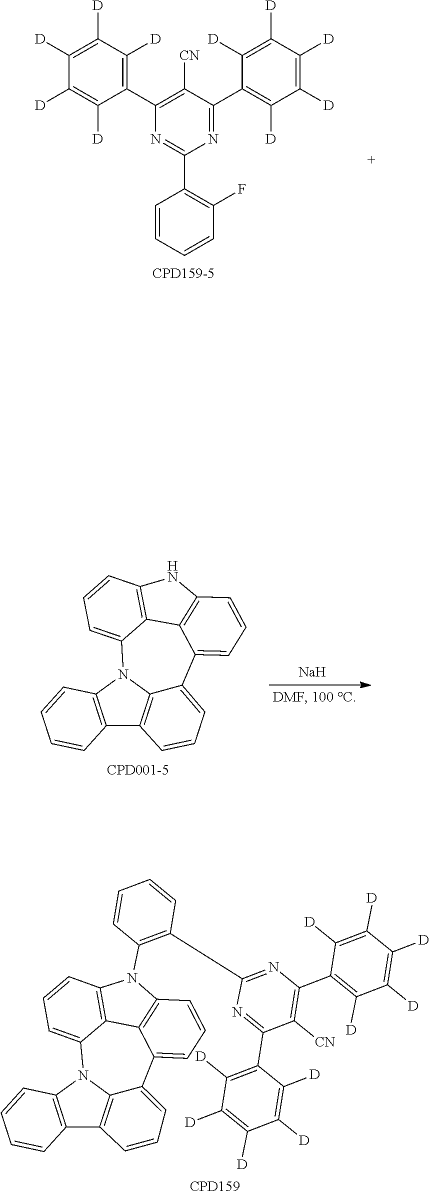

### Synthesis of Compound CPD159-3

With reference to the synthesis and purification methods for the compound CPD019-4, only the corresponding starting materials were replaced, to provide the target compound CPD159-3 as a white solid (18.88 g, purity: 99.21%, yield: 62.74%, mass spectrum: 284.16 (M+H)).

### Synthesis of Compound CPD159-4

With reference to the synthesis and purification methods for the compound CPD001-4, only the corresponding starting materials were replaced, to provide the target compound CPD159-4 as a white solid (17.60 g, purity: 99.50%, yield: 87.54%, mass spectrum: 302.24 (M+H)).

### Synthesis of Compound CPD159-5

With reference to the synthesis and purification methods for the compound CPD053-2, only the corresponding starting materials were replaced, to provide the target compound CPD159-5 as a white solid (16.32 g, purity: 99.75%, yield: 82.95%, mass spectrum: 362.02 (M+H)).

### Synthesis of Compound CPD159

With reference to the synthesis and purification methods for the compound CPD001, only the corresponding starting materials were replaced, to provide the target compound CPD159 as a yellow solid (14.44 g, purity: 99.92%, yield: 67.50%). 14.44 g of the crude CPD159 was purified by sublimation to provide sublimation-purified CPD159 (11.31 g, purity: 99.95%, yield: 78.32%, mass spectrum: 672.29 (M+H)). ¹H NMR (400 MHz, CDCl₃) δ 8.23-8.16 (m, 2H), 7.94-7.89 (m, 2H), 7.84-7.68 (m, 2H), 7.56-7.45 (m, 2H), 7.45-7.36 (m, 3H), 7.26-7.08 (m, 4H), 7.07-6.98 (m, 2H).

### Synthesis of Compound CPD165

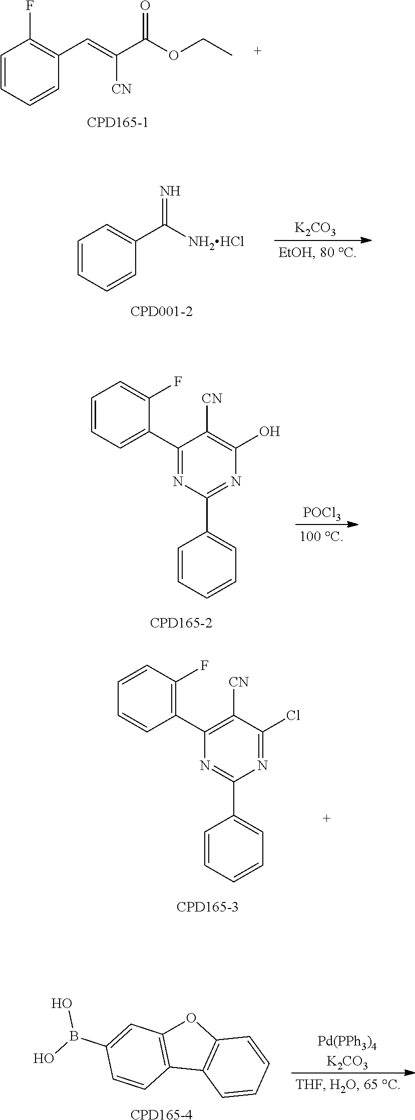

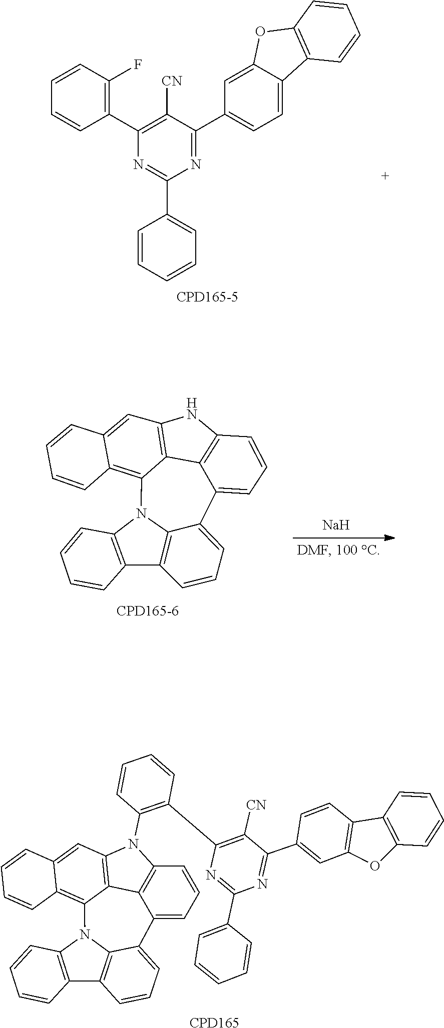

### Synthesis of Compound CPD165-2

With reference to the synthesis and purification methods for the compound CPD001-3, only the corresponding starting materials were replaced, to provide the target compound CPD165-2 as a white solid (20.63 g, purity: 99.50%, yield: 84.21%, mass spectrum: 292.07 (M+H)).

### Synthesis of Compound CPD165-3

With reference to the synthesis and purification methods for the compound CPD001-4, only the corresponding starting materials were replaced, to provide the target compound CPD165-3 as a white solid (18.95 g, purity: 99.57%, yield: 86.46%, mass spectrum: 310.06 (M+H)).

### Synthesis of Compound CPD165-5

With reference to the synthesis and purification methods for the compound CPD053-2, only the corresponding starting materials were replaced, to provide the target compound CPD165-5 as a white solid (20.08 g, purity: 99.64%, yield: 81.07%, mass spectrum: 442.13 (M+H)).

### Synthesis of Compound CPD165

With reference to the synthesis and purification methods for the compound CPD001, only the corresponding starting materials were replaced, to provide the target compound CPD165 as a yellow solid (17.41 g, purity: 99.93%, yield: 66.48%). 17.41 g of the crude CPD165 was purified by sublimation to provide sublimation-purified CPD165 (14.63 g, purity: 99.95%, yield: 84.03%, mass spectrum: 802.25 (M+H)). ¹H NMR (400 MHz, CDCl₃) δ 8.51 (dd, J=14.9, 3.0 Hz, 1H), 8.40-8.29 (m, 2H), 8.24-8.11 (m, 2H), 8.04-7.88 (m, 5H), 7.88-7.77 (m, 2H), 7.75-7.69 (m, 3H), 7.62-7.29 (m, 12H), 7.29-7.14 (m, 2H), 7.10-7.02 (m, 2H).

### Synthesis of Compound CPD180

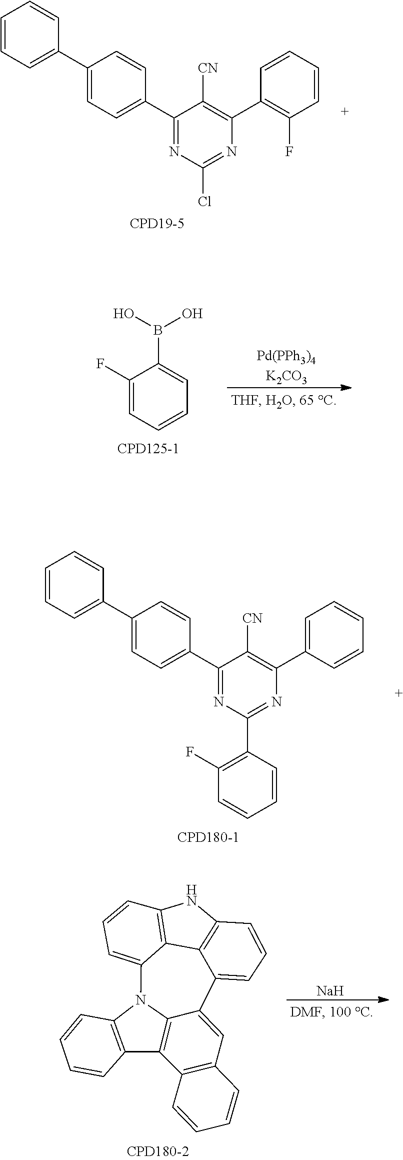

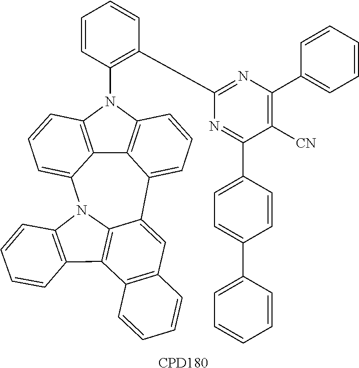

### Synthesis of Compound CPD180-1

With reference to the synthesis and purification methods for the compound CPD053-2, only the corresponding starting materials were replaced, to provide the target compound CPD180-1 as a white solid (24.16 g, purity: 99.74%, yield: 80.43%, mass spectrum: 428.15 (M+H)).

### Synthesis of Compound CPD180

With reference to the synthesis and purification methods for the compound CPD001, only the corresponding starting materials were replaced, to provide the target compound CPD180 as a yellow solid (18.96 g, purity: 99.94%, yield: 65.36%). 18.96 g of the crude CPD180 was purified by sublimation to provide sublimation-purified CPD180 (15.19 g, purity: 99.94%, yield: 80.11%, mass spectrum: 788.06 (M+H)). ¹H NMR (400 MHz, CDCl₃) δ 8.67 (d, J=2.9 Hz, 1H), 8.54 (dd, J=14.8, 3.1 Hz, 1H), 8.42-8.10 (m, 3H), 7.94-7.90 (m, 2H), 7.89-7.77 (m, 6H), 7.76-7.72 (m, 2H), 7.67-7.34 (m, 13H), 7.21-7.15 (m, 3H), 7.10-6.95 (m, 2H).

### Synthesis of Compound CPD220

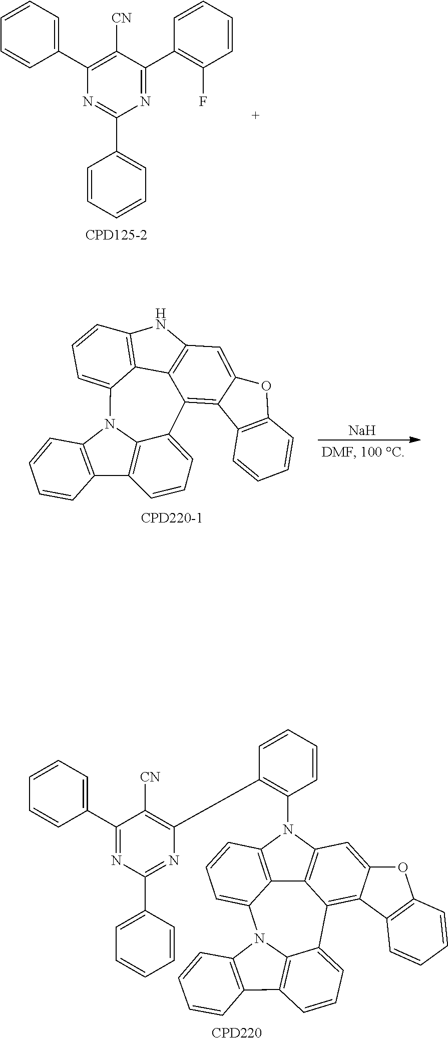

### Synthesis of Compound CPD220

With reference to the synthesis and purification methods for the compound CPD001, only the corresponding starting materials were replaced, to provide the target compound CPD220 as a yellow solid (17.53 g, purity: 99.95%, yield: 61.23%). 17.53 g of the crude CPD220 was purified by sublimation to provide sublimation-purified CPD220 (13.94 g, purity: 99.95%, yield: 79.52%, mass spectrum: 752.24 (M+H)). ¹H NMR (400 MHz, CDCl₃) δ 8.41-8.26 (m, 3H), 8.24-8.14 (m, 2H), 8.02-7.88 (m, 3H), 7.88-7.76 (m, 3H), 7.72 (dd, J=15.0, 2.9 Hz, 1H), 7.59-7.44 (m, 9H), 7.44-7.26 (m, 3H), 7.25-7.12 (m, 3H), 7.12-6.98 (m, 2H).

### Application Example: Preparation of Organic Electroluminescent Device

A glass substrate of 50 mm*50 mm*1.0 mm with an ITO (100 nm) transparent electrode was ultrasonically cleaned in ethanol for 10 min, dried at 150° C., and then treated with N₂ plasma for 30 min. The washed glass substrate was mounted on a substrate holder in a vacuum evaporation apparatus. Compound NDP-9 and compound HTM 1 were evaporated at a weight ratio of 97:3 to form a hole injection layer with a film thickness of 10 nm. Subsequently, a layer of HTM1 was evaporated to form a film with a film thickness of 60 nm as HTL1. Then, a layer of HTM2 was evaporated on the HTM1 film to form a film with a film thickness of 10 nm as HTL2. Finally, a light emitting layer with a film thickness of 40 nm was evaporated by co-evaporation on the HTM2 film layer (host material:dopant material=97%:3%), wherein the host materials were the compound in the present disclosure and the comparative compounds 1-5, respectively. Then, ETL and LiQ were co-evaporated (35 nm) on the light emitting layer at a weight ratio of 50:50 as an electron transport material. Then, LiQ (1 nm) was evaporated above the electron transport material layer as an electron injection material, and then Mg/Ag (100 nm, 1:9) was co-evaporated as a cathode material.

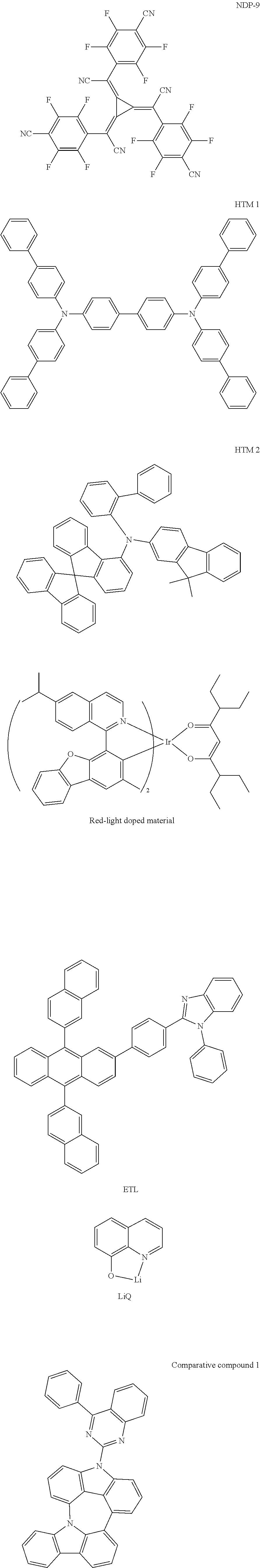

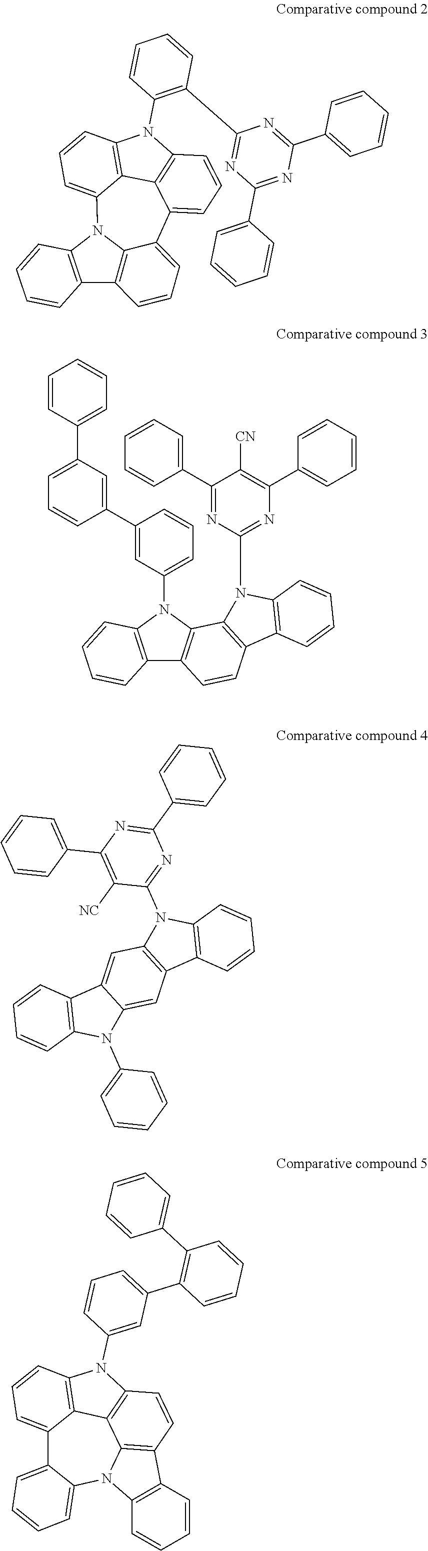

### Evaluation:

Performance test was performed on the above device by comparison between the compounds in the Examples and Comparative Examples 1-5 of the present disclosure respectively as host materials. The luminescence spectrum was measured using a spectroradiometric luminance meter (CS 2000) with a constant current source (Keithley 2400) and with a constant current density flowing through a light emitting element. The voltage value, current efficiency, and spectrum were measured simultaneously, and the time (LT95) for the luminance to reach 9500 of the initial luminance was also measured. The result is shown in Table 1:

| | | | Light emitting | Emission | |
|---|---|---|---|---|---|
| | | Voltage | efficiency | wavelength | LT95@5000 nits |
| | Host material | (V) | (cd/a) | (nm) | (h) |
| Example 1 | CPD001 | 3.54 | 16.7 | 623 | 602 |
| Example 2 | CPD009 | 3.57 | 16.4 | 624 | 579 |
| Example 3 | CPD019 | 3.60 | 16.8 | 624 | 611 |
| Example 4 | CPD033 | 3.64 | 16.6 | 624 | 566 |
| Example 5 | CPD053 | 3.65 | 16.9 | 624 | 609 |
| Example 6 | CPD074 | 3.68 | 17.3 | 623 | 587 |
| Example 7 | CPD089 | 3.62 | 16.5 | 623 | 573 |
| Example 8 | CPD100 | 3.56 | 16.4 | 624 | 542 |
| Example 9 | CPD125 | 3.62 | 17.2 | 624 | 624 |
| Example 10 | CPD127 | 3.66 | 16.8 | 623 | 569 |
| Example 11 | CPD143 | 3.61 | 16.7 | 624 | 598 |
| Example 12 | CPD159 | 3.64 | 17.1 | 624 | 627 |
| Example 13 | CPD165 | 3.67 | 16.5 | 623 | 545 |
| Example 14 | CPD180 | 3.69 | 16.5 | 623 | 557 |
| Example 15 | CPD220 | 3.71 | 17.0 | 624 | 583 |
| Comparative | Comparative | 4.65 | 14.8 | 624 | 205 |
| Example 1 | compound 1 | | | | |
| Comparative | Comparative | 4.43 | 15.6 | 624 | 214 |
| Example 2 | compound 2 | | | | |
| Comparative | Comparative | 4.79 | 13.7 | 622 | 169 |
| Example 3 | compound 3 | | | | |
| Comparative | Comparative | 4.72 | 14.3 | 625 | 191 |
| Example 4 | compound 4 | | | | |
| Comparative | Comparative | 4.82 | 13.4 | 623 | 144 |
| Example 5 | compound 5 | | | | |
| Example 15 | CPD43: | 3.69 | 16.5 | 624 | 532 |
| | Comparative | | | | |
| | compound 5 = | | | | |
| | 50%:50% | | | | |

Comparison of sublimation temperature: the sublimation temperature is defined as: a temperature corresponding to a sublimation rate of 1 angstrom per second under a vacuum degree of 10⁻⁷ Torr. The test result is as follows:

| Material | Sublimation temperature/° C. |
|---|---|
| CPD001 | 254 |
| CPD125 | 261 |
| CPD143 | 263 |
| Comparative compound 2 | 278 |
| Comparative compound 3 | 274 |
| Comparative compound 4 | 281 |

As can be seen from the comparison of the data in the above table, the host material in the present disclosure has low sublimation temperature, which is conducive to industrial application.

The material in the present disclosure has the advantages of high optical and electrical stability, low sublimation temperature, low driving voltage, high light emitting efficiency, and long device service life, can be used as a host material in an OLED device, further has a low melting point, and is conductive to material evaporation stability as a molten material. As a host material, this compound has the possibility of being applied to AMOLED industry.

1. An organic electroluminescent material, having a structure of formula (1), 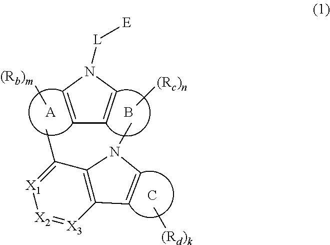
wherein L is selected from any one of a single bond, a substituted or unsubstituted C₆-C₃₀ aryl, or a substituted or unsubstituted C₃-C₂₀ heteroaryl;
X₁ to X₃ each independently represent N or CRₐ; ring A, ring B, and ring C are, identically or differently during each occurrence, selected from an aromatic ring with 5-18 carbon atoms or a heteroaromatic ring with 3-18 carbon atoms;
wherein E has a structure of formula (2): 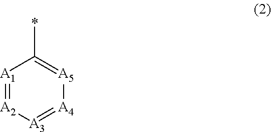
wherein A₁ to A₅ are identical or different; A₁ to A₅ are each independently selected from N or CR₁; at least one of A₁ to A₅ is N, at least one thereof is C—CN; and * represents a linking site with formula (1);
wherein Rₐ, R_{b}, R_{c}, R_{d}, and R₁ are identical or different during each occurrence, and are each independently selected from hydrogen, deuterium, halogen, a substituted or unsubstituted alkyl with 1-10 carbon atoms, a substituted or unsubstituted cycloalkyl with 3-ring carbon atoms, a substituted or unsubstituted heteroalkyl with 1-10 carbon atoms, a substituted or unsubstituted heterocyclyl with 3-20 ring atoms, a substituted or unsubstituted arylalkyl with 7-30 carbon atoms, a substituted or unsubstituted alkoxy with 1-20 carbon atoms, a substituted or unsubstituted aryloxy with 6-30 carbon atoms, a substituted or unsubstituted alkenyl with 2-10 carbon atoms, a substituted or unsubstituted aryl with 6-30 carbon atoms, a substituted or unsubstituted heteroaryl with 3-30 carbon atoms, a substituted or unsubstituted alkylsilyl with 3-20 carbon atoms, a substituted or unsubstituted arylsilyl with 6-20 carbon atoms, a substituted or unsubstituted amino with 0-20 carbon atoms, a substituted or unsubstituted acyl with 0-20 carbon atoms, carbonyl, carboxyl, a substituted or unsubstituted ester group with 0-20 carbon atoms, cyano, isocyano, hydroxyl, sulfydryl, a substituted or unsubstituted sulfinyl with 0-20 carbon atoms, a substituted or unsubstituted sulfonyl with 0-20 carbon atoms, or phosphino; adjacent Rₐ, adjacent R_{b}, adjacent R_{c}, and adjacent R_{d} are respectively capable of being linked to form a ring, and the substitution refers to a substitution with deuterium, halogen, cyano, an alkyl with 1-6 carbon atoms, or an aryl with 6-30 carbon atoms,
wherein m, n, and k are each independently selected from an integer of 1 to 4; and
heteroatoms in the heteroaromatic ring, the heteroalkyl, the heterocyclyl, and the heteroaryl are each independently selected from O, S, N, Si, Ge, or P.

2. The organic electroluminescent material of claim 1, having a structure of formula (3), 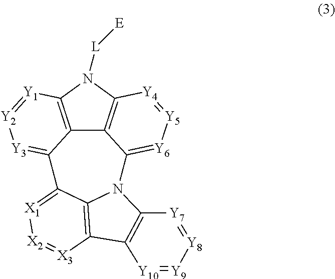
wherein X₁ to X₃ each independently represent N or CRₐ; Y₁ to Y₁₀ are identical or different; Y₁ to Y₁₀ are each independently selected from N or CR; and Rₐ is as defined in claim 1;
R is as defined as R_{b}, R_{c}, and R_{d} in claim 1; and R is identical or different during each occurrence, and is each independently selected from hydrogen, deuterium, halogen, a substituted or unsubstituted alkyl with 1-10 carbon atoms, a substituted or unsubstituted cycloalkyl with 3-20 ring carbon atoms, a substituted or unsubstituted heteroalkyl with 1-10 carbon atoms, a substituted or unsubstituted heterocyclyl with 3-20 ring atoms, a substituted or unsubstituted arylalkyl with 7-30 carbon atoms, a substituted or unsubstituted alkoxy with 1-20 carbon atoms, a substituted or unsubstituted aryloxy with 6-30 carbon atoms, a substituted or unsubstituted alkenyl with 2-10 carbon atoms, a substituted or unsubstituted aryl with 6-30 carbon atoms, a substituted or unsubstituted heteroaryl with 3-30 carbon atoms, a substituted or unsubstituted alkylsilyl with 3-20 carbon atoms, a substituted or unsubstituted arylsilyl with 6-20 carbon atoms, a substituted or unsubstituted amino with 0-carbon atoms, a substituted or unsubstituted acyl with 0-20 carbon atoms, carbonyl, carboxyl, a substituted or unsubstituted ester group with 0-20 carbon atoms, cyano, isocyano, hydroxyl, sulfydryl, a substituted or unsubstituted sulfinyl with 0-20 carbon atoms, a substituted or unsubstituted sulfonyl with 0-20 carbon atoms, or phosphino; adjacent R and the adjacent Rₐ are respectively capable of being linked to form a ring, and the substitution refers to a substitution with deuterium, halogen, cyano, an alkyl with 1-6 carbon atoms, or an aryl with 6-30 carbon atoms.

3. The organic electroluminescent material of claim 2, wherein Y₁ to Y₁₀ each independently represent CR; and R is as defined in claim 2.

4. The organic electroluminescent material of claim 3, having one of structures represented by formulas (4)-(27):

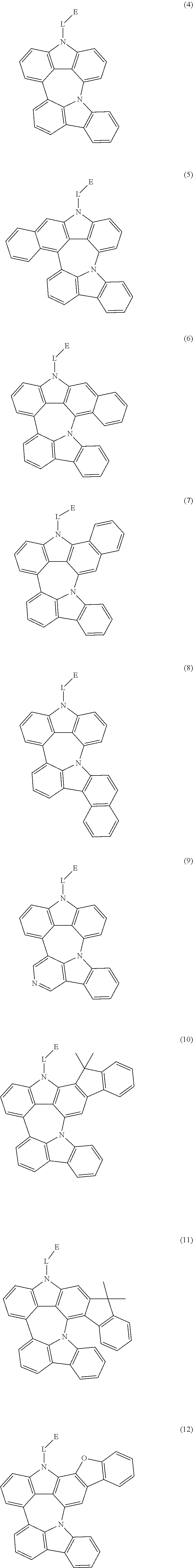

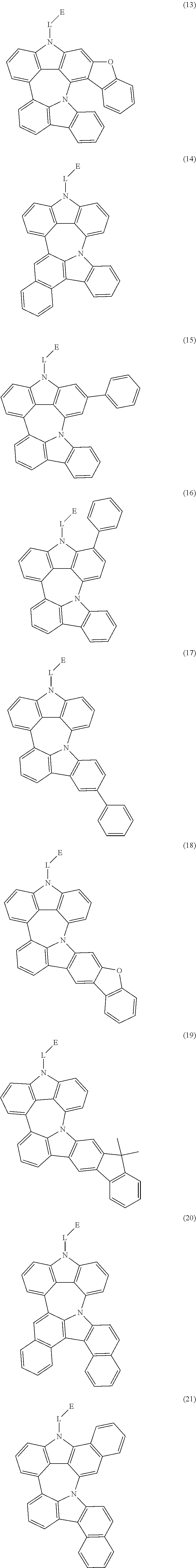

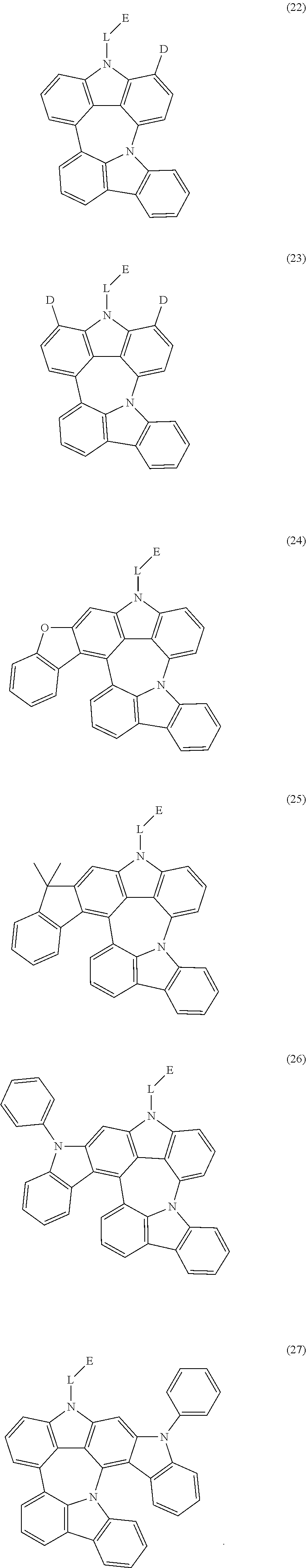

5. The organic electroluminescent material of claim 4, wherein L is a single bond or a substituted or unsubstituted C₆-C₁₀ aryl.

6. The organic electroluminescent material of claim 5, wherein L is a substituted or unsubstituted phenyl.

7. The organic electroluminescent material according to claim 1, wherein A₁ to A₅ in the formula (2) comprise two N; and at least one of A₁ to A₅ is C—CN.

8. The organic electroluminescent material of claim 71, wherein E has a structure represented by one of formulas (28)-(34): 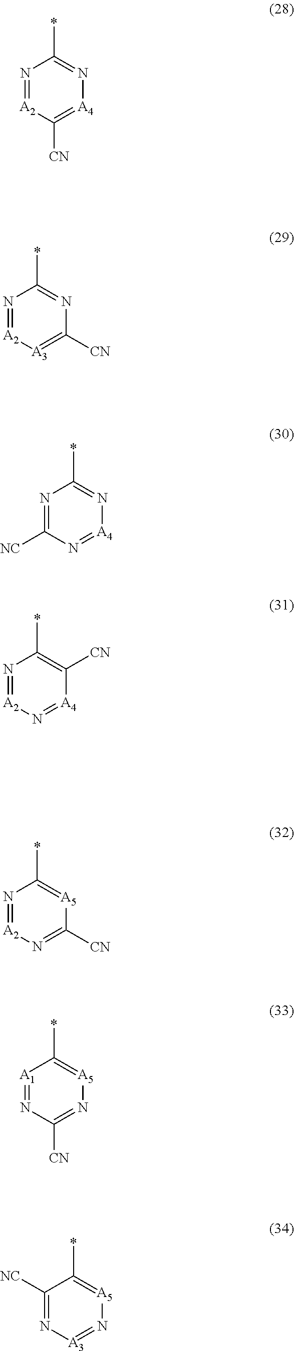
wherein A₁ to A₅ in the formulas (28)-(34) are CR₁; and R₁ is as defined in claim 1.

9. The organic electroluminescent material of claim 8, wherein R₁ is a substituted or unsubstituted aryl with 6-18 carbon atoms, or a substituted or unsubstituted heteroaryl with 3-18 carbon atoms.

10. The organic electroluminescent material of claim 9, wherein R₁ is, identically or differently during each occurrence, selected from a group consisting of phenyl, deuterated phenyl, methylphenyl, fluorophenyl, tert-butylphenyl, trideuterated methylphenyl, biphenyl, naphthyl, deuterated naphthyl, dibenzofuryl, dibenzothiophenyl, 9,9-dimethylfluorenyl, carbazolyl, pyridyl, pyrimidinyl, 4-cyanophenyl, 3-cyanophenyl, and triphenylene.

11. The organic electroluminescent material of claim 1, being one of following structural formulas, or being one of the following structural formulas in which hydrogen is partially or completely replaced by deuterium or fluorine.

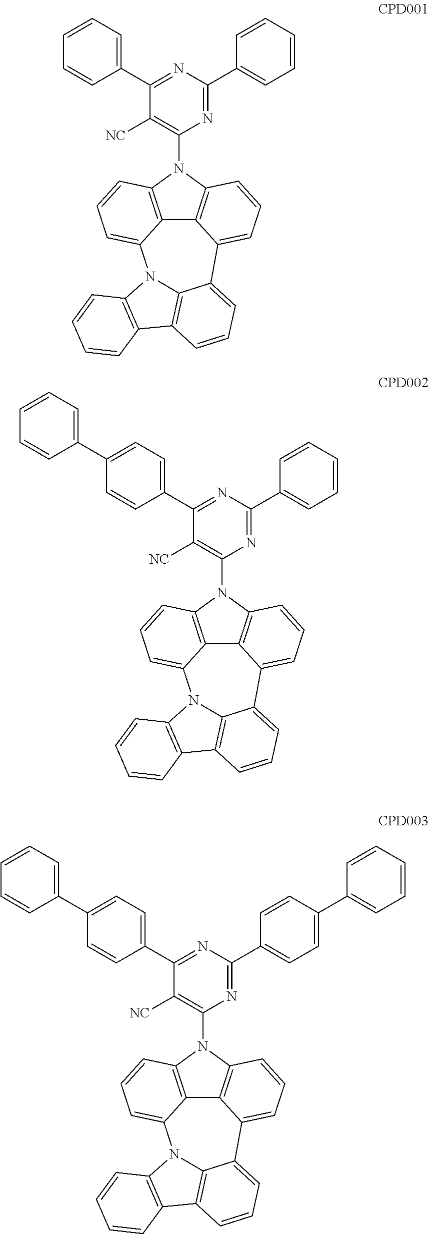 [SMILES: N#Cc1c(-c2ccccc2)nc(-c2ccccc2)nc1-n1c2cccc3c4cccc5c6ccccc6n(c6cccc1c6c32)c45 ; N#Cc1c(-c2ccc(-c3ccccc3)cc2)nc(-c2ccccc2)nc1-n1c2cccc3c4cccc5c6ccccc6n(c6cccc1c6c32)c45 ; N#Cc1c(-c2ccc(-c3ccccc3)cc2)nc(-c2ccc(-c3ccccc3)cc2)nc1-n1c2cccc3c4cccc5c6ccccc6n(c6cccc1c6c32)c45]

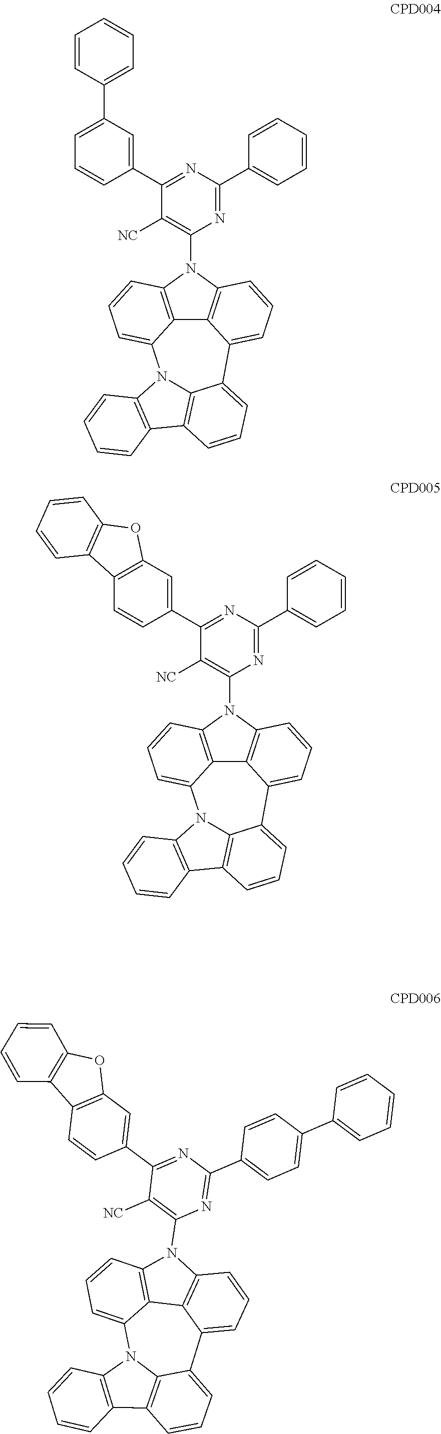 [SMILES: N#Cc1c(-c2cccc(-c3ccccc3)c2)nc(-c2ccccc2)nc1-n1c2cccc3c4cccc5c6ccccc6n(c6cccc1c6c32)c45 ; N#Cc1c(-c2ccc3c(c2)oc2ccccc23)nc(-c2ccccc2)nc1-n1c2cccc3c4cccc5c6ccccc6n(c6cccc1c6c32)c45 ; N#Cc1c(-c2ccc3c(c2)oc2ccccc23)nc(-c2ccc(-c3ccccc3)cc2)nc1-n1c2cccc3c4cccc5c6ccccc6n(c6cccc1c6c32)c45]

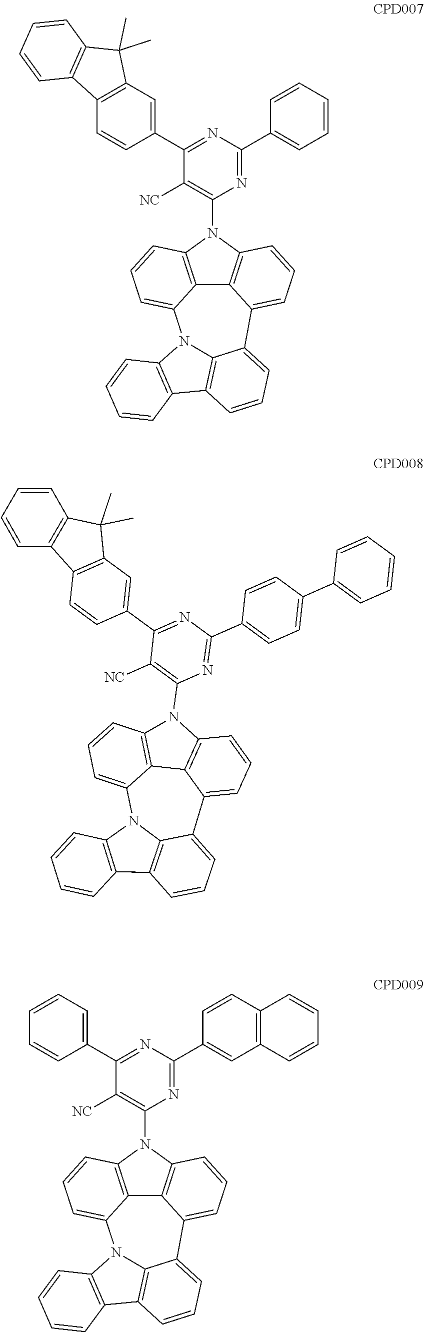 [SMILES: CC1(C)c2ccccc2-c2ccc(-c3nc(-c4ccccc4)nc(-n4c5cccc6c7cccc8c9ccccc9n(c9cccc4c9c65)c78)c3C#N)cc21 ; CC1(C)c2ccccc2-c2ccc(-c3nc(-c4ccc(-c5ccccc5)cc4)nc(-n4c5cccc6c7cccc8c9ccccc9n(c9cccc4c9c65)c78)c3C#N)cc21 ; N#Cc1c(-c2ccccc2)nc(-c2ccc3ccccc3c2)nc1-n1c2cccc3c4cccc5c6ccccc6n(c6cccc1c6c32)c45]

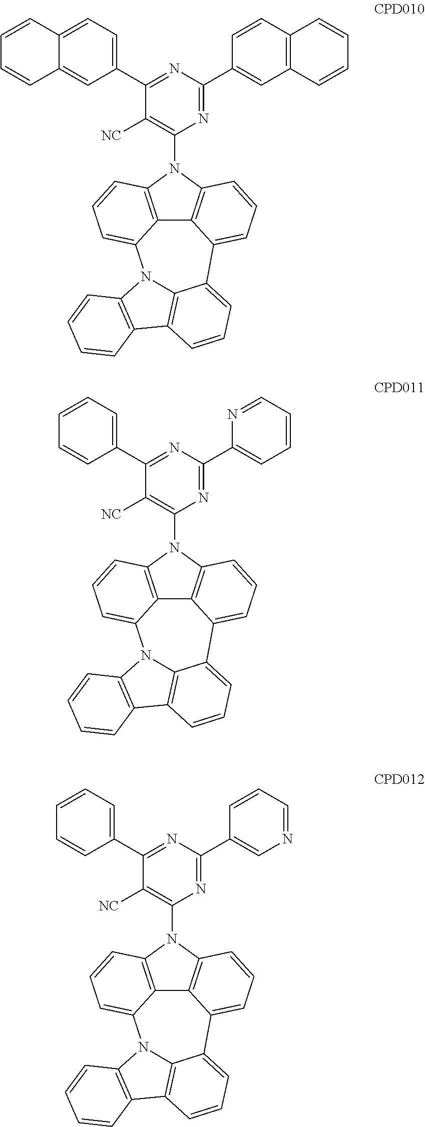

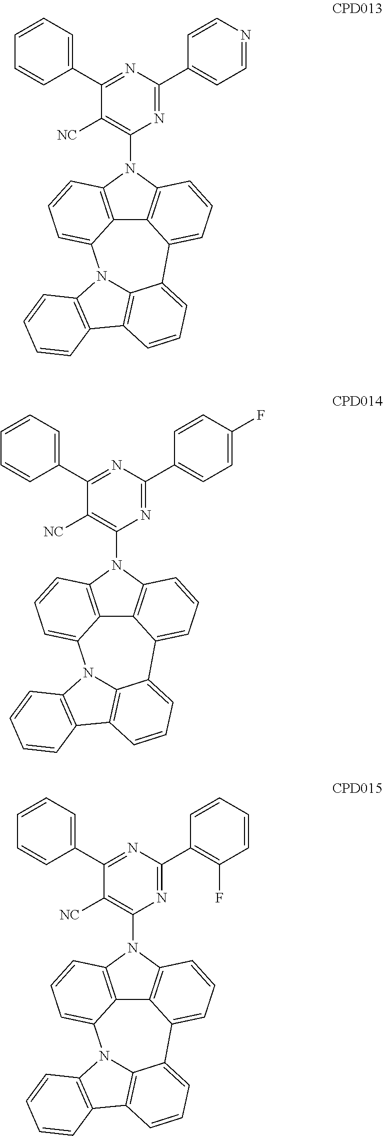 [SMILES: N#Cc1c(-c2ccccc2)nc(-c2ccncc2)nc1-n1c2cccc3c4cccc5c6ccccc6n(c6cccc1c6c32)c45 ; N#Cc1c(-c2ccccc2)nc(-c2ccc(F)cc2)nc1-n1c2cccc3c4cccc5c6ccccc6n(c6cccc1c6c32)c45 ; N#Cc1c(-c2ccccc2)nc(-c2ccccc2F)nc1-n1c2cccc3c4cccc5c6ccccc6n(c6cccc1c6c32)c45]

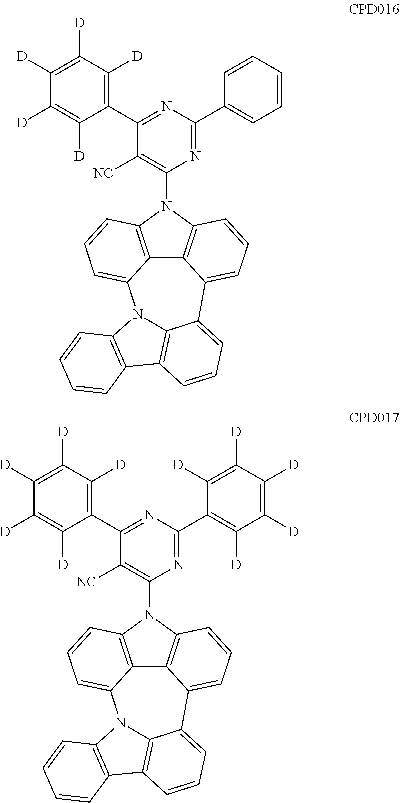

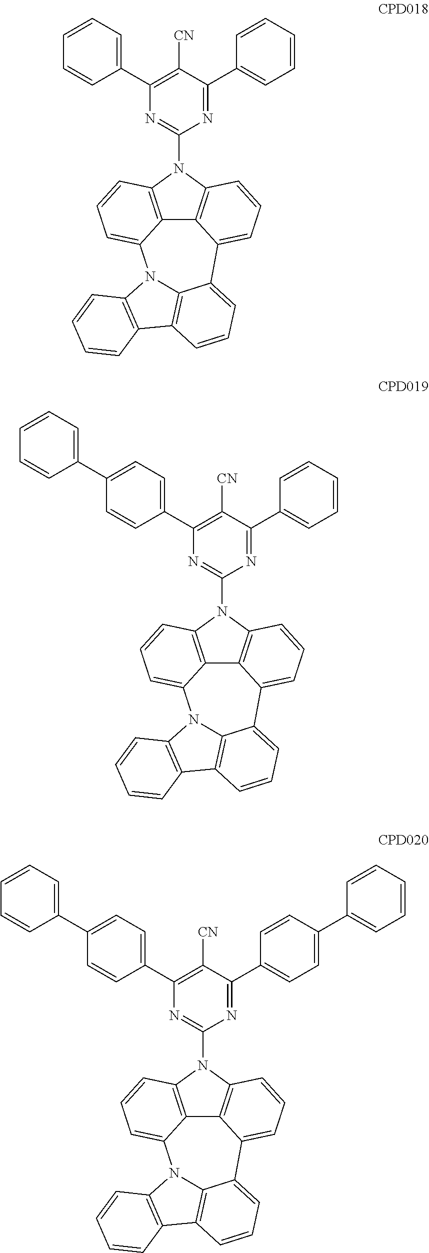 [SMILES: N#Cc1c(-c2ccccc2)nc(-n2c3cccc4c5cccc6c7ccccc7n(c7cccc2c7c43)c56)nc1-c1ccccc1 ; N#Cc1c(-c2ccccc2)nc(-n2c3cccc4c5cccc6c7ccccc7n(c7cccc2c7c43)c56)nc1-c1ccc(-c2ccccc2)cc1 ; N#Cc1c(-c2ccc(-c3ccccc3)cc2)nc(-n2c3cccc4c5cccc6c7ccccc7n(c7cccc2c7c43)c56)nc1-c1ccc(-c2ccccc2)cc1]

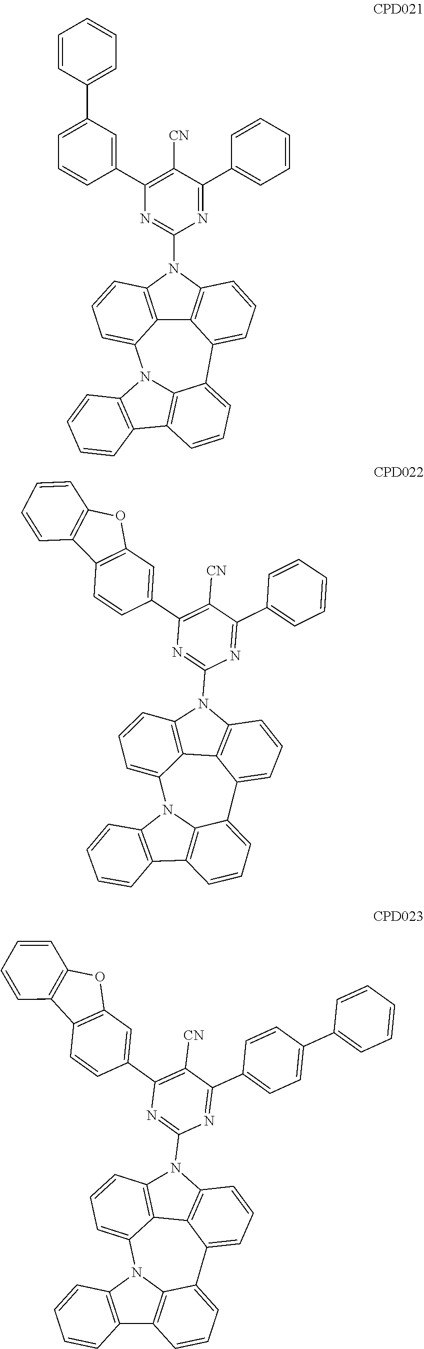

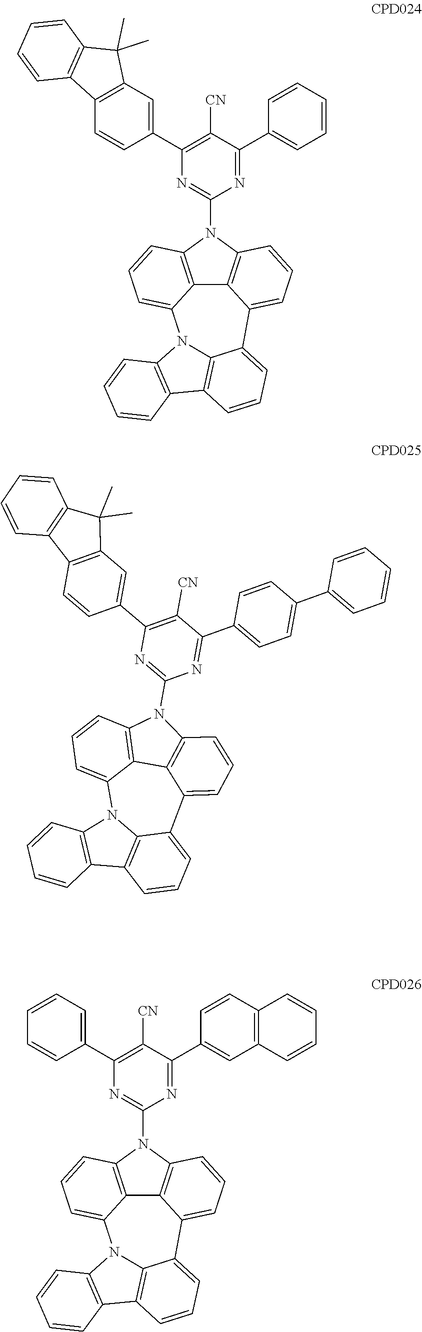 [SMILES: CC1(C)c2ccccc2-c2ccc(-c3nc(-n4c5cccc6c7cccc8c9ccccc9n(c9cccc4c9c65)c78)nc(-c4ccccc4)c3C#N)cc21 ; CC1(C)c2ccccc2-c2ccc(-c3nc(-n4c5cccc6c7cccc8c9ccccc9n(c9cccc4c9c65)c78)nc(-c4ccc(-c5ccccc5)cc4)c3C#N)cc21 ; N#Cc1c(-c2ccccc2)nc(-n2c3cccc4c5cccc6c7ccccc7n(c7cccc2c7c43)c56)nc1-c1ccc2ccccc2c1]

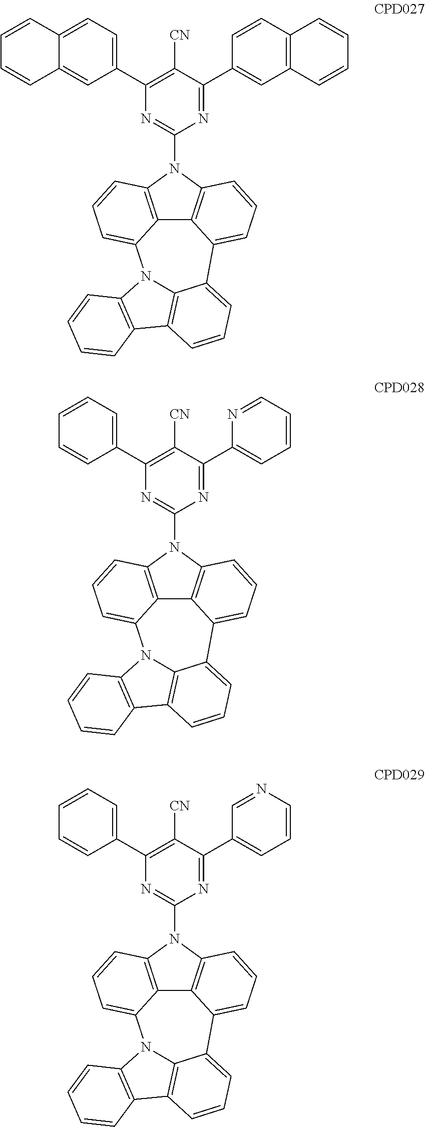

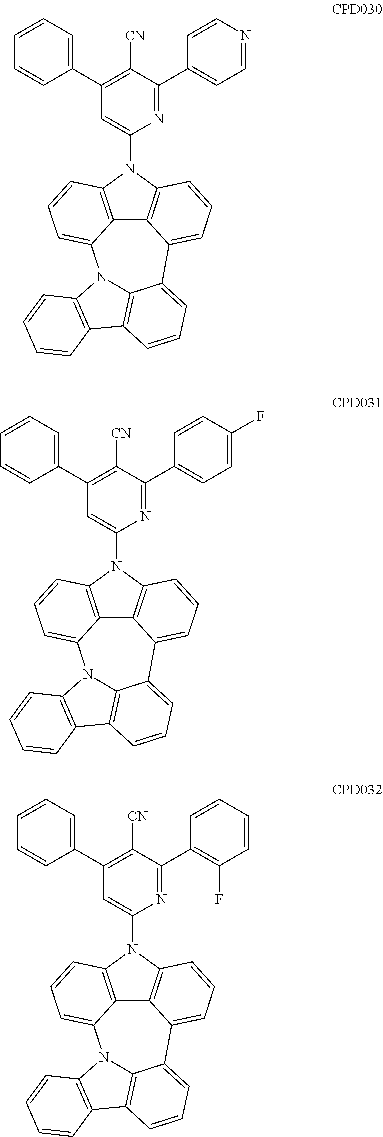 [SMILES: N#Cc1c(-c2ccccc2)cc(-n2c3cccc4c5cccc6c7ccccc7n(c7cccc2c7c43)c56)nc1-c1ccncc1 ; N#Cc1c(-c2ccccc2)cc(-n2c3cccc4c5cccc6c7ccccc7n(c7cccc2c7c43)c56)nc1-c1ccc(F)cc1 ; N#Cc1c(-c2ccccc2)cc(-n2c3cccc4c5cccc6c7ccccc7n(c7cccc2c7c43)c56)nc1-c1ccccc1F]

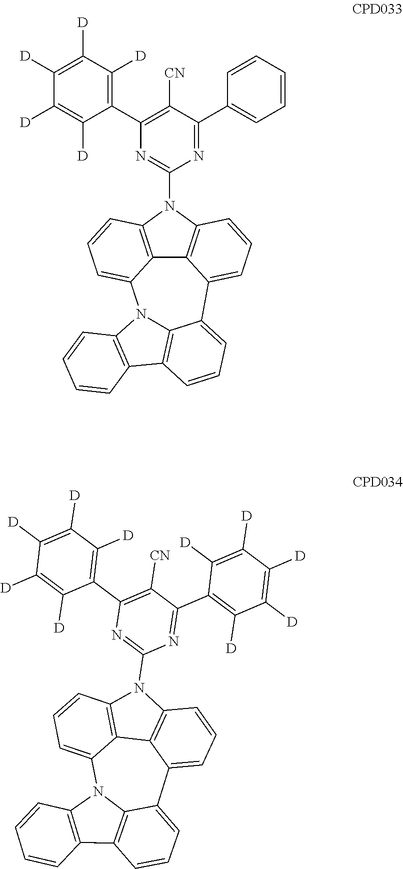 [SMILES: [2H]c1c([2H])c([2H])c(-c2nc(-n3c4cccc5c6cccc7c8ccccc8n(c8cccc3c8c54)c67)nc(-c3ccccc3)c2C#N)c([2H])c1[2H] ; [2H]c1c([2H])c([2H])c(-c2nc(-n3c4cccc5c6cccc7c8ccccc8n(c8cccc3c8c54)c67)nc(-c3c([2H])c([2H])c([2H])c([2H])c3[2H])c2C#N)c([2H])c1[2H]]

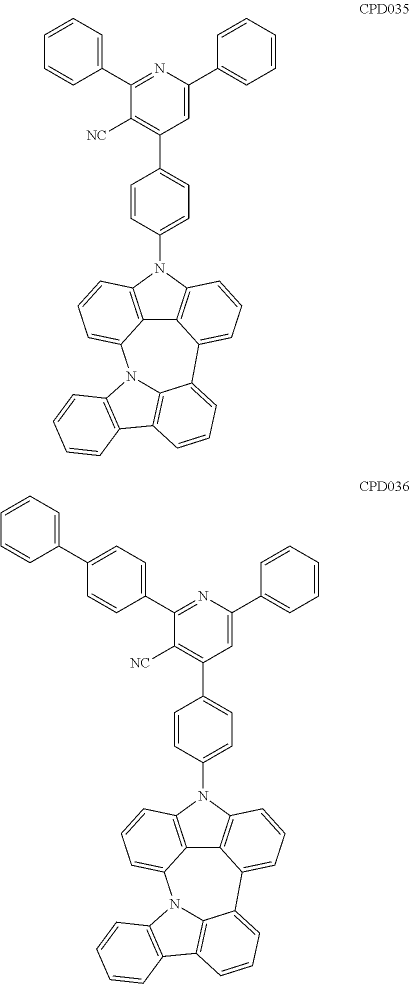 [SMILES: N#Cc1c(-c2ccc(-n3c4cccc5c6cccc7c8ccccc8n(c8cccc3c8c54)c67)cc2)cc(-c2ccccc2)nc1-c1ccccc1 ; N#Cc1c(-c2ccc(-n3c4cccc5c6cccc7c8ccccc8n(c8cccc3c8c54)c67)cc2)cc(-c2ccccc2)nc1-c1ccc(-c2ccccc2)cc1]

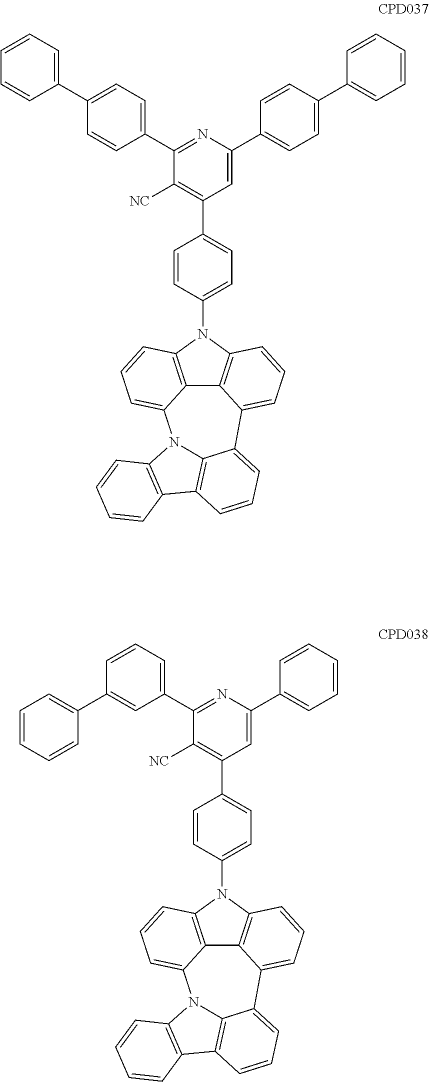 [SMILES: N#Cc1c(-c2ccc(-n3c4cccc5c6cccc7c8ccccc8n(c8cccc3c8c54)c67)cc2)cc(-c2ccc(-c3ccccc3)cc2)nc1-c1ccc(-c2ccccc2)cc1 ; N#Cc1c(-c2ccc(-n3c4cccc5c6cccc7c8ccccc8n(c8cccc3c8c54)c67)cc2)cc(-c2ccccc2)nc1-c1cccc(-c2ccccc2)c1]

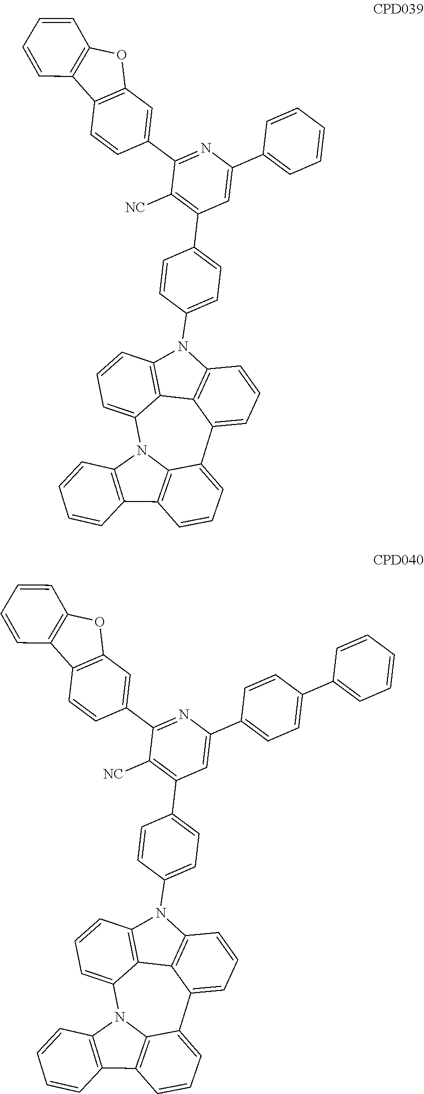 [SMILES: N#Cc1c(-c2ccc(-n3c4cccc5c6cccc7c8ccccc8n(c8cccc3c8c54)c67)cc2)cc(-c2ccccc2)nc1-c1ccc2c(c1)oc1ccccc12 ; N#Cc1c(-c2ccc(-n3c4cccc5c6cccc7c8ccccc8n(c8cccc3c8c54)c67)cc2)cc(-c2ccc(-c3ccccc3)cc2)nc1-c1ccc2c(c1)oc1ccccc12]

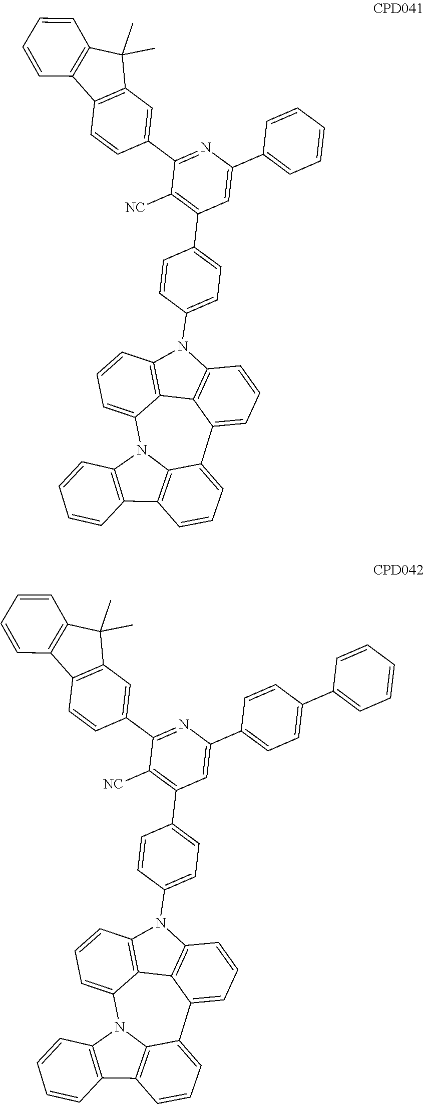 [SMILES: CC1(C)c2ccccc2-c2ccc(-c3nc(-c4ccccc4)cc(-c4ccc(-n5c6cccc7c8cccc9c%10ccccc%10n(c%10cccc5c%10c76)c89)cc4)c3C#N)cc21 ; CC1(C)c2ccccc2-c2ccc(-c3nc(-c4ccc(-c5ccccc5)cc4)cc(-c4ccc(-n5c6cccc7c8cccc9c%10ccccc%10n(c%10cccc5c%10c76)c89)cc4)c3C#N)cc21]

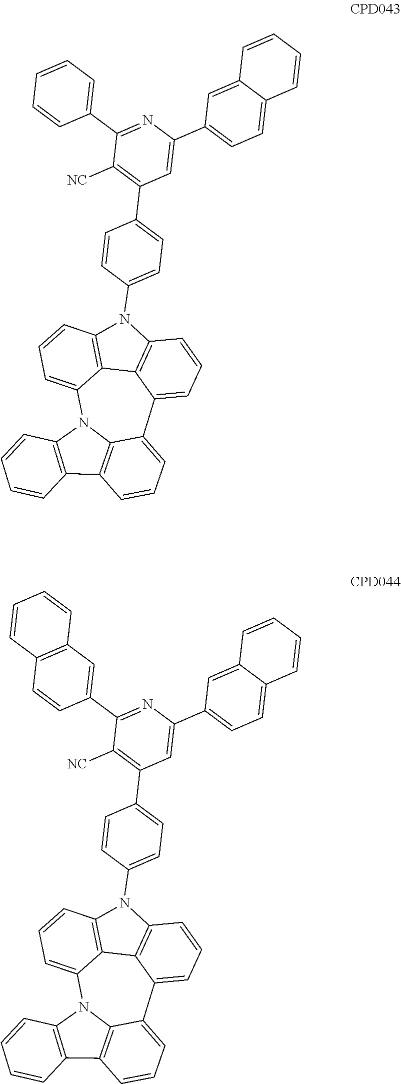 [SMILES: N#Cc1c(-c2ccc(-n3c4cccc5c6cccc7c8ccccc8n(c8cccc3c8c54)c67)cc2)cc(-c2ccc3ccccc3c2)nc1-c1ccccc1 ; N#Cc1c(-c2ccc(-n3c4cccc5c6cccc7c8ccccc8n(c8cccc3c8c54)c67)cc2)cc(-c2ccc3ccccc3c2)nc1-c1ccc2ccccc2c1]

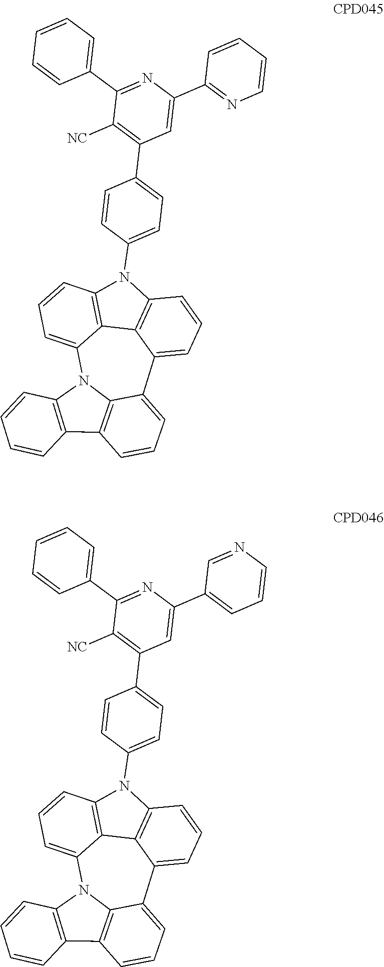 [SMILES: N#Cc1c(-c2ccc(-n3c4cccc5c6cccc7c8ccccc8n(c8cccc3c8c54)c67)cc2)cc(-c2ccccn2)nc1-c1ccccc1 ; N#Cc1c(-c2ccc(-n3c4cccc5c6cccc7c8ccccc8n(c8cccc3c8c54)c67)cc2)cc(-c2cccnc2)nc1-c1ccccc1]

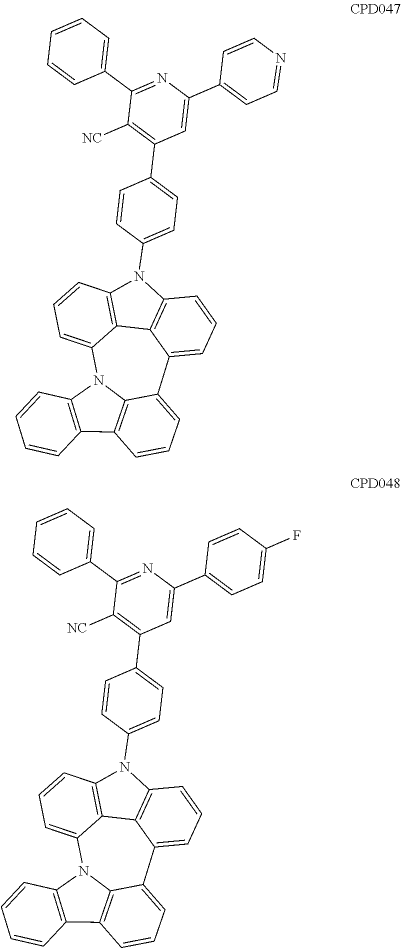 [SMILES: N#Cc1c(-c2ccc(-n3c4cccc5c6cccc7c8ccccc8n(c8cccc3c8c54)c67)cc2)cc(-c2ccncc2)nc1-c1ccccc1 ; N#Cc1c(-c2ccc(-n3c4cccc5c6cccc7c8ccccc8n(c8cccc3c8c54)c67)cc2)cc(-c2ccc(F)cc2)nc1-c1ccccc1]

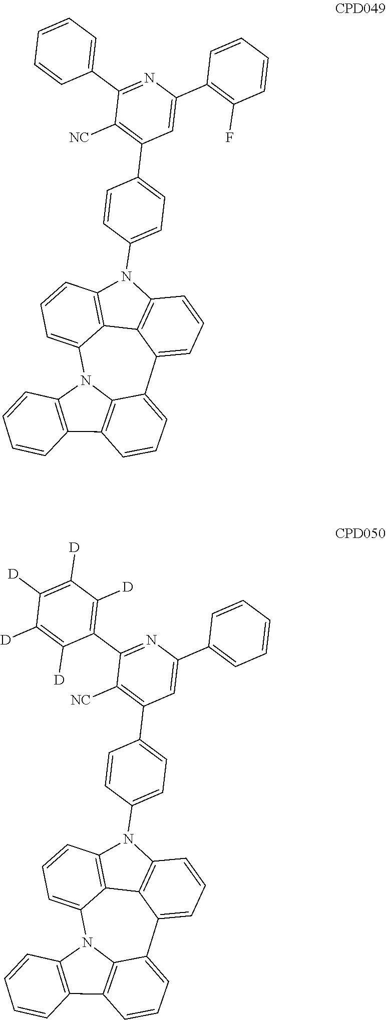 [SMILES: N#Cc1c(-c2ccc(-n3c4cccc5c6cccc7c8ccccc8n(c8cccc3c8c54)c67)cc2)cc(-c2ccccc2F)nc1-c1ccccc1 ; [2H]c1c([2H])c([2H])c(-c2nc(-c3ccccc3)cc(-c3ccc(-n4c5cccc6c7cccc8c9ccccc9n(c9cccc4c9c65)c78)cc3)c2C#N)c([2H])c1[2H]]

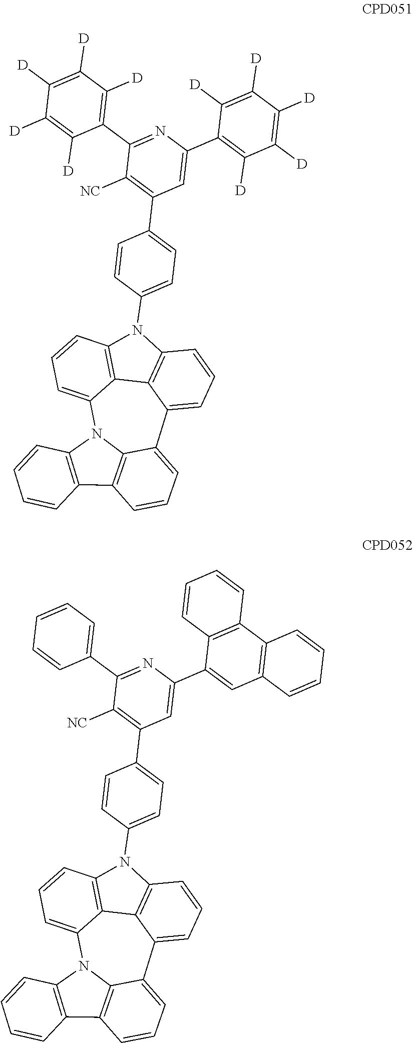 [SMILES: [2H]c1c([2H])c([2H])c(-c2cc(-c3ccc(-n4c5cccc6c7cccc8c9ccccc9n(c9cccc4c9c65)c78)cc3)c(C#N)c(-c3c([2H])c([2H])c([2H])c([2H])c3[2H])n2)c([2H])c1[2H] ; N#Cc1c(-c2ccc(-n3c4cccc5c6cccc7c8ccccc8n(c8cccc3c8c54)c67)cc2)cc(-c2cc3ccccc3c3ccccc23)nc1-c1ccccc1]

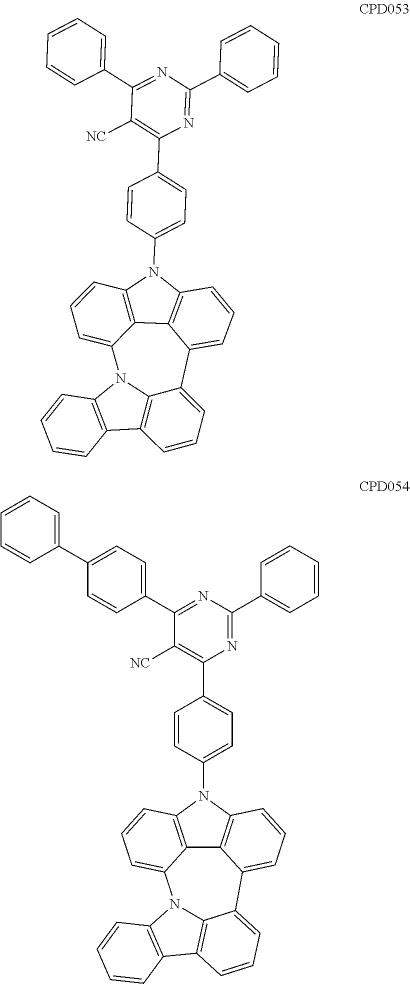 [SMILES: N#Cc1c(-c2ccccc2)nc(-c2ccccc2)nc1-c1ccc(-n2c3cccc4c5cccc6c7ccccc7n(c7cccc2c7c43)c56)cc1 ; N#Cc1c(-c2ccc(-c3ccccc3)cc2)nc(-c2ccccc2)nc1-c1ccc(-n2c3cccc4c5cccc6c7ccccc7n(c7cccc2c7c43)c56)cc1]

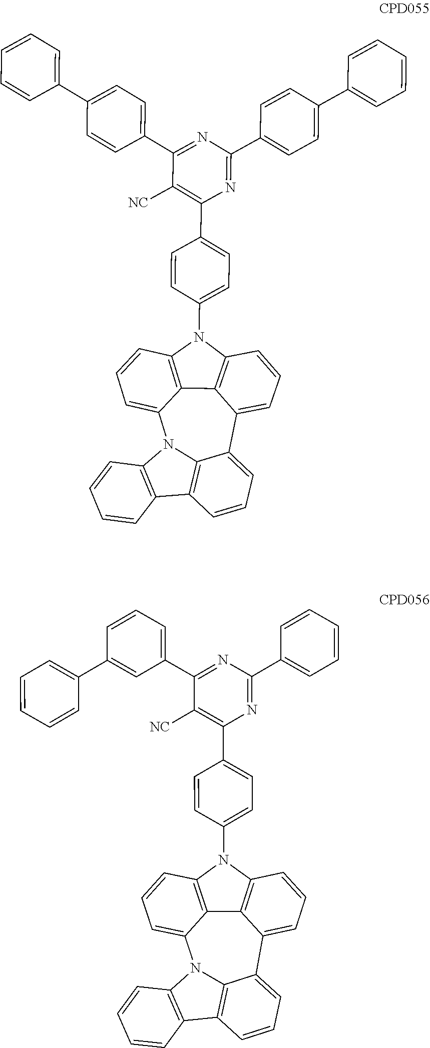 [SMILES: N#Cc1c(-c2ccc(-c3ccccc3)cc2)nc(-c2ccc(-c3ccccc3)cc2)nc1-c1ccc(-n2c3cccc4c5cccc6c7ccccc7n(c7cccc2c7c43)c56)cc1 ; N#Cc1c(-c2ccc(-n3c4cccc5c6cccc7c8ccccc8n(c8cccc3c8c54)c67)cc2)nc(-c2ccccc2)nc1-c1cccc(-c2ccccc2)c1]

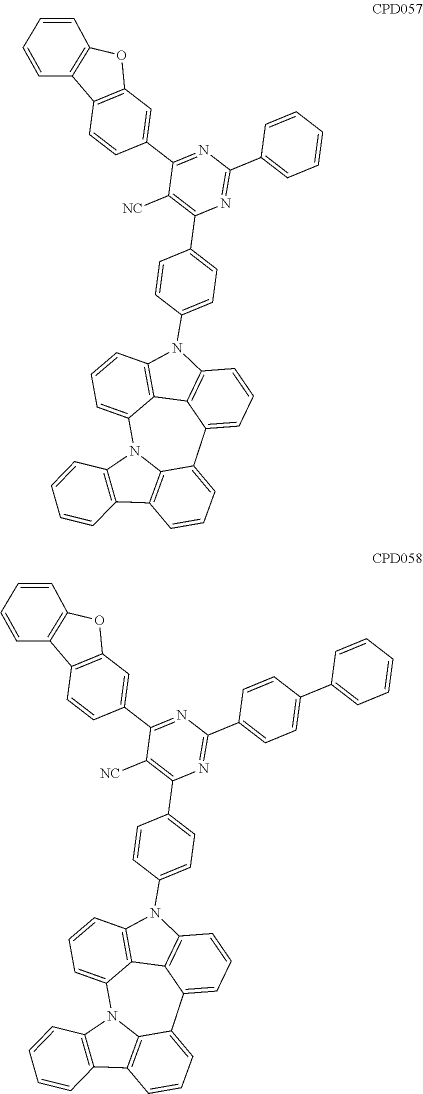 [SMILES: N#Cc1c(-c2ccc(-n3c4cccc5c6cccc7c8ccccc8n(c8cccc3c8c54)c67)cc2)nc(-c2ccccc2)nc1-c1ccc2c(c1)oc1ccccc12 ; N#Cc1c(-c2ccc(-n3c4cccc5c6cccc7c8ccccc8n(c8cccc3c8c54)c67)cc2)nc(-c2ccc(-c3ccccc3)cc2)nc1-c1ccc2c(c1)oc1ccccc12]

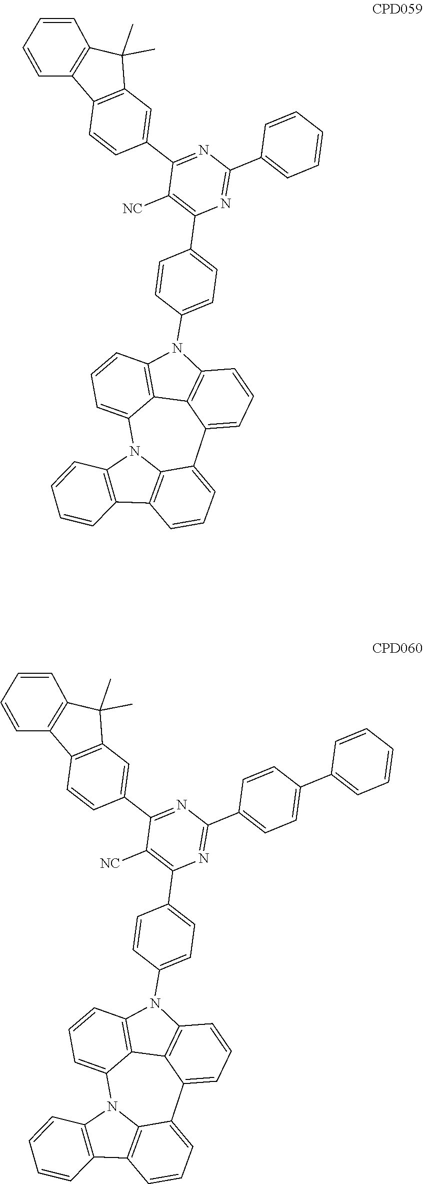 [SMILES: CC1(C)c2ccccc2-c2ccc(-c3nc(-c4ccccc4)nc(-c4ccc(-n5c6cccc7c8cccc9c%10ccccc%10n(c%10cccc5c%10c76)c89)cc4)c3C#N)cc21 ; CC1(C)c2ccccc2-c2ccc(-c3nc(-c4ccc(-c5ccccc5)cc4)nc(-c4ccc(-n5c6cccc7c8cccc9c%10ccccc%10n(c%10cccc5c%10c76)c89)cc4)c3C#N)cc21]

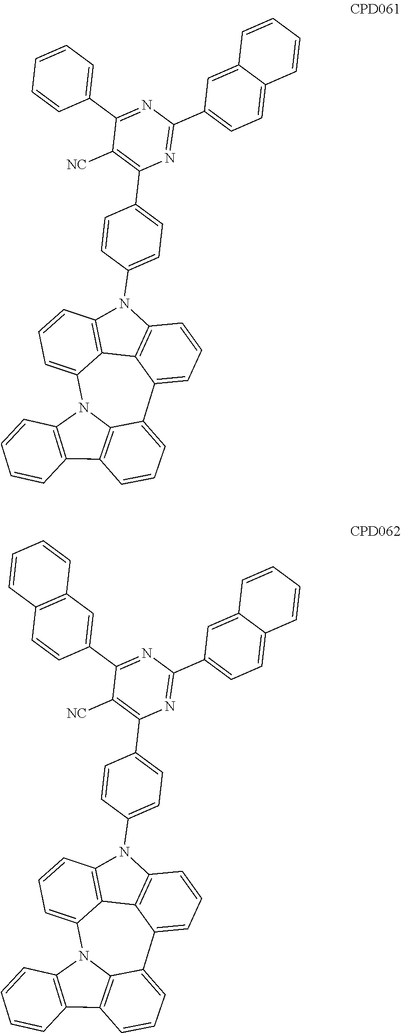 [SMILES: N#Cc1c(-c2ccccc2)nc(-c2ccc3ccccc3c2)nc1-c1ccc(-n2c3cccc4c5cccc6c7ccccc7n(c7cccc2c7c43)c56)cc1 ; N#Cc1c(-c2ccc(-n3c4cccc5c6cccc7c8ccccc8n(c8cccc3c8c54)c67)cc2)nc(-c2ccc3ccccc3c2)nc1-c1ccc2ccccc2c1]

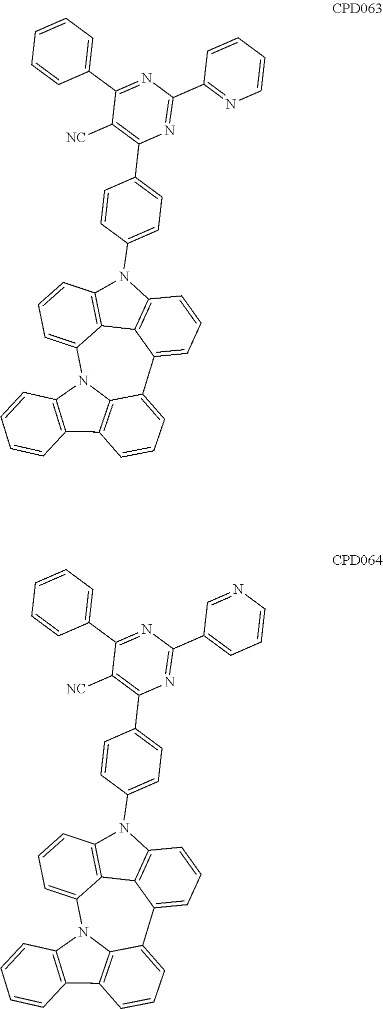 [SMILES: N#Cc1c(-c2ccccc2)nc(-c2ccccn2)nc1-c1ccc(-n2c3cccc4c5cccc6c7ccccc7n(c7cccc2c7c43)c56)cc1 ; N#Cc1c(-c2ccccc2)nc(-c2cccnc2)nc1-c1ccc(-n2c3cccc4c5cccc6c7ccccc7n(c7cccc2c7c43)c56)cc1]

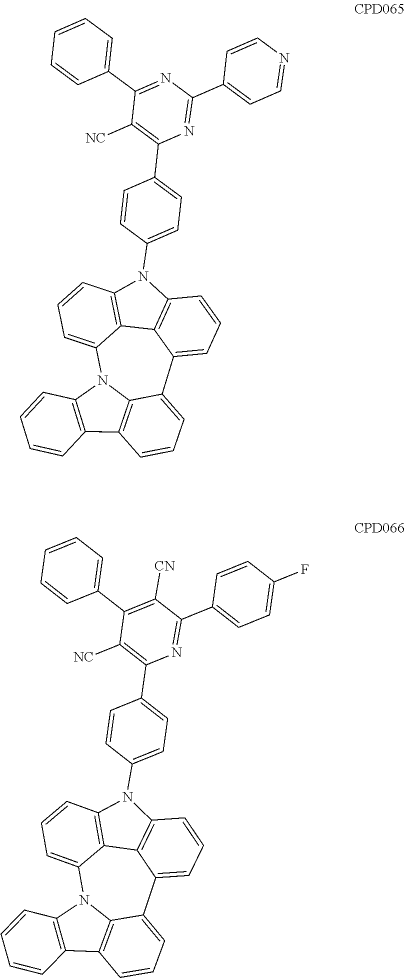 [SMILES: N#Cc1c(-c2ccccc2)nc(-c2ccncc2)nc1-c1ccc(-n2c3cccc4c5cccc6c7ccccc7n(c7cccc2c7c43)c56)cc1 ; N#Cc1c(-c2ccc(F)cc2)nc(-c2ccc(-n3c4cccc5c6cccc7c8ccccc8n(c8cccc3c8c54)c67)cc2)c(C#N)c1-c1ccccc1]

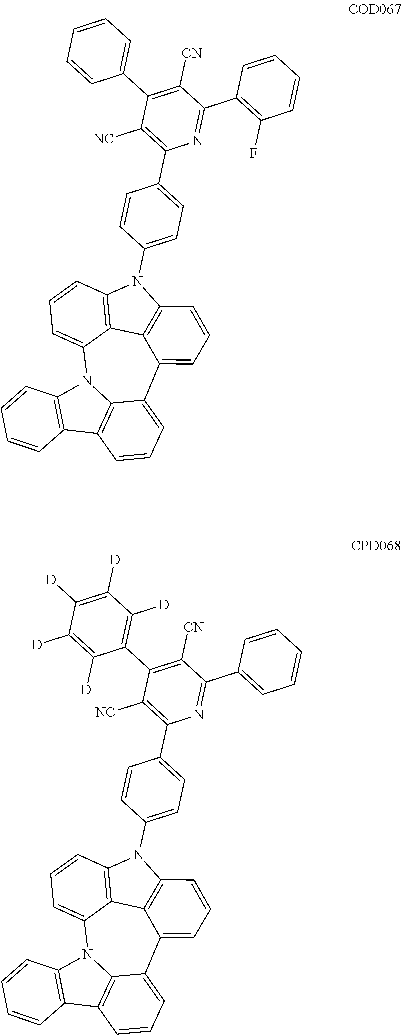 [SMILES: N#Cc1c(-c2ccc(-n3c4cccc5c6cccc7c8ccccc8n(c8cccc3c8c54)c67)cc2)nc(-c2ccccc2F)c(C#N)c1-c1ccccc1 ; [2H]c1c([2H])c([2H])c(-c2c(C#N)c(-c3ccccc3)nc(-c3ccc(-n4c5cccc6c7cccc8c9ccccc9n(c9cccc4c9c65)c78)cc3)c2C#N)c([2H])c1[2H]]

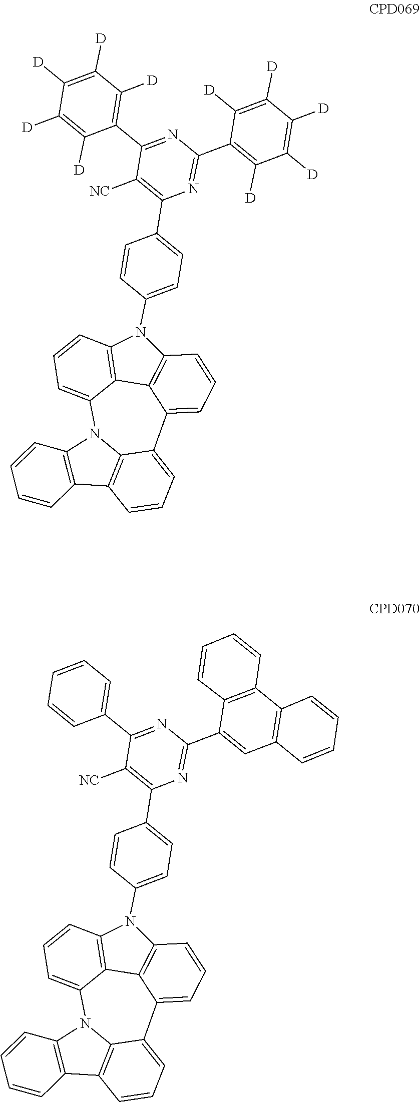 [SMILES: [2H]c1c([2H])c([2H])c(-c2nc(-c3ccc(-n4c5cccc6c7cccc8c9ccccc9n(c9cccc4c9c65)c78)cc3)c(C#N)c(-c3c([2H])c([2H])c([2H])c([2H])c3[2H])n2)c([2H])c1[2H] ; N#Cc1c(-c2ccccc2)nc(-c2cc3ccccc3c3ccccc23)nc1-c1ccc(-n2c3cccc4c5cccc6c7ccccc7n(c7cccc2c7c43)c56)cc1]

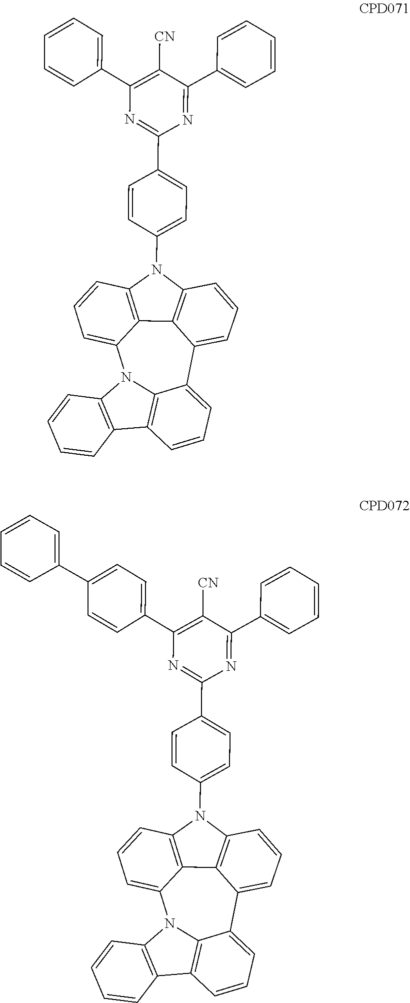 [SMILES: N#Cc1c(-c2ccccc2)nc(-c2ccc(-n3c4cccc5c6cccc7c8ccccc8n(c8cccc3c8c54)c67)cc2)nc1-c1ccccc1 ; N#Cc1c(-c2ccccc2)nc(-c2ccc(-n3c4cccc5c6cccc7c8ccccc8n(c8cccc3c8c54)c67)cc2)nc1-c1ccc(-c2ccccc2)cc1]

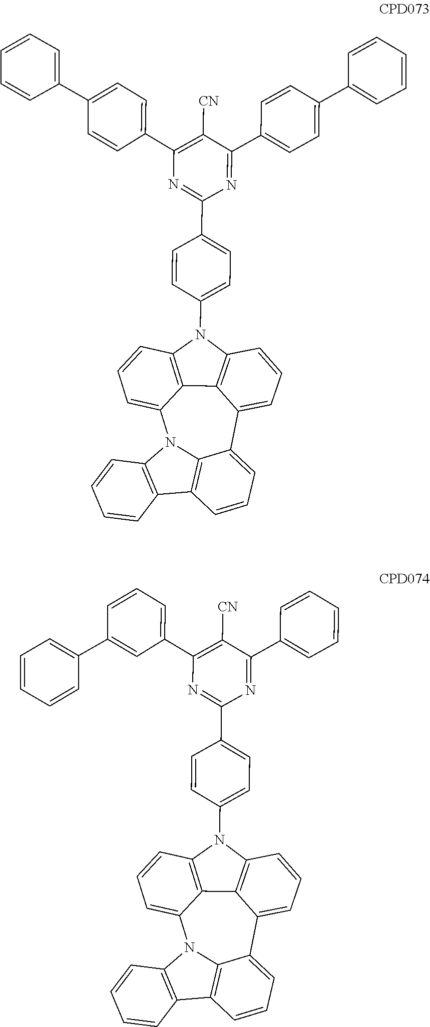 [SMILES: N#Cc1c(-c2ccc(-c3ccccc3)cc2)nc(-c2ccc(-n3c4cccc5c6cccc7c8ccccc8n(c8cccc3c8c54)c67)cc2)nc1-c1ccc(-c2ccccc2)cc1 ; N#Cc1c(-c2ccccc2)nc(-c2ccc(-n3c4cccc5c6cccc7c8ccccc8n(c8cccc3c8c54)c67)cc2)nc1-c1cccc(-c2ccccc2)c1]

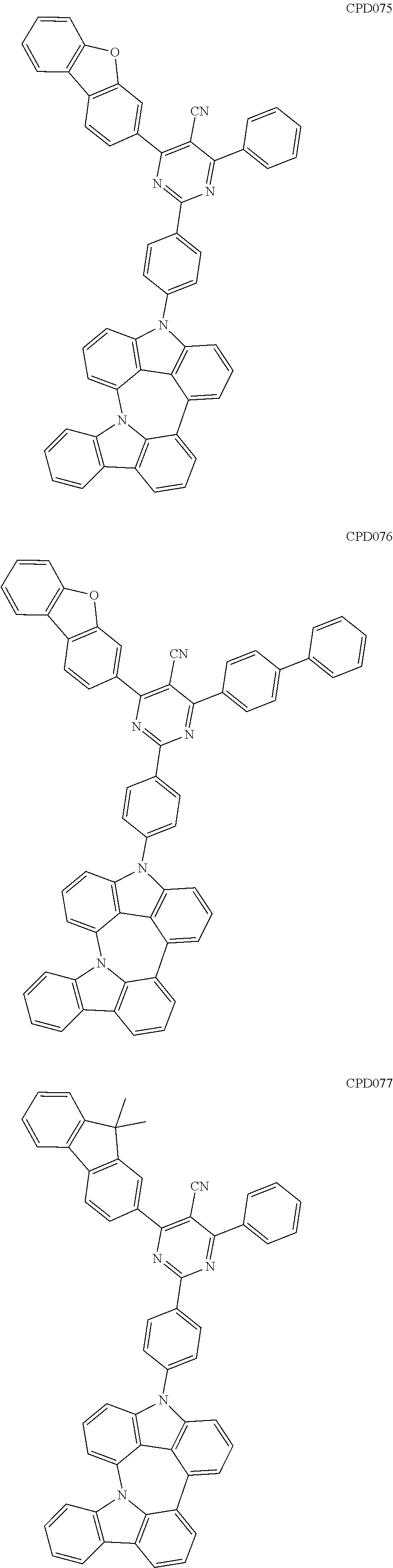 [SMILES: N#Cc1c(-c2ccccc2)nc(-c2ccc(-n3c4cccc5c6cccc7c8ccccc8n(c8cccc3c8c54)c67)cc2)nc1-c1ccc2c(c1)oc1ccccc12 ; N#Cc1c(-c2ccc(-c3ccccc3)cc2)nc(-c2ccc(-n3c4cccc5c6cccc7c8ccccc8n(c8cccc3c8c54)c67)cc2)nc1-c1ccc2c(c1)oc1ccccc12 ; CC1(C)c2ccccc2-c2ccc(-c3nc(-c4ccc(-n5c6cccc7c8cccc9c%10ccccc%10n(c%10cccc5c%10c76)c89)cc4)nc(-c4ccccc4)c3C#N)cc21]

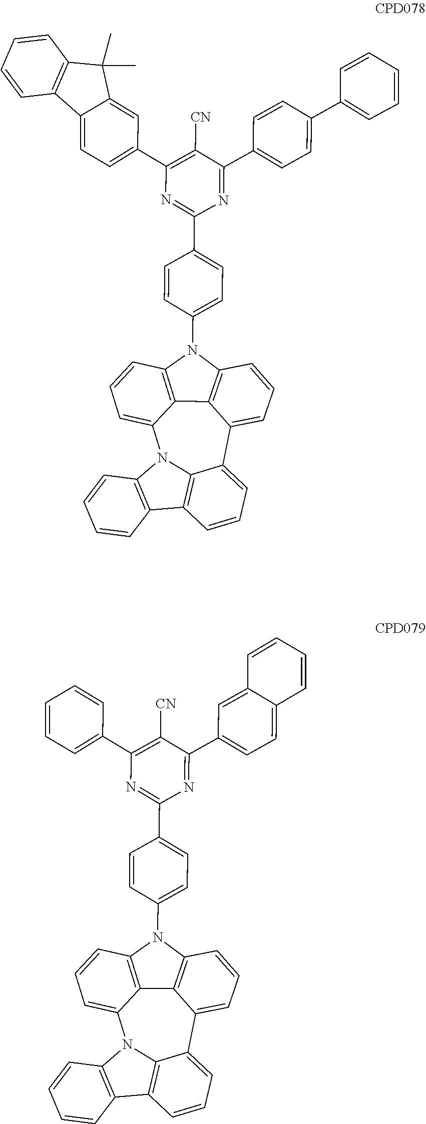 [SMILES: CC1(C)c2ccccc2-c2ccc(-c3nc(-c4ccc(-n5c6cccc7c8cccc9c%10ccccc%10n(c%10cccc5c%10c76)c89)cc4)nc(-c4ccc(-c5ccccc5)cc4)c3C#N)cc21 ; N#Cc1c(-c2ccccc2)nc(-c2ccc(-n3c4cccc5c6cccc7c8ccccc8n(c8cccc3c8c54)c67)cc2)nc1-c1ccc2ccccc2c1]

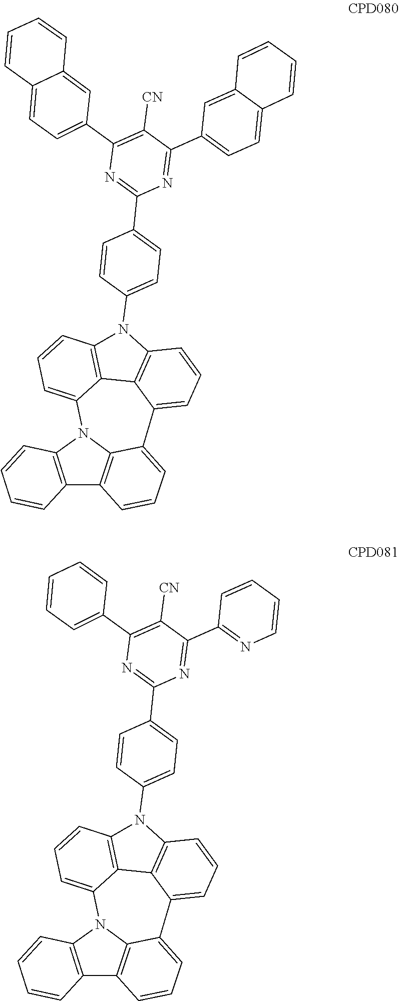 [SMILES: N#Cc1c(-c2ccc3ccccc3c2)nc(-c2ccc(-n3c4cccc5c6cccc7c8ccccc8n(c8cccc3c8c54)c67)cc2)nc1-c1ccc2ccccc2c1 ; N#Cc1c(-c2ccccc2)nc(-c2ccc(-n3c4cccc5c6cccc7c8ccccc8n(c8cccc3c8c54)c67)cc2)nc1-c1ccccn1]

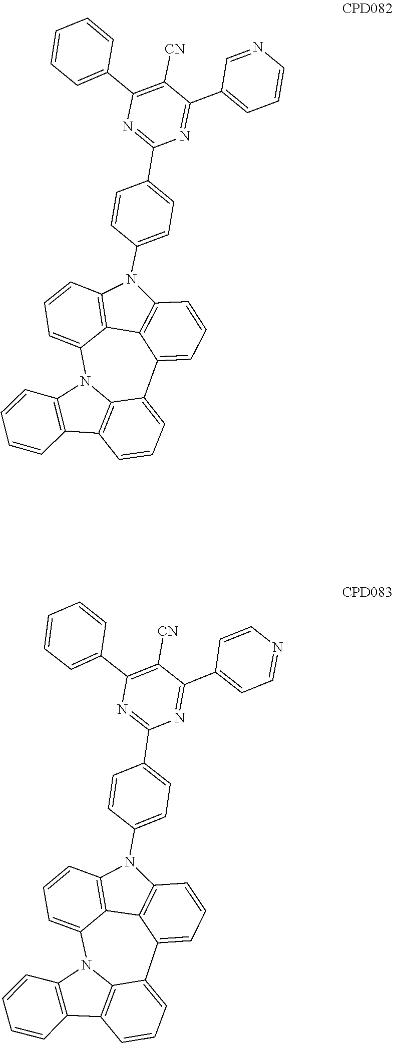 [SMILES: N#Cc1c(-c2ccccc2)nc(-c2ccc(-n3c4cccc5c6cccc7c8ccccc8n(c8cccc3c8c54)c67)cc2)nc1-c1cccnc1 ; N#Cc1c(-c2ccccc2)nc(-c2ccc(-n3c4cccc5c6cccc7c8ccccc8n(c8cccc3c8c54)c67)cc2)nc1-c1ccncc1]

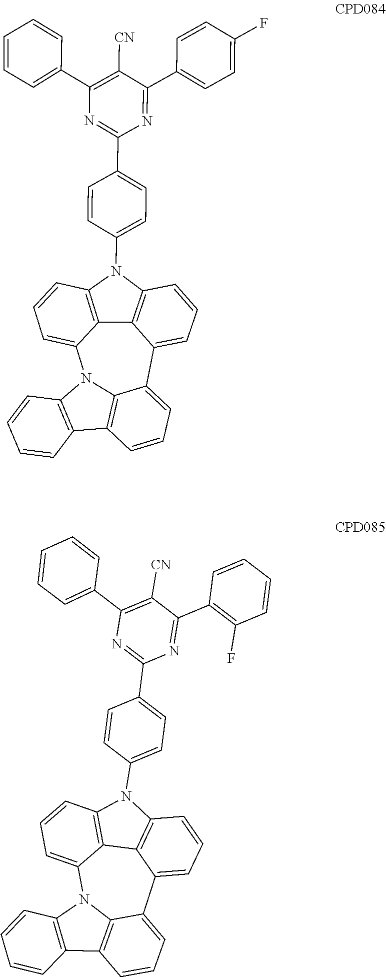 [SMILES: N#Cc1c(-c2ccccc2)nc(-c2ccc(-n3c4cccc5c6cccc7c8ccccc8n(c8cccc3c8c54)c67)cc2)nc1-c1ccc(F)cc1 ; N#Cc1c(-c2ccccc2)nc(-c2ccc(-n3c4cccc5c6cccc7c8ccccc8n(c8cccc3c8c54)c67)cc2)nc1-c1ccccc1F]

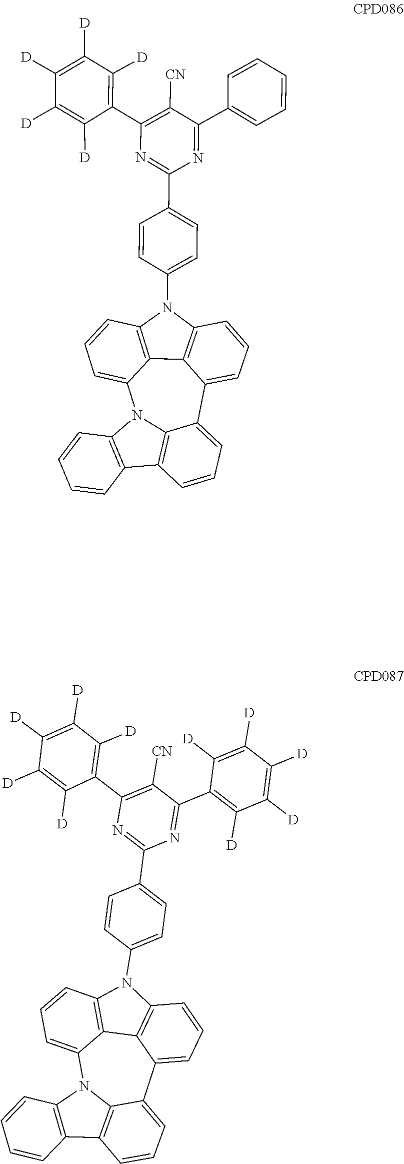 [SMILES: [2H]c1c([2H])c([2H])c(-c2nc(-c3ccc(-n4c5cccc6c7cccc8c9ccccc9n(c9cccc4c9c65)c78)cc3)nc(-c3ccccc3)c2C#N)c([2H])c1[2H] ; [2H]c1c([2H])c([2H])c(-c2nc(-c3ccc(-n4c5cccc6c7cccc8c9ccccc9n(c9cccc4c9c65)c78)cc3)nc(-c3c([2H])c([2H])c([2H])c([2H])c3[2H])c2C#N)c([2H])c1[2H]]

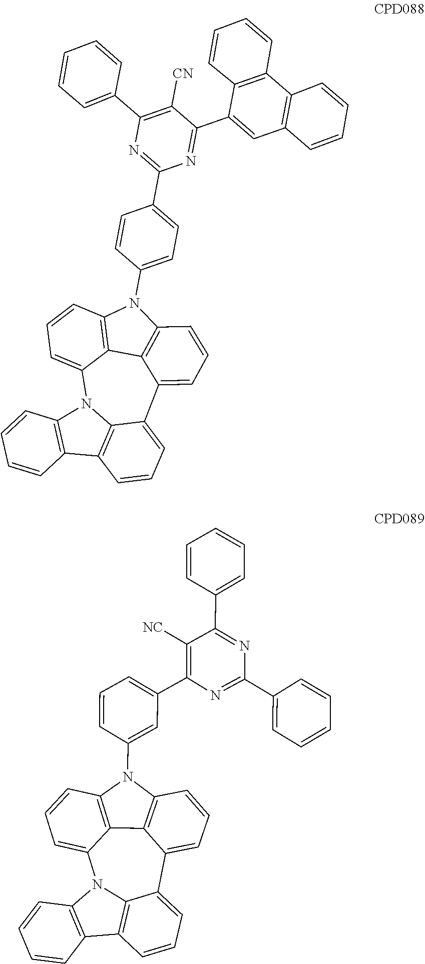 [SMILES: N#Cc1c(-c2ccccc2)nc(-c2ccc(-n3c4cccc5c6cccc7c8ccccc8n(c8cccc3c8c54)c67)cc2)nc1-c1cc2ccccc2c2ccccc12 ; N#Cc1c(-c2ccccc2)nc(-c2ccccc2)nc1-c1cccc(-n2c3cccc4c5cccc6c7ccccc7n(c7cccc2c7c43)c56)c1]

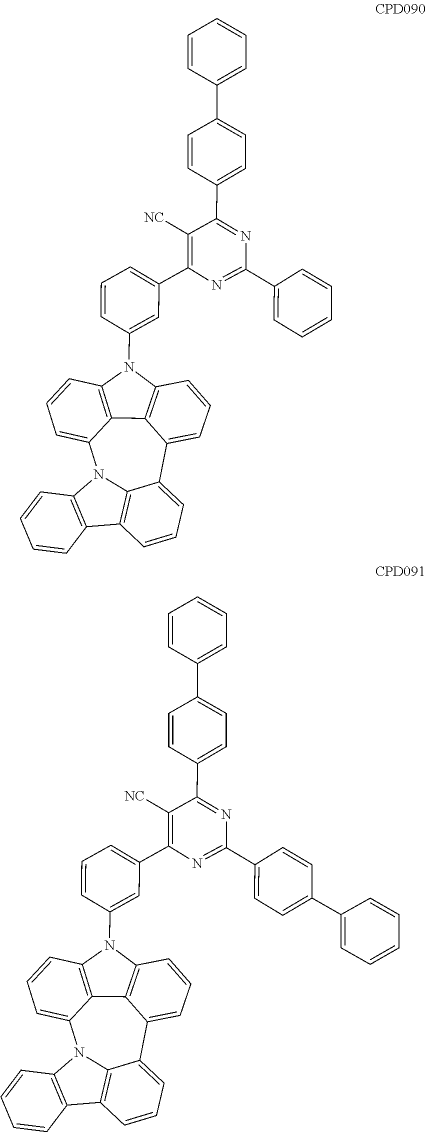 [SMILES: N#Cc1c(-c2ccc(-c3ccccc3)cc2)nc(-c2ccccc2)nc1-c1cccc(-n2c3cccc4c5cccc6c7ccccc7n(c7cccc2c7c43)c56)c1 ; N#Cc1c(-c2ccc(-c3ccccc3)cc2)nc(-c2ccc(-c3ccccc3)cc2)nc1-c1cccc(-n2c3cccc4c5cccc6c7ccccc7n(c7cccc2c7c43)c56)c1]

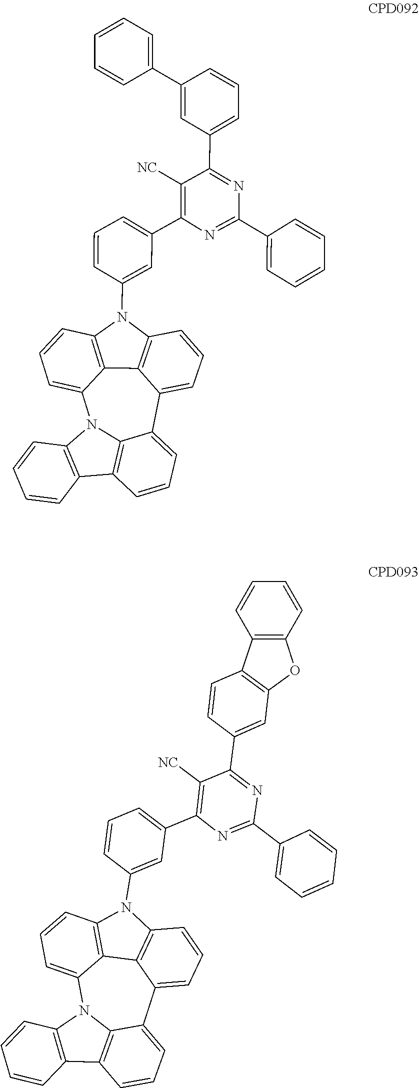 [SMILES: N#Cc1c(-c2cccc(-c3ccccc3)c2)nc(-c2ccccc2)nc1-c1cccc(-n2c3cccc4c5cccc6c7ccccc7n(c7cccc2c7c43)c56)c1 ; N#Cc1c(-c2cccc(-n3c4cccc5c6cccc7c8ccccc8n(c8cccc3c8c54)c67)c2)nc(-c2ccccc2)nc1-c1ccc2c(c1)oc1ccccc12]

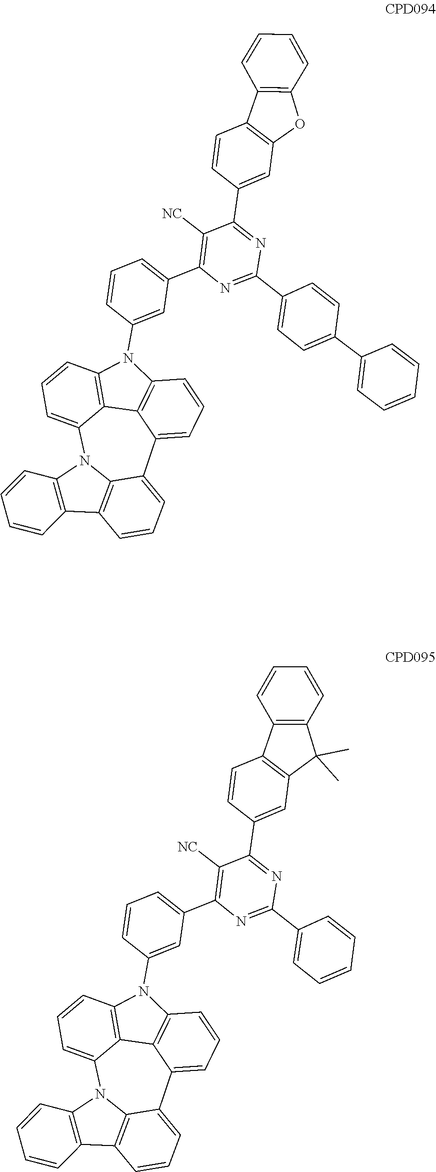 [SMILES: N#Cc1c(-c2cccc(-n3c4cccc5c6cccc7c8ccccc8n(c8cccc3c8c54)c67)c2)nc(-c2ccc(-c3ccccc3)cc2)nc1-c1ccc2c(c1)oc1ccccc12 ; CC1(C)c2ccccc2-c2ccc(-c3nc(-c4ccccc4)nc(-c4cccc(-n5c6cccc7c8cccc9c%10ccccc%10n(c%10cccc5c%10c76)c89)c4)c3C#N)cc21]

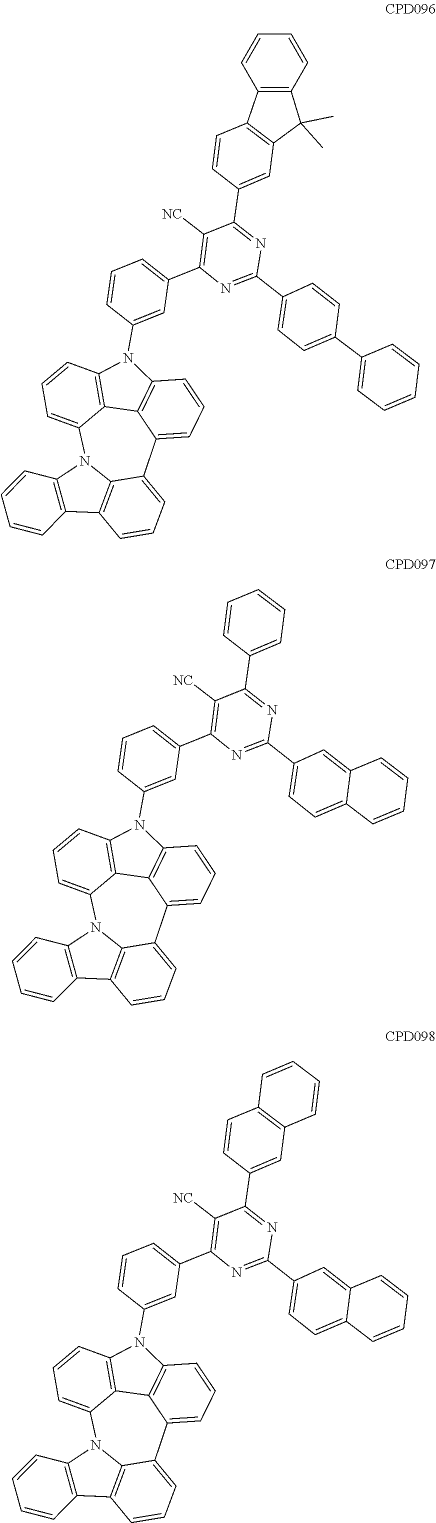 [SMILES: CC1(C)c2ccccc2-c2ccc(-c3nc(-c4ccc(-c5ccccc5)cc4)nc(-c4cccc(-n5c6cccc7c8cccc9c%10ccccc%10n(c%10cccc5c%10c76)c89)c4)c3C#N)cc21 ; N#Cc1c(-c2ccccc2)nc(-c2ccc3ccccc3c2)nc1-c1cccc(-n2c3cccc4c5cccc6c7ccccc7n(c7cccc2c7c43)c56)c1 ; N#Cc1c(-c2cccc(-n3c4cccc5c6cccc7c8ccccc8n(c8cccc3c8c54)c67)c2)nc(-c2ccc3ccccc3c2)nc1-c1ccc2ccccc2c1]

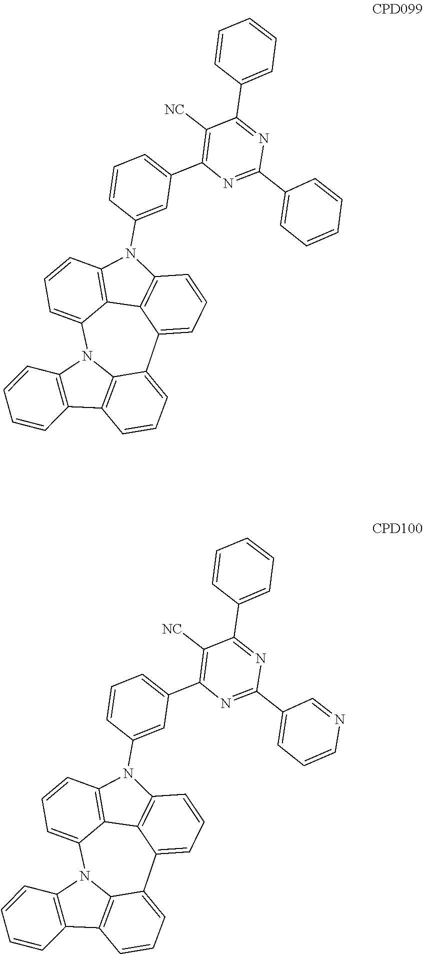 [SMILES: N#Cc1c(-c2ccccc2)nc(-c2ccccc2)nc1-c1cccc(-n2c3cccc4c5cccc6c7ccccc7n(c7cccc2c7c43)c56)c1 ; N#Cc1c(-c2ccccc2)nc(-c2cccnc2)nc1-c1cccc(-n2c3cccc4c5cccc6c7ccccc7n(c7cccc2c7c43)c56)c1]

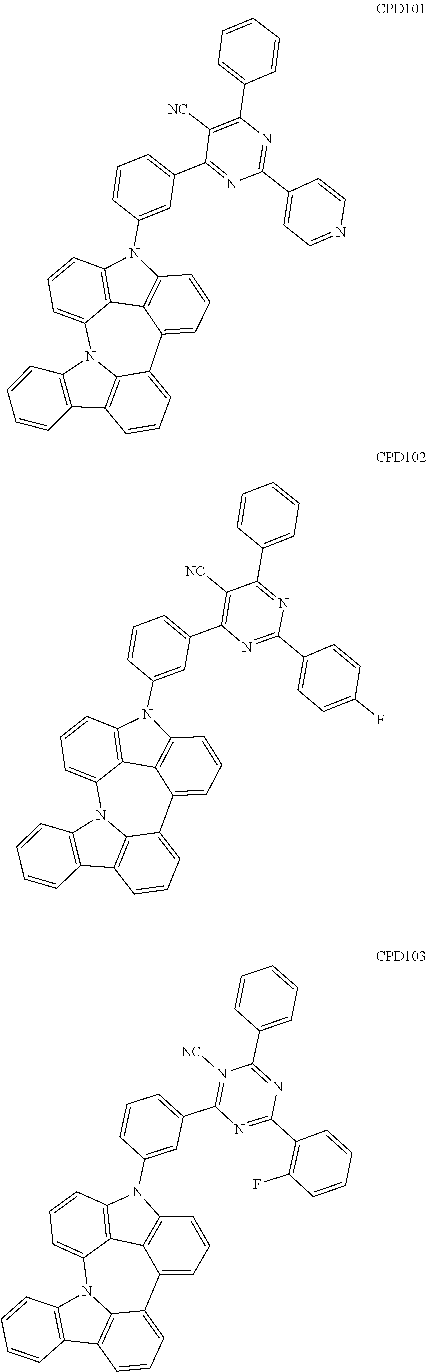

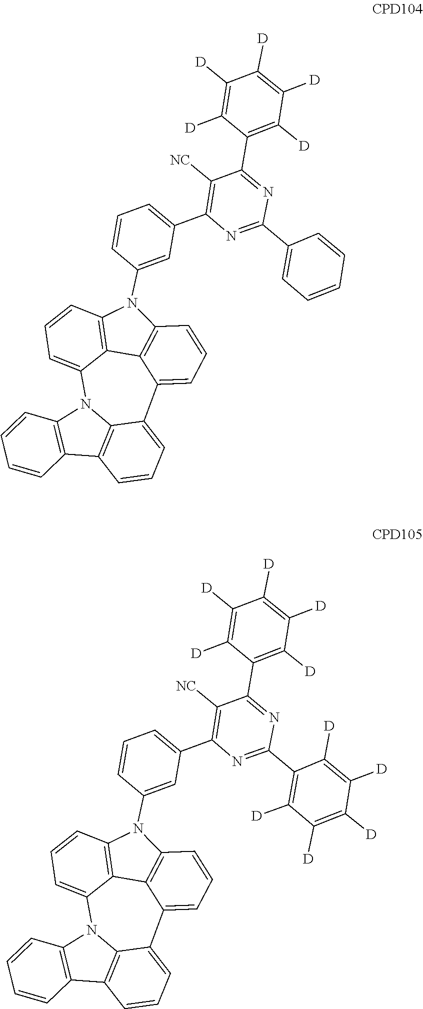 [SMILES: [2H]c1c([2H])c([2H])c(-c2nc(-c3ccccc3)nc(-c3cccc(-n4c5cccc6c7cccc8c9ccccc9n(c9cccc4c9c65)c78)c3)c2C#N)c([2H])c1[2H] ; [2H]c1c([2H])c([2H])c(-c2nc(-c3cccc(-n4c5cccc6c7cccc8c9ccccc9n(c9cccc4c9c65)c78)c3)c(C#N)c(-c3c([2H])c([2H])c([2H])c([2H])c3[2H])n2)c([2H])c1[2H]]

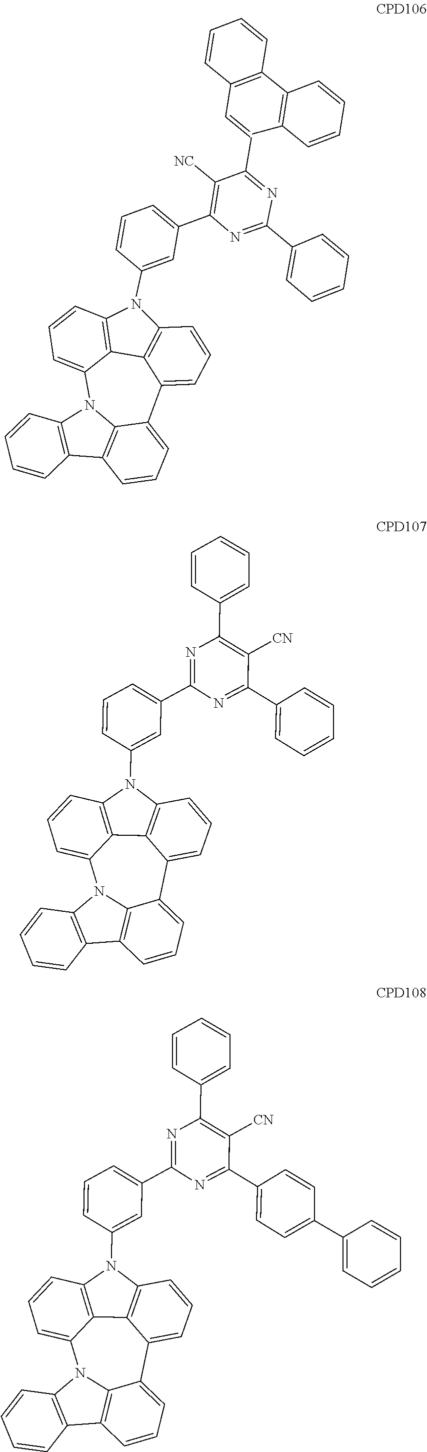 [SMILES: Cc1ccccc1-c1ccccc1/C=C/c1nc(-c2ccccc2)nc(-c2cccc(-n3c4cccc5c6cccc7c8ccccc8n(c8cccc3c8c54)c67)c2)c1C#N ; N#Cc1c(-c2ccccc2)nc(-c2cccc(-n3c4cccc5c6cccc7c8ccccc8n(c8cccc3c8c54)c67)c2)nc1-c1ccccc1 ; N#Cc1c(-c2ccccc2)nc(-c2cccc(-n3c4cccc5c6cccc7c8ccccc8n(c8cccc3c8c54)c67)c2)nc1-c1ccc(-c2ccccc2)cc1]

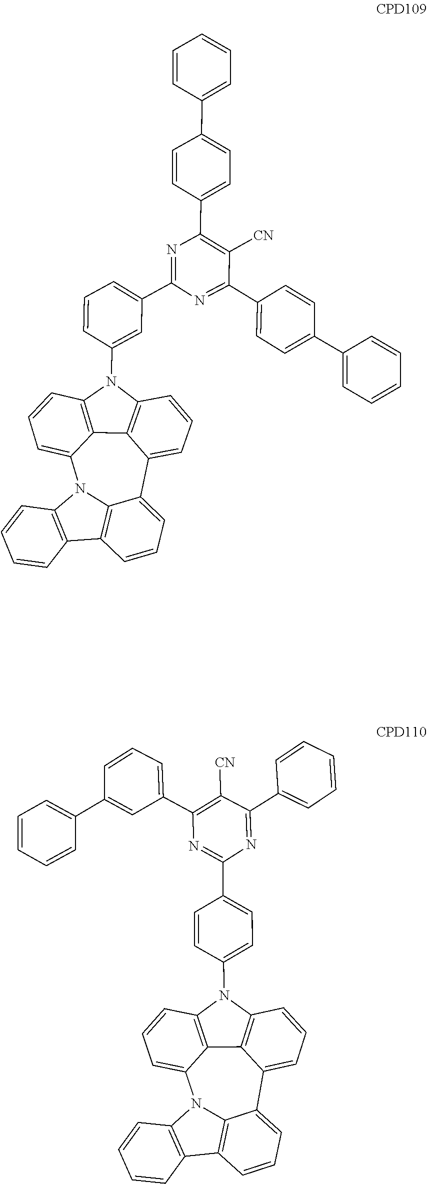 [SMILES: N#Cc1c(-c2ccc(-c3ccccc3)cc2)nc(-c2cccc(-n3c4cccc5c6cccc7c8ccccc8n(c8cccc3c8c54)c67)c2)nc1-c1ccc(-c2ccccc2)cc1 ; N#Cc1c(-c2ccccc2)nc(-c2ccc(-n3c4cccc5c6cccc7c8ccccc8n(c8cccc3c8c54)c67)cc2)nc1-c1cccc(-c2ccccc2)c1]

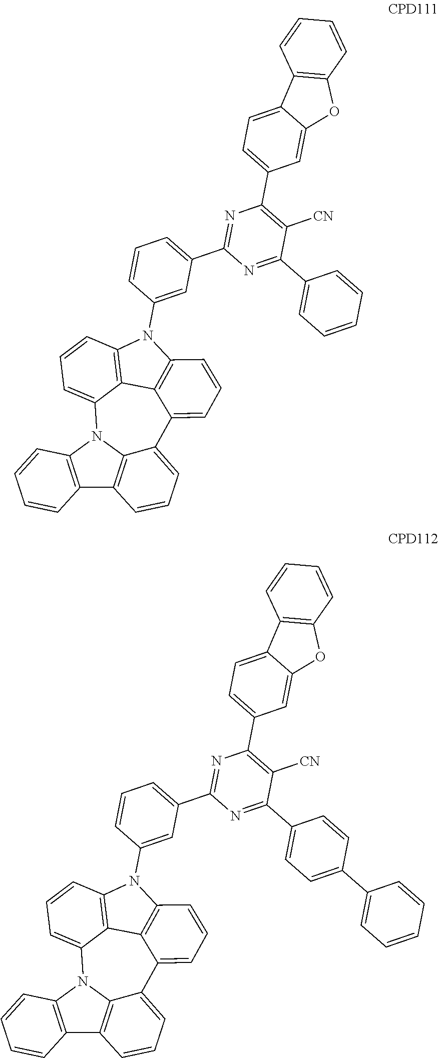 [SMILES: N#Cc1c(-c2ccccc2)nc(-c2cccc(-n3c4cccc5c6cccc7c8ccccc8n(c8cccc3c8c54)c67)c2)nc1-c1ccc2c(c1)oc1ccccc12 ; N#Cc1c(-c2ccc(-c3ccccc3)cc2)nc(-c2cccc(-n3c4cccc5c6cccc7c8ccccc8n(c8cccc3c8c54)c67)c2)nc1-c1ccc2c(c1)oc1ccccc12]

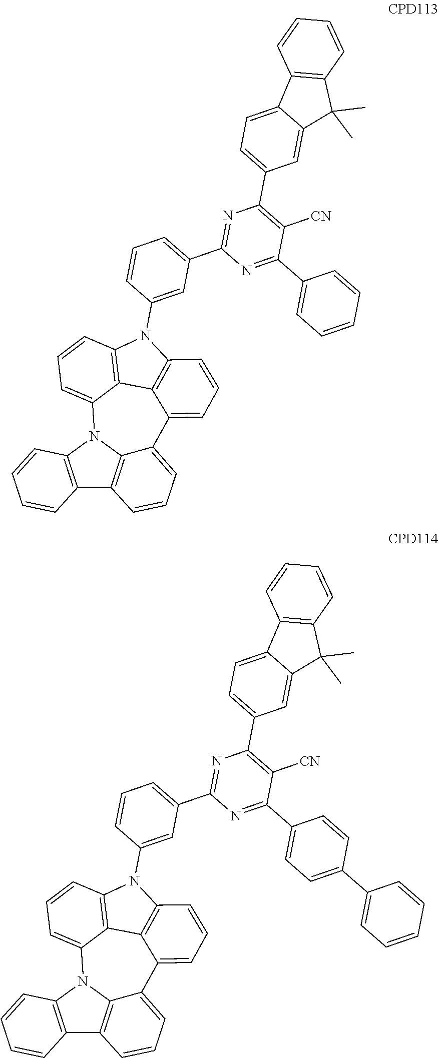 [SMILES: CC1(C)c2ccccc2-c2ccc(-c3nc(-c4cccc(-n5c6cccc7c8cccc9c%10ccccc%10n(c%10cccc5c%10c76)c89)c4)nc(-c4ccccc4)c3C#N)cc21 ; CC1(C)c2ccccc2-c2ccc(-c3nc(-c4cccc(-n5c6cccc7c8cccc9c%10ccccc%10n(c%10cccc5c%10c76)c89)c4)nc(-c4ccc(-c5ccccc5)cc4)c3C#N)cc21]

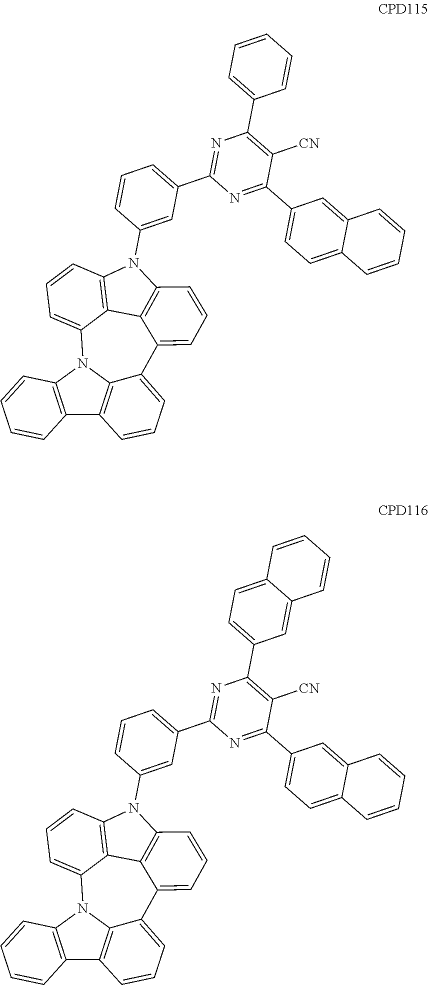 [SMILES: N#Cc1c(-c2ccccc2)nc(-c2cccc(-n3c4cccc5c6cccc7c8ccccc8n(c8cccc3c8c54)c67)c2)nc1-c1ccc2ccccc2c1 ; N#Cc1c(-c2ccc3ccccc3c2)nc(-c2cccc(-n3c4cccc5c6cccc7c8ccccc8n(c8cccc3c8c54)c67)c2)nc1-c1ccc2ccccc2c1]

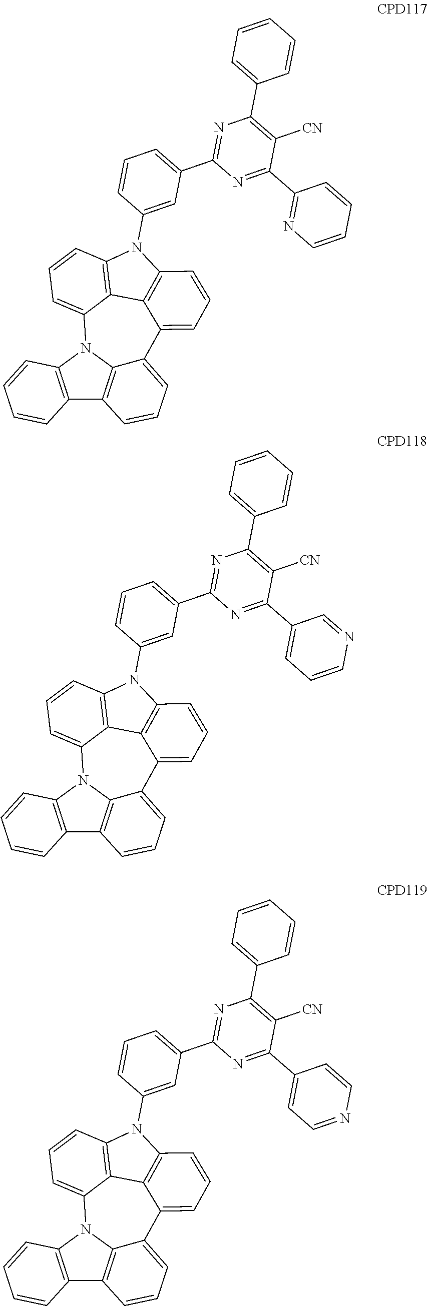 [SMILES: N#Cc1c(-c2ccccc2)nc(-c2cccc(-n3c4cccc5c6cccc7c8ccccc8n(c8cccc3c8c54)c67)c2)nc1-c1ccccn1 ; N#Cc1c(-c2ccccc2)nc(-c2cccc(-n3c4cccc5c6cccc7c8ccccc8n(c8cccc3c8c54)c67)c2)nc1-c1cccnc1 ; N#Cc1c(-c2ccccc2)nc(-c2cccc(-n3c4cccc5c6cccc7c8ccccc8n(c8cccc3c8c54)c67)c2)nc1-c1ccncc1]

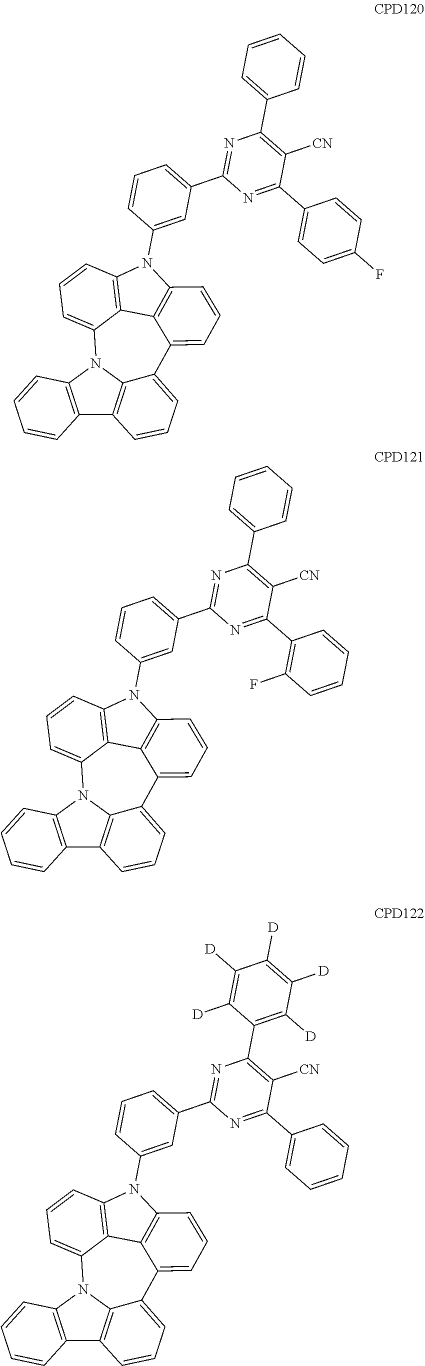 [SMILES: N#Cc1c(-c2ccccc2)nc(-c2cccc(-n3c4cccc5c6cccc7c8ccccc8n(c8cccc3c8c54)c67)c2)nc1-c1ccc(F)cc1 ; N#Cc1c(-c2ccccc2)nc(-c2cccc(-n3c4cccc5c6cccc7c8ccccc8n(c8cccc3c8c54)c67)c2)nc1-c1ccccc1F ; [2H]c1c([2H])c([2H])c(-c2nc(-c3cccc(-n4c5cccc6c7cccc8c9ccccc9n(c9cccc4c9c65)c78)c3)nc(-c3ccccc3)c2C#N)c([2H])c1[2H]]

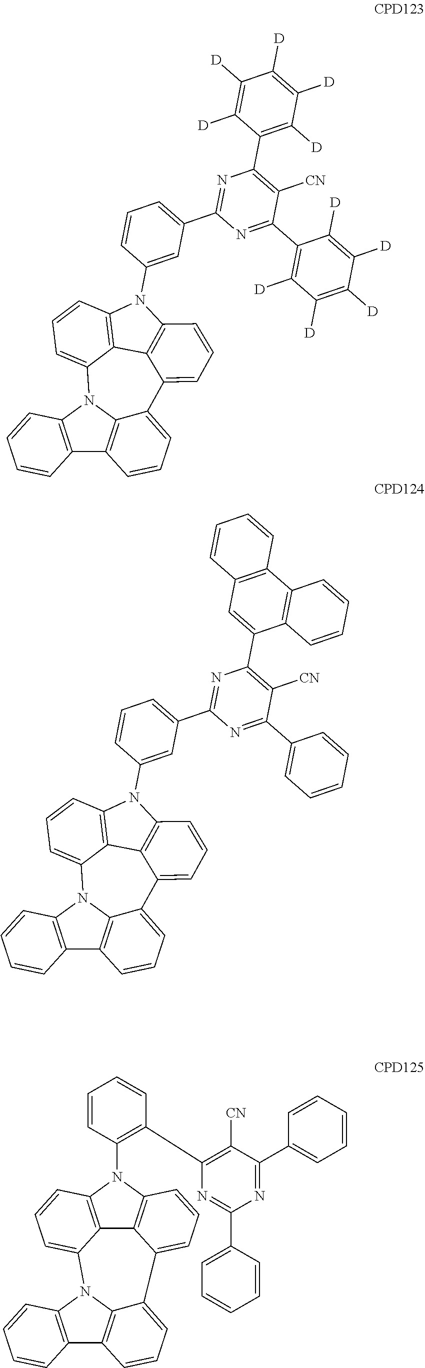 [SMILES: [2H]c1c([2H])c([2H])c(-c2nc(-c3cccc(-n4c5cccc6c7cccc8c9ccccc9n(c9cccc4c9c65)c78)c3)nc(-c3c([2H])c([2H])c([2H])c([2H])c3[2H])c2C#N)c([2H])c1[2H] ; N#Cc1c(-c2ccccc2)nc(-c2cccc(-n3c4cccc5c6cccc7c8ccccc8n(c8cccc3c8c54)c67)c2)nc1-c1cc2ccccc2c2ccccc12 ; N#Cc1c(-c2ccccc2)nc(-c2ccccc2)nc1-c1ccccc1-n1c2cccc3c4cccc5c6ccccc6n(c6cccc1c6c32)c45]

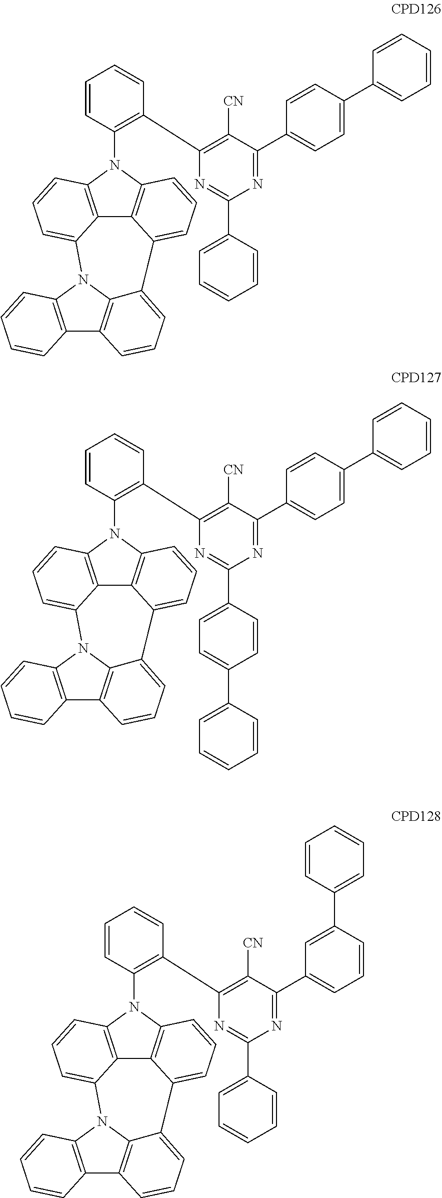 [SMILES: N#Cc1c(-c2ccc(-c3ccccc3)cc2)nc(-c2ccccc2)nc1-c1ccccc1-n1c2cccc3c4cccc5c6ccccc6n(c6cccc1c6c32)c45 ; N#Cc1c(-c2ccc(-c3ccccc3)cc2)nc(-c2ccc(-c3ccccc3)cc2)nc1-c1ccccc1-n1c2cccc3c4cccc5c6ccccc6n(c6cccc1c6c32)c45 ; N#Cc1c(-c2cccc(-c3ccccc3)c2)nc(-c2ccccc2)nc1-c1ccccc1-n1c2cccc3c4cccc5c6ccccc6n(c6cccc1c6c32)c45]

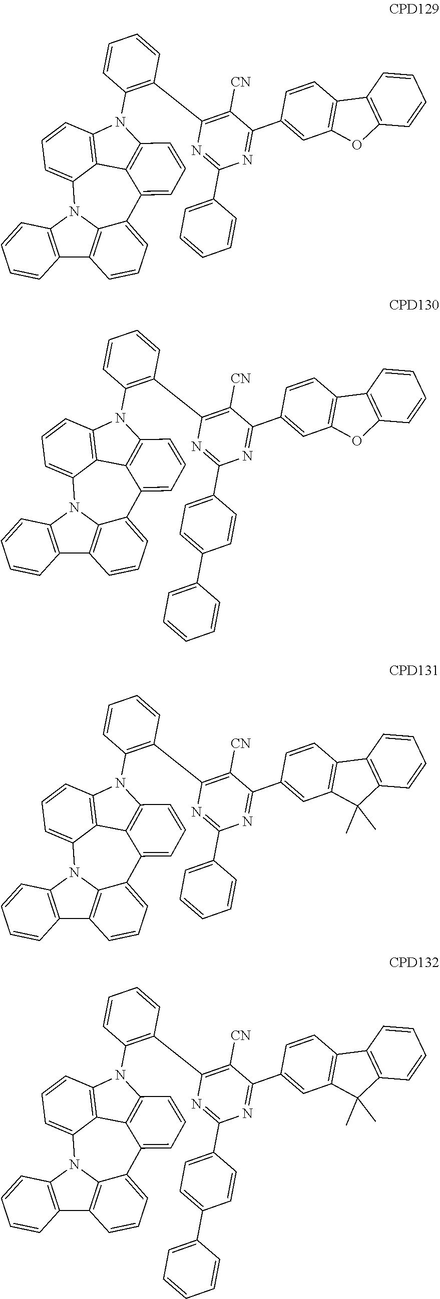 [SMILES: N#Cc1c(-c2ccc3c(c2)oc2ccccc23)nc(-c2ccccc2)nc1-c1ccccc1-n1c2cccc3c4cccc5c6ccccc6n(c6cccc1c6c32)c45 ; N#Cc1c(-c2ccc3c(c2)oc2ccccc23)nc(-c2ccc(-c3ccccc3)cc2)nc1-c1ccccc1-n1c2cccc3c4cccc5c6ccccc6n(c6cccc1c6c32)c45 ; CC1(C)c2ccccc2-c2ccc(-c3nc(-c4ccccc4)nc(-c4ccccc4-n4c5cccc6c7cccc8c9ccccc9n(c9cccc4c9c65)c78)c3C#N)cc21 ; CC1(C)c2ccccc2-c2ccc(-c3nc(-c4ccc(-c5ccccc5)cc4)nc(-c4ccccc4-n4c5cccc6c7cccc8c9ccccc9n(c9cccc4c9c65)c78)c3C#N)cc21]

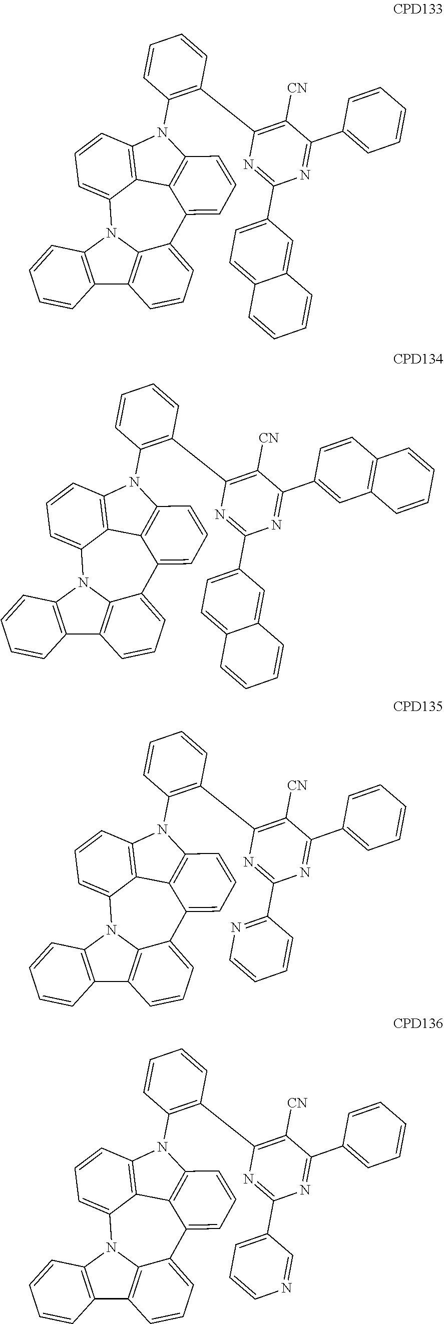 [SMILES: N#Cc1c(-c2ccccc2)nc(-c2ccc3ccccc3c2)nc1-c1ccccc1-n1c2cccc3c4cccc5c6ccccc6n(c6cccc1c6c32)c45 ; N#Cc1c(-c2ccc3ccccc3c2)nc(-c2ccc3ccccc3c2)nc1-c1ccccc1-n1c2cccc3c4cccc5c6ccccc6n(c6cccc1c6c32)c45 ; N#Cc1c(-c2ccccc2)nc(-c2ccccn2)nc1-c1ccccc1-n1c2cccc3c4cccc5c6ccccc6n(c6cccc1c6c32)c45 ; N#Cc1c(-c2ccccc2)nc(-c2cccnc2)nc1-c1ccccc1-n1c2cccc3c4cccc5c6ccccc6n(c6cccc1c6c32)c45]

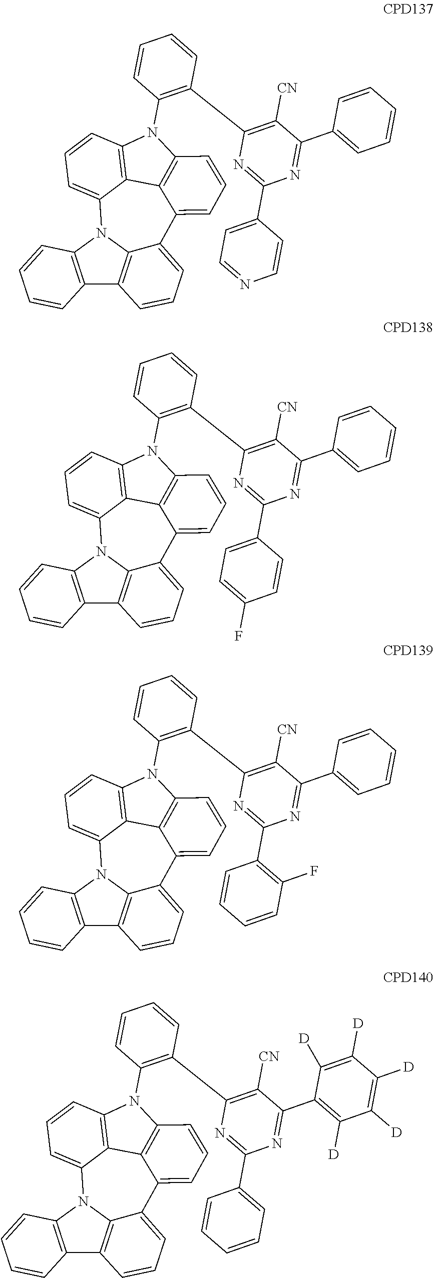 [SMILES: N#Cc1c(-c2ccccc2)nc(-c2ccncc2)nc1-c1ccccc1-n1c2cccc3c4cccc5c6ccccc6n(c6cccc1c6c32)c45 ; N#Cc1c(-c2ccccc2)nc(-c2ccc(F)cc2)nc1-c1ccccc1-n1c2cccc3c4cccc5c6ccccc6n(c6cccc1c6c32)c45 ; N#Cc1c(-c2ccccc2)nc(-c2ccccc2F)nc1-c1ccccc1-n1c2cccc3c4cccc5c6ccccc6n(c6cccc1c6c32)c45 ; [2H]c1c([2H])c([2H])c(-c2nc(-c3ccccc3)nc(-c3ccccc3-n3c4cccc5c6cccc7c8ccccc8n(c8cccc3c8c54)c67)c2C#N)c([2H])c1[2H]]

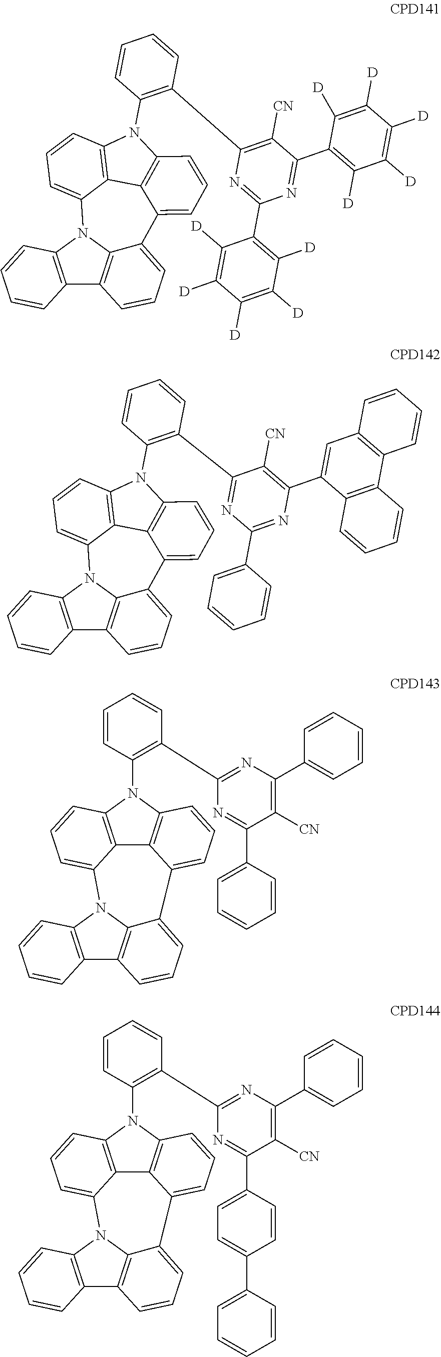

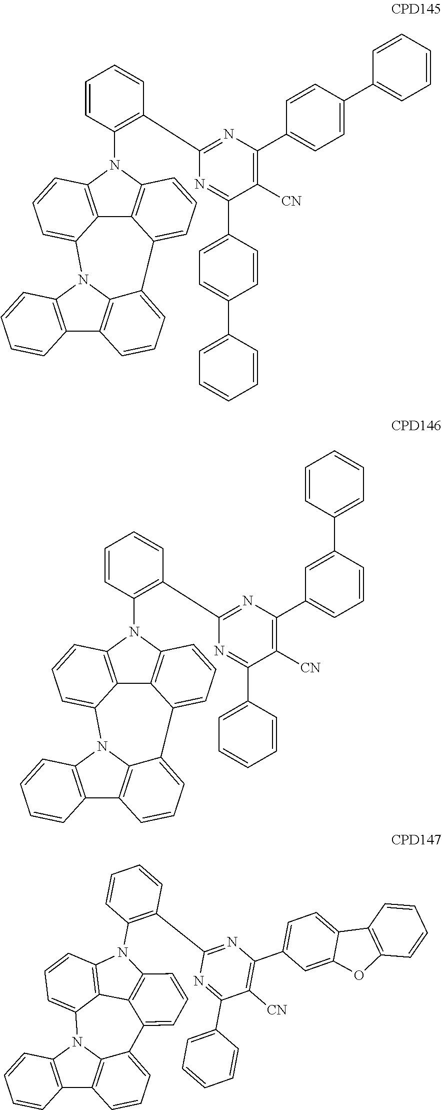

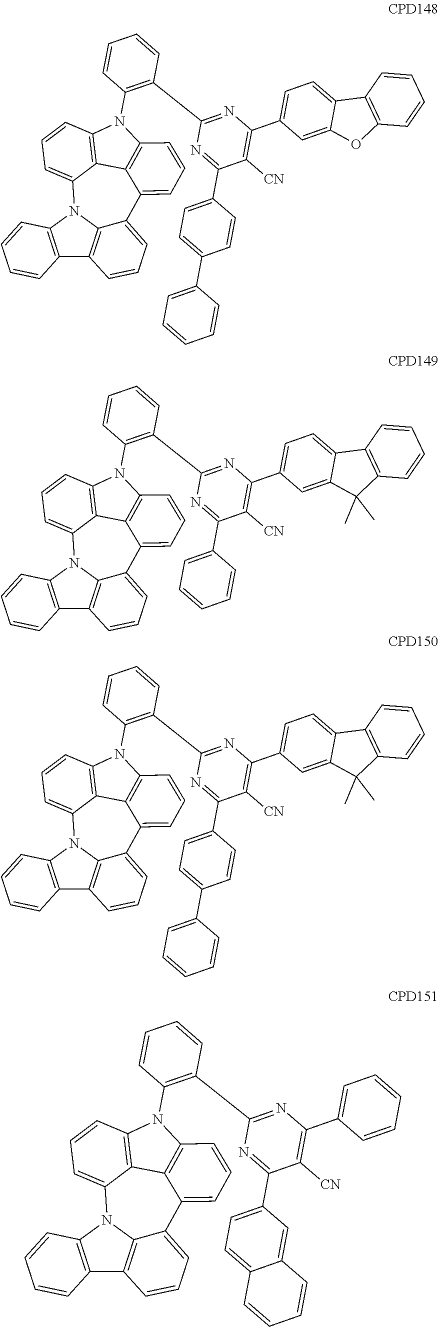 [SMILES: N#Cc1c(-c2ccc(-c3ccccc3)cc2)nc(-c2ccccc2-n2c3cccc4c5cccc6c7ccccc7n(c7cccc2c7c43)c56)nc1-c1ccc2c(c1)oc1ccccc12 ; CC1(C)c2ccccc2-c2ccc(-c3nc(-c4ccccc4-n4c5cccc6c7cccc8c9ccccc9n(c9cccc4c9c65)c78)nc(-c4ccccc4)c3C#N)cc21 ; CC1(C)c2ccccc2-c2ccc(-c3nc(-c4ccccc4-n4c5cccc6c7cccc8c9ccccc9n(c9cccc4c9c65)c78)nc(-c4ccc(-c5ccccc5)cc4)c3C#N)cc21 ; N#Cc1c(-c2ccccc2)nc(-c2ccccc2-n2c3cccc4c5cccc6c7ccccc7n(c7cccc2c7c43)c56)nc1-c1ccc2ccccc2c1]

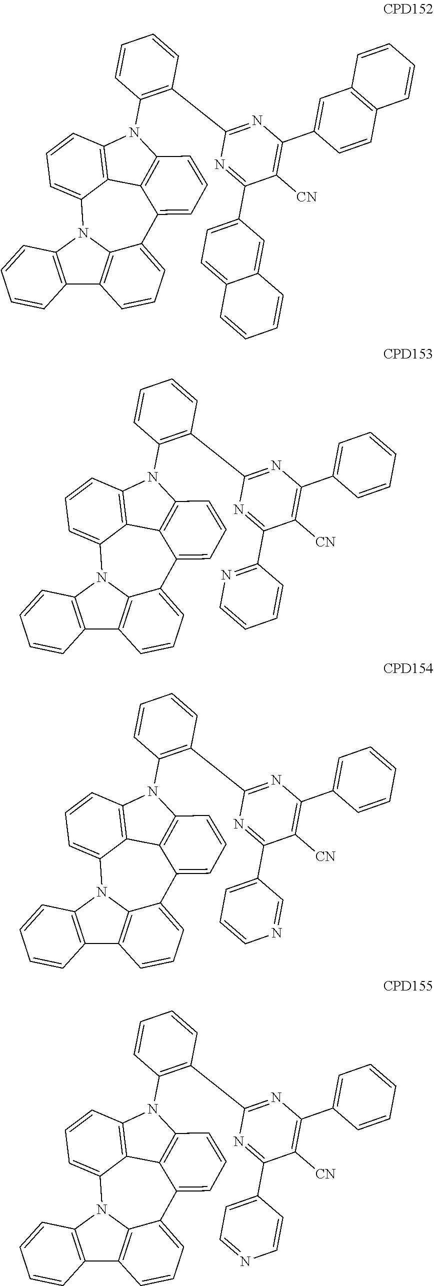 [SMILES: N#Cc1c(-c2ccc3ccccc3c2)nc(-c2ccccc2-n2c3cccc4c5cccc6c7ccccc7n(c7cccc2c7c43)c56)nc1-c1ccc2ccccc2c1 ; N#Cc1c(-c2ccccc2)nc(-c2ccccc2-n2c3cccc4c5cccc6c7ccccc7n(c7cccc2c7c43)c56)nc1-c1ccccn1 ; N#Cc1c(-c2ccccc2)nc(-c2ccccc2-n2c3cccc4c5cccc6c7ccccc7n(c7cccc2c7c43)c56)nc1-c1cccnc1 ; N#Cc1c(-c2ccccc2)nc(-c2ccccc2-n2c3cccc4c5cccc6c7ccccc7n(c7cccc2c7c43)c56)nc1-c1ccncc1]

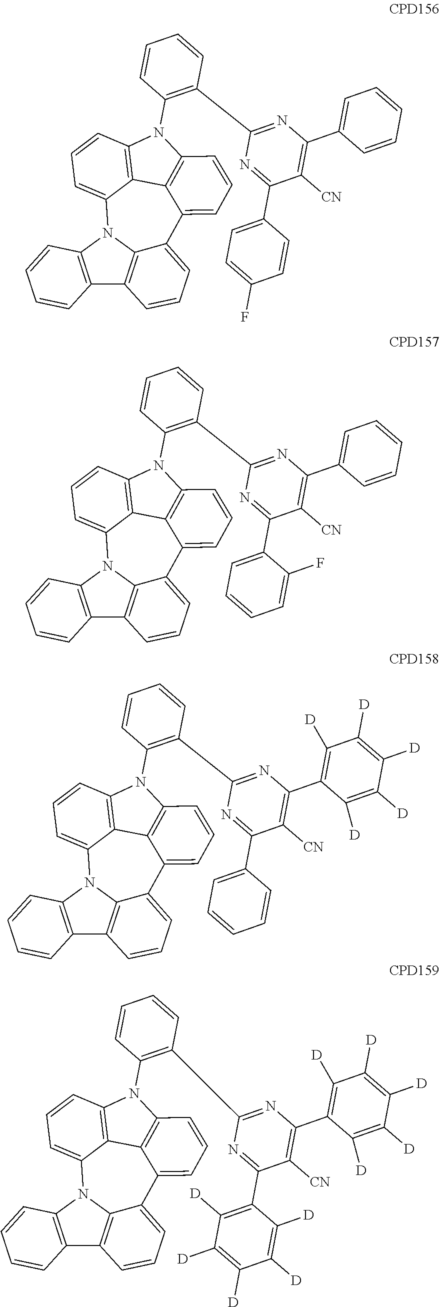

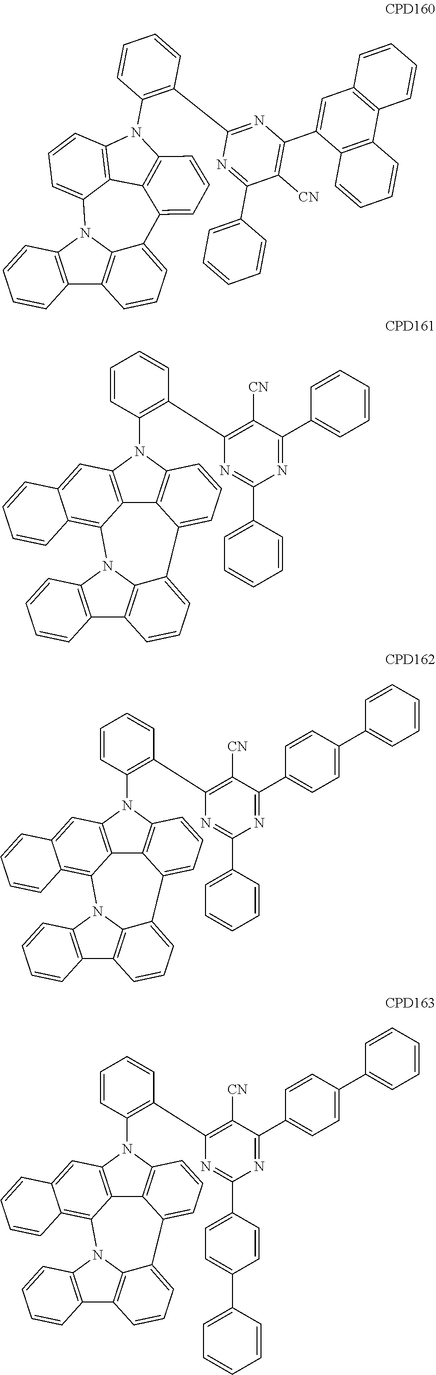

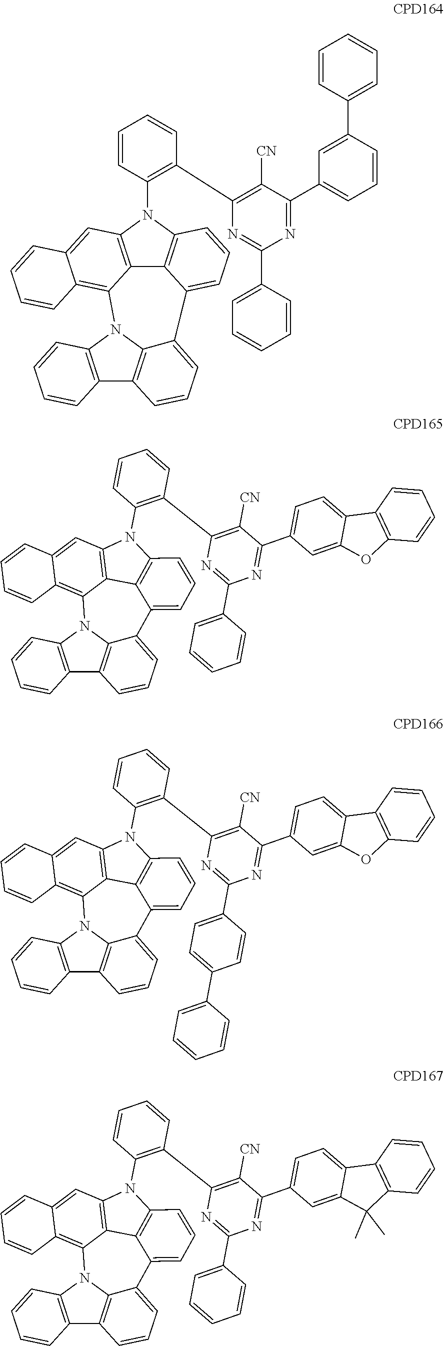 [SMILES: N#Cc1c(-c2cccc(-c3ccccc3)c2)nc(-c2ccccc2)nc1-c1ccccc1-n1c2cccc3c4cccc5c6ccccc6n(c45)c4c5ccccc5cc1c4c32 ; N#Cc1c(-c2ccc3c(c2)oc2ccccc23)nc(-c2ccccc2)nc1-c1ccccc1-n1c2cccc3c4cccc5c6ccccc6n(c45)c4c5ccccc5cc1c4c32 ; N#Cc1c(-c2ccc3c(c2)oc2ccccc23)nc(-c2ccc(-c3ccccc3)cc2)nc1-c1ccccc1-n1c2cccc3c4cccc5c6ccccc6n(c45)c4c5ccccc5cc1c4c32 ; CC1(C)c2ccccc2-c2ccc(-c3nc(-c4ccccc4)nc(-c4ccccc4-n4c5cccc6c7cccc8c9ccccc9n(c78)c7c8ccccc8cc4c7c65)c3C#N)cc21]

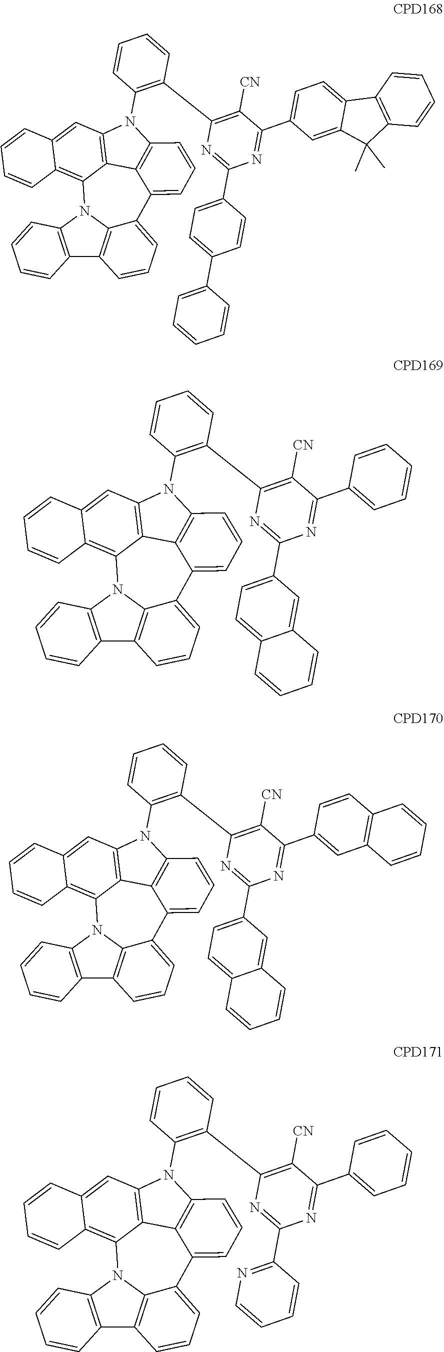 [SMILES: CC1(C)c2ccccc2-c2ccc(-c3nc(-c4ccc(-c5ccccc5)cc4)nc(-c4ccccc4-n4c5cccc6c7cccc8c9ccccc9n(c78)c7c8ccccc8cc4c7c65)c3C#N)cc21 ; N#Cc1c(-c2ccccc2)nc(-c2ccc3ccccc3c2)nc1-c1ccccc1-n1c2cccc3c4cccc5c6ccccc6n(c45)c4c5ccccc5cc1c4c32 ; N#Cc1c(-c2ccc3ccccc3c2)nc(-c2ccc3ccccc3c2)nc1-c1ccccc1-n1c2cccc3c4cccc5c6ccccc6n(c45)c4c5ccccc5cc1c4c32 ; N#Cc1c(-c2ccccc2)nc(-c2ccccn2)nc1-c1ccccc1-n1c2cccc3c4cccc5c6ccccc6n(c45)c4c5ccccc5cc1c4c32]

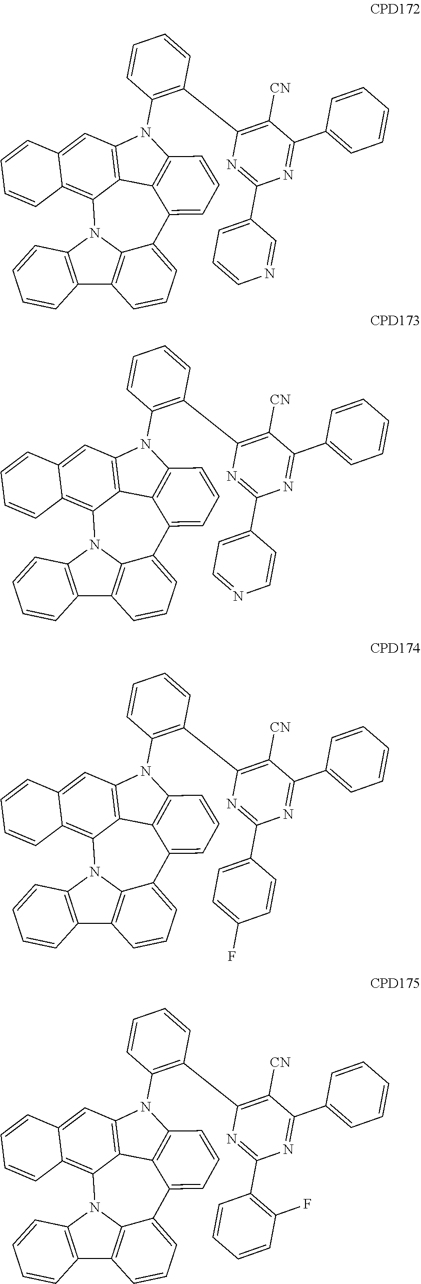

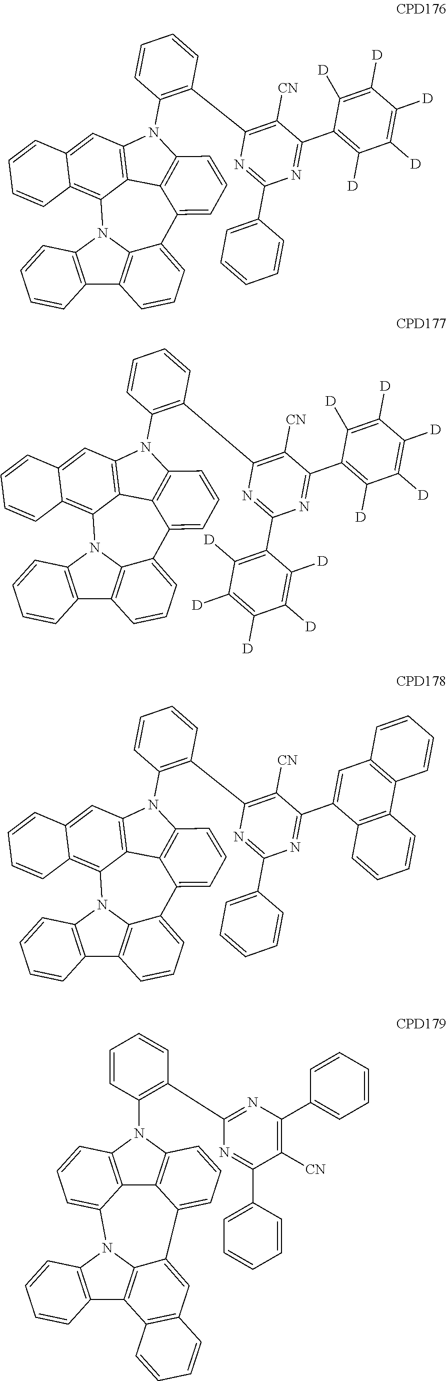

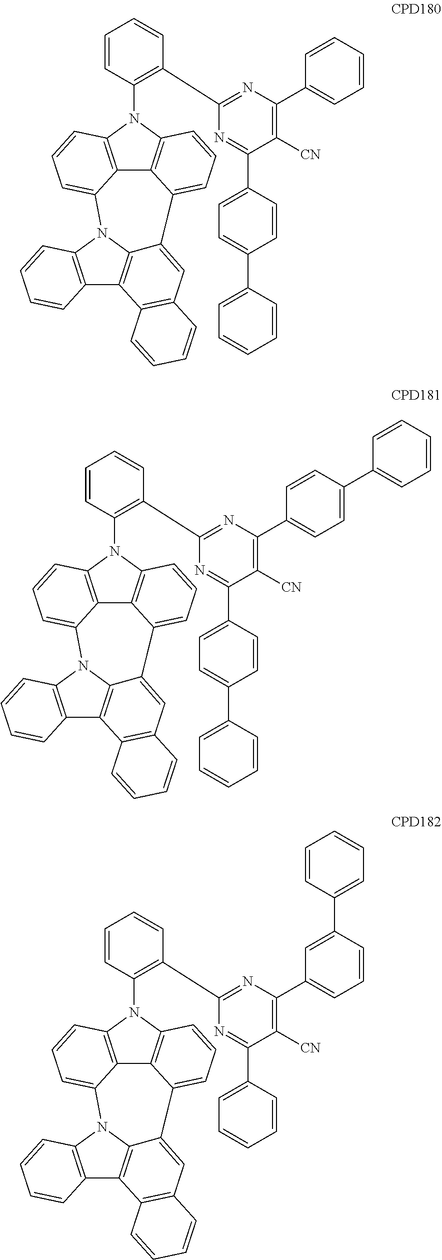 [SMILES: N#Cc1c(-c2ccccc2)nc(-c2ccccc2-n2c3cccc4c5cc6ccccc6c6c7ccccc7n(c7cccc2c7c43)c56)nc1-c1ccc(-c2ccccc2)cc1 ; N#Cc1c(-c2ccc(-c3ccccc3)cc2)nc(-c2ccccc2-n2c3cccc4c5cc6ccccc6c6c7ccccc7n(c7cccc2c7c43)c56)nc1-c1ccc(-c2ccccc2)cc1 ; N#Cc1c(-c2ccccc2)nc(-c2ccccc2-n2c3cccc4c5cc6ccccc6c6c7ccccc7n(c7cccc2c7c43)c56)nc1-c1cccc(-c2ccccc2)c1]

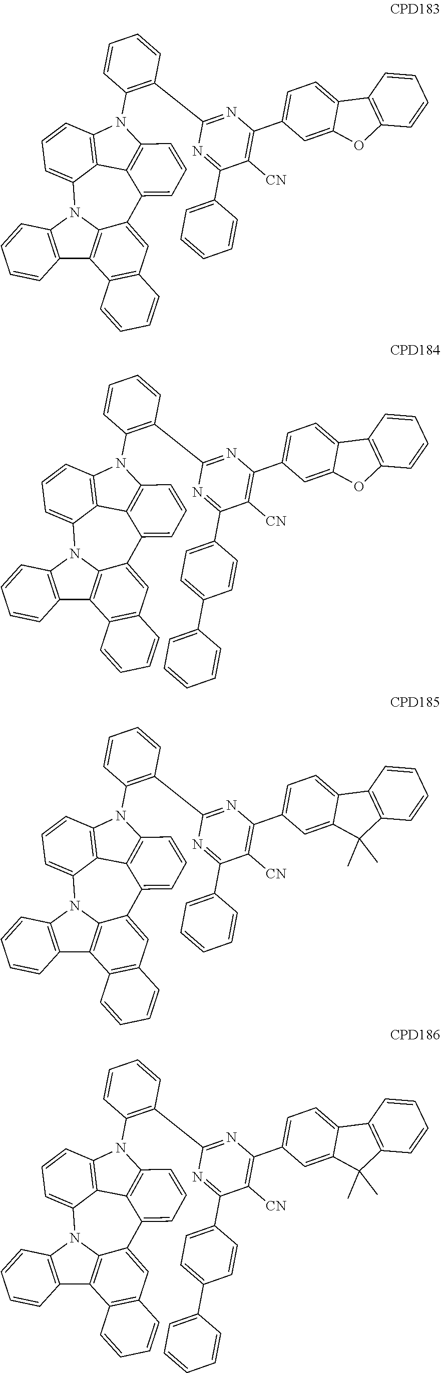

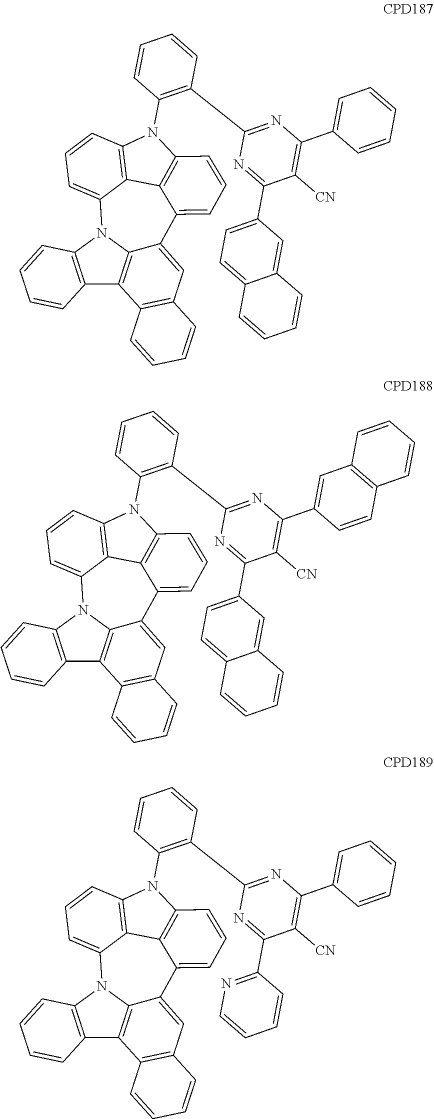

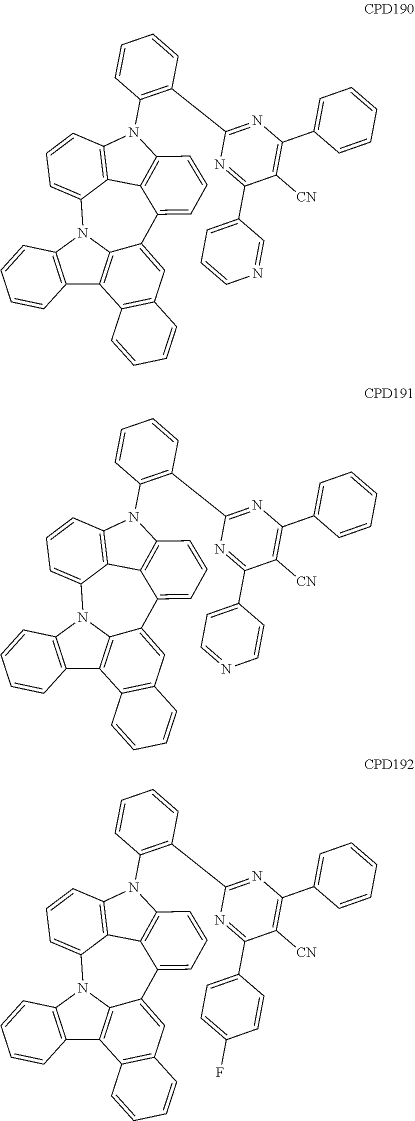

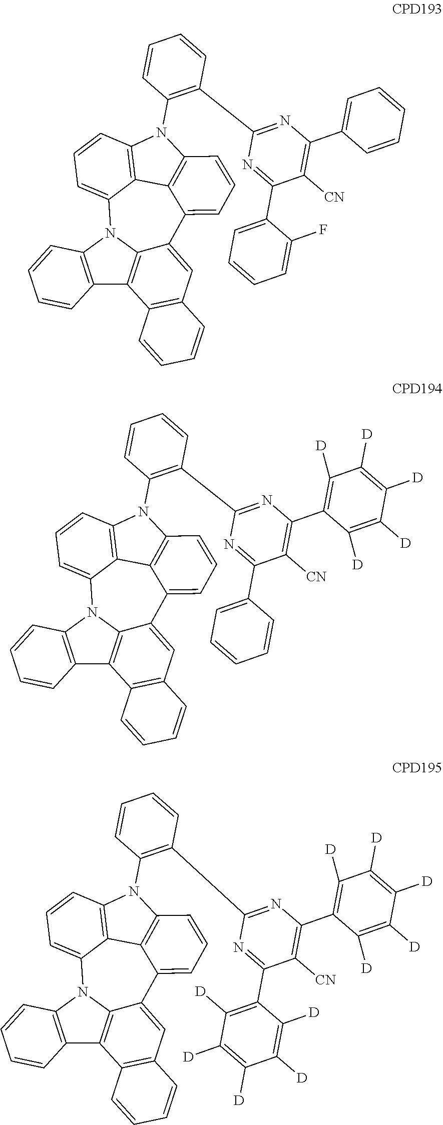 [SMILES: N#Cc1c(-c2ccccc2)nc(-c2ccccc2-n2c3cccc4c5cc6ccccc6c6c7ccccc7n(c7cccc2c7c43)c56)nc1-c1ccccc1F ; [2H]c1c([2H])c([2H])c(-c2nc(-c3ccccc3-n3c4cccc5c6cc7ccccc7c7c8ccccc8n(c8cccc3c8c54)c67)nc(-c3ccccc3)c2C#N)c([2H])c1[2H] ; [2H]c1c([2H])c([2H])c(-c2nc(-c3ccccc3-n3c4cccc5c6cc7ccccc7c7c8ccccc8n(c8cccc3c8c54)c67)nc(-c3c([2H])c([2H])c([2H])c([2H])c3[2H])c2C#N)c([2H])c1[2H]]

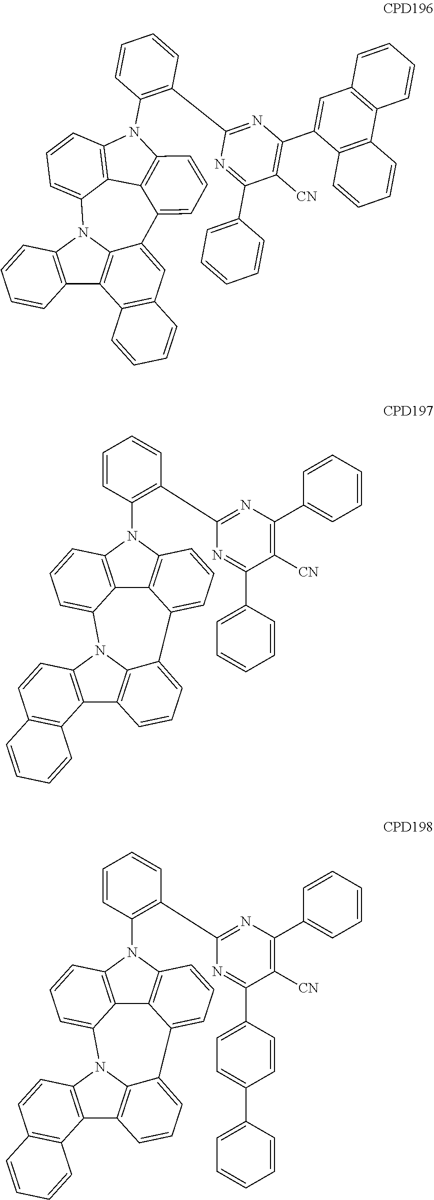 [SMILES: N#Cc1c(-c2ccccc2)nc(-c2ccccc2-n2c3cccc4c5cc6ccccc6c6c7ccccc7n(c7cccc2c7c43)c56)nc1-c1cc2ccccc2c2ccccc12 ; N#Cc1c(-c2ccccc2)nc(-c2ccccc2-n2c3cccc4c5cccc6c7c8ccccc8ccc7n(c7cccc2c7c43)c56)nc1-c1ccccc1 ; N#Cc1c(-c2ccccc2)nc(-c2ccccc2-n2c3cccc4c5cccc6c7c8ccccc8ccc7n(c7cccc2c7c43)c56)nc1-c1ccc(-c2ccccc2)cc1]

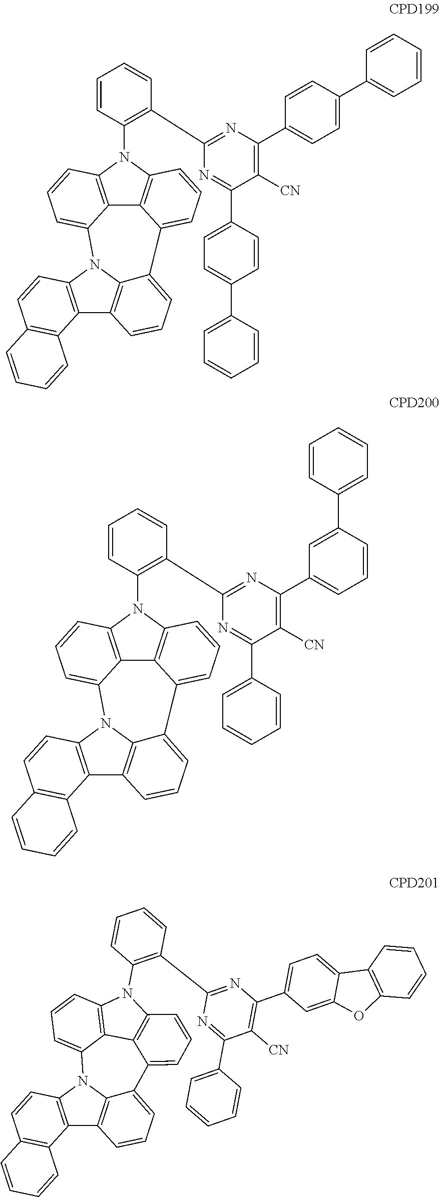 [SMILES: N#Cc1c(-c2ccc(-c3ccccc3)cc2)nc(-c2ccccc2-n2c3cccc4c5cccc6c7c8ccccc8ccc7n(c7cccc2c7c43)c56)nc1-c1ccc(-c2ccccc2)cc1 ; N#Cc1c(-c2ccccc2)nc(-c2ccccc2-n2c3cccc4c5cccc6c7c8ccccc8ccc7n(c7cccc2c7c43)c56)nc1-c1cccc(-c2ccccc2)c1 ; N#Cc1c(-c2ccccc2)nc(-c2ccccc2-n2c3cccc4c5cccc6c7c8ccccc8ccc7n(c7cccc2c7c43)c56)nc1-c1ccc2c(c1)oc1ccccc12]

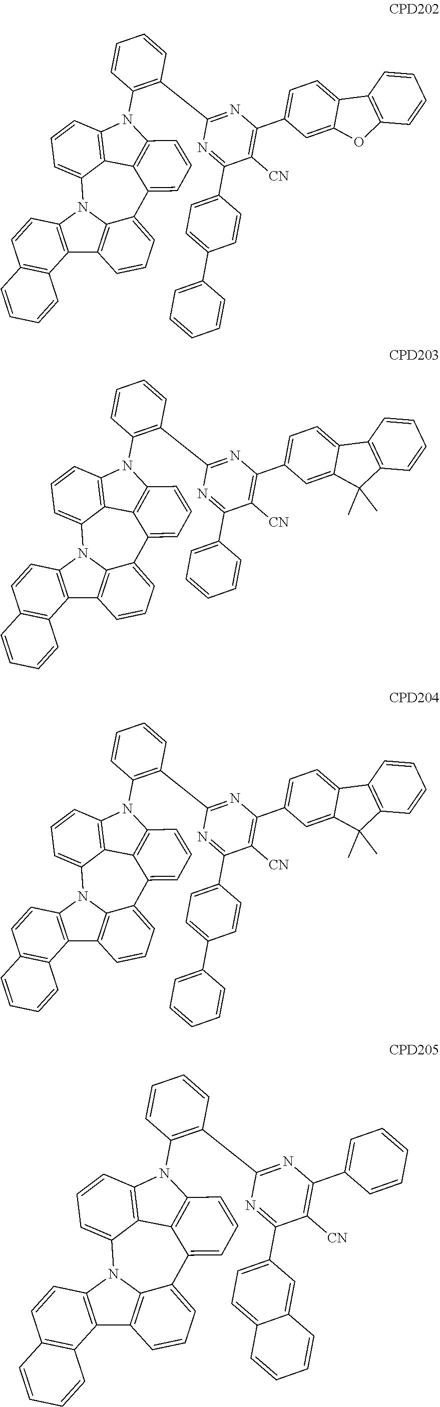 [SMILES: N#Cc1c(-c2ccc(-c3ccccc3)cc2)nc(-c2ccccc2-n2c3cccc4c5cccc6c7c8ccccc8ccc7n(c7cccc2c7c43)c56)nc1-c1ccc2c(c1)oc1ccccc12 ; CC1(C)c2ccccc2-c2ccc(-c3nc(-c4ccccc4-n4c5cccc6c7cccc8c9c%10ccccc%10ccc9n(c9cccc4c9c65)c78)nc(-c4ccccc4)c3C#N)cc21 ; CC1(C)c2ccccc2-c2ccc(-c3nc(-c4ccccc4-n4c5cccc6c7cccc8c9c%10ccccc%10ccc9n(c9cccc4c9c65)c78)nc(-c4ccc(-c5ccccc5)cc4)c3C#N)cc21 ; N#Cc1c(-c2ccccc2)nc(-c2ccccc2-n2c3cccc4c5cccc6c7c8ccccc8ccc7n(c7cccc2c7c43)c56)nc1-c1ccc2ccccc2c1]

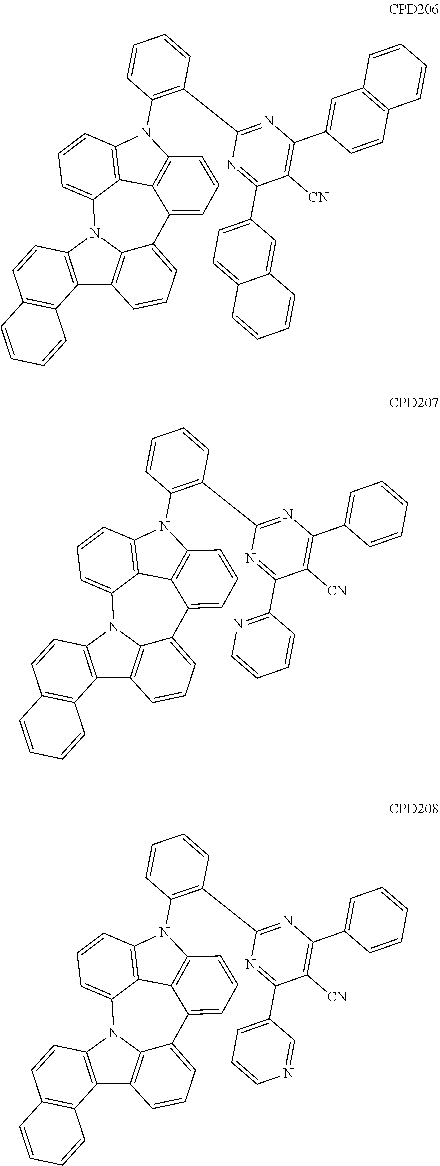 [SMILES: N#Cc1c(-c2ccc3ccccc3c2)nc(-c2ccccc2-n2c3cccc4c5cccc6c7c8ccccc8ccc7n(c7cccc2c7c43)c56)nc1-c1ccc2ccccc2c1 ; N#Cc1c(-c2ccccc2)nc(-c2ccccc2-n2c3cccc4c5cccc6c7c8ccccc8ccc7n(c7cccc2c7c43)c56)nc1-c1ccccn1 ; N#Cc1c(-c2ccccc2)nc(-c2ccccc2-n2c3cccc4c5cccc6c7c8ccccc8ccc7n(c7cccc2c7c43)c56)nc1-c1cccnc1]

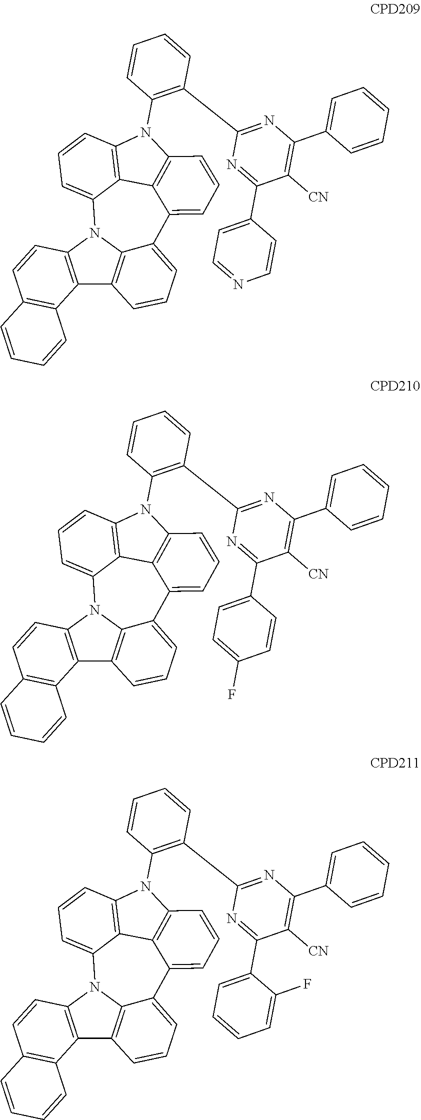 [SMILES: N#Cc1c(-c2ccccc2)nc(-c2ccccc2-n2c3cccc4c5cccc6c7c8ccccc8ccc7n(c7cccc2c7c43)c56)nc1-c1ccncc1 ; N#Cc1c(-c2ccccc2)nc(-c2ccccc2-n2c3cccc4c5cccc6c7c8ccccc8ccc7n(c7cccc2c7c43)c56)nc1-c1ccc(F)cc1 ; N#Cc1c(-c2ccccc2)nc(-c2ccccc2-n2c3cccc4c5cccc6c7c8ccccc8ccc7n(c7cccc2c7c43)c56)nc1-c1ccccc1F]

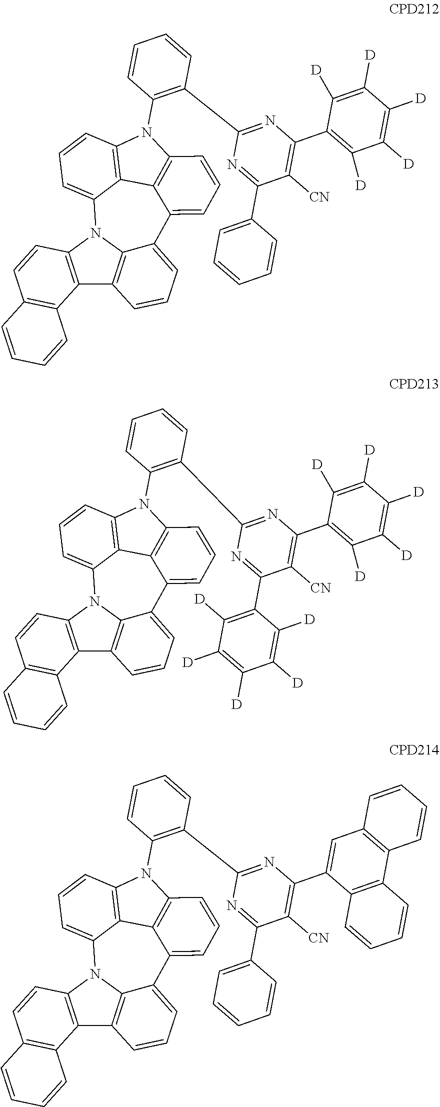 [SMILES: [2H]c1c([2H])c([2H])c(-c2nc(-c3ccccc3-n3c4cccc5c6cccc7c8c9ccccc9ccc8n(c8cccc3c8c54)c67)nc(-c3ccccc3)c2C#N)c([2H])c1[2H] ; [2H]c1c([2H])c([2H])c(-c2nc(-c3ccccc3-n3c4cccc5c6cccc7c8c9ccccc9ccc8n(c8cccc3c8c54)c67)nc(-c3c([2H])c([2H])c([2H])c([2H])c3[2H])c2C#N)c([2H])c1[2H] ; N#Cc1c(-c2ccccc2)nc(-c2ccccc2-n2c3cccc4c5cccc6c7c8ccccc8ccc7n(c7cccc2c7c43)c56)nc1-c1cc2ccccc2c2ccccc12]

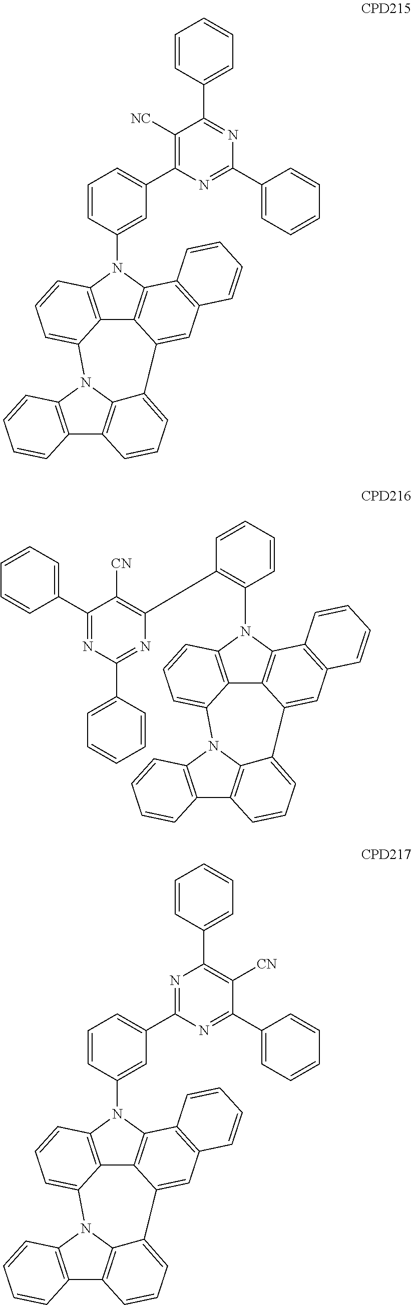 [SMILES: N#Cc1c(-c2ccccc2)nc(-c2ccccc2)nc1-c1cccc(-n2c3cccc4c3c3c(cc5ccccc5c32)c2cccc3c5ccccc5n4c23)c1 ; N#Cc1c(-c2ccccc2)nc(-c2ccccc2)nc1-c1ccccc1-n1c2cccc3c2c2c(cc4ccccc4c21)c1cccc2c4ccccc4n3c12 ; N#Cc1c(-c2ccccc2)nc(-c2cccc(-n3c4cccc5c4c4c(cc6ccccc6c43)c3cccc4c6ccccc6n5c34)c2)nc1-c1ccccc1]

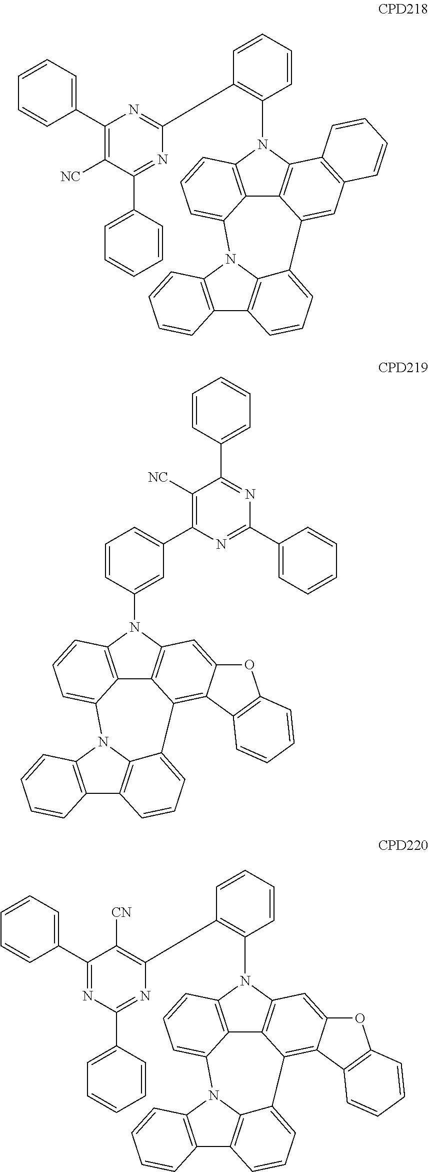 [SMILES: N#Cc1c(-c2ccccc2)nc(-c2ccccc2-n2c3cccc4c3c3c(cc5ccccc5c32)c2cccc3c5ccccc5n4c23)nc1-c1ccccc1 ; N#Cc1c(-c2ccccc2)nc(-c2ccccc2)nc1-c1cccc(-n2c3cc4oc5ccccc5c4c4c5cccc6c7ccccc7n(c7cccc2c7c43)c65)c1 ; N#Cc1c(-c2ccccc2)nc(-c2ccccc2)nc1-c1ccccc1-n1c2cc3oc4ccccc4c3c3c4cccc5c6ccccc6n(c6cccc1c6c32)c54]

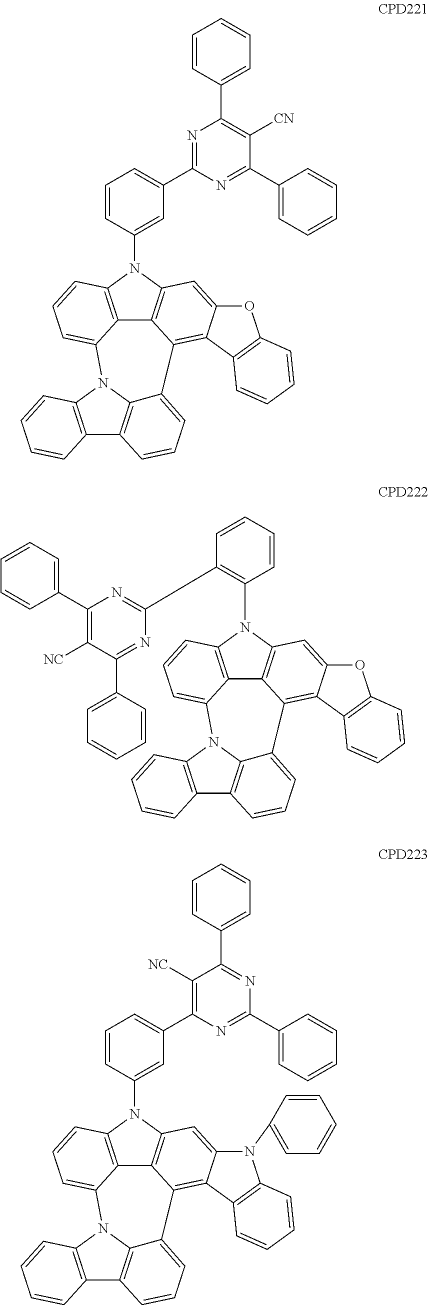 [SMILES: N#Cc1c(-c2ccccc2)nc(-c2cccc(-n3c4cc5oc6ccccc6c5c5c6cccc7c8ccccc8n(c8cccc3c8c54)c76)c2)nc1-c1ccccc1 ; N#Cc1c(-c2ccccc2)nc(-c2ccccc2-n2c3cc4oc5ccccc5c4c4c5cccc6c7ccccc7n(c7cccc2c7c43)c65)nc1-c1ccccc1 ; N#Cc1c(-c2ccccc2)nc(-c2ccccc2)nc1-c1cccc(-n2c3cc4c(c5ccccc5n4-c4ccccc4)c4c5cccc6c7ccccc7n(c7cccc2c7c43)c65)c1]

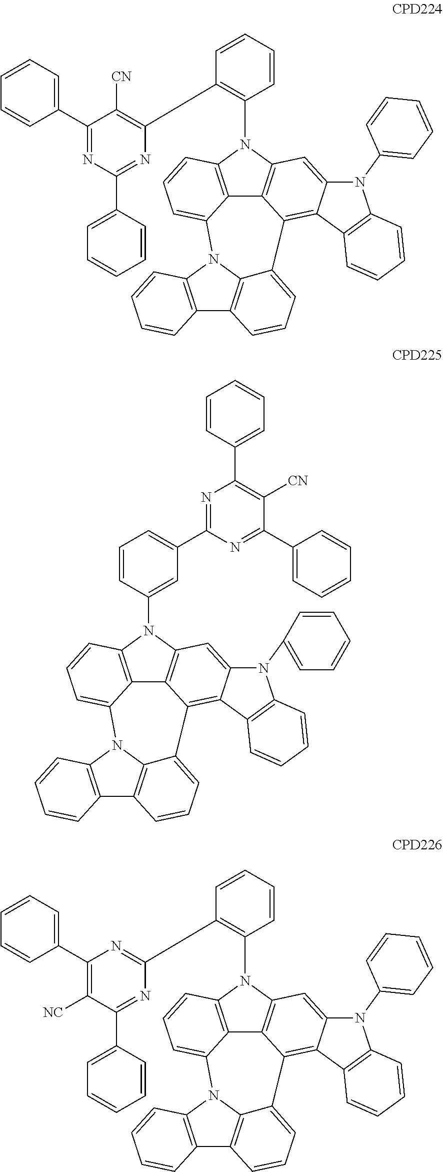 [SMILES: N#Cc1c(-c2ccccc2)nc(-c2ccccc2)nc1-c1ccccc1-n1c2cc3c(c4ccccc4n3-c3ccccc3)c3c4cccc5c6ccccc6n(c6cccc1c6c32)c54 ; N#Cc1c(-c2ccccc2)nc(-c2cccc(-n3c4cc5c(c6ccccc6n5-c5ccccc5)c5c6cccc7c8ccccc8n(c8cccc3c8c54)c76)c2)nc1-c1ccccc1 ; N#Cc1c(-c2ccccc2)nc(-c2ccccc2-n2c3cc4c(c5ccccc5n4-c4ccccc4)c4c5cccc6c7ccccc7n(c7cccc2c7c43)c65)nc1-c1ccccc1]

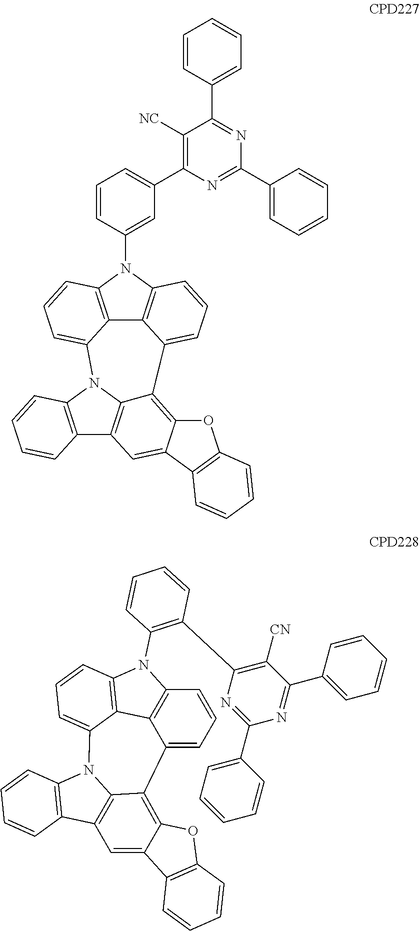 [SMILES: N#Cc1c(-c2ccccc2)nc(-c2ccccc2)nc1-c1cccc(-n2c3cccc4c3c3c2cccc3n2c3ccccc3c3cc5c6ccccc6oc5c4c32)c1 ; N#Cc1c(-c2ccccc2)nc(-c2ccccc2)nc1-c1ccccc1-n1c2cccc3c2c2c1cccc2n1c2ccccc2c2cc4c5ccccc5oc4c3c21]

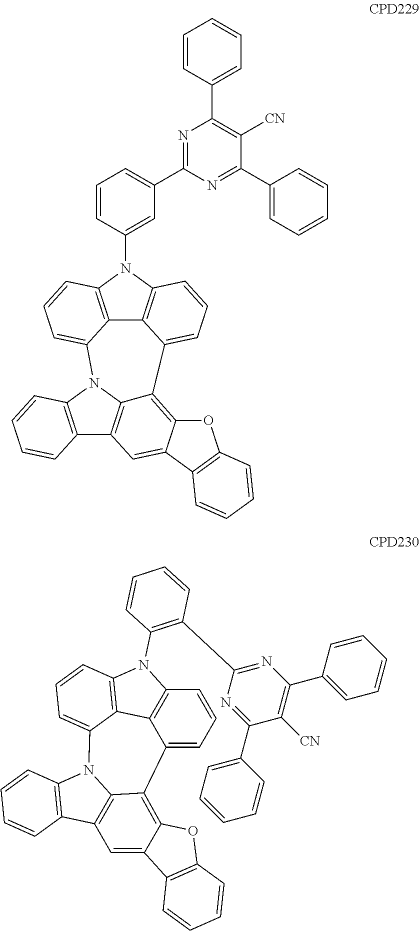 [SMILES: N#Cc1c(-c2ccccc2)nc(-c2cccc(-n3c4cccc5c4c4c3cccc4n3c4ccccc4c4cc6c7ccccc7oc6c5c43)c2)nc1-c1ccccc1 ; N#Cc1c(-c2ccccc2)nc(-c2ccccc2-n2c3cccc4c3c3c2cccc3n2c3ccccc3c3cc5c6ccccc6oc5c4c32)nc1-c1ccccc1]

12. An organic electroluminescent device, comprising the organic electroluminescent material according to claim 1.

13. The organic electroluminescent device of claim 12, comprising a light emitting layer, wherein the light emitting layer comprises the organic electroluminescent material.
